(19) United States
(12) Patent Application Publication
(10) Pub. No.: US 2014/0249143 A1
(43) Pub. Date: Sep. 4, 2014

(54) Compounds And Methods For Enhancing Innate Immune Responses

(71) Applicant: GlaxoSmithKline LLC, Philadelphia, PA (US)

(72) Inventors: Anna Lindsey Banka, Research Triangle Park, NC (US); Janos Botyanszki, Research Triangle Park, NC (US); Eric Gregory Burroughs, Research Triangle Park, NC (US); John George Catalano, Research Triangle Park, NC (US); Wendy Huang Chern, Research Triangle Park, NC (US); Hamilton D. Dickson, Research Triangle Park, NC (US); Margaret J. Gartland, Research Triangle Park, NC (US); Robert Hamatake, Research Triangle Park, NC (US); Hans Hofland, Research Triangle Park, NC (US); Jesse Daniel Keicher, Research Triangle Park, NC (US); Christopher Brooks Moore, Research Triangle Park, NC (US); John Bradford Shotwell, Research Triangle Park, NC (US); Matthew David Tallant, Research Triangle Park, NC (US); Jean-Philippe Therrien, Research Triangle Park, NC (US); Shihyun You, Research Triangle Park, NC (US)

(73) Assignee: GlaxoSmithKline LLC, Philadelphia, PA (US)

(21) Appl. No.: 14/353,067

(22) PCT Filed: Oct. 19, 2012
(86) PCT No.: PCT/US2012/060971

§ 371 Date: Apr. 21, 2014

Related U.S. Application Data
(60) Provisional application No. 61/549,784, filed on Oct. 21, 2011.
(60) Provisional application No. 61/692,431, filed on Aug. 23, 2012.

(51) Int. Cl. C07D 471/14; C07D 498/14; C07D 491/048; C07D 513/04; C07D 471/04; C07D 498/04

(52) U.S. Cl. CPC C07D 471/14; C07D 471/04; C07D 498/04; C07D 491/048; C07D 513/04; C07D 498/14
USPC 514/230.2; 546/82; 544/251; 514/293; 514/267; 546/81; 514/292; 546/83; 514/291; 544/346; 514/250; 544/126; 514/233.2; 544/101

(57) ABSTRACT

Provided are certain compounds and pharmaceutically acceptable salts thereof, their pharmaceutical compositions, their methods of preparation, and their use for treating viral infections.

### CROSS-REFERENCE TO RELATED PATENTS AND PATENT APPLICATIONS

This is a Patent Cooperation Treaty application and claims the benefit of U.S. Provisional Application No. 61/549,784, filed Oct. 21, 2011 and U.S. Provisional Application No. 61/692,431, filed Aug. 23, 2012, both of which are hereby incorporated by reference in their entireties.

### FIELD OF THE INVENTION

Provided are compounds, pharmaceutical compositions, their methods of preparation, and methods for their use in treating and/or preventing viral infections, and in particular, to certain compounds that can enhance one or more innate immune responses within a subject.

### BACKGROUND OF THE INVENTION

A virus is a small infectious agent that invades a living cell in order to replicate. Viruses cause many familiar infectious diseases ranging from the common cold and influenza to more severe illnesses such as HIV/AIDS and hepatitis C. Virus-caused illnesses affect many people. For example, each year in the US there are approximately 62 million cases of the common cold and approximately 50 thousand people are newly infected with HIV. (National Center for Health Statistics, Health Data Interactive, www.cdc.gov/nchs/hdi.htm. Accessed on Sep. 9, 2011).

The market offers few drugs to combat viral infections. Antiviral drugs can work by interacting with the virus to reduce its pathogenicity or by targeting the host in order to improve the host's defense against the virus. Most antiviral drugs on the market, like zanamivir for treating influenza and zidovudine for treating HIV, interact directly with the virus to reduce pathogenicity. However, viruses can mutate and, thereby, develop resistance to these types of antiviral drugs. Consequently, antiviral drugs aimed at directly targeting a virus are prone to decreased efficacy over time. As a result, there is a strong, unmet need for an antiviral drug that targets the host rather than the virus directly.

Infectious virus-associated diseases remain a leading cause of premature death and disability due to disease. The World Health Organization (WHO) reports respiratory viral infections alone account for over 4 million deaths annually. Significantly, a number of other virus-associated diseases make significant contributions to deaths as well, including AIDS (2 million), HCV (54,000), HBV (105,000), measles (424,000), and Dengue (18,000). Large populations of carriers (HCV: 350,000,000; HBV: 170,000,000) remain within the population and will continue to propagate the crisis without the development of novel treatments paradigms. (see: J. Yewdell and J. Bennick. The Immune Response to Infection. (2011), p. 133-141).

Infectious virus-associated diseases remain a leading cause of premature death and disability years due to disease (DALYs). The World Health Organization (WHO) reports respiratory viral infections alone account for over 4 million deaths (1.6 million children) and 97 million DALYs annually. Significantly, a number of other virus-associated diseases make significant contributions to deaths and DALYs as well, including AIDS (2 million/58 million), HCV (54,000/955,000), HBV (105,000/2,068,000), measles (424,000, 14.8 million), and Dengue (18,000/681,000). Large populations of carriers (HCV: 350,000,000; HBV: 170,000,000) remain within the population and will continue to propagate the crisis without the development of novel treatments paradigms. See J. Yewdell and J. Bennick. The Immune Response to Infection. (2011), p. 133-141. The development of agents acting directly on critical viral enzymes/structural proteins has become an advanced field, with potent treatment cocktails approved for HIV and in late-stage development for HCV. However, all direct acting antiviral agents carry the risk of selecting for mutant viruses which can tremendously limit the efficacy of treatment. This problem, coupled with a myriad of unique replication strategies represented by known infectious viruses, has made the identification of agents suitable for treatment of multiple virus-associated diseases extremely challenging and largely unsuccessful. Therapeutic agents that bolster existing host immune mechanisms of viral defense, specifically the host innate immune response to infection, hold potential as inroads to the treatment of multiple infections with a single agent.

The innate immune system is capable of the rapid recognition of invading viruses via a set of pattern recognition receptors (PRRs): toll-like receptors (TLRs), retinoic acid-inducible gene I like receptors (RLRs) and nucleotide oligomerization domain like receptors (NODs) (for review: O. Takeuchi and S. Akira, Immunological Reviews, (2009), p. 75-86). For example, the recognition of dsRNA and 5′-triphosphate capped RNAs by RLRs and TLRs leads directly to downstream signaling effecting a type-I interferon (IFN) response, upregulating expression of IFN-inducible genes involved in the elimination of the virus from infected host cells. STATs are essential downstream effectors of these IFNs. Binding of IFNs to their corresponding receptors (for example, IFNα to INFAR1/INFAR2) leads to activation of constitutively bound JAK family kinases (for example, TYK2 and JAK1), subsequent phosphorylation of the receptor affording a STAT binding site (binding via an SH2 domain for example), and then phosphorylation of STATs (for example phosphorylation of STAT1 on tyrosine 701) promoting STAT dimerization, translocation to the nucleus, and initiation of transcription of proteins critical for a host's antiviral machinery and response (see: K. Shuai and B. Liu, Nature Reviews Immunology, (2003), p. 900-911).

To successfully infect organisms pathogens (viral in addition to bacterial and parasitic pathogens) must overcome the activation of STATs and the ensuing transcription of host antiviral genes. Indeed, most pathogens have evolved some means of blocking one or more steps in the host's innate immune response (see: I. Najar and R. Fagard, Biochimie, (2010), p. 425-444). Therapeutics which activate the innate immune response via the JAK/STAT pathway either (1) via a mechanism downstream of a particular viral blocking mechanism or (2) in a manner robust enough to overcome the virus's means of circumvention hold potential as treatments for the elimination of these viral infections, and should not suffer from virus resistance mutations as the therapeutics target host proteins under no selection pressure.

However, all direct acting antiviral agents carry the risk of selecting for resistant viruses which can tremendously limit the efficacy of treatment. This problem, coupled with a myriad of unique replication strategies represented by known infectious viruses, has made the identification of agents suitable for treatment of multiple virus-associated diseases extremely challenging and largely unsuccessful. Therapeutic agents that bolster existing host immune mechanisms of viral defense, specifically the host innate immune response to infection, hold potential as inroads to the treatment of multiple infections with a single agent.

Virus-infected cells secrete a broad range of interferon (IFN) subtypes which in turn trigger the synthesis of antiviral factors that confer host resistance. IFN-alpha, IFN-beta and other type I IFNs signal through a common universally expressed cell surface receptor, See Mordsten, et al., PLoS Pathoa. 2008 Sep. 12; 4(9):e1000151. Interferon-lambda contributes to innate immunity of mice against influenza A virus.

In particular, one virus that is a source of world-wide concern is the Human papillomavirus (“HPV”). Human papillomavirus is a double-stranded DNA virus, and is responsible for the appearance of warts. Virus particles reside in the basal layer of epithelia, but replicate only in the well-differentiated, superficial layer. The ensuing cellular proliferation gives rise to the characteristic morphology of warts. Human papillomavirus may be transmitted indirectly through contact with the skin of an infected individual or by transmission of virus that has survived in warm, moist environments. The virus may also be transferred from one site to another when autoinoculation occurs upon traumatizing warts by scratching or biting. The incubation period is unknown, but may be several months or years.

Warts are a widespread medical problem that cause pain and discomfort, and may lead to complications if untreated or improperly treated. Warts are benign growths of the skin caused by a virus that involves the epidermis. Five different types of warts are classified by their clinical presentation. (1) Verrucae vulgares are common warts that display hyperkeratosis and may occur anywhere except the genital and mucous membranes and plantar surfaces (soles of the feet); (2) Verrucae planae are flat warts that usually occur on the face, trunk and extremities; (3) Verrucae plantares are warts that occur only on the soles of the feet; (4) Condylomata acuminata are venereal warts that occur on the genitals and mucous membranes; (5) premalignant warts (Epidermoldysplasia verruciformis) usually occur on the hands and feet and are rare in occurrence.

Currently, there are no completely successful, treatments for warts. Current treatments of verrucae involve physical destruction of the infected cells. Choice of treatment depends on the location, size, number, type of wart, age and co-operation of the patient. No one treatment modality is uniformally effective or directly antiviral.

Wart treatments include cryotherapy with liquid nitrogen, caustics and acids such as salicylic acid, lactic acid and trichloroacetic acid which destroy and peel off infected skin. Retinoic acid has been used topically to treat flat warts. Cantharidin is an extract of the green blister beetle that leads to blistering and focal destruction of the epidermis. Induction of allergic contact dermatitis with dinitrochlorobenzene (DNCB) produces local inflammation to warts on which this chemical has been applied.

Based on the foregoing, there exists a significant need to identify synthetic or biological compounds for their ability to enhance a host's innate immune response, specifically its Type I Interferon response, and subsequently inhibit replication of multiple viral infections. Likewise, there also exists a significant need to identify synthetic or biological compounds for their ability to enhance a host's innate immune response, specifically its Type I Interferon response, and subsequently inhibit replication of multiple viral infections. Very few examples of small molecules with such properties have been reported (in addition to molecules acting via TLR-7, see Am. J. Respir. Cell. Mol. Biol., 2011, p. 480-488).

### SUMMARY OF THE INVENTION

The present invention relates to compounds that act as enhancers of the host's immune response. The compounds are believed to up-regulate expression and/or activity of one or more of these proteins, thereby leading to better viral defense and/or treatment.

In accordance with one embodiment of the present invention, there is provided a compound of Formula (I):

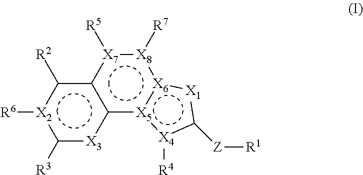

or a pharmaceutically acceptable salt thereof, wherein:
X₁, X₄, X₇, and X₈, are independently selected from N, NH, S, O, C, CH, or CH₂;
X₂, X₃, X₅, and X₆ are independently selected from N, NH, C, CH, or CH₂;
Z is selected from a bond, —C(O), or (C₁-C₆)alkylene;
R¹ is selected from the group consisting of hydrogen, —R¹², —R¹⁴, —R⁹(R¹⁵)ₘ, —OR(R¹⁵)ₘ, and halo;
R² is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, OR¹³, —R¹²S(O)₂, —S(O)₂R¹², halo, nitrile, sulfonamide, sulfone, sulfoxide, (C₄-C₁₄)aryl, and (C₃-C₁₂)Cycloalkyl, wherein said R² group may be optionally substituted with one to three R¹¹ groups;
R³ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, halo, nitrile, sulfonamide, sulfone, sulfoxide, (C₄-C₁₄)aryl, and (C₃-C₁₂)Cycloalkyl, wherein said R³ group may be optionally substituted with one to three R¹¹ groups;
R⁴ is optionally absent or is selected from the group consisting of hydrogen, (C₁-C₆)alkyl, —R⁹(R¹⁵)ₘ, —OR(R¹⁵)ₘ, —R⁹R¹⁰, —C(O)R⁹, —C(O)R¹³, halo, and (C₃-C₁₂)Cycloalkyl;
R⁵ is selected from the group consisting of hydrogen, (C₁-C₆)alkyl, —C(O)R¹², —R⁹R¹², —R⁹(R¹⁵)ₘ, —OR⁹(R¹⁵)ₘ, —R¹⁴, halo, and nitrile;
R⁶ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, halo, nitrile, sulfonamide, sulfone, sulfoxide, (C₄-C₁₄)aryl, and (C₃-C₁₂)Cycloalkyl, wherein said R⁶ group may be optionally substituted with one to three R¹¹ groups;
R⁷ is selected from the group consisting of hydrogen, (C₁-C₆)alkyl, —R⁹(R¹⁵)ₘ, —OR(R¹⁵)ₘ, halo, —C(O)R¹², —R⁹R¹², nitrile, and —R¹⁴;
R⁸ is independently selected from the group consisting of hydrogen and (C₁-C₆)alkyl;
R⁹ is (C₁-C₆)alkyl;
R¹⁰ is (C₄-C₁₄)aryl;
R¹¹ is selected from the group consisting of (C₁-C₆)alkyl, dimethyl, sulfonamide, —OR⁸, —C(O)R¹², oxo, nitrile, —R¹², halo, —R⁹(R¹⁵)ₘ, and —OR(R¹⁵)ₘ;
R¹² is —NR^{x}R^{y}, wherein R^{x} and R^{y} are independently selected from the group consisting of hydrogen and (C₁-C₆)alkyl; wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring independently have one to four heteroatoms selected from N, S and O, and wherein said heterocyclic ring or heteroaryl ring may be also optionally substituted with one to three R¹¹ groups;
R¹³ is (C₃-C₂)Cycloalkyl;
R¹⁴ is selected from the group consisting of (C₁-C₁₁)heteroaryl or (C₁-C₁₁)heterocyclic, wherein said (C₁-C₁₁)heterocyclic or (C₁-C₁₁)heteroaryl each may have one to three heteroatoms selected from N, S, or O, and wherein said (C₁-C₁₁)heteroaryl or (C₁-C₁₁)heterocyclic may also be optionally substituted by one to three independent R¹¹ groups;
R¹⁵ is halo; and
m is independently 0 or an integer from 1 to 3.

There is also provided a pharmaceutical composition comprising a pharmaceutically acceptable diluent and a therapeutically effective amount of a compound as defined in any of the formulas described herein.

There is also provided a method for treating a viral infection in a subject that has been diagnosed with said viral infection or is at risk of developing said viral infection comprising administering to said subject, a compound of any of the formulas described herein.

There is also provided a method for enhancing the immune response in a subject that has been diagnosed with a viral infection or is at risk of developing said viral infection comprising administering to said subject, a compound as defined in any of the formulas described herein.

There is also provided a method for enhancing the immune response to a viral infection in a subject that is immunocompromised or is at risk of developing an immunocomprised immune system comprising administering to said subject, a compound as defined in any of the formulas described herein.

### BRIEF DESCRIPTION OF THE FIGURES

FIG. 1 shows the expression of secreted alkaline phosphatase (SEAP) upon treatment.

FIG. 2 shows STAT1 phosphorylation for IFNα and Example 1 for 1 hour, 6 hours, and 24 hours.

FIG. 3 shows the induction of various known interferon stimulated genes (ISGs) in a time-dependent manner for Example 1, IFNα, and an inactive analog compound.

FIG. 4 shows the correlation of antiviral activity in HCV replication and the induction of M×1 RNA and indicates the activation of phosphor-STAT1 in a dose response of Example 1.

FIG. 5 shows the antiviral activity of Example 1 by small interfering RNAs (siRNAs).

FIG. 6 shows the induction of interferon stimulated genes (ISGs) in mice in vivo following treatment with Example 1.

FIG. 7 shows the dose response of interferon stimulated genes (ISGs) induction in vivo following treatment with Example 11.

FIG. 8 shows that a broad spectrum of antiviral activity of Example 1 was accessed by testing other viruses.

FIG. 9 shows the reduction of the number of RSV plaques after treatment with Example 1 and IFNα.

FIG. 10 shows a protein Western Blot and Taqman gene expression analysis on the ability of various compounds of the present invention and IFNα to upregulate pSTAT1 and ISG expression.

FIG. 11 shows a protein Western Blot of pSTAT1 and ISG activation in human keratinocytes after their treatment with various compounds of the present invention and IFNα.

FIG. 12 shows Taqman gene expression analysis patterns for pSTAT1 and ISG after treatment of reconstructed human epidermis (“RHE”) with various compounds of the present invention and IFNα. In RHE tissue culture these agents stimulate production of pSTAT1 and induction of IFN-stimulated genes (ISGs), MX1, OAS2 and IL6.

FIG. 13 shows a bar graph representing gene expression analysis at 8 and 72 hours post treatment with JAK/Stat activators (Ex. 1, 2, and 11) having significant upregulation of ISG (MX1) expression.

FIG. 14 shows a bar graph representing gene expression analysis at 8 and 72 hours post treatment with JAK/Stat activators (Ex. 1, 2, and 11) having significant upregulation of ISG (OAS2) expression.

FIG. 15 shows a bar graph representing gene expression analysis at 8 and 72 hours post treatment with JAK/Stat activators (Ex. 1, 2, and 11) having significant upregulation of ISG (IL-6) expression.

### DETAILED DESCRIPTION OF REPRESENTATIVE EMBODIMENTS

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings.

“Alkyl” refers to monovalent saturated aliphatic hydrocarbyl groups having from 1 to 14 carbon atoms and, in some embodiments, from 1 to 6 carbon atoms. “(Cₓ₋C_{y})alkyl” refers to alkyl groups having from x to y carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkylidene” or “alkylene” refers to divalent saturated aliphatic hydrocarbyl groups having from 1 to 10 carbon atoms and, in some embodiments, from 1 to 6 carbon atoms. “(Cᵤ₋ᵥ)alkylene” refers to alkylene groups having from u to v carbon atoms. The alkylidene and alkylene groups include branched and straight chain hydrocarbyl groups. For example “(C₁₋₆)alkylene” is meant to include methylene, ethylene, propylene, 2-methypropylene, pentylene, and so forth.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, isopropylene, 1,3-butadienyl, and the like.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Acyl” refers to the groups H—C(O)—, alkyl-C(O)—, alkenyl-C(O)—, alkynyl-C(O)—, cycloalkyl-C(O)—, aryl-C(O)—, heteroaryl-C(O)—, and heterocyclic-C(O)—. Acyl includes the “acetyl” group CH₃C(O)—.

“Acylamino” refers to the groups —NR²⁰C(O)alkyl, —NR²⁰C(O)cycloalkyl, —NR²⁰C(O)alkenyl, —NR²⁰C(O)alkynyl, —NR²⁰C(O)aryl, —NR²⁰C(O)heteroaryl, and —NR²⁰C(O)heterocyclic, wherein R²⁰ is hydrogen or alkyl.

“Acyloxy” refers to the groups alkyl-C(O)O—, alkenyl-C(O)O—, alkynyl-C(O)O—, aryl-C(O)O—, cycloalkyl-C(O)O—, heteroaryl-C(O)O—, and heterocyclic-C(O)O—.

“Amino” refers to the group —NR²¹R²² where R²¹ and R² are independently selected from hydrogen, alkyl, alkenyl, alkynyl, aryl, cycloalkyl, heteroaryl, heterocyclic, —SO₂-alkyl, —SO₂-alkenyl, —SO₂-cycloalkyl, —SO₂-aryl, —SO₂-heteroaryl, and —SO₂-heterocyclic, and wherein R²¹ and R²² are optionally joined together with the nitrogen bound thereto to form a heterocyclic group. When R²¹ is hydrogen and R²² is alkyl, the amino group is sometimes referred to herein as alkylamino. When R²¹ and R²² are alkyl, the amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²⁶R²⁷ where R²⁶ and R²⁷ are independently selected from hydrogen, alkyl, alkenyl, alkynyl, aryl, cycloalkyl, heteroaryl, heterocyclic, hydroxy, alkoxy, amino, and acylamino, and where R²⁶ and R²⁷ are optionally joined together with the nitrogen bound thereto to form a heterocyclic group.

“Aryl” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“Cyano” or “nitrile” refers to the group —CN.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups, such as cyclohexenyl. Examples of cycloalkyl groups include, for instance, adamantyl, cyclopropyl, cyclobutyl, cyclohexyl, cyclopentyl, cyclooctyl, cyclopentenyl, and cyclohexenyl. Examples of cycloalkyl groups that include multiple bicycloalkyl ring systems are bicyclohexyl, bicyclopentyl, bicyclooctyl, and the like. Two such bicycloalkyl multiple ring structures are exemplified and named below:

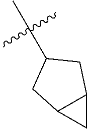 [SMILES: *C1CC2CC2C1]

bicyclohexyl, and

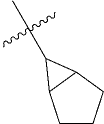 [SMILES: *C1C2CCCC12]

bicyclohexyl.

“(Cᵤ-Cᵥ)cycloalkyl” refers to cycloalkyl groups having u to v carbon atoms.

“Spiro cycloalkyl” refers to a 3 to 10 member cyclic substituent formed by replacement of two hydrogen atoms at a common carbon atom in a cyclic ring structure or in an alkylene group having 2 to 9 carbon atoms, as exemplified by the following structure wherein the group shown here attached to bonds marked with wavy lines is substituted with a spiro cycloalkyl group:

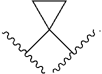 [SMILES: *C1(*)CC1]

“Fused cycloalkyl” refers to a 3 to 10 member cyclic substituent formed by the replacement of two hydrogen atoms at different carbon atoms in a cycloalkyl ring structure, as exemplified by the following structure wherein the cycloalkyl group shown here contains bonds marked with wavy lines which are bonded to carbon atoms that are substituted with a fused cycloalkyl group:

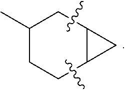

“Halo” or “halogen” refers to fluoro, chloro, bromo, and iodo.

“Haloalkoxy” refers to substitution of alkoxy groups with 1 to 5 (e.g. when the alkoxy group has at least 2 carbon atoms) or in some embodiments 1 to 3 halo groups (e.g. trifluoromethoxy).

“Hydroxy” or “hydroxyl” refers to the group —OH.

“Heteroaryl” refers to an aromatic group of from 1 to 14 carbon atoms and 1 to 6 heteroatoms selected from oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In some embodiments, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, imidazolinyl, isoxazolyl, pyrrolyl, pyrazolyl, pyridazinyl, pyrimidinyl, purinyl, phthalazyl, naphthylpryidyl, benzofuranyl, tetrahydrobenzofuranyl, isobenzofuranyl, benzothiazolyl, benzoisothiazolyl, benzotriazolyl, indolyl, isoindolyl, indolizinyl, dihydroindolyl, indazolyl, indolinyl, benzoxazolyl, quinolyl, isoquinolyl, quinolizyl, quianazolyl, quinoxalyl, tetrahydroquinolinyl, isoquinolyl, quinazolinonyl, benzimidazolyl, benzisoxazolyl, benzothienyl, benzopyridazinyl, pteridinyl, carbazolyl, carbolinyl, phenanthridinyl, acridinyl, phenanthrolinyl, phenazinyl, phenoxazinyl, phenothiazinyl, and phthalimidyl.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from nitrogen, sulfur, phosphorus or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen, phosphorus and/or sulfur atom(s) of the heterocyclic group are optionally oxidized to provide for the N-oxide, phosphinane oxide, sulfinyl, sulfonyl moieties. More specifically the heterocyclyl includes, but is not limited to, tetrahydropyranyl, piperidinyl, piperazinyl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

Examples of heterocycle and heteroaryl groups include, but are not limited to, azetidine, pyrrole, imidazole, pyrazole, pyridine, pyrazine, pyrimidine, pyridazine, pyridone, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholine, thiomorpholine (also referred to as thiamorpholine), piperidine, pyrrolidine, and tetrahydrofuranyl.

“Fused heterocyclic” refers to a 3 to 10 member cyclic substituent formed by the replacement of two hydrogen atoms at different carbon atoms in a cycloalkyl ring structure, as exemplified by the following structure wherein the cycloalkyl group shown here contains bonds marked with wavy lines which are bonded to carbon atoms that are substituted with a fused heterocyclic group:

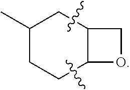

“Compound”, “compounds”, “chemical entity”, and “chemical entities” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Oxo” refers to a (═O) group.

“Oxazolidinone” refers to a 5-membered heterocyclic ring containing one nitrogen and one oxygen as heteroatoms and also contains two carbons and is substituted at one of the two carbons by a carbonyl group as exemplified by any of the following structures, wherein the oxazolidinone groups shown here are bonded to a parent molecule, which is indicated by a wavy line in the bond to the parent molecule:

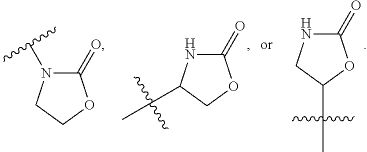 [SMILES: *N1CCOC1=O ; *C1COC(=O)N1 ; *C1CNC(=O)O1]

“Racemates” refers to a mixture of enantiomers. In an embodiment of the invention, the compounds of Formula I, or pharmaceutically acceptable salts thereof, are enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

“Solvate” or “solvates” of a compound refer to those compounds, as defined above, which are bound to a stoichiometric or non-stoichiometric amount of a solvent. Solvates of a compound includes solvates of all forms of the compound. In certain embodiments, solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Subject” refers to mammals and includes humans and non-human mammals. In some embodiments, the subject is a human. In other embodiments, the subject is an animal such as dogs, cats, horses, cows, and livestock animals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Wherever dashed lines occur adjacent to single bonds denoted by solid lines, then the dashed line represents an optional double bond at that position. Likewise, wherever dashed circles appear within ring structures denoted by solid lines or solid circles, then the dashed circles represent one to three optional double bonds arranged according to their proper valence taking into account whether the ring has any optional substitutions around the ring as will be known by one of skill in the art. For example, the dashed line in the structure below could either indicate a double bond at that position or a single bond at that position:

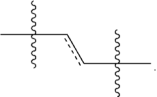

Similarly, ring A below could be a cyclohexyl ring without any double bonds or it could also be a phenyl ring having three double bonds arranged in any position that still depicts the proper valence for a phenyl ring. Likewise, in ring B below, any of X¹-X⁵ could be selected from: C, CH, or CH₂, N, or NH, and the dashed circle means that ring B could be a cyclohexyl or phenyl ring or a N-containing heterocycle with no double bonds or a N-containing heteroaryl ring with one to three double bonds arranged in any position that still depicts the proper valence:

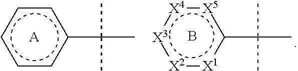

Where specific compounds or generic formulas are drawn that have aromatic rings, such as aryl or heteroaryl rings, then it will understood by one of still in the art that the particular aromatic location of any double bonds are a blend of equivalent positions even if they are drawn in different locations from compound to compound or from formula to formula. For example, in the two pyridine rings (A and B) below, the double bonds are drawn in different locations, however, they are known to be the same structure and compound:

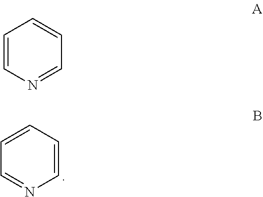

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—. In a term such as “C(R^{x})₂”, it should be understood that the two R^{x} groups can be the same, or they can be different if R^{x} is defined as having more than one possible identity. In addition, certain substituents are drawn as —R^{x}R^{y}, where the “—” indicates a bond adjacent to the parent molecule and R^{y} being the terminal portion of the functionality. Similarly, it is understood that the above definitions are not intended to include impermissible substitution patterns (e.g., methyl substituted with 5 fluoro groups). Such impermissible substitution patterns are well known to the skilled artisan.

In accordance with one embodiment of the present invention, there is provided a compound of Formula (I):

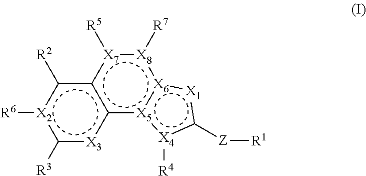

or a pharmaceutically acceptable salt thereof, wherein:
X₁, X₄, X₇, and X₈, are independently selected from N, NH, S, O, C, CH, or CH₂;
X₂, X₃, X₅, and X₆ are independently selected from N, NH, C, CH, or CH₂;
Z is selected from a bond, —C(O), or (C₁-C₆)alkylene;
R¹ is selected from the group consisting of hydrogen, —R¹², —R¹⁴, —R⁹(R¹⁵)ₘ, —OR(R¹⁵)ₘ, and halo;
R² is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, —R¹²S(O)₂, —S(O)₂R¹², halo, nitrile, sulfonamide, sulfone, sulfoxide, (C₄-C₁₄)aryl, and (C₃-C₁₂)cycloalkyl, wherein said R² group may be optionally substituted with one to three R¹¹ groups;
R³ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, halo, nitrile, sulfonamide, sulfone, sulfoxide, (C₄-C₁₄)aryl, and (C₃-C₁₂)cycloalkyl, wherein said R³ group may be optionally substituted with one to three R¹¹ groups;
R⁴ is optionally absent or is selected from the group consisting of hydrogen, (C₁-C₆)alkyl, —R⁹(R¹⁵)ₘ, —OR(R¹⁵)ₘ, —R⁹R¹⁰, —C(O)R⁹, —C(O)R¹³, halo, and (C₃-C₁₂)cycloalkyl;
R⁵ is selected from the group consisting of hydrogen, (C₁-C₆)alkyl, —C(O)R¹², —R⁹R¹², —R⁹(R¹⁵)ₘ, —OR⁹(R¹⁵)ₘ, —R¹⁴, halo, and nitrile;
R⁶ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, halo, nitrile, sulfonamide, sulfone, sulfoxide, (C₄-C₁₄)aryl, and (C₃-C₁₂)cycloalkyl, wherein said R⁶ group may be optionally substituted with one to three R¹¹ groups;
R⁷ is selected from the group consisting of hydrogen, (C₁-C₆)alkyl, —R⁹(R¹⁵)ₘ, —OR(R¹⁵), halo, —C(O)R¹², —R⁹R¹², nitrile, and —R¹⁴;
R⁸ is independently selected from the group consisting of hydrogen and (C₁-C₆)alkyl;
R⁹ is (C₁-C₆)alkyl;
R¹⁰ is (C₄-C₁₄)aryl;
R¹¹ is selected from the group consisting of (C₁-C₆)alkyl, dimethyl, sulfonamide, —OR⁸, —C(O)R¹², oxo, nitrile, —R¹², halo, —R⁹(R¹⁵)ₘ, and —OR(R¹⁵)ₘ;
R¹² is —NR^{x}R^{y}, wherein R^{x} and R^{y} are independently selected from the group consisting of hydrogen and (C₁-C₆)alkyl; wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring independently have one to four heteroatoms selected from N, S and O, and wherein said heterocyclic ring or heteroaryl ring may be also optionally substituted with one to three R¹¹ groups;
R¹³ is (C₃-C₁₂)cycloalkyl;
R¹⁴ is selected from the group consisting of (C₁-C₁₁)heteroaryl or (C₁-C₁₁)heterocyclic, wherein said (C₁-C₁₁)heterocyclic or (C₁-C₁₁)heteroaryl each may have one to three heteroatoms selected from N, S, or O, and wherein said (C₁-C₁₁)heteroaryl or (C₁-C₁₁)heterocyclic may also be optionally substituted by one to three independent R¹¹ groups;
R¹⁵ is halo; and
m is independently 0 or an integer from 1 to 3.

In certain embodiments, with regards to the formulas described herein and throughout, m is an integer that ranges from 2 to 3. In other embodiments m is 2. In still other embodiments, m is 3.

In accordance with another embodiment of the present invention, there is provided a compound of Formula (I):

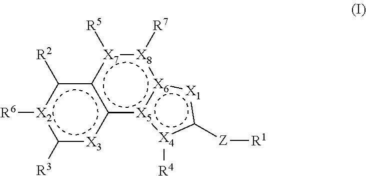

or a pharmaceutically acceptable salt thereof, wherein:
X₁, X₄, X₇, and X₈, are independently selected from N, NH, S, O, C, CH, or CH₂;
X₂, X₃, X₅, and X₆ are independently selected from N, NH, C, CH, or CH₂;
Z is selected from the group consisting of a bond, —C(O), and methylene;
R¹ is selected from the group consisting of hydrogen, —R¹², chloro, bromo, fluoro, difluoromethyl, trifluoromethyl, difluoromethoxy, trifluoromethoxy, oxazolyl, furanyl, oxolanyl, oxadiazolyl, oxazolidinyl, imidazolidinyl, imidazolyl, oxanyl, piperidinyl, morpholinyl, dihydropyranyl, pyranyl, tetrahydropyridinyl, pyridinyl, and pyrrolidinyl, wherein said R¹ group may be optionally substituted with one to three R¹¹ groups;
R² is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R² may be optionally substituted by one to three independent R¹¹ groups;
R³ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R³ may be optionally substituted by one to three independent R¹¹ groups;
R⁴ is optionally absent or is selected from the group consisting of hydrogen, methyl, ethyl, propyl, butyl, —C(O)R⁹, —(CO)R¹³, chloro, bromo, fluoro, difluoromethyl, trifluoromethyl, difluoromethoxy, trifluoromethoxy, cyclopropyl, cyclobutyl, cyclopentyl, and cyclohexyl;
R⁵ is selected from the group consisting of hydrogen, methyl, ethyl, propyl, butyl, —C(O)R¹², —R⁹R¹², nitrile, chloro, bromo, fluoro, difluoromethyl, trifluoromethyl, difluoromethoxy, trifluoromethoxy, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R⁶ may be optionally substituted by one to three independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;

R¹² is —NR^{x}R^{y}, wherein R^{x} and R^{y} are independently selected from the group consisting of hydrogen and methyl; and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, each independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, imidazolyl, thiophenyl, oxanyl, pyrrolidinyl, furanyl, morpholinyl, oxazolyl, oxadiazolyl, oxazolidinyl, dihydropyranyl, tetrahydropyridinyl, imidazolidinyl, and pyridinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (I):

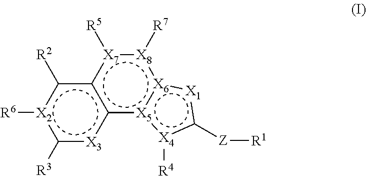

or a pharmaceutically acceptable salt thereof, wherein:

X₁, X₂ X₃, X₄, X₅, X₆ X₇, and X₈, are independently selected from N, C, or CH;
Z is selected from a bond or methylene;
R¹ is selected from the group consisting of oxazolyl, oxanyl, oxolanyl, oxadiazolyl, oxazolidinyl, dihydropyranyl, tetrahydropyridinyl, pyrrolidinyl, morpholinyl, imidazolidinyl, and furanyl, wherein said R¹ group may be optionally substituted with one to two R¹¹ groups;
R² is selected from the group consisting of hydrogen, methyl, ethyl, propyl, isopropyl, butyl, isobutyl, methoxy, ethoxy, propoxy, chloro, bromo, fluoro, nitrile, difluoromethyl, trifluoromethyl, difluoromethoxy, trifluoromethoxy, cyclopropyl, cyclobutyl, cyclopentyl, cyclohexyl, imidazolyl, phenyl, and oxolanyl, wherein R² may be optionally substituted by one to two independent R¹¹ groups;
R³ is selected from the group consisting of hydrogen, methyl, ethyl, propyl, isopropyl, butyl, isobutyl, methoxy, ethoxy, propoxy, nitrile, difluoromethyl, trifluoromethyl, —C(O)R¹², oxanyl, oxolanyl, pyridinyl, phenyl, thiophenyl, piperidinyl, pyrrolidinyl, wherein R³ may be optionally substituted by one to two independent R¹¹ groups;
R⁴ is optionally absent or is selected from the group consisting of hydrogen, methoxy, ethoxy, propoxy, methyl, ethyl, propyl, butyl, nitrile, —C(O)R⁹, —C(O)R¹³, chloro, bromo, and fluoro;
R⁵ is selected from the group consisting of hydrogen, methyl, ethyl, propyl, butyl, —C(O)R¹², —R⁹R¹², nitrile, methoxy, ethoxy, propoxy, nitrile, chloro, bromo, fluoro, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, difluoromethoxy, trifluoromethoxy, piperidinyl, morpholinyl, oxolanyl, wherein R⁶ may be optionally substituted by one to two independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl; and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, each independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (I):

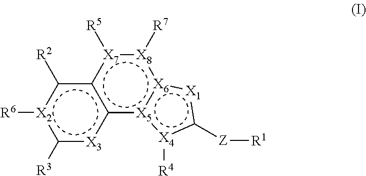

or a pharmaceutically acceptable salt thereof, wherein:

X₁, X₂ X₃, X₄, X₅, X₆ X₇, and X₈, are independently selected from N or CH;
Z is selected from a bond or methylene;
R¹ is selected from the group consisting of oxazolyl, oxanyl, oxolanyl, oxadiazolyl, oxazolidinyl, dihydropyranyl, tetrahydropyridinyl, pyrrolidinyl, morpholinyl, imidazolidinyl, and furanyl, wherein said R¹ group may be optionally substituted with one to two R¹¹ groups;
R² is selected from the group consisting of hydrogen, methyl, ethyl, propyl, isopropyl, butyl, isobutyl, methoxy, ethoxy, propoxy, chloro, bromo, fluoro, nitrile, difluoromethyl, trifluoromethyl, difluoromethoxy, trifluoromethoxy, cyclopropyl, cyclobutyl, cyclopentyl, cyclohexyl, imidazolyl, phenyl, and oxolanyl, wherein R² may be optionally substituted by one to two independent R¹¹ groups;
R³ is selected from the group consisting of hydrogen, methyl, ethyl, propyl, isopropyl, butyl, isobutyl, methoxy, ethoxy, propoxy, nitrile, difluoromethyl, trifluoromethyl, —C(O)R¹², oxanyl, oxolanyl, pyridinyl, phenyl, thiophenyl, piperidinyl, pyrrolidinyl, wherein R³ may be optionally substituted by one to two independent R¹¹ groups;
R⁴ is optionally absent or is selected from the group consisting of hydrogen, methoxy, ethoxy, propoxy, methyl, ethyl, propyl, butyl, nitrile, —C(O)R⁹, —C(O)R¹³, chloro, bromo, and fluoro;
R⁵ is selected from the group consisting of hydrogen, methyl, ethyl, propyl, butyl, —C(O)R¹², —R⁹R¹², nitrile, methoxy, ethoxy, propoxy, nitrile, chloro, bromo, fluoro, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, difluoromethoxy, trifluoromethoxy, piperidinyl, morpholinyl, oxolanyl, wherein R⁶ may be optionally substituted by one to two independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from hydrogen or methyl;
R¹³ is selected from the group consisting of cyclopropyl, cyclobutyl, cyclopentyl, and cyclohexyl;
R¹⁴ is selected from the group consisting of piperidinyl, oxolanyl, morpholinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (I), wherein R¹ is selected from the group consisting of thiophenyl, furanyl, pyridinyl, tetrahydrofuranyl, tetrahydropyranyl, methylpyrrolidinyl, methylpiperdidinyl,

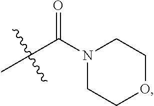 [SMILES: *C(=O)N1CCOCC1]

and methyl-morpholinyl.

In accordance with another embodiment of the present invention, there is provided a compound of Formula (I), wherein R² is selected from the group consisting of morpholinyl, methylpiperidinyl, and tetrahydrofuranyl.

In accordance with another embodiment of the present invention, there is provided a compound of Formula (I), wherein R³ is selected from the group consisting of tetrahydrofuranyl, piperidinyl, pyrrolidinyl, 1H-imidazolyl, propanyloxy, and carbonyl-morpholinyl.

In accordance with another embodiment of the present invention, there is provided a compound of Formula (I), wherein R⁴ is pyrrolidinyl.

In accordance with another embodiment of the present invention, there is provided a compound of Formula (I), wherein R⁵ is pyrrolidinyl.

In accordance with another embodiment of the present invention, there is provided a compound of Formula (I), wherein R⁶ is selected from the group consisting of oxadiazolyl, furanyl, oxazolyl, methyl-pyrrolidyl, methyl-pyrrolidinol, methyl-morpholinyl, oxazolidinone), pyrrolidinone, imidazolidinone, imidazolidinedione, and methyl-oxazole.

In accordance with another embodiment of the present invention, there is provided a compound of Formula (I), wherein:
X₁, X₂, X₃, X₄, X₅, X₆ X₇, and X₈ are selected from N and CH;
R¹ is selected from the group consisting of hydrogen, cyclopentyl, cyclopropyl, propan-2-yl, methyl, ethyl, 2-methylpropyl, thiophen-3-yl, furan-3-yl, pyridine-3-yl, ethoxy, phenyl, difluoromethoxy, chloride, tetrahydrofuran-(2 or 3)-yl, tetrahydropyran-(3 or 4)-yl, 1-methylpyrrolidin-(2 or 3)-yl, 1-methyl-(3 or 4)-piperdidinyl, carboxamide,

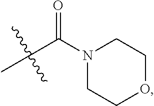 [SMILES: *C(=O)N1CCOCC1]

N,N-dimethyl-carboxamide, N-methyl-carboxamide, methyl-dimethylamine, 4-methyl-morpholinyl, 4-carbonyl-morpholinyl, cyclopentyl-methyl, and trifluoromethyl;
R² is selected from the group consisting of hydrogen, trifluoromethyl, propan-2-yl, morpholin-4-yl, 1-methylpiperidin-4-yl, and tetrahydrofuran-3-yl;
R³ is selected from the group consisting of hydrogen, trifluoromethyl, chloride, methyl, propan-2-yl, 2-methylpropyl, phenyl, cyclopropyl, cyclobutyl, cyclopentyl, cyclohexyl, tetrahydrofuran-(2 or 3)-yl, and piperidin-1-yl, pyrrolidin-1-yl, 1H-imidazol-(2 or 5)-yl, propan-2-yloxy, ethoxy, cyano, carboxamide, and carbonyl-morpholinyl;
R⁴ is optionally absent or is selected from the group consisting of hydrogen, pyrrolidin-1-yl, cyano, carboxamide, and dimethyl-methylamine;
R⁵ is selected from the group consisting of hydrogen, pyrrolidin-1-yl, cyano, carboxamide, and dimethyl-methylamine;
R⁶ is selected from the group consisting of hydrogen, 1,3,4-oxadiazol-2-yl, furan-2-yl, 1,3-oxazol-2-yl, methyl-dimethylamine, 1-methyl-pyrrolidyl, 1-methyl-pyrrolidin-3-ol, 4-methyl-morpholinyl, 3-(1,3-oxazolidin-2-one), 1-pyrrolidin-2-one, 1-imidazolidin-2-one, 1-imidazolidine-2,4-dione, 4-methyl-1,3-oxazol-5-yl, 4-(propan-2-yl)-1,3-oxazol-5-yl, 5-(4,4-dimethyl-4,5-dihydro-1,3-oxazol-5-yl), 5-(1,3-oxazol-4-amine), 5-(1,3-oxazole-4-carbonitrile), 5-(1,3-oxazole-4-carboxamide); and
R⁷ is selected from the group consisting of hydrogen and chloro.

In accordance with another embodiment of the present invention, there is provided a compound of Formula (I), wherein:
X₁, X₂, X₃, X₄, X₅, X₆ X₇, and X₈ are selected from N or CH;
R¹ is selected from the group consisting of hydrogen, cyclopentyl, cyclopropyl, propan-2-yl, methyl, ethyl, 2-methylpropyl, thiophen-3-yl, furan-3-yl, pyridine-3-yl, ethoxy, phenyl, difluoromethoxy, chloride, tetrahydrofuran-(2 or 3)-yl, tetrahydropyran-(3 or 4)-yl, 1-methylpyrrolidin-(2 or 3)-yl, 1-methyl-(3 or 4)-piperdidinyl, carboxamide,

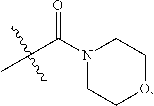 [SMILES: *C(=O)N1CCOCC1]

N,N-dimethyl-carboxamide, N-methyl-carboxamide, methyl-dimethylamine, 4-methyl-morpholinyl, 4-carbonyl-morpholinyl, cyclopentyl-methyl, and trifluoromethyl;
R² is selected from the group consisting of hydrogen, trifluoromethyl, propan-2-yl, morpholin-4-yl, 1-methylpiperidin-4-yl, and tetrahydrofuran-3-yl;
R³ is selected from the group consisting of hydrogen, trifluoromethyl, chloride, methyl, propan-2-yl, 2-methylpropyl, phenyl, cyclopropyl, cyclobutyl, cyclopentyl, cyclohexyl, tetrahydrofuran-(2 or 3)-yl, and piperidin-1-yl, pyrrolidin-1-yl, 1H-imidazol-(2 or 5)-yl, propan-2-yloxy, ethoxy, cyano, carboxamide, and carbonyl-morpholinyl;
R⁴ is optionally absent or is selected from the group consisting of hydrogen, pyrrolidin-1-yl, cyano, carboxamide, and dimethyl-methylamine;
R⁵ is selected from the group consisting of hydrogen, pyrrolidin-1-yl, cyano, carboxamide, and dimethyl-methylamine;
R⁶ is selected from the group consisting of hydrogen, 1,3,4-oxadiazol-2-yl, furan-2-yl, 1,3-oxazol-2-yl, methyl-dimethylamine, 1-methyl-pyrrolidyl, 1-methyl-pyrrolidin-3-ol, 4-methyl-morpholinyl, 3-(1,3-oxazolidin-2-one), 1-pyrrolidin-2-one, 1-imidazolidin-2-one, 1-imidazolidine-2,4-dione, 4-methyl-1,3-oxazol-5-yl, 4-(propan-2-yl)-1,3-oxazol-5-yl, 5-(4,4-dimethyl-4,5-dihydro-1,3-oxazol-5-yl), 5-(1,3-oxazol-4-amine), 5-(1,3-oxazole-4-carbonitrile), 5-(1,3-oxazole-4-carboxamide); and
R⁷ is selected from the group consisting of hydrogen and chloride.

In accordance with another embodiment of the present invention, there is provided a compound of Formula (II):

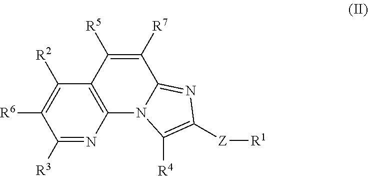

or a pharmaceutically acceptable salt thereof, wherein:
Z is selected from a bond, —C(O), or (C₁-C₆)alkylene;
R¹ is selected from the group consisting of hydrogen, —R¹², —R¹⁴, —R⁹(R¹⁵)ₘ, —OR(R¹⁵)ₘ, and halo;
R² is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, OR¹³, —R¹²S(O)₂, —S(O)₂R¹², halo, nitrile, sulfonamide, sulfone, sulfoxide, (C₄-C₁₄)aryl, and (C₃-C₁₂)cycloalkyl, wherein said R² group may be optionally substituted with one to three R¹¹ groups;
R³ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, halo, nitrile, sulfonamide, sulfone, sulfoxide, (C₄-C₁₄)aryl, and (C₃-C₁₂)cycloalkyl, wherein said R³ group may be optionally substituted with one to three R¹¹ groups;
R⁴ is selected from the group consisting of hydrogen, (C₁-C₆)alkyl, —R⁹(R¹⁵)ₘ, —OR(R¹⁵)ₘ, —C(O)R⁹, —C(O)R¹³, halo, and (C₃-C₁₂)cycloalkyl;
R⁵ is selected from the group consisting of hydrogen, (C₁-C₆)alkyl, —C(O)R¹², —R⁹R¹², —R⁹(R¹⁵)ₘ, —OR⁹(R¹⁵)ₘ, —R¹⁴, halo, and nitrile;
R⁶ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, halo, nitrile, sulfonamide, sulfone, sulfoxide, (C₄-C₁₄)aryl, and (C₃-C₁₂)cycloalkyl, wherein said R⁶ group may be optionally substituted with one to three R¹¹ groups;
R⁷ is selected from the group consisting of hydrogen, (C₁-C₆)alkyl, —R⁹(R¹⁵)ₘ, —OR(R¹⁵), halo, —C(O)R¹², —R⁹R¹², nitrile, and —R¹⁴;
R⁸ is independently selected from the group consisting of hydrogen and (C₁-C₆)alkyl;
R⁹ is (C₁-C₆)alkyl;
R¹⁰ is (C₄-C₁₄)aryl;
R¹¹ is selected from the group consisting of (C₁-C₆)alkyl, dimethyl, sulfonamide, —OR⁸, —C(O)R¹², Oxo, nitrile, —R¹², halo, —R⁹(R¹⁵)ₘ, and —OR(R¹⁵)ₘ;
R¹² is —NR^{x}R^{y}, wherein R^{x} and R^{y} are independently selected from the group consisting of hydrogen and (C₁-C₆)alkyl, and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring independently have one to four heteroatoms selected from N, S and O, and wherein said heterocyclic ring or heteroaryl ring may be also optionally substituted with one to three R¹¹ groups;
R¹³ is (C₃-C₁₂)cycloalkyl;
R¹⁴ is selected from the group consisting of (C₁-C₁₁)heteroaryl or (C₁-C₁₁)heterocyclic, wherein said (C₁-C₁₁)heterocyclic or (C₁-C₁₁)heteroaryl each may have one to three heteroatoms selected from N, S, or O, and wherein said (C₁-C₁₁)heteroaryl or (C₁-C₁₁)heterocyclic may also be optionally substituted by one to three independent R¹¹ groups;
R¹⁵ is halo; and
m is independently 0 or an integer from 1 to 3.

In accordance with another embodiment of the present invention, there is provided a compound of Formula (II):

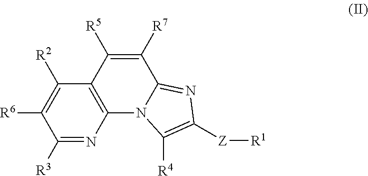

or a pharmaceutically acceptable salt thereof, wherein:
Z is selected from the group consisting of a bond, —C(O), and methylene;
R¹ is selected from the group consisting of hydrogen, —R¹², chloro, bromo, fluoro, difluoromethyl, trifluoromethyl, difluoromethoxy, trifluoromethoxy, oxazolyl, furanyl, oxolanyl, oxadiazolyl, oxazolidinyl, imidazolidinyl, imidazolyl, oxanyl, piperidinyl, morpholinyl, dihydropyranyl, pyranyl, tetrahydropyridinyl, pyridinyl, and pyrrolidinyl, wherein said R¹ group may be optionally substituted with one to three R¹¹ groups;
R² is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R² may be optionally substituted by one to three independent R¹¹ groups;
R³ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R³ may be optionally substituted by one to three independent R¹¹ groups;
R⁴ is selected from the group consisting of hydrogen, methyl, ethyl, propyl, butyl, —C(O)R⁹, —C(O)R¹³, chloro, bromo, fluoro, difluoromethyl, trifluoromethyl, difluoromethoxy, trifluoromethoxy, cyclopropyl, cyclobutyl, cyclopentyl, and cyclohexyl;
R⁵ is selected from the group consisting of hydrogen, methyl, ethyl, propyl, butyl, —C(O)R¹², —R⁹R¹², nitrile, chloro, bromo, fluoro, difluoromethyl, trifluoromethyl, difluoromethoxy, trifluoromethoxy, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R⁶ may be optionally substituted by one to three independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl; and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, imidazolyl, thiophenyl, oxanyl, pyrrolidinyl, furanyl, morpholinyl, oxazolyl, oxadiazolyl, oxazolidinyl, dihydropyranyl, tetrahydropyridinyl, imidazolidinyl, and pyridinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (II):

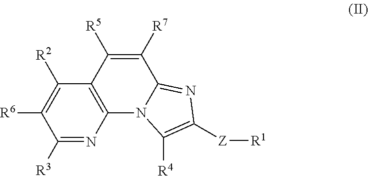

or a pharmaceutically acceptable salt thereof, wherein:
Z is selected from a bond or methylene;
R¹ is selected from the group consisting of oxazolyl, oxanyl, oxolanyl, oxadiazolyl, oxazolidinyl, dihydropyranyl, tetrahydropyridinyl, pyrrolidinyl, morpholinyl, imidazolidinyl, and furanyl, wherein said R¹ group may be optionally substituted with one to two R¹¹ groups;
R² is selected from the group consisting of hydrogen, methyl, ethyl, propyl, isopropyl, butyl, isobutyl, methoxy, ethoxy, propoxy, chloro, bromo, fluoro, nitrile, difluoromethyl, trifluoromethyl, difluoromethoxy, trifluoromethoxy, cyclopropyl, cyclobutyl, cyclopentyl, cyclohexyl, imidazolyl, phenyl, and oxolanyl, wherein R² may be optionally substituted by one to two independent R¹¹ groups;
R³ is selected from the group consisting of hydrogen, methyl, ethyl, propyl, isopropyl, butyl, isobutyl, methoxy, ethoxy, propoxy, nitrile, difluoromethyl, trifluoromethyl, —C(O)R¹², oxanyl, oxolanyl, pyridinyl, phenyl, thiophenyl, piperidinyl, pyrrolidinyl, wherein R³ may be optionally substituted by one to two independent R¹¹ groups;
R⁴ is selected from the group consisting of hydrogen, methoxy, ethoxy, propoxy, methyl, ethyl, propyl, butyl, nitrile, —C(O)R⁹, —C(O)R¹³, chloro, bromo, and fluoro;
R⁵ is selected from the group consisting of hydrogen, methyl, ethyl, propyl, butyl, —C(O)R¹², —R⁹R¹², nitrile, methoxy, ethoxy, propoxy, nitrile, chloro, bromo, fluoro, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, difluoromethoxy, trifluoromethoxy, piperidinyl, morpholinyl, oxolanyl, wherein R⁶ may be optionally substituted by one to two independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl; and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (II):

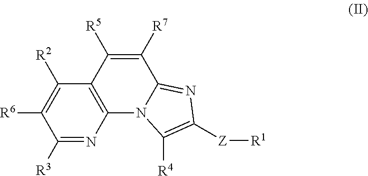

or a pharmaceutically acceptable salt thereof, wherein:
Z is selected from a bond or methylene;
R¹ is selected from the group consisting of oxazolyl, oxanyl, oxolanyl, oxadiazolyl, oxazolidinyl, dihydropyranyl, tetrahydropyridinyl, pyrrolidinyl, morpholinyl, imidazolidinyl, and furanyl, wherein said R¹ group may be optionally substituted with one to two R¹¹ groups;
R² is selected from the group consisting of hydrogen, methyl, ethyl, propyl, isopropyl, butyl, isobutyl, methoxy, ethoxy, propoxy, chloro, bromo, fluoro, nitrile, difluoromethyl, trifluoromethyl, difluoromethoxy, trifluoromethoxy, cyclopropyl, cyclobutyl, cyclopentyl, cyclohexyl, imidazolyl, phenyl, and oxolanyl, wherein R² may be optionally substituted by one to two independent R¹¹ groups;
R³ is selected from the group consisting of hydrogen, methyl, ethyl, propyl, isopropyl, butyl, isobutyl, methoxy, ethoxy, propoxy, nitrile, difluoromethyl, trifluoromethyl, —C(O)R¹², oxanyl, oxolanyl, pyridinyl, phenyl, thiophenyl, piperidinyl, pyrrolidinyl, wherein R³ may be optionally substituted by one to two independent R¹¹ groups;
R⁴ is selected from the group consisting of hydrogen, methoxy, ethoxy, propoxy, methyl, ethyl, propyl, butyl, nitrile, —C(O)R⁹, —C(O)R¹³, chloro, bromo, and fluoro;
R⁵ is selected from the group consisting of hydrogen, methyl, ethyl, propyl, butyl, —C(O)R¹², —R⁹R¹², nitrile, methoxy, ethoxy, propoxy, nitrile, chloro, bromo, fluoro, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, difluoromethoxy, trifluoromethoxy, piperidinyl, morpholinyl, oxolanyl, wherein R⁶ may be optionally substituted by one to two independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from hydrogen or methyl;
R¹³ is selected from the group consisting of cyclopropyl, cyclobutyl, cyclopentyl, and cyclohexyl;
R¹⁴ is selected from the group consisting of piperidinyl, oxolanyl, and morpholinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (III):

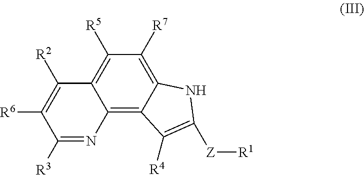

or a pharmaceutically acceptable salt thereof, wherein:
Z is selected from a bond, —C(O), or (C₁-C₆)alkylene;
R¹ is selected from the group consisting of hydrogen, —R¹², —R¹⁴, —R⁹(R¹⁵)ₘ, —OR(R¹⁵)ₘ, and halo;
R² is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, —R¹²S(O)₂, —S(O)₂R¹², halo, nitrile, sulfonamide, sulfone, sulfoxide, (C₄-C₁₄)aryl, and (C₃-C₁₂)cycloalkyl, wherein said R² group may be optionally substituted with one to three R¹¹ groups;
R³ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, OR¹³, halo, nitrile, sulfonamide, sulfone, sulfoxide, (C₄-C₁₄)aryl, and (C₃-C₁₂)cycloalkyl, wherein said R³ group may be optionally substituted with one to three R¹¹ groups;
R⁴ is selected from the group consisting of hydrogen, (C₁-C₆)alkyl, —R⁹(R¹⁵)ₘ, —OR(R¹⁵)ₘ, —C(O)R⁹, —(CO)R¹³, halo, and (C₃-C₁₂)cycloalkyl;
R⁵ is selected from the group consisting of hydrogen, —C(O)R¹², —R⁹R¹², —R⁹(R¹⁵)ₘ, —OR⁹(R¹⁵)ₘ, —R¹⁴, halo, and nitrile;
R⁶ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, halo, nitrile, sulfonamide, sulfone, sulfoxide, (C₄-C₁₄)aryl, and (C₃-C₁₂)cycloalkyl, wherein said R⁶ group may be optionally substituted with one to three R¹¹ groups;
R⁷ is selected from the group consisting of hydrogen, (C₁-C₆)alkyl, —R⁹(R¹⁵)ₘ, —OR(R¹⁵), halo, —C(O)R¹², —R⁹R¹², nitrile, and —R¹⁴;
R⁸ is independently selected from the group consisting of hydrogen and (C₁-C₆)alkyl;
R⁹ is (C₁-C₆)alkyl;
R¹⁰ is (C₄-C₁₄)aryl;
R¹¹ is selected from the group consisting of (C₁-C₆)alkyl, dimethyl, sulfonamide, —OR⁸, —C(O)R¹², oxo, nitrile, —R¹², halo, —R⁹(R¹⁵)ₘ, and —OR(R¹⁵)ₘ;
R¹² is —NR^{x}R^{y}, wherein R^{x} and R^{y} are independently selected from the group consisting of hydrogen and (C₁-C₆)alkyl, and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring independently have one to four heteroatoms selected from N, S and O, and wherein said heterocyclic ring or heteroaryl ring may be also optionally substituted with one to three R¹¹ groups;
R¹³ is (C₃-C₁₂)cycloalkyl;
R¹⁴ is selected from the group consisting of (C₁-C₁₁)heteroaryl or (C₁-C₁₁)heterocyclic, wherein said (C₁-C₁₁)heterocyclic or (C₁-C₁₁)heteroaryl each may have one to three heteroatoms selected from N, S, or O, and wherein said (C₁-C₁₁)heteroaryl or (C₁-C₁₁)heterocyclic may also be optionally substituted by one to three independent R¹¹ groups;
R¹⁵ is halo; and
m is independently 0 or an integer from 1 to 3.

In accordance with another embodiment of the present invention, there is provided a compound of Formula (III):

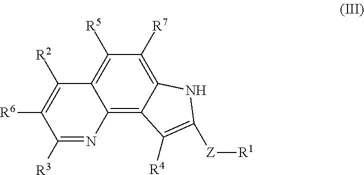

or a pharmaceutically acceptable salt thereof, wherein:
Z is selected from the group consisting of a bond, —C(O), and methylene;
R¹ is selected from the group consisting of hydrogen, —R¹², chloro, bromo, fluoro, difluoromethyl, trifluoromethyl, difluoromethoxy, trifluoromethoxy, oxazolyl, furanyl, oxolanyl, oxadiazolyl, oxazolidinyl, imidazolidinyl, imidazolyl, oxanyl, pipendinyl, morpholinyl, dihydropyranyl, pyranyl, tetrahydropyridinyl, pyridinyl, and pyrrolidinyl, wherein said R¹ group may be optionally substituted with one to three R¹¹ groups;
R² is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R² may be optionally substituted by one to three independent R¹¹ groups;
R³ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R³ may be optionally substituted by one to three independent R¹¹ groups;
R⁴ is selected from the group consisting of hydrogen, methyl, ethyl, propyl, butyl, —C(O)R⁹, —C(O)R¹³, chloro, bromo, fluoro, difluoromethyl, trifluoromethyl, difluoromethoxy, trifluoromethoxy, cyclopropyl, cyclobutyl, cyclopentyl, and cyclohexyl;
R⁵ is selected from the group consisting of hydrogen, methyl, ethyl, propyl, butyl, —C(O)R¹², —R⁹R¹², nitrile, chloro, bromo, fluoro, difluoromethyl, trifluoromethyl, difluoromethoxy, trifluoromethoxy, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R⁶ may be optionally substituted by one to three independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl; and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, imidazolyl, thiophenyl, oxanyl, pyrrolidinyl, furanyl, morpholinyl, oxazolyl, oxadiazolyl, oxazolidinyl, dihydropyranyl, tetrahydropyridinyl, imidazolidinyl, and pyridinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (III):

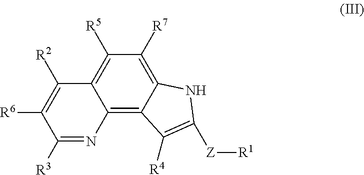

or a pharmaceutically acceptable salt thereof, wherein:
Z is selected from a bond or methylene;
R¹ is selected from the group consisting of oxazolyl, oxanyl, oxolanyl, oxadiazolyl, oxazolidinyl, dihydropyranyl, tetrahydropyridinyl, pyrrolidinyl, morpholinyl, imidazolidinyl, and furanyl, wherein said R¹ group may be optionally substituted with one to two R¹¹ groups;
R² is selected from the group consisting of hydrogen, methyl, ethyl, propyl, isopropyl, butyl, isobutyl, methoxy, ethoxy, propoxy, chloro, bromo, fluoro, nitrile, difluoromethyl, trifluoromethyl, difluoromethoxy, trifluoromethoxy, cyclopropyl, cyclobutyl, cyclopentyl, cyclohexyl, imidazolyl, phenyl, and oxolanyl, wherein R² may be optionally substituted by one to two independent R¹¹ groups;
R³ is selected from the group consisting of hydrogen, methyl, ethyl, propyl, isopropyl, butyl, isobutyl, methoxy, ethoxy, propoxy, nitrile, difluoromethyl, trifluoromethyl, —C(O)R¹², oxanyl, oxolanyl, pyridinyl, phenyl, thiophenyl, piperidinyl, pyrrolidinyl, wherein R³ may be optionally substituted by one to two independent R¹¹ groups;
R⁴ is selected from the group consisting of hydrogen, methoxy, ethoxy, propoxy, methyl, ethyl, propyl, butyl, nitrile, —C(O)R⁹, —C(O)R¹³, chloro, bromo, and fluoro;
R⁵ is selected from the group consisting of hydrogen, methyl, ethyl, propyl, butyl, —C(O)R¹², —R⁹R¹², nitrile, methoxy, ethoxy, propoxy, nitrile, chloro, bromo, fluoro, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, difluoromethoxy, trifluoromethoxy, piperidinyl, morpholinyl, oxolanyl, wherein R⁶ may be optionally substituted by one to two independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl; and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, each independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (III):

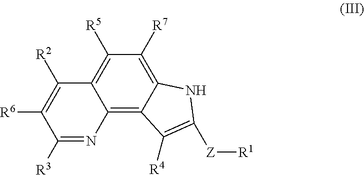

or a pharmaceutically acceptable salt thereof, wherein:
Z is selected from a bond or methylene;
R¹ is selected from the group consisting of oxazolyl, oxanyl, oxolanyl, oxadiazolyl, oxazolidinyl, dihydropyranyl, tetrahydropyridinyl, pyrrolidinyl, morpholinyl, imidazolidinyl, and furanyl, wherein said R¹ group may be optionally substituted with one to two R¹¹ groups;
R² is selected from the group consisting of hydrogen, methyl, ethyl, propyl, isopropyl, butyl, isobutyl, methoxy, ethoxy, propoxy, chloro, bromo, fluoro, nitrile, difluoromethyl, trifluoromethyl, difluoromethoxy, trifluoromethoxy, cyclopropyl, cyclobutyl, cyclopentyl, cyclohexyl, imidazolyl, phenyl, and oxolanyl, wherein R² may be optionally substituted by one to two independent R¹¹ groups;
R³ is selected from the group consisting of hydrogen, methyl, ethyl, propyl, isopropyl, butyl, isobutyl, methoxy, ethoxy, propoxy, nitrile, difluoromethyl, trifluoromethyl, —C(O)R¹², oxanyl, oxolanyl, pyridinyl, phenyl, thiophenyl, piperidinyl, pyrrolidinyl,
wherein R³ may be optionally substituted by one to two independent R¹¹ groups;
R⁴ is selected from the group consisting of hydrogen, methoxy, ethoxy, propoxy, methyl, ethyl, propyl, butyl, nitrile, —C(O)R⁹, —(CO)R¹³, chloro, bromo, and fluoro;
R⁵ is selected from the group consisting of hydrogen, methyl, ethyl, propyl, butyl, —C(O)R¹², —R⁹R¹², nitrile, methoxy, ethoxy, propoxy, nitrile, chloro, bromo, fluoro, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, difluoromethoxy, trifluoromethoxy, piperidinyl, morpholinyl, oxolanyl, wherein R⁶ may be optionally substituted by one to two independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from hydrogen or methyl;
R¹³ is selected from the group consisting of cyclopropyl, cyclobutyl, cyclopentyl, and cyclohexyl;
R¹⁴ is selected from the group consisting of piperidinyl, oxolanyl, morpholinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (III):

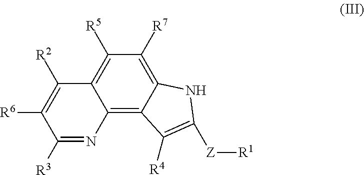

or a pharmaceutically acceptable salt thereof, wherein:
Z is selected from a bond or methylene;
R¹ is selected from oxadiazolyl or oxazolyl;
R² is selected from the group consisting of hydrogen, methyl, ethyl, propyl, isopropyl, butyl, isobutyl, methoxy, ethoxy, propoxy, chloro, bromo, fluoro, nitrile, difluoromethyl, trifluoromethyl, difluoromethoxy, and trifluoromethoxy;
R³ is selected from the group consisting of hydrogen, methyl, ethyl, propyl, isopropyl, butyl, isobutyl, methoxy, ethoxy, propoxy, nitrile, difluoromethyl, trifluoromethyl; and
R⁴, R⁵, R⁶, and R⁷ are hydrogen.

In accordance with another embodiment of the present invention, there is provided a compound of Formula (IV):

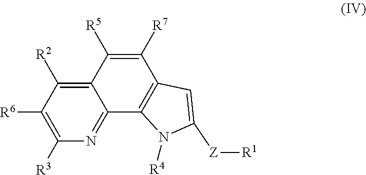

or a pharmaceutically acceptable salt thereof, wherein:
Z is selected from a bond, —C(O), or (C₁-C₆)alkylene;
R¹ is selected from the group consisting of hydrogen, —R¹², —R¹⁴, —R⁹(R¹⁵)ₘ, —OR(R¹⁵)ₘ, and halo;
R² is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, OR¹³, —R¹²S(O)₂, —S(O)₂R¹², halo, nitrile, sulfonamide, sulfone, sulfoxide, (C₄-C₁₄)aryl, and (C₃-C₁₂)cycloalkyl, wherein said R² group may be optionally substituted with one to three R¹¹ groups;
R³ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R(R¹⁵)ₘ, —OR(R¹⁵)ₘ, —OR¹³, halo, nitrile, sulfonamide, sulfone, sulfoxide, (C₄-C₁₄)aryl, and (C₃-C₁₂)cycloalkyl, wherein said R³ group may be optionally substituted with one to three R¹¹ groups;
R⁴ is selected from the group consisting of hydrogen, (C₁-C₆)alkyl, —R⁹(R¹⁵)ₘ, —OR(R¹⁵), —C(O)R⁹, —C(O)R¹³, halo, and (C₃-C₁₂)cycloalkyl;
R⁵ is selected from the group consisting of hydrogen, (C₁-C₆)alkyl, —C(O)R¹², —R⁹R¹² —OR⁹(R¹⁵)ₘ, —R¹⁴, halo, and nitrile;
R⁶ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, halo, nitrile, sulfonamide, sulfone, sulfoxide, (C₄-C₁₄)aryl, and (C₃-C₁₂)cycloalkyl, wherein said R⁶ group may be optionally substituted with one to three R¹¹ groups;
R⁷ is selected from the group consisting of hydrogen, (C₁-C₆)alkyl, —R⁹(R¹⁵)ₘ, —OR(R¹⁵)ₘ, halo, —C(O)R¹², —R⁹R¹², nitrile, and —R¹⁴;
R⁸ is independently selected from the group consisting of hydrogen and (C₁-C₆)alkyl;
R⁹ is (C₁-C₆)alkyl;

R¹⁰ is (C₄-C₁₄)aryl;
R¹¹ is selected from the group consisting of (C₁-C₆)alkyl, dimethyl, sulfonamide, —OR⁸, —C(O)R¹², oxo, nitrile, —R¹², halo, —R⁹(R¹⁵)ₘ, and —OR(R¹⁵)ₘ;
R¹² is —NR^{x}R^{y}, wherein R^{x} and R^{y} are independently selected from the group consisting of hydrogen, (C₁-C₆)alkyl, and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring independently have one to four heteroatoms selected from N, S and O, and wherein said heterocyclic ring or heteroaryl ring may be also optionally substituted with one to three R¹¹ groups;
R¹³ is (C₃-C₁₂)cycloalkyl;
R¹⁴ is selected from the group consisting of (C₁-C₁₁)heteroaryl or (C₁-C₁₁)heterocyclic, wherein said (C₁-C₁₁)heterocyclic or (C₁-C₁₁)heteroaryl each may have one to three heteroatoms selected from N and O, and wherein said (C₁-C₁₁)heteroaryl or (C₁-C₁₁)heterocyclic may also be optionally substituted by one to three independent R¹¹ groups;
R¹⁵ is halo; and
m is independently 0 or an integer from 1 to 3.

In accordance with another embodiment of the present invention, there is provided a compound of Formula (IV):

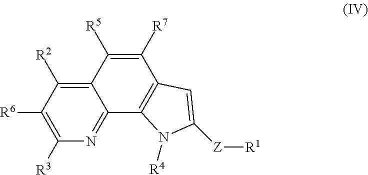

or a pharmaceutically acceptable salt thereof, wherein:
Z is selected from the group consisting of a bond, —C(O), and methylene;
R¹ is selected from the group consisting of hydrogen, —R¹², chloro, bromo, fluoro, difluoromethyl, trifluoromethyl, difluoromethoxy, trifluoromethoxy, oxazolyl, furanyl, oxolanyl, oxadiazolyl, oxazolidinyl, imidazolidinyl, imidazolyl, oxanyl, piperidinyl, morpholinyl, dihydropyranyl, pyranyl, tetrahydropyridinyl, pyridinyl, and pyrrolidinyl, wherein said R¹ group may be optionally substituted with one to three R¹¹ groups;
R² is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R² may be optionally substituted by one to three independent R¹¹ groups;
R³ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R³ may be optionally substituted by one to three independent R¹¹ groups;
R⁴ is selected from the group consisting of hydrogen, methyl, ethyl, propyl, butyl, —C(O)R⁹, —C(O)R¹³, chloro, bromo, fluoro, difluoromethyl, trifluoromethyl, difluoromethoxy, trifluoromethoxy, cyclopropyl, cyclobutyl, cyclopentyl, and cyclohexyl;
R⁵ is selected from the group consisting of hydrogen, —C(O)R¹², —R⁹R¹², nitrile, chloro, bromo, fluoro, difluoromethyl, trifluoromethyl, difluoromethoxy, trifluoromethoxy, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R⁶ may be optionally substituted by one to three independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen, methyl; and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, each independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, imidazolyl, thiophenyl, oxanyl, pyrrolidinyl, furanyl, morpholinyl, oxazolyl, oxadiazolyl, oxazolidinyl, dihydropyranyl, tetrahydropyridinyl, imidazolidinyl, and pyridinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (IV):

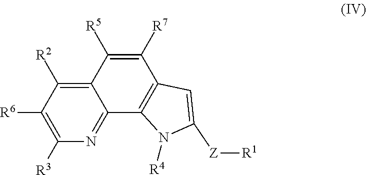

or a pharmaceutically acceptable salt thereof, wherein:
Z is selected from a bond or methylene;
R¹ is selected from the group consisting of oxazolyl, oxanyl, oxolanyl, oxadiazolyl, oxazolidinyl, dihydropyranyl, tetrahydropyridinyl, pyrrolidinyl, morpholinyl, imidazolidinyl, and furanyl, wherein said R¹ group may be optionally substituted with one to two R¹¹ groups;
R² is selected from the group consisting of hydrogen, methyl, ethyl, propyl, isopropyl, butyl, isobutyl, methoxy, ethoxy, propoxy, chloro, bromo, fluoro, nitrile, difluoromethyl, trifluoromethyl, difluoromethoxy, trifluoromethoxy, cyclopropyl, cyclobutyl, cyclopentyl, cyclohexyl, imidazolyl, phenyl, and oxolanyl, wherein R² may be optionally substituted by one to two independent R¹¹ groups;
R³ is selected from the group consisting of hydrogen, methyl, ethyl, propyl, isopropyl, butyl, isobutyl, methoxy, ethoxy, propoxy, nitrile, difluoromethyl, trifluoromethyl, —C(O)R¹², oxanyl, oxolanyl, pyridinyl, phenyl, thiophenyl, piperidinyl, pyrrolidinyl, wherein R³ may be optionally substituted by one to two independent R¹¹ groups;
R⁴ is selected from the group consisting of hydrogen, methoxy, ethoxy, propoxy, methyl, ethyl, propyl, butyl, nitrile, —C(O)R⁹, —C(O)R¹³, chloro, bromo, and fluoro;
R⁵ is selected from the group consisting of hydrogen, methyl, ethyl, propyl, butyl, —C(O)R¹², —R⁹R¹², nitrile, methoxy, ethoxy, propoxy, nitrile, chloro, bromo, fluoro, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, difluoromethoxy, trifluoromethoxy, piperidinyl, morpholinyl, oxolanyl, wherein R⁶ may be optionally substituted by one to two independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from hydrogen or methyl;
R¹³ is selected from the group consisting of cyclopropyl, cyclobutyl, cyclopentyl, and cyclohexyl;
R¹⁴ is selected from the group consisting of piperidinyl, oxolanyl, morpholinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (V):

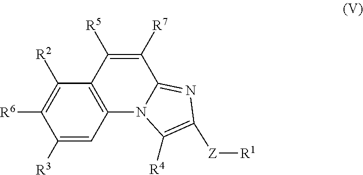

or a pharmaceutically acceptable salt thereof, wherein:
Z is selected from a bond, —C(O), or (C₁-C₆)alkylene;
R¹ is selected from the group consisting of hydrogen, —R¹², —R¹⁴, —R⁹(R¹⁵)ₘ, —OR(R¹⁵)ₘ, and halo;
R² is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, —R¹²S(O)₂, —S(O)₂R¹², halo, nitrile, sulfonamide, sulfone, sulfoxide, (C₄-C₁₄)aryl, and (C₃-C₁₂)cycloalkyl, wherein said R² group may be optionally substituted with one to three R¹¹ groups;
R³ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, OR¹³, halo, nitrile, sulfonamide, sulfone, sulfoxide, (C₄-C₁₄)aryl, and (C₃-C₁₂)cycloalkyl, wherein said R³ group may be optionally substituted with one to three R¹¹ groups;
R⁴ is selected from the group consisting of hydrogen, (C₁-C₆)alkyl, —R⁹(R¹⁵)ₘ, —OR⁹(R¹⁵)ₘ, —C(O)R⁹, —C(O)R¹³, halo, and (C₃-C₁₂)cycloalkyl;
R⁵ is selected from the group consisting of hydrogen, (C₁-C₆)alkyl, —C(O)R¹², —R⁹R¹², —R⁹(R¹⁵)ₘ, —OR⁹(R¹⁵)ₘ, —R¹⁴, halo, and nitrile;
R⁶ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, halo, nitrile, sulfonamide, sulfone, sulfoxide, (C₄-C₁₄)aryl, and (C₃-C₁₂)cycloalkyl, wherein said R⁶ group may be optionally substituted with one to three R¹¹ groups;
R⁷ is selected from the group consisting of hydrogen, (C₁-C₆)alkyl, —R⁹(R¹⁵)ₘ, —OR(R¹⁵), halo, —C(O)R¹², —R⁹R¹², nitrile, and —R¹⁴;
R⁸ is independently selected from the group consisting of hydrogen and (C₁-C₆)alkyl;
R⁹ is (C₁-C₆)alkyl;
R¹⁰ is (C₄-C₁₄)aryl;
R¹¹ is selected from the group consisting of (C₁-C₆)alkyl, dimethyl, sulfonamide, —OR⁸, —C(O)R¹², Oxo, nitrile, —R¹², halo, —R⁹(R¹⁵)ₘ, and —OR(R¹⁵)ₘ;
R¹² is —NR^{x}R^{y}, wherein R^{x} and R^{y} are independently selected from the group consisting of hydrogen, (C₁-C₆)alkyl, and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, each independently have one to four heteroatoms selected from N, S and O, and wherein said heterocyclic ring or heteroaryl ring may be also optionally substituted with one to three R¹¹ groups;
R¹³ is (C₃-C₁₂)cycloalkyl;
R¹⁴ is selected from the group consisting of (C₁-C₁₁)heteroaryl or (C₁-C₁₁)heterocyclic, wherein said (C₁-C₁₁)heterocyclic or (C₁-C₁₁)heteroaryl each may have one to three heteroatoms selected from N and O, and wherein said (C₁-C₁₁)heteroaryl or (C₁-C₁₁)heterocyclic may also be optionally substituted by one to three independent R¹¹ groups;
R¹⁵ is halo; and
m is independently 0 or an integer from 1 to 3.

In accordance with another embodiment of the present invention, there is provided a compound of Formula (V):

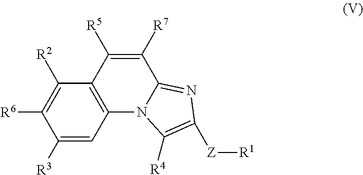

or a pharmaceutically acceptable salt thereof, wherein:
Z is selected from the group consisting of a bond, —C(O), and methylene;
R¹ is selected from the group consisting of hydrogen, —R¹², chloro, bromo, fluoro, difluoromethyl, trifluoromethyl, difluoromethoxy, trifluoromethoxy, oxazolyl, furanyl, oxolanyl, oxadiazolyl, oxazolidinyl, imidazolidinyl, imidazolyl, oxanyl, pipendinyl, morpholinyl, dihydropyranyl, pyranyl, tetrahydropyridinyl, pyridinyl, and pyrrolidinyl, wherein said R¹ group may be optionally substituted with one to three R¹¹ groups;
R² is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R² may be optionally substituted by one to three independent R¹¹ groups;
R³ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R³ may be optionally substituted by one to three independent R¹¹ groups;
R⁴ is selected from the group consisting of hydrogen, methyl, ethyl, propyl, butyl, —C(O)R⁹, —C(O)R¹³, chloro, bromo, fluoro, difluoromethyl, trifluoromethyl, difluoromethoxy, trifluoromethoxy, cyclopropyl, cyclobutyl, cyclopentyl, and cyclohexyl;
R⁵ is selected from the group consisting of hydrogen, —C(O)R¹², —R⁹R¹², nitrile, chloro, bromo, fluoro, difluoromethyl, trifluoromethyl, difluoromethoxy, trifluoromethoxy, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R⁶ may be optionally substituted by one to three independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen, methyl; and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, each independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, imidazolyl, thiophenyl, oxanyl, pyrrolidinyl, furanyl, morpholinyl, oxazolyl, oxadiazolyl, oxazolidinyl, dihydropyranyl, tetrahydropyridinyl, imidazolidinyl, and pyridinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (VI):

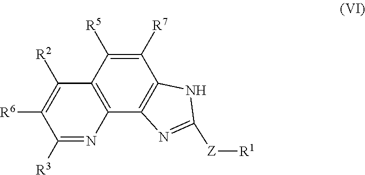

or a pharmaceutically acceptable salt thereof, wherein:
Z is selected from a bond, —C(O), or (C₁-C₆)alkylene;
R¹ is selected from the group consisting of hydrogen, —R¹², —R¹⁴, —R⁹(R¹⁵)ₘ, —OR(R¹⁵)ₘ, and halo;
R² is selected from the group consisting of hydrogen, (C₁-C₆)alkyl, (C₁-C₆)alkoxy, —R¹², R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, —OR¹³, —R¹²S(O)₂, —S(O)₂R¹², halo, nitrile, sulfonamide, sulfone, sulfoxide, (C₄-C₁₄)aryl, and (C₃-C₁₂)cycloalkyl, wherein said R² group may be optionally substituted with one to three R¹¹ groups;
R³ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵)ₘ, OR¹³, halo, nitrile, sulfonamide, sulfone, sulfoxide, (C₄-C₁₄)aryl, and (C₃-C₁₂)cycloalkyl, wherein said R³ group may be optionally substituted with one to three R¹¹ groups;
R⁵ is selected from the group consisting of hydrogen, —C(O)R¹², —R⁹R¹², —R⁹(R¹⁵)ₘ, —OR⁹(R¹⁵)ₘ, —R¹⁴, halo, and nitrile;

R⁶ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR(R¹⁵), —OR¹³, halo, nitrile, sulfonamide, sulfone, sulfoxide, (C₄-C₁₄)aryl, and (C₃-C₁₂)cycloalkyl, wherein said R⁶ group may be optionally substituted with one to three R¹¹ groups;
R⁷ is selected from the group consisting of hydrogen, (C₁-C₆)alkyl, —R⁹(R¹⁵)ₘ, —OR(R¹⁵)ₘ, halo, —C(O)R¹², —R⁹R¹², nitrile, and —R¹⁴;
R⁸ is independently selected from the group consisting of hydrogen and (C₁-C₆)alkyl;
R⁹ is (C₁-C₆)alkyl;
R¹⁰ is (C₄-C₁₄)aryl;
R¹¹ is selected from the group consisting of (C₁-C₆)alkyl, dimethyl, sulfonamide, —OR⁸, —C(O)R¹², oxo, nitrile, —R¹², halo, —R⁹(R¹⁵)ₘ, and —OR(R¹⁵)ₘ;
R¹² is —NR^{x}R^{y}, wherein R^{x} and R^{y} are independently selected from the group consisting of hydrogen, (C₁-C₆)alkyl, and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, each independently have one to four heteroatoms selected from N, S and O, and wherein said heterocyclic ring or heteroaryl ring may be also optionally substituted with one to three R¹¹ groups;
R¹³ is (C₃-C₁₂)cycloalkyl;
R¹⁴ is selected from the group consisting of (C₁-C₁₁)heteroaryl or (C₁-C₁₁)heterocyclic, wherein said (C₁-C₁₁)heterocyclic or (C₁-C₁₁)heteroaryl each may have one to three heteroatoms selected from N and O, and wherein said (C₁-C₁₁)heteroaryl or (C₁-C₁₁)heterocyclic may also be optionally substituted by one to three independent R¹¹ groups;
R¹⁵ is halo; and
m is independently 0 or an integer from 1 to 3.

In accordance with another embodiment of the present invention, there is provided a compound of Formula (VI):

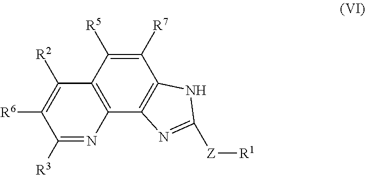

or a pharmaceutically acceptable salt thereof, wherein:
Z is selected from the group consisting of a bond, —C(O), and methylene;
R¹ is selected from the group consisting of hydrogen, —R¹², chloro, bromo, fluoro, difluoromethyl, trifluoromethyl, difluoromethoxy, trifluoromethoxy, oxazolyl, furanyl, oxolanyl, oxadiazolyl, oxazolidinyl, imidazolidinyl, imidazolyl, oxanyl, piperidinyl, morpholinyl, dihydropyranyl, pyranyl, tetrahydropyridinyl, pyridinyl, and pyrrolidinyl, wherein said R¹ group may be optionally substituted with one to three R¹¹ groups;
R² is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R² may be optionally substituted by one to three independent R¹¹ groups;
R³ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R³ may be optionally substituted by one to three independent R¹¹ groups;
R⁵ is selected from the group consisting of hydrogen, methyl, ethyl, propyl, butyl, —C(O)R¹², —R⁹R¹², nitrile, chloro, bromo, fluoro, difluoromethyl, trifluoromethyl, difluoromethoxy, trifluoromethoxy, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R⁶ may be optionally substituted by one to three independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl;
R¹³ is selected from the group consisting of cyclopropyl, cyclobutyl, cyclopentyl, and cyclohexyl;
R¹⁴ is selected from the group consisting of piperidinyl, oxolanyl, morpholinyl, imidazolyl, thiophenyl, oxanyl, pyrrolidinyl, furanyl, morpholinyl, oxazolyl, oxadiazolyl, oxazolidinyl, dihydropyranyl, tetrahydropyridinyl, imidazolidinyl, and pyridinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (VII):

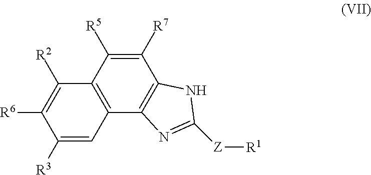

or a pharmaceutically acceptable salt thereof, wherein:
Z is selected from the group consisting of a bond, —C(O), and methylene;
R¹ is selected from the group consisting of hydrogen, —R¹², chloro, bromo, fluoro, difluoromethyl, trifluoromethyl, difluoromethoxy, trifluoromethoxy, oxazolyl, furanyl, oxolanyl, oxadiazolyl, oxazolidinyl, imidazolidinyl, imidazolyl, oxanyl, piperidinyl, morpholinyl, dihydropyranyl, pyranyl, tetrahydropyridinyl, pyridinyl, and pyrrolidinyl, wherein said R¹ group may be optionally substituted with one to three R¹¹ groups;
R² is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R² may be optionally substituted by one to three independent R¹¹ groups;
R³ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R³ may be optionally substituted by one to three independent R¹¹ groups;
R⁵ is selected from the group consisting of hydrogen, methyl, ethyl, propyl, butyl, —C(O)R¹², —R⁹R¹², nitrile, chloro, bromo, fluoro, difluoromethyl, trifluoromethyl, difluoromethoxy, trifluoromethoxy, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R⁶ may be optionally substituted by one to three independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl;
R¹³ is selected from the group consisting of cyclopropyl, cyclobutyl, cyclopentyl, and cyclohexyl;
R¹⁴ is selected from the group consisting of piperidinyl, oxolanyl, morpholinyl, imidazolyl, thiophenyl, oxanyl, pyrrolidinyl, furanyl, morpholinyl, oxazolyl, oxadiazolyl, oxazolidinyl, dihydropyranyl, tetrahydropyridinyl, imidazolidinyl, and pyridinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (VIII):

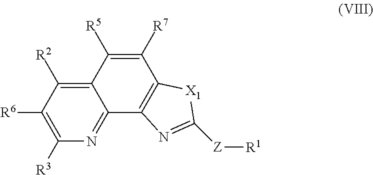

or a pharmaceutically acceptable salt thereof, wherein:
X₁ is selected from O or S;
Z is selected from the group consisting of a bond, —C(O), and methylene;
R¹ is selected from the group consisting of hydrogen, —R¹², chloro, bromo, fluoro, difluoromethyl, trifluoromethyl, difluoromethoxy, trifluoromethoxy, oxazolyl, furanyl, oxolanyl, oxadiazolyl, oxazolidinyl, imidazolidinyl, imidazolyl, oxanyl, piperidinyl, morpholinyl, dihydropyranyl, pyranyl, tetrahydropyridinyl, pyridinyl, and pyrrolidinyl, wherein said R¹ group may be optionally substituted with one to three R¹¹ groups;
R² is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R² may be optionally substituted by one to three independent R¹¹ groups;
R³ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R³ may be optionally substituted by one to three independent R¹¹ groups;
R⁵ is selected from the group consisting of hydrogen, methyl, ethyl, propyl, butyl, —C(O)R¹², —R⁹R¹², nitrile, chloro, bromo, fluoro, difluoromethyl, trifluoromethyl, difluoromethoxy, trifluoromethoxy, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R⁶ may be optionally substituted by one to three independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl;
R¹³ is selected from the group consisting of cyclopropyl, cyclobutyl, cyclopentyl, and cyclohexyl;
R¹⁴ is selected from the group consisting of piperidinyl, oxolanyl, morpholinyl, imidazolyl, thiophenyl, oxanyl, pyrrolidinyl, furanyl, morpholinyl, oxazolyl, oxadiazolyl, oxazolidinyl, dihydropyranyl, tetrahydropyridinyl, imidazolidinyl, and pyridinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (VIV):

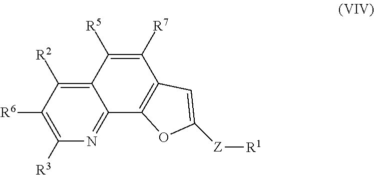

or a pharmaceutically acceptable salt thereof, wherein:
Z is selected from the group consisting of a bond, —C(O), and methylene;
R¹ is selected from the group consisting of hydrogen, —R¹², chloro, bromo, fluoro, difluoromethyl, trifluoromethyl, difluoromethoxy, trifluoromethoxy, oxazolyl, furanyl, oxolanyl, oxadiazolyl, oxazolidinyl, imidazolidinyl, imidazolyl, oxanyl, piperidinyl, morpholinyl, dihydropyranyl, pyranyl, tetrahydropyridinyl, pyridinyl, and pyrrolidinyl, wherein said R¹ group may be optionally substituted with one to three R¹¹ groups;
R² is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R² may be optionally substituted by one to three independent R¹¹ groups;
R³ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R³ may be optionally substituted by one to three independent R¹¹ groups;
R⁵ is selected from the group consisting of hydrogen, methyl, ethyl, propyl, butyl, —C(O)R¹², —R⁹R¹², nitrile, chloro, bromo, fluoro, difluoromethyl, trifluoromethyl, difluoromethoxy, trifluoromethoxy, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R⁶ may be optionally substituted by one to three independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl;
R¹³ is selected from the group consisting of cyclopropyl, cyclobutyl, cyclopentyl, and cyclohexyl;
R¹⁴ is selected from the group consisting of piperidinyl, oxolanyl, morpholinyl, imidazolyl, thiophenyl, oxanyl, pyrrolidinyl, furanyl, morpholinyl, oxazolyl, oxadiazolyl, oxazolidinyl, dihydropyranyl, tetrahydropyridinyl, imidazolidinyl, and pyridinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

In accordance with another embodiment of the present invention, there is provided a compound of Formula (X):

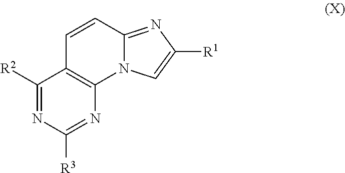

or a pharmaceutically acceptable salt thereof, wherein:
R¹ is selected from the group consisting of hydrogen and (C₁-C₁₁)heteroaryl;
R² is selected from the group consisting of hydrogen and (C₁-C₆)haloalkyl; and
R³ is selected from the group consisting of hydrogen and (C₁-C₆)haloalkyl.

In accordance with another embodiment of the present invention, there is provided a compound of Formula (X), wherein R¹ is selected from the group consisting of hydrogen and oxadiazolyl.

In accordance with another embodiment of the present invention, there is provided a compound of Formula (X), wherein:
R¹ is selected from the group consisting of hydrogen and oxadiazolyl;
R² is selected from the group consisting of hydrogen and trifluoromethyl; and
R³ is selected from the group consisting of hydrogen and trifluoromethyl.

In accordance with another embodiment of the present invention, there is provided a compound of Formula (X), wherein:
R¹ is selected from the group consisting of hydrogen, and 1,3,4-oxadiazol-2-yl;
R² is selected from the group consisting of hydrogen, and trifluoromethyl; and
R³ is selected from the group consisting of hydrogen, and trifluoromethyl.

In accordance with another embodiment of the present invention, there is provided a compound of Formula (XI):

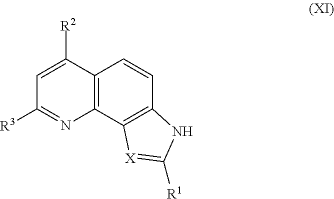

or a pharmaceutically acceptable salt thereof, wherein:
X is selected from the group consisting of N and CH;
R¹ is selected from the group consisting of hydrogen, and (C₁-C₁₁)heteroaryl;
R² is selected from the group consisting of hydrogen, and (C₁-C₆)haloalkyl; and
R³ is selected from the group consisting of hydrogen, and (C₁-C₆)haloalkyl.

In accordance with another embodiment of the present invention, there is provided a compound of Formula (XI), wherein R¹ is selected from the group consisting of hydrogen, oxadiazolyl, and oxazolyl.

In accordance with another embodiment of the present invention, there is provided a compound of Formula (XI), wherein:
X is selected from the group consisting of nitrogen and carbon.
R¹ is selected from the group consisting of hydrogen, 1,3,4-oxadiazol-2-yl, and 1,3-oxazol-5-yl;
R² is selected from the group consisting of hydrogen, and trifluoromethyl; and
R³ is selected from the group consisting of hydrogen, and trifluoromethyl.

In accordance with another embodiment of the present invention, there is provided a compound of Formula (XIII):

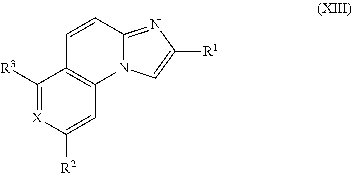

or a pharmaceutically acceptable salt thereof, wherein:
X is selected from the group consisting of N and CH;
R¹ is selected from the group consisting of hydrogen, and (C₁-C₁₁)heteroaryl;
R² is selected from the group consisting of hydrogen, and (C₁-C₆)haloalkyl; and
R³ is selected from the group consisting of hydrogen, and (C₁-C₆)haloalkyl.

In accordance with another embodiment of the present invention, there is provided a compound of Formula (XIII), wherein R¹ is selected from the group consisting of hydrogen, and oxadiazolyl.

In accordance with another embodiment of the present invention, there is provided a compound of Formula (XIII), wherein:
X is selected from the group consisting of nitrogen and carbon;
R¹ is selected from the group consisting of hydrogen, and 1,3,4-oxadiazol-2-yl;
R² is selected from the group consisting of hydrogen, and trifluoromethyl; and
R³ is selected from the group consisting of hydrogen, and trifluoromethyl.

In accordance with another embodiment of the present invention, there is provided a compound of Formula (XIV):

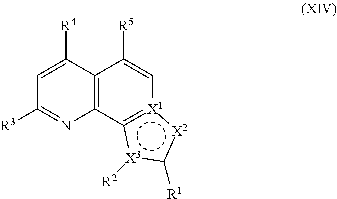

or a pharmaceutically acceptable salt thereof, wherein:

X¹ is selected from the group consisting of N and C;

X² is selected from the group consisting of S, C, and CH;

X³ is selected from the group consisting of N and O;
R¹ is selected from the group consisting of hydrogen, (C₁-C₁₁)heteroaryl, and (C₁-C₁₁)heterocycle;
R² is selected from the group consisting of hydrogen, benzyl, (C₁-C₆)alkyl, acetyl, and cycloalkylcarbonyl;
R³ is selected from the group consisting of hydrogen, and (C₁-C₆)haloalkyl;
R⁴ is selected from the group consisting of hydrogen, and (C₁-C₆)haloalkyl; and
R⁵ is hydrogen.

In accordance with another embodiment of the present invention, there is provided a compound of Formula (XIV), wherein R¹ is oxadiazolyl.

In accordance with another embodiment of the present invention, there is provided a compound of Formula (XIV), wherein:
X¹ is selected from the group consisting of N and C;
X² is selected from the group consisting of S, C, and CH;
X³ is selected from the group consisting of N and O;
R¹ is selected from the group consisting of hydrogen, 3,4-oxadiazol-2-yl, tetrahydropyran-(3 or 4)-yl, 1-methylpiperidin-(3 or 4)-yl, 3,6-dihydro-2H-pyran-4-yl, 5,6-dihydro-2H-pyran-3-yl, and 1-methyl-1,2,3,6-tetrahydropyridin-(4 or 5)-yl;
R² is selected from the group consisting of hydrogen, benzyl, methyl, acetyl, and cyclobutylcarbonyl;
R³ is selected from the group consisting of hydrogen, and trifluoromethyl;
R⁴ is selected from the group consisting of hydrogen, and trifluoromethyl; and
R⁵ is hydrogen.

In accordance with another embodiment of the present invention, there is provided a compound of Formula (XV):

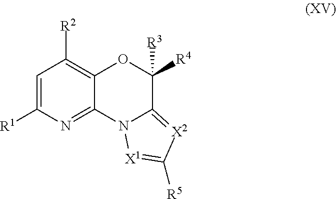

or a pharmaceutically acceptable salt thereof, wherein:
X¹ and X² are independently selected from the group consisting of N and CH;
R¹ is selected from the group consisting of hydrogen and (C₁-C₆)haloalkyl;
R² is selected from the group consisting of hydrogen and (C₁-C₆)haloalkyl;
R³ is selected from the group consisting of hydrogen and (C₁-C₆)alkyl;
R⁴ is selected from the group consisting of hydrogen and (C₁-C₆)alkyl; and
R⁵ is selected from the group consisting of hydrogen and (C₁-C₁₁)heteroaryl.

In accordance with another embodiment of the present invention, there is provided a compound of Formula (XV), wherein R⁵ is oxadiazolyl.

In accordance with another embodiment of the present invention, there is provided a compound of Formula (XV), wherein:
X¹ is selected from the group consisting of N and CH;
X² is selected from the group consisting of N and CH;
R¹ is selected from the group consisting of hydrogen, and trifluoromethyl;
R² is selected from the group consisting of hydrogen, and trifluoromethyl;
R³ is selected from the group consisting of hydrogen, and methyl;
R⁴ is selected from the group consisting of hydrogen, and methyl; and
R⁵ is selected from the group consisting of hydrogen, and 1,3,4-oxadiazol-2-yl.

In accordance with another embodiment of the present invention, there is provided a compound of Formula (XVI):

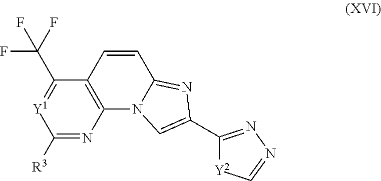

or a pharmaceutically acceptable salt thereof, wherein:
Y¹ is selected from the group consisting of N and CH;
Y² is selected from the group consisting of O and S; and
R³ is selected from the group consisting of trifluoromethyl and cyclopentyl.

In accordance with another embodiment of the present invention, there is provided a compound of Formula (XVII):

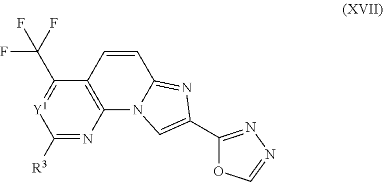

or a pharmaceutically acceptable salt thereof, wherein:
Y¹ is selected from the group consisting of N and CH;
R³ is selected from the group consisting of trifluoromethyl and cyclopentyl.

In accordance with another embodiment of the present invention, there is provided a compound having the structure:

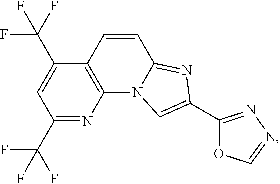 [SMILES: FC(F)(F)c1cc(C(F)(F)F)c2ccc3nc(-c4nnco4)cn3c2n1]

or a pharmaceutically acceptable salt thereof.

In accordance with another embodiment of the present invention, there is provided a compound having the structure:

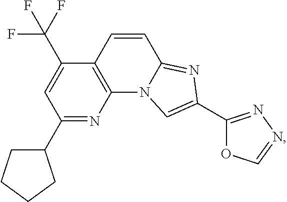 [SMILES: FC(F)(F)c1cc(C2CCCC2)nc2c1ccc1nc(-c3nnco3)cn12]

or a pharmaceutically acceptable salt thereof.

In accordance with another embodiment of the present invention, there is provided a compound having the structure:

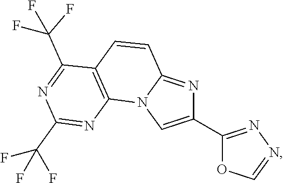 [SMILES: FC(F)(F)c1nc(C(F)(F)F)c2ccc3nc(-c4nnco4)cn3c2n1]

or a pharmaceutically acceptable salt thereof.

In accordance with another embodiment of the present invention, there is provided a compound selected from the group consisting of those compounds in Tables 1 and 2.

In accordance with another embodiment of the present invention, there is provided a compound selected from the group consisting of those compounds in Table 1.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g., D₂O, d₆-acetone, d₆-DMSO.

Compounds of formula (I) containing one or more asymmetric carbon atoms can exist as two or more stereoisomers. Where a compound of formula (I) contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds present invention are all stereoisomers, geometric isomers and tautomeric forms of the compounds of Formula (I) or Formula (II), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of any of the formulas described herein contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of any of the formulas described herein, wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of any of the formulas described herein, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labelled compounds of any of the formulas described herein can generally be prepared by conventional techniques known to those skilled in the art or by processes analogous to those described in the accompanying Examples using an appropriate isotopically-labelled reagents in place of the non-labelled reagent previously employed.

The compounds of the present invention may be administered as prodrugs. Thus, certain derivatives of compounds of any of the formulas described herein, which may have little or no pharmacological activity themselves can, when administered into or onto the body, be converted into compounds having the desired activity, for example, by hydrolytic cleavage. Such derivatives are referred to as ‘prodrugs’.

In accordance with another embodiment of the present invention, there is provided the use of a compound or salt as defined in any of the formulas described herein in the manufacture of a medicament for use in the treatment of a viral infection in a human.

In accordance with another embodiment of the present invention, there is provided a pharmaceutical composition comprising a pharmaceutically acceptable diluent and a therapeutically effective amount of a compound as defined in any of the formulas described herein.

Antiviral response through interferon-alpha (IFNα) pathway activation, mainly via activation of JAK1/STAT pathway, has been described recently to be inhibited by human papillomavirus proteins E6 and E7 (See Stanley, M., Clinical Microbiology Revs. 25:2 215-222 (2012)), suggesting that the restoration/upregulation of the JAK1/STAT pathway activation as potentially being an effective antiviral approach for treating human papillomavirus infections and ameliorating the resultant symptoms, such as warts. Therefore, without intending to be bound by any particular theory, activation of the JAK1/STAT pathway in such physiological tissues as skin keratinocytes, is expected to lead to effective therapies for treating warts caused by the human papillomavirus. By activating the JAK1/STAT pathway and thereby the IFNα pathway within and/or near the site of a wart in a subject, it is believed that this could lead to shrinkage of the wart over time or eventually the complete eridication of the wart from the skin of the subject.

Thus, in accordance with one embodiment of the present invention, there is provided a method for treating a viral infection in a subject that has been diagnosed with said viral infection or is at risk of developing said viral infection comprising administering to said subject, any one of the compounds from any of the formula (s) or Tables 1 or 2 described herein.

In accordance with another embodiment of the present invention, there is provided a method for enhancing the immune response in a subject that has been diagnosed with a viral infection or is at risk of developing said viral infection comprising administering to said subject, a compound as defined in any of the formulas described herein.

In accordance with another embodiment of the present invention, there is provided a method for enhancing the immune response to a viral infection in a subject that is immunocompromised or is at risk of developing an immunocomprised immune system comprising administering to said subject, a compound as defined in any of the formulas described herein.

In accordance with another embodiment of the present invention, there is provided a method for enhancing the immune response to a viral infection in a subject that is immunocompromised or is at risk of developing an immunocomprised immune system comprising administering to said subject, a compound as defined in any of the formulas described herein, wherein the immunocomprised subject is a subject diagnosed with an HIV infection.

In accordance with another embodiment of the present invention, there is provided a method for enhancing the immune response to a viral infection in a subject that is immunocompromised or is at risk of developing an immunocomprised immune system comprising administering to said subject, a compound as defined in any of the formulas described herein, wherein the immunocomprised subject is a pre-term infant.

In accordance with another embodiment of the present invention, there is provided a method for enhancing the immune response to a viral infection in a subject that is immunocompromised or is at risk of developing an immunocomprised immune system comprising administering to said subject, a compound as defined in any of the formulas described herein, wherein the immunocomprised subject is a subject that has had an organ transplant or is at risk for having an organ transplant.

In another embodiment of the present invention, there is provided a method for treating and/or preventing a viral infection in a subject comprising administering to the subject an activator of the subject's JAK/STAT pathway. In some embodiments, the activator is a chemical activator. In some embodiments, the chemical activator is administered topically to the subject's skin and/or mucous membranes.

In accordance with another embodiment of the present invention, there is provided a method for upregulating the JAK/STAT immune pathway in a subject that has been diagnosed with a viral infection or is at risk of developing said viral infection comprising administering to said subject, a compound as defined in any of the Formula's described herein.

In accordance with another embodiment of the present invention, there are provided compounds and methods for treating viral infections, wherein the viral infection comprises one or more viruses from a viral family selected from the group consisting of Picornaviruses, Togaviruses, Flaviruses, Filoviruses, Paramixoviruses, Bunya viruses, Polyomaviruses, Adenoviruses, Herpes viruses, and Poxviruses.

In accordance with another embodiment of the present invention, there are provided compounds and methods for treating viral infections, wherein the viral infection comprises one or more viruses from the Picornavirus family.

In accordance with another embodiment of the present invention, there are provided compounds and methods for treating viral infection, wherein said viral infection comprises one or more viruses from the Picornavirus family selected from the group consisting of rhinovirus, poliovirus, Coxsackie virus, enteroviruses, Foot and Mouth Disease virus, hepatitis A virus, and Norovirus.

In accordance with another embodiment of the present invention, there are provided compounds and methods for treating viral infections, wherein said viral infection comprises one or more viruses from the Togavirus family.

In accordance with another embodiment of the present invention, there are provided compounds and methods for treating viral infections, wherein said viral infection comprises one or more viruses from the Togavirus family selected from the group consisting of Eastern Equine Encephalitis virus, Western Equine Encephalitis virus, Venezuelan Equine Encephalitis virus, Chikungunya virus, Ross River virus, Semliki Forest virus, and Sindbis virus.

In accordance with another embodiment of the present invention, there are provided compounds and methods for treating viral infections, wherein said viral infection comprises one or more viruses from the Flavivirus family.

In accordance with another embodiment of the present invention, there are provided compounds and methods for treating viral infections, wherein said viral infection comprises one or more viruses from the Flavivirus family selected from the group consisting of Dengue virus, Yellow fever virus, Japanese Encephalitis virus, St. Louis Encephalitis virus, West Nile virus, Tickbome encephalitis virus, and Hepatitis C virus.

In accordance with another embodiment of the present invention, there are provided compounds and methods for treating viral infections, wherein said viral infection comprises one or more viruses from the Filovirus family.

In accordance with another embodiment of the present invention, there are provided compounds and methods for treating viral infections, wherein said viral infection comprises one or more viruses from the Filovirus family selected from the group consisting of Marburg virus and Ebola virus.

In accordance with another embodiment of the present invention, there are provided compounds and methods for treating viral infections, wherein said viral infection comprises one or more viruses from the Paramixovirus family.

In accordance with another embodiment of the present invention, there are provided compounds and methods for treating viral infections, wherein said viral infection comprises one or more viruses from the negative strand RNA viruses selected from the group consisting of Mumps virus, Parainfluenza virus, Newcastle Disease virus, Measles virus, Nipah virus, Respiratory Syncytial virus, Metapneumovirus, and Influenza virus.

In accordance with another embodiment of the present invention, there are provided compounds and methods for treating viral infections, wherein said viral infection comprises one or more viruses from the Bunya virus family.

In accordance with another embodiment of the present invention, there are provided compounds and methods for treating viral infections, wherein said viral infection comprises one or more viruses from the Bunya virus family selected from the group consisting of Orthobunya viruses, Phleboviruses, Hanta virus, and Rotavirus.

In accordance with another embodiment of the present invention, there are provided compounds and methods for treating viral infections, wherein said viral infection comprises one or more viruses from the Polyomavirus family.

In accordance with another embodiment of the present invention, there are provided compounds and methods for treating viral infections, wherein said viral infection comprises one or more viruses from the Polyomavirus family selected from the group consisting of JC virus and BK virus.

In accordance with another embodiment of the present invention, there are provided compounds and methods for treating viral infections, wherein said viral infection comprises one or more viruses from the Adenovirus family.

In accordance with another embodiment of the present invention, there are provided compounds and methods for treating viral infections, wherein said viral infection comprises one or more viruses from the Herpes virus family.

In accordance with another embodiment of the present invention, there are provided compounds and methods for treating viral infections, wherein said viral infection comprises one or more viruses from the Herpes virus family selected from the group consisting of HHV-1 (HSV-1), HHV-2 (HSV-2), HHV-3 (VZV), HHV-4 (EBV), HHV-5 (CMV), HHV-8 (KSV), and B virus.

In accordance with another embodiment of the present invention, there are provided compounds and methods for treating viral infections.

In accordance with another embodiment of the present invention, there are provided compounds and methods for treating viral infections, wherein said viral infection comprises one or more viruses from the Poxvirus family.

In accordance with another embodiment of the present invention, there are provided compounds and methods for treating viral infections, wherein said viral infection comprises one or more viruses from the Poxvirus family selected from the group consisting of monkeypox and Variola virus (smallpox).

In accordance with another embodiment of the present invention, there are provided compounds and methods for treating and preventing viral infections, wherein said viral infection comprises one or more viruses from the Papillomavirus family. Human papillomavirus (HPV) is a virus from the papillomavirus family that is capable of infecting humans. Like all papillomaviruses, HPVs establish productive infections in keratinocytes of the skin or mucous membranes. While the majority of the known types of HPV cause no symptoms in most people, some types can cause warts (verrucae), while others can lead to cancers of the cervix, vulva, vagina, penis, oropharynx and anus. In addition, HPV 16 and 18 infections are strongly associated with an increased odds ratio of developing oropharyngeal (throat) cancer. These two types are responsible for close to 70% of cervical cancers, 90% of vaginal and anal cancers and 40% of cancers of the vulva and penis. More than 30 to 40 types of HPV are typically transmitted through sexual contact and infect the anogenital region. Some sexually transmitted HPV types may cause genital warts. Persistent infection with “high-risk” HPV types—different from the ones that cause skin warts—may progress to precancerous lesions and invasive cancer. HPV infection is a cause of nearly all cases of cervical cancer.

Some “cutaneous” HPV types cause common skin warts. Common warts are often found on the hands and feet, but can also occur in other areas, such as the elbows or knees. Common warts have a characteristic cauliflower-like surface and are typically slightly raised above the surrounding skin. Plantar warts are found on the soles of the feet. Plantar warts grow inward, generally causing pain when walking. Subungual or periungual warts form under the fingemail (subungual), around the fingemail or on the cuticle (periungual). Flat warts are most commonly found on the arms, face or forehead. Like common warts, flat warts occur most frequently in children and teens.

Over 120 HPV types have been identified and are referred to by number. Types 16, 18, 31, 33, 35, 39, 45, 51, 52, 56, 58, 59, 68, 73, and 82 are carcinogenic “high-risk” sexually transmitted HPVs and may lead to the development of cervical intraepithelial neoplasia, vulvar intraepithelial neoplasia, penile intraepithelial neoplasia, and/or anal intraepithelial neoplasia. For example, the chart provided below lists several diseases encompassed by the methods of prevention and/or treatment described herein, which are associated with HPV, and in particular, the HPV type.

| Disease | HPV type |
|---|---|
| Common warts | 2, 7 |
| Plantar warts | 1, 2, 4, 63 |
| Flat warts | 3, 10, 8 |
| Anogenital warts | 6, 11, 42, 44 |
| Anal lesions | 6, 16, 18, 31, 53, 58 |
| Genital cancers | Highest risk: 16, 18, 31, 45 |
| | Other high-risk: 33, 35, 39, 51, 52, 56, 58, 59 |
| | Probably high-risk: 26, 53, 66, 68, 73, 82 |
| Epidermodysplasia | more than 15 types |
| verruciformis | |
| Focal epithelial | 13, 32 |
| hyperplasia (oral) | |
| Oral papillomas | 6, 7, 11, 16, 32 |
| Oropharyngeal cancer | 16 |
| Verucous cyst | 60 |
| Laryngeal papillomatosis | 6, 11 |

Therefore, in accordance with another embodiment of the present invention, there are provided compounds and methods for treating human papilloma virus associated skin diseases including common warts, flat warts, plantar warts, inguinal warts and venereal warts and pre-cancerous lesions.

In accordance with another embodiment of the present invention, there are provided compounds and methods for treating high risk human Papillomavirus infections of the cervix, vulva, vagina, penis, oropharynx and anus.

In accordance with another embodiment of the present invention, there are provided compounds and methods for topically treating human papilloma virus warts (verrucae) in and on human skin or mucous membranes.

In accordance with another embodiment of the present invention, there are provided compounds and methods for treating a common wart on a subject comprising administering to the subject any one of the compounds from any of the formula (s) or Tables 1 or 2 described herein.

In accordance with another embodiment of the present invention, there are provided compounds and methods for preventing and/or treating common wart(s) on a subject comprising contacting any one of the compounds from any of the formula (s) or Tables 1 or 2 described herein to the common wart on the subject. In some embodiments, the compound can be formulated into a topical formulation for treating and/or preventing a dermatological condition resulting from a human papillomavirus. One such condition is the common wart, which may appear on the skin or on a mucous membrane. By way of example, the compound(s) described herein can be added to formulations such as film-forming liquids or gels that would cover and dry to form a thin film over the wart area, thus keeping the compound in contact with the wart for an extended period of time and could also optionally be covered afterwards with an occlusive dressing. Therefore, in other embodiments, the compound(s) of the present invention could be included in a topical formulation along with a kit with occlusive dressings or adhesives and also applicators to coat the surface of the wart.

In accordance with one embodiment of the present invention, there is provided a method for treating a wart on the skin or mucous membrane of a subject comprising contacting a compound having the structure:

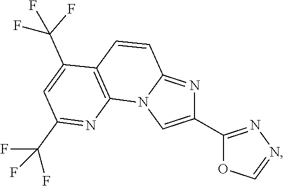 [SMILES: FC(F)(F)c1cc(C(F)(F)F)c2ccc3nc(-c4nnco4)cn3c2n1]

or a pharmaceutically acceptable salt thereof,
to the wart on the skin or mucous membrane of the subject.

In accordance with one embodiment of the present invention, there is provided a method for treating a wart on the skin or mucous membrane of a subject comprising contacting a compound having the structure:

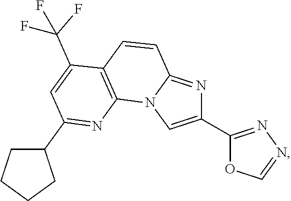 [SMILES: FC(F)(F)c1cc(C2CCCC2)nc2c1ccc1nc(-c3nnco3)cn12]

or a pharmaceutically acceptable salt thereof,
to the wart on the skin or mucous membrane of the subject.

In accordance with one embodiment of the present invention, there is provided a method for treating a wart on the skin or mucous membrane of a subject comprising contacting a compound having the structure:

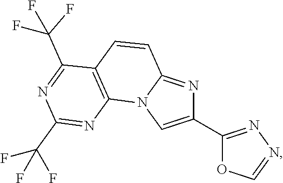 [SMILES: FC(F)(F)c1nc(C(F)(F)F)c2ccc3nc(-c4nnco4)cn3c2n1]

or a pharmaceutically acceptable salt thereof,
to the wart on the skin or mucous membrane of the subject.

In accordance with another embodiment of the present invention, there is provided a method for treating a viral infection in a subject that has been diagnosed with said viral infection or is at risk of developing said viral infection comprising administering to said subject, any one of the compounds from any of the formula (s) or Tables 1 or 2 described herein.

In accordance with another embodiment of the present invention, there is provided a method for enhancing the immune response in a subject that has been diagnosed with a viral infection or is at risk of developing said viral infection comprising administering to said subject, any one of the compounds from any of the formula (s) or Tables 1 or 2 described herein.

In accordance with another embodiment of the present invention, there is provided a method for enhancing the immune response to a viral infection in a subject that is immunocompromised or is at risk of developing an immunocomprised immune system comprising administering to said subject any one of the compounds from any of the formula (s) or Tables 1 or 2 described herein.

In accordance with another embodiment of the present invention, there is provided a method for upregulating the JAK/STAT immune pathway in a subject that has been diagnosed with a viral infection or is at risk of developing said viral infection comprising administering to said subject any one of the compounds from any of the formula (s) or Tables 1 or 2 described herein.

In accordance with another embodiment of the present invention, there is provided a method for treating a common wart on a subject comprising administering to the subject any one of the compounds from any of the formula (s) or Tables 1 or 2 described herein.

In accordance with another embodiment of the present invention, there is provided a method for treating a common wart on a subject comprising contacting any one of the compounds from any of the formula (s) or Tables 1 or 2 described herein.

In accordance with another embodiment of the present invention, there are provided compounds and methods of treating precancerous and cancerous skin lesions, including actinic keratoses, basal cell carcinoma, and squamous cell carcinoma.

In accordance with another embodiment of the present invention, there are provided compounds and methods of treating viral skin infections including Molloscum contagiosum. Molluscum contagiosum (MC) is a viral infection of the skin or occasionally of the mucous membranes, sometimes called water warts. It is caused by a DNA poxvirus called the molluscum contagiosum virus (MCV). There are four types of MCV, MCV-1 to −4; MCV-1 is the most prevalent and MCV-2 is seen usually in adults and often sexually transmitted. This common viral disease has a higher incidence in children, sexually active adults, and those who are immunodeficient, and the infection is most common in children aged one to ten years old. MC can affect any area of the skin but is most common on the trunk of the body, arms, and legs.

In further embodiments, the compound of the present invention, or a pharmaceutically acceptable salt thereof, is chosen from the compounds set forth in Table 1.

TABLE 1

| Compound | | |
|---|---|---|
| Number | | |
| and | | |
| Example | | |
| Number | Structure | Chemical Name |
| 1 | 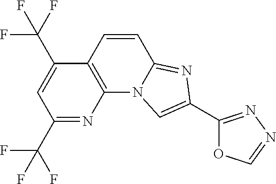 [SMILES: FC(F)(F)c1cc(C(F)(F)F)c2ccc3nc(-c4nnco4)cn3c2n1] | 2-[2,4-bis(trifluoromethyl)imidazo[1,2- a]1,8-naphthyridin-8-yl]-1,3,4- oxadiazole |
| 2 | 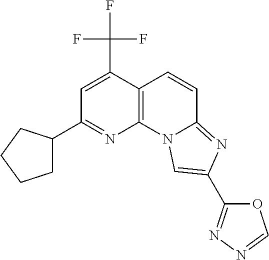 [SMILES: FC(F)(F)c1cc(C2CCCC2)nc2c1ccc1nc(-c3nnco3)cn12] | 2-[2-cyclopentyl-4- (trifluoromethyl)imidazo[1,2-a]1,8- naphthyridin-8-yl]-1,3,4-oxadiazole |
| 3 | 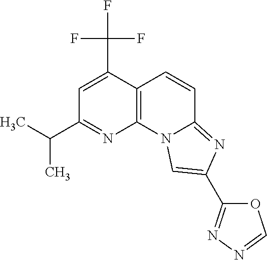 [SMILES: CC(C)c1cc(C(F)(F)F)c2ccc3nc(-c4nnco4)cn3c2n1] | 2-[2-(propan-2-yl)-4- (trifluoromethyl)imidazo[1,2-a]1,8- naphthyridin-8-yl]-1,3,4-oxadiazole |
| 4 | 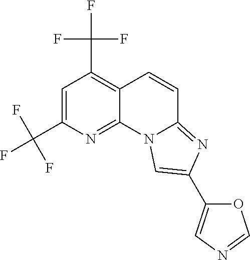 [SMILES: FC(F)(F)c1cc(C(F)(F)F)c2ccc3nc(-c4cnco4)cn3c2n1] | 5-[2,4-bis(trifluoromethyl)imidazo[1,2- a]1,8-naphthyridin-8-yl]-1,3-oxazole |
| 5 | 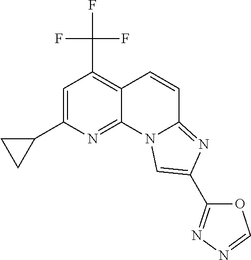 [SMILES: FC(F)(F)c1cc(C2CC2)nc2c1ccc1nc(-c3nnco3)cn12] | 2[2-cyclopropyl-4- (trifluoromethyl)imidazo[1,2-a]1,8- naphthyridin-8-yl]-1,3,4-oxadiazole |
| 6 | 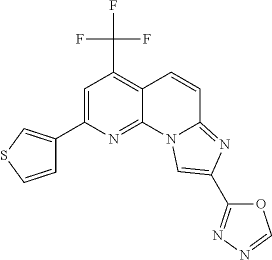 [SMILES: FC(F)(F)c1cc(-c2ccsc2)nc2c1ccc1nc(-c3nnco3)cn12] | 2-[2-(thiophen-3-yl)-4- (trifluoromethyl)imidazo[1,2-a]1,8- naphthyridin-8-yl]-1,3,4-oxadiazole |
| 7 | 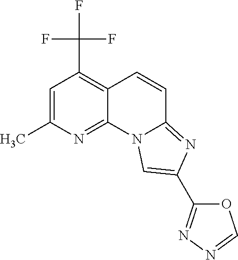 [SMILES: Cc1cc(C(F)(F)F)c2ccc3nc(-c4nnco4)cn3c2n1] | 2-[2-methyl-4- (trifluoromethyl)imidazo[1,2-a]1,8- naphthyridin-8-yl]-1,3,4-oxadiazole |
| 8 | 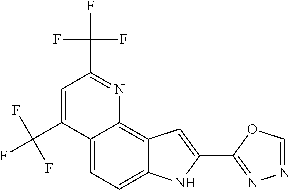 [SMILES: FC(F)(F)c1cc(C(F)(F)F)c2ccc3[nH]c(-c4nnco4)cc3c2n1] | 2-[2,4-bis(trifluoromethyl)-7H- pyrrolo[2,3-h]quinolin-8-yl]-1,3,4- oxadiazole |
| 9 | 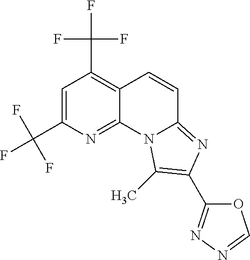 [SMILES: Cc1c(-c2nnco2)nc2ccc3c(C(F)(F)F)cc(C(F)(F)F)nc3n12] | 2-[9-methyl-2,4- bis(trifluoromethyl)imidazo[1,2-a]1,8- naphthyridin-8-yl]-1,3,4-oxadiazole |
| 10 | 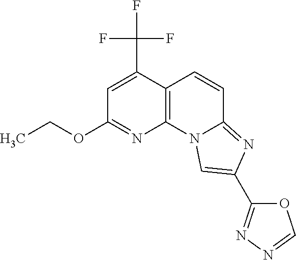 [SMILES: CCOc1cc(C(F)(F)F)c2ccc3nc(-c4nnco4)cn3c2n1] | 2-[2-ethoxy-4- (trifluoromethyl)imidazo[1,2-a]1,8- naphthyridin-8-yl]-1,3,4-oxadiazole |
| 11 | 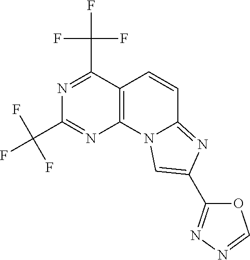 [SMILES: FC(F)(F)c1nc(C(F)(F)F)c2ccc3nc(-c4nnco4)cn3c2n1] | 4-(1,3,4-oxadiazol-2-yl)-10,12- bis(trifluoromethyl)-2,5,11,13- tetraazatricyclo[7.4.0.0²,⁶]trideca- 1(9),3,5,7,10,12-hexaene |
| 12 | 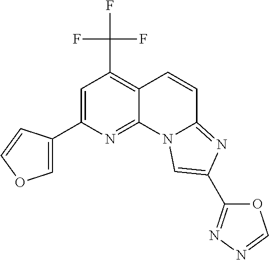 [SMILES: FC(F)(F)c1cc(-c2ccoc2)nc2c1ccc1nc(-c3nnco3)cn12] | 2-[2-(furan-3-yl)-4- (trifluoromethyl)imidazo[1,2-a]1,8- naphthyridin-8-yl]-1,3,4-oxadiazole |
| 13 | 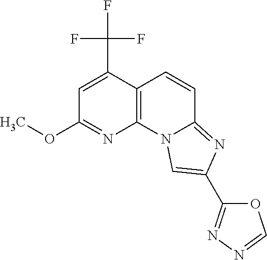 [SMILES: COc1cc(C(F)(F)F)c2ccc3nc(-c4nnco4)cn3c2n1] | 2-[2-ethyl-4- (trifluoromethyl)imidazo[1,2-a]1,8- naphthyridin-8-yl]-1,3,4-oxadiazole |
| 14 | 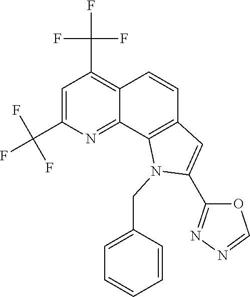 [SMILES: FC(F)(F)c1cc(C(F)(F)F)c2ccc3cc(-c4nnco4)n(Cc4ccccc4)c3c2n1] | 2-[1-benzyl-6,8-bis(trifluoromethyl)-1H- pyrrolo[3,2-h]quinolin-2-yl]-1,3,4- oxadiazole |
| 15 | 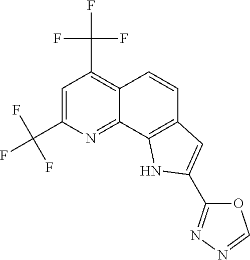 [SMILES: FC(F)(F)c1cc(C(F)(F)F)c2ccc3cc(-c4nnco4)[nH]c3c2n1] | 2-[6,8-bis(trifluoromethyl)-1H- pyrrolo[3,2-h]quinolin-2-yl]-1,3,4- oxadiazole |
| 16 | 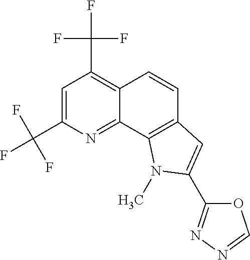 [SMILES: Cn1c(-c2nnco2)cc2ccc3c(C(F)(F)F)cc(C(F)(F)F)nc3c21] | 2-[1-methyl-6,8-bis(trifluoromethyl)-1H- pyrrolo[3,2-h]quinolin-2-yl]-1,3,4- oxadiazole |
| 17 | 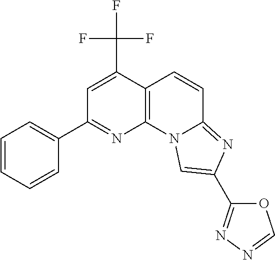 [SMILES: FC(F)(F)c1cc(-c2ccccc2)nc2c1ccc1nc(-c3nnco3)cn12] | 2-[2-phenyl-4- (trifluoromethyl)imidazo[1,2-a]1,8- naphthyridin-8-yl]-1,3,4-oxadiazole |
| 18 | 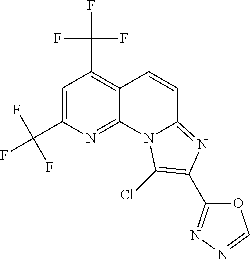 [SMILES: FC(F)(F)c1cc(C(F)(F)F)c2ccc3nc(-c4nnco4)c(Cl)n3c2n1] | 2-[9-chloro-2,4- bis(trifluoromethyl)imidazo[1,2-a]1,8- naphthyridin-8-yl]-1,3,4-oxadiazole |
| 19 | 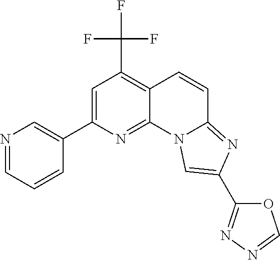 [SMILES: FC(F)(F)c1cc(-c2cccnc2)nc2c1ccc1nc(-c3nnco3)cn12] | 3-[8-(1,3,4-oxadiazol-2-yl)-4- (trifluoromethyl)imidazo[1,2-a]1,8- naphthyridin-2-yl]pyridine |
| 20 | 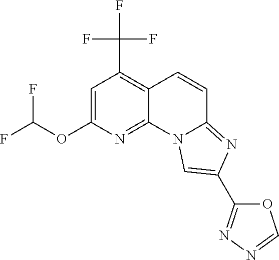 [SMILES: FC(F)Oc1cc(C(F)(F)F)c2ccc3nc(-c4nnco4)cn3c2n1] | 2-[2-(difluoromethoxy)-4- (trifluoromethyl)imidazo[1,2-a]1,8- naphthyridin-8-yl]-1,3,4-oxadiazole |
| 21 | 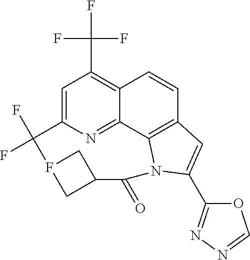 | 2-[1-cyclobutanecarbonyl-6,8- bis(trifluoromethyl)-1H-pyrrolo[3,2- h]quinolin-2-yl]-1,3,4-oxadiazole |
| 22 | 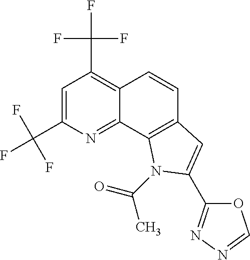 [SMILES: CC(=O)n1c(-c2nnco2)cc2ccc3c(C(F)(F)F)cc(C(F)(F)F)nc3c21] | 1-[2-(1,3,4-oxadiazol-2-yl)-6,8- bis(trifluoromethyl)-1H-pyrrolo[3,2- h]quinolin-1-yl]ethan-1-one |
| 23 | 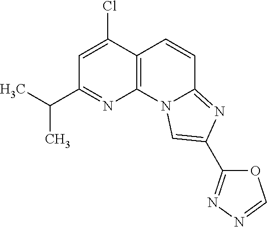 [SMILES: CC(C)c1cc(Cl)c2ccc3nc(-c4nnco4)cn3c2n1] | 2-[4-chloro-2-(propan-2-yl)imidazo[1,2- a]1,8-naphthyridin-8-yl]-1,3,4- oxadiazole |
| 24 | 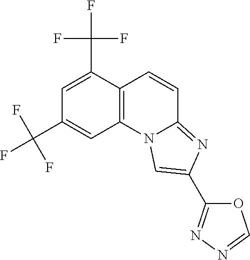 [SMILES: FC(F)(F)c1cc(C(F)(F)F)c2ccc3nc(-c4nnco4)cn3c2c1] | 2-[6,8-bis(trifluoromethyl)imidazo[1,2- a]quinolin-2-yl]-1,3,4-oxadiazole |
| 25 | 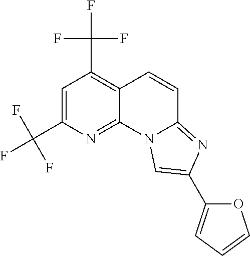 [SMILES: FC(F)(F)c1cc(C(F)(F)F)c2ccc3nc(-c4ccco4)cn3c2n1] | 8-(furan-2-yl)-2,4- bis(trifluoromethyl)imidazo[1,2-a]1,8- naphthyridine |
| 26 | 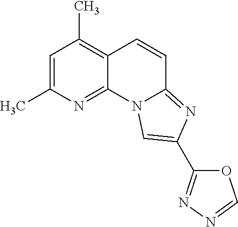 [SMILES: Cc1cc(C)c2ccc3nc(-c4nnco4)cn3c2n1] | 2-{2,4-dimethylimidazo[1,2-a]1,8- naphthyridin-8-yl}-1,3,4-oxadiazole |
| 27 | 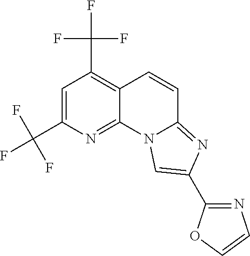 [SMILES: FC(F)(F)c1cc(C(F)(F)F)c2ccc3nc(-c4ncco4)cn3c2n1] | 2-[2,4-bis(trifluoromethyl)imidazo[1,2- a]1,8-naphthyridin-8-yl]-1,3-oxazole |
| 28 | 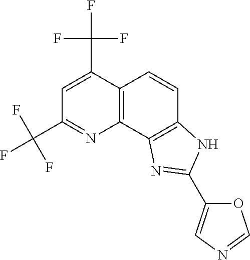 [SMILES: FC(F)(F)c1cc(C(F)(F)F)c2ccc3[nH]c(-c4cnco4)nc3c2n1] | 5-[6,8-bis(trifluoromethyl)-3H- imidazo[4,5-h]quinolin-2-yl]-1,3- oxazole |
| 29 | 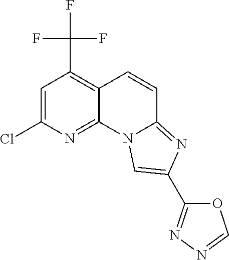 [SMILES: FC(F)(F)c1cc(Cl)nc2c1ccc1nc(-c3nnco3)cn12] | 2-[2-chloro-4- (trifluoromethyl)imidazo[1,2-a]1,8- naphthyridin-8-yl]-1,3,4-oxadiazole |
| 30 | 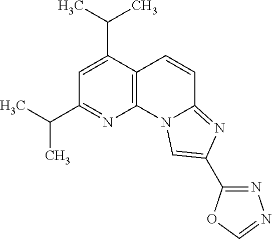 [SMILES: CC(C)c1cc(C(C)C)c2ccc3nc(-c4nnco4)cn3c2n1] | 2-[2,4-bis(propan-2-yl)imidazo[1,2- a]1,8-naphthyridin-8-yl]-1,3,4- oxadiazole |
| 31 | 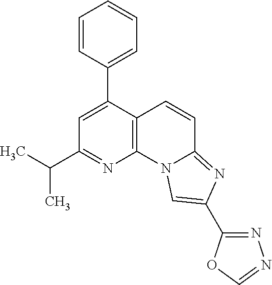 [SMILES: CC(C)c1cc(-c2ccccc2)c2ccc3nc(-c4nnco4)cn3c2n1] | 2-[4-phenyl-2-(propan-2-yl)imidazo[1,2- a]1,8-naphthyridin-8-yl]-1,3,4- oxadiazole |

In yet further embodiments, the compound of the present invention, or a pharmaceutically acceptable salt thereof, is chosen from the compounds set forth in Table 2.

TABLE 2

| Compound | | |
|---|---|---|
| Number | Structure | Chemical Name |
| 32 | 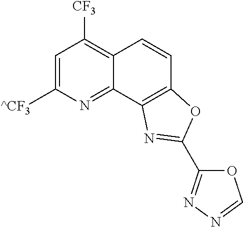 [SMILES: FC(F)(F)c1cc(C(F)(F)F)c2ccc3oc(-c4nnco4)nc3c2n1] | 2-[6,8-bis(trifluoromethyl)- [1,3]oxazolo[5,4-h]quinolin-2- yl]-1,3,4-oxadiazole |
| 33 | 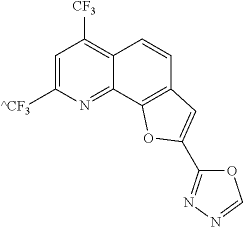 [SMILES: FC(F)(F)c1cc(C(F)(F)F)c2ccc3cc(-c4nnco4)oc3c2n1] | 2-[6,8- bis(trifluoromethyl)furo[3,2- h]quinolin-2-yl]-1,3,4- oxadiazole |
| 34 | 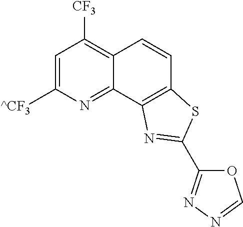 [SMILES: FC(F)(F)c1cc(C(F)(F)F)c2ccc3sc(-c4nnco4)nc3c2n1] | 2-[6,8-bis(trifluoromethyl)- [1,3]thiazolo[5,4-h]quinolin-2- yl]-1,3,4-oxadiazole |
| 35 | 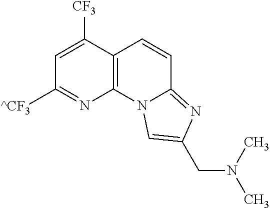 [SMILES: CN(C)Cc1cn2c(ccc3c(C(F)(F)F)cc(C(F)(F)F)nc32)n1] | {[2,4- bis(trifluoromethyl)imidazo[1,2- a]1,8-naphthyridin-8- yl]methyl}dimethylamine |
| 36 | 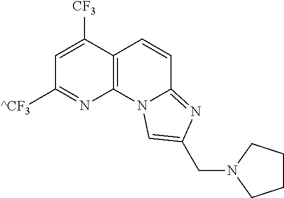 [SMILES: FC(F)(F)c1cc(C(F)(F)F)c2ccc3nc(CN4CCCC4)cn3c2n1] | 1-{[2,4- bis(trifluoromethyl)imidazo[1,2- a]1,8-naphthyridin-8- yl]methyl}pyrrolidine |
| 37 | 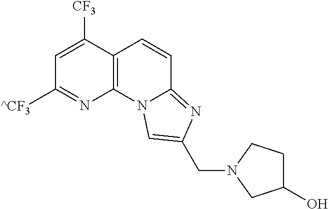 [SMILES: OC1CCN(Cc2cn3c(ccc4c(C(F)(F)F)cc(C(F)(F)F)nc43)n2)C1] | 1-{[2,4- bis(trifluoromethyl)imidazo[1,2- a]1,8-naphthyridin-8- yl]methyl}pyrrolidin-3-ol |
| 38 | 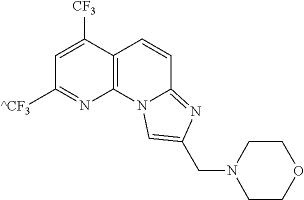 [SMILES: FC(F)(F)c1cc(C(F)(F)F)c2ccc3nc(CN4CCOCC4)cn3c2n1] | 4-{[2,4- bis(trifluoromethyl)imidazo[1,2- a]1,8-naphthyridin-8- yl]methyl}morpholine |
| 39 | 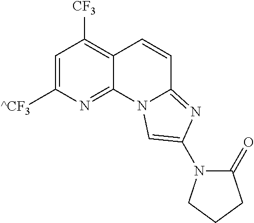 [SMILES: O=C1CCCN1c1cn2c(ccc3c(C(F)(F)F)cc(C(F)(F)F)nc32)n1] | 1-[2,4- bis(trifluoromethyl)imidazo[1,2- a]1,8-naphthyridin-8- yl]pyrrolidin-2-one |
| 40 | 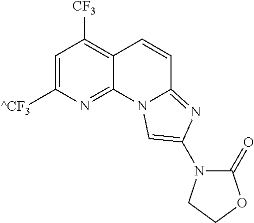 [SMILES: O=C1OCCN1c1cn2c(ccc3c(C(F)(F)F)cc(C(F)(F)F)nc32)n1] | 3-[2,4- bis(trifluoromethyl)imidazo[1,2- a]1,8-naphthyridin-8-yl]-1,3- oxazolidin-2-one |
| 41 | 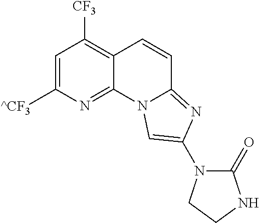 [SMILES: O=C1NCCN1c1cn2c(ccc3c(C(F)(F)F)cc(C(F)(F)F)nc32)n1] | 1-[2,4- bis(trifluoromethyl)imidazo[1,2- a]1,8-naphthyridin-8- yl]imidazolidin-2-one |
| 42 | 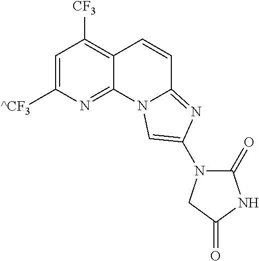 [SMILES: O=C1CN(c2cn3c(ccc4c(C(F)(F)F)cc(C(F)(F)F)nc43)n2)C(=O)N1] | 1-[2,4- bis(trifluoromethyl)imidazo[1,2- a]1,8-naphthyridin-8- yl]imidazolidine-2,4-dione |
| 43 | 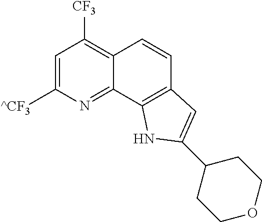 [SMILES: FC(F)(F)c1cc(C(F)(F)F)c2ccc3cc(C4CCOCC4)[nH]c3c2n1] | 2-(oxan-4-yl)-6,8- bis(trifluoromethyl)-1H- pyrrolo[3,2-h]quinoline |
| 44 | 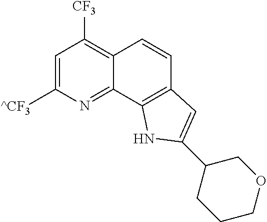 [SMILES: FC(F)(F)c1cc(C(F)(F)F)c2ccc3cc(C4CCCOC4)[nH]c3c2n1] | 2-(oxan-3-yl)-6,8- bis(trifluoromethyl)-1H- pyrrolo[3,2-h]quinoline |
| 45 | 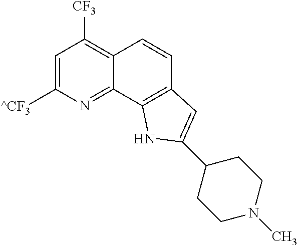 [SMILES: CN1CCC(c2cc3ccc4c(C(F)(F)F)cc(C(F)(F)F)nc4c3[nH]2)CC1] | 4-[6,8-bis(trifluoromethyl)-1H- pyrrolo[3,2-h]quinolin-2-yl]-1- methylpiperidine |
| 46 | 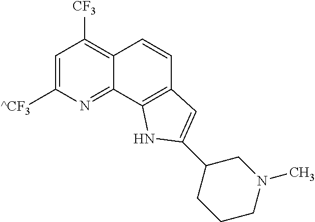 [SMILES: CN1CCCC(c2cc3ccc4c(C(F)(F)F)cc(C(F)(F)F)nc4c3[nH]2)C1] | 3-[6,8-bis(trifluoromethyl)-1H- pyrrolo[3,2-h]quinolin-2-yl]-1- methylpiperidine |
| 47 | 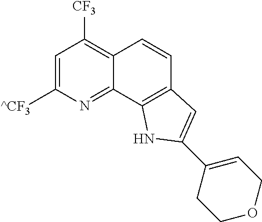 [SMILES: FC(F)(F)c1cc(C(F)(F)F)c2ccc3cc(C4=CCOCC4)[nH]c3c2n1] | 2-(3,6-dihydro-2H-pyran-4-yl)- 6,8-bis(trifluoromethyl)-1H- pyrrolo[3,2-h]quinoline |
| 48 | 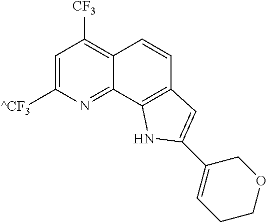 [SMILES: FC(F)(F)c1cc(C(F)(F)F)c2ccc3cc(C4=CCCOC4)[nH]c3c2n1] | 2-(5,6-dihydro-2H-pyran-3-yl)- 6,8-bis(trifluoromethyl)-1H- pyrrolo[3,2-h]quinoline |
| 49 | 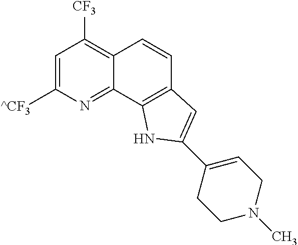 [SMILES: CN1CC=C(c2cc3ccc4c(C(F)(F)F)cc(C(F)(F)F)nc4c3[nH]2)CC1] | 4-[6,8-bis(trifluoromethyl)-1H- pyrrolo[3,2-h]quinolin-2-yl]-1- methyl-1,2,3,6- tetrahydropyridine |
| 50 | 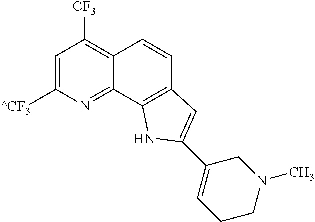 [SMILES: CN1CCC=C(c2cc3ccc4c(C(F)(F)F)cc(C(F)(F)F)nc4c3[nH]2)C1] | 5-[6,8-bis(trifluoromethyl)-1H- pyrrolo[3,2-h]quinolin-2-yl]-1- methyl-1,2,3,6- tetrahydropyridine |
| 51 | 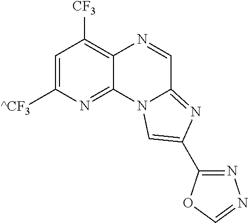 [SMILES: FC(F)(F)c1cc(C(F)(F)F)c2ncc3nc(-c4nnco4)cn3c2n1] | 4-(1,3,4-oxadiazol-2-yl)-10,12- bis(trifluoromethyl)-2,5,8,13- tetraazatricyclo[7.4.0.0^{2, 6}]trideca- 1(13),3,5,7,9,11-hexaene |
| 52 | 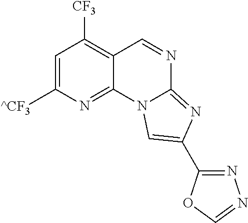 [SMILES: FC(F)(F)c1cc(C(F)(F)F)c2cnc3nc(-c4nnco4)cn3c2n1] | 4-(1,3,4-oxadiazol-2-yl)-10,12- bis(trifluoromethyl)-2,5,7,13- tetraazatricyclo[7.4.0.0^{2, 6}]trideca- 1(13),3,5,7,9,11-hexaene |
| 53 | 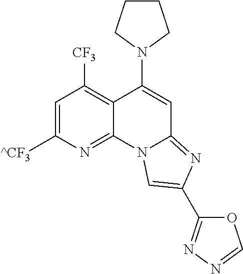 [SMILES: FC(F)(F)c1cc(C(F)(F)F)c2c(N3CCCC3)cc3nc(-c4nnco4)cn3c2n1] | 2-[5-(pyrrolidin-1-yl)-2,4- bis(trifluoromethyl)imidazo[1,2- a]1,8-naphthyridin-8-yl]-1,3,4- oxadiazole |
| 54 | 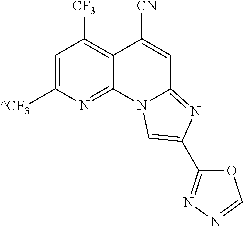 [SMILES: N#Cc1cc2nc(-c3nnco3)cn2c2nc(C(F)(F)F)cc(C(F)(F)F)c12] | 8-(1,3,4-oxadiazol-2-yl)-2,4- bis(trifluoromethyl)imidazo[1,2- a]1,8-naphthyridine-5- carbonitrile |
| 55 | 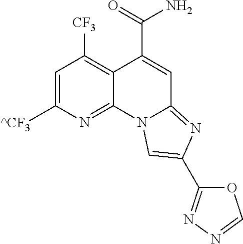 [SMILES: NC(=O)c1cc2nc(-c3nnco3)cn2c2nc(C(F)(F)F)cc(C(F)(F)F)c12] | 8-(1,3,4-oxadiazol-2-yl)-2,4- bis(trifluoromethyl)imidazo[1,2- a]1,8-naphthyridine-5- carboxamide |
| 56 | 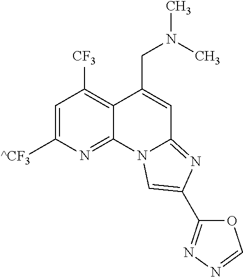 [SMILES: CN(C)Cc1cc2nc(-c3nnco3)cn2c2nc(C(F)(F)F)cc(C(F)(F)F)c12] | dimethyl({[8-(1,3,4-oxadiazol-2- yl)-2,4- bis(trifluoromethyl)imidazo[1,2- a]1,8-naphthyridin-5- yl]methyl})amine |
| 57 | 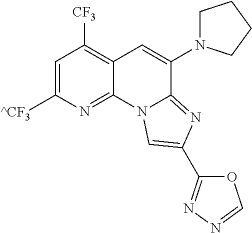 [SMILES: FC(F)(F)c1cc(C(F)(F)F)c2cc(N3CCCC3)c3nc(-c4nnco4)cn3c2n1] | 2-[6-(pyrrolidin-1-yl)-2,4- bis(trifluoromethyl)imidazo[1,2- a]1,8-naphthyridin-8-yl]-1,3,4- oxadiazole |
| 58 | 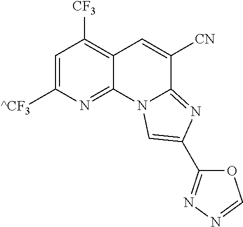 [SMILES: N#Cc1cc2c(C(F)(F)F)cc(C(F)(F)F)nc2n2cc(-c3nnco3)nc12] | 8-(1,3,4-oxadiazol-2-yl)-2,4- bis(trifluoromethyl)imidazo[1,2- a]1,8-naphthyridine-6- carbonitrile |
| 59 | 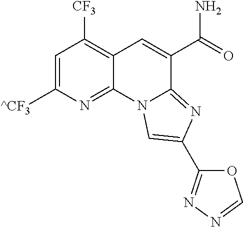 [SMILES: NC(=O)c1cc2c(C(F)(F)F)cc(C(F)(F)F)nc2n2cc(-c3nnco3)nc12] | 8-(1,3,4-oxadiazol-2-yl)-2,4- bis(trifluoromethyl)imidazo[1,2- a]1,8-naphthyridine-6- carboxamide |
| 60 | 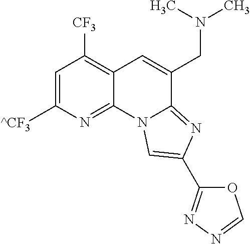 [SMILES: CN(C)Cc1cc2c(C(F)(F)F)cc(C(F)(F)F)nc2n2cc(-c3nnco3)nc12] | dimethyl({[8-(1,3,4-oxadiazol-2- yl)-2,4- bis(trifluoromethyl)imidazo[1,2- a]1,8-naphthyridin-6- yl]methyl})amine |
| 61 | 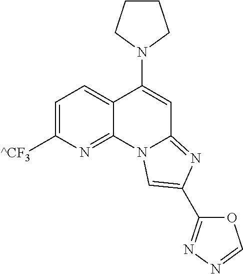 [SMILES: FC(F)(F)c1ccc2c(N3CCCC3)cc3nc(-c4nnco4)cn3c2n1] | 2-[5-(pyrrolidin-1-yl)-2- (trifluoromethyl)imidazo[1,2- a]1,8-naphthyridin-8-yl]-1,3,4- oxadiazole |
| 62 | 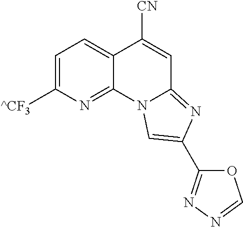 [SMILES: N#Cc1cc2nc(-c3nnco3)cn2c2nc(C(F)(F)F)ccc12] | 8-(1,3,4-oxadiazol-2-yl)-2- (trifluoromethyl)imidazo[1,2- a]1,8-naphthyridine-5- carbonitrile |
| 63 | 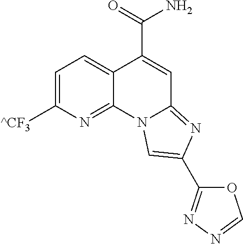 [SMILES: NC(=O)c1cc2nc(-c3nnco3)cn2c2nc(C(F)(F)F)ccc12] | 8-(1,3,4-oxadiazol-2-yl)-2- (trifluoromethyl)imidazo[1,2- a]1,8-naphthyridine-5- carboxamide |
| 64 | 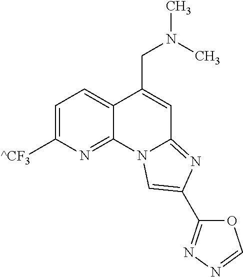 [SMILES: CN(C)Cc1cc2nc(-c3nnco3)cn2c2nc(C(F)(F)F)ccc12] | dimethyl({[8-(1,3,4-oxadiazol-2- yl)-2- (trifluoromethyl)imidazo[1,2- a]1,8-naphthyridin-5- yl]methyl})amine |
| 65 | 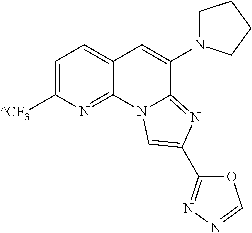 [SMILES: FC(F)(F)c1ccc2cc(N3CCCC3)c3nc(-c4nnco4)cn3c2n1] | 2-[6-(pyrrolidin-1-yl)-2- (trifluoromethyl)imidazo[1,2- a]1,8-naphthyridin-8-yl]-1,3,4- oxadiazole |
| 66 | 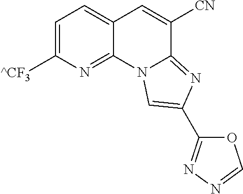 [SMILES: N#Cc1cc2ccc(C(F)(F)F)nc2n2cc(-c3nnco3)nc12] | 8-(1,3,4-oxadiazol-2-yl)-2- (trifluoromethyl)imidazo[1,2- a]1,8-naphthyridine-6- carbonitrile |
| 67 | 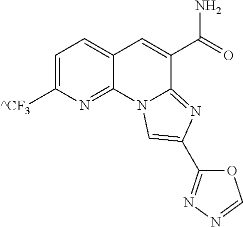 [SMILES: NC(=O)c1cc2ccc(C(F)(F)F)nc2n2cc(-c3nnco3)nc12] | 8-(1,3,4-oxadiazol-2-yl)-2- (trifluoromethyl)imidazo[1,2- a]1,8-naphthyridine-6- carboxamide |
| 68 | 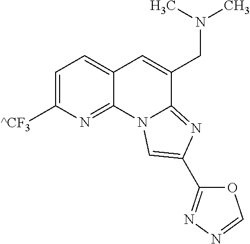 [SMILES: CN(C)Cc1cc2ccc(C(F)(F)F)nc2n2cc(-c3nnco3)nc12] | dimethyl({[8-(1,3,4-oxadiazol-2- yl)-2- (trifluoromethyl)imidazo[1,2- a]1,8-naphthyridin-6- yl]methyl})amine |
| 69 | 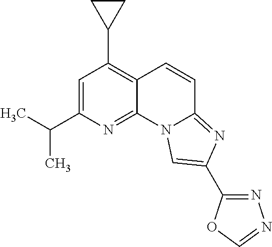 [SMILES: CC(C)c1cc(C2CC2)c2ccc3nc(-c4nnco4)cn3c2n1] | 2-[4-cyclopropyl-2-(propan-2- yl)imidazo[1,2-a]1,8- naphthyridin-8-yl]-1,3,4- oxadiazole |
| 70 | 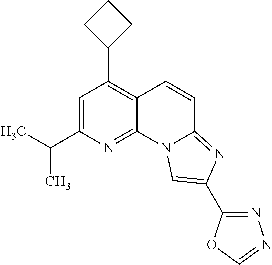 [SMILES: CC(C)c1cc(C2CCC2)c2ccc3nc(-c4nnco4)cn3c2n1] | 2-[4-cyclobutyl-2-(propan-2- yl)imidazo[1,2-a]1,8- naphthyridin-8-yl]-1,3,4- oxadiazole |
| 71 | 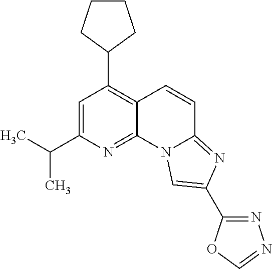 [SMILES: CC(C)c1cc(C2CCCC2)c2ccc3nc(-c4nnco4)cn3c2n1] | 2-[4-cyclopentyl-2-(propan-2- yl)imidazo[1,2-a]1,8- naphthyridin-8-yl]-1,3,4- oxadiazole |
| 72 | 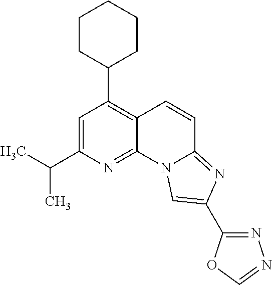 [SMILES: CC(C)c1cc(C2CCCCC2)c2ccc3nc(-c4nnco4)cn3c2n1] | 2-[4-cyclohexyl-2-(propan-2- yl)imidazo[1,2-a]1,8- naphthyridin-8-yl]-1,3,4- oxadiazole |
| 73 | 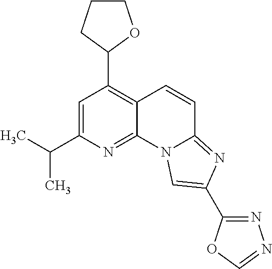 [SMILES: CC(C)c1cc(C2CCCO2)c2ccc3nc(-c4nnco4)cn3c2n1] | 2-[4-(oxolan-2-yl)-2-(propan-2- yl)imidazo[1,2-a]1,8- naphthyridin-8-yl]-1,3,4- oxadiazole |
| 74 | 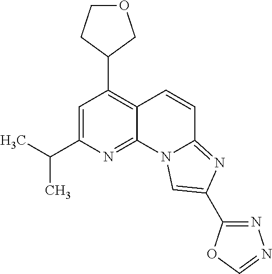 [SMILES: CC(C)c1cc(C2CCOC2)c2ccc3nc(-c4nnco4)cn3c2n1] | 2-[4-(oxolan-3-yl)-2-(propan-2- yl)imidazo[1,2-a]1,8- naphthyridin-8-yl]-1,3,4- oxadiazole |
| 75 | 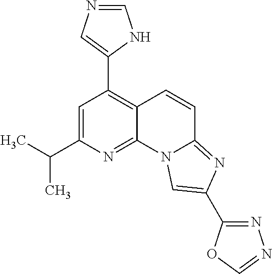 [SMILES: CC(C)c1cc(-c2cnc[nH]2)c2ccc3nc(-c4nnco4)cn3c2n1] | 2-[4-(1H-imidazol-5-yl)-2- (propan-2-yl)imidazo[1,2-a]1,8- naphthyridin-8-yl]-1,3,4- oxadiazole |
| 76 | 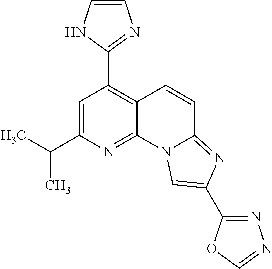 [SMILES: CC(C)c1cc(-c2ncc[nH]2)c2ccc3nc(-c4nnco4)cn3c2n1] | 2-[4-(1H-imidazol-2-yl)-2- (propan-2-yl)imidazo[1,2-a]1,8- naphthyridin-8-yl]-1,3,4- oxadiazole |
| 77 | 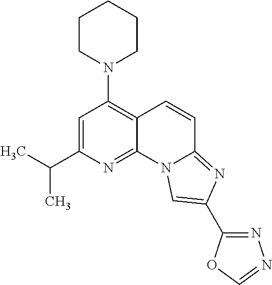 [SMILES: CC(C)c1cc(N2CCCCC2)c2ccc3nc(-c4nnco4)cn3c2n1] | 1-[8-(1,3,4-oxadiazol-2-yl)-2- (propan-2-yl)imidazo[1,2-a]1,8- naphthyridin-4-yl]piperidine |
| 78 | 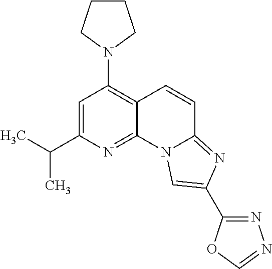 [SMILES: CC(C)c1cc(N2CCCC2)c2ccc3nc(-c4nnco4)cn3c2n1] | 2-[2-(propan-2-yl)-4-(pyrrolidin- 1-yl)imidazo[1,2-a]1,8- naphthyridin-8-yl]-1,3,4- oxadiazole |
| 79 | 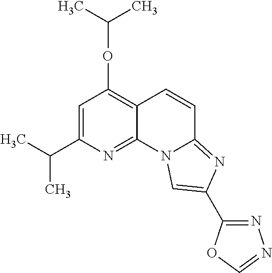 [SMILES: CC(C)Oc1cc(C(C)C)nc2c1ccc1nc(-c3nnco3)cn12] | 2-[2-(propan-2-yl)-4-(propan-2- yloxy)imidazo[1,2-a]1,8- naphthyridin-8-yl]-1,3,4- oxadiazole |
| 80 | 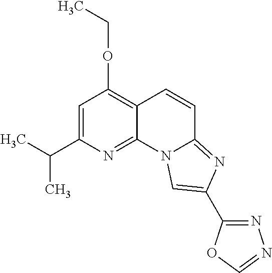 [SMILES: CCOc1cc(C(C)C)nc2c1ccc1nc(-c3nnco3)cn12] | 2-[4-ethoxy-2-(propan-2- yl)imidazo[1,2-a]1,8- naphthyridin-8-yl]-1,3,4- oxadiazole |
| 51 | 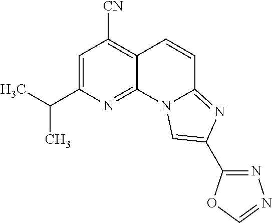 [SMILES: CC(C)c1cc(C#N)c2ccc3nc(-c4nnco4)cn3c2n1] | 8-(1,3,4-oxadiazol-2-yl)-2- (propan-2-yl)imidazo[1,2-a]1,8- naphthyridine-4-carbonitrile |
| 52 | 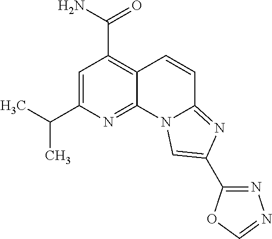 [SMILES: CC(C)c1cc(C(N)=O)c2ccc3nc(-c4nnco4)cn3c2n1] | 8-(1,3,4-oxadiazol-2-yl)-2- (propan-2-yl)imidazo[1,2-a]1,8- naphthyridine-4-carboxamide |
| 53 | 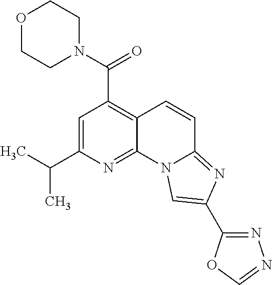 [SMILES: CC(C)c1cc(C(=O)N2CCOCC2)c2ccc3nc(-c4nnco4)cn3c2n1] | 4-{[8-(1,3,4-oxadiazol-2-yl)-2- (propan-2-yl)imidazo[1,2-a]1,8- naphthyridin-4- yl]carbonyl}morpholine |
| 54 | 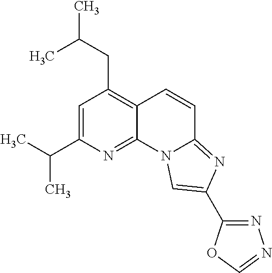 [SMILES: CC(C)Cc1cc(C(C)C)nc2c1ccc1nc(-c3nnco3)cn12] | 2-[4-(2-methylpropyl)-2- (propan-2-yl)imidazo[1,2-a]1,8- naphthyridin-8-yl]-1,3,4- oxadiazole |
| 55 | 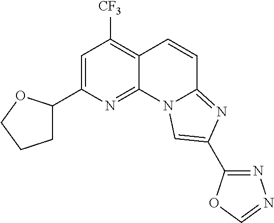 [SMILES: FC(F)(F)c1cc(C2CCCO2)nc2c1ccc1nc(-c3nnco3)cn12] | 2-[2-(oxolan-2-yl)-4- (trifluoromethyl)imidazo[1,2- a]1,8-naphthyridin-8-yl]-1,3,4- oxadiazole |
| 56 | 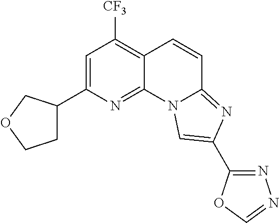 [SMILES: FC(F)(F)c1cc(C2CCOC2)nc2c1ccc1nc(-c3nnco3)cn12] | 2-[2-(oxolan-3-yl)-4- (trifluoromethyl)imidazo[1,2- a]1,8-naphthyridin-8-yl]-1,3,4- oxadiazole |
| 57 | 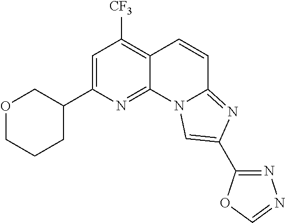 [SMILES: FC(F)(F)c1cc(C2CCCOC2)nc2c1ccc1nc(-c3nnco3)cn12] | 2-[2-(oxan-3-yl)-4- (trifluoromethyl)imidazo[1,2- a]1,8-naphthyridin-8-yl]-1,3,4- oxadiazole |
| 58 | 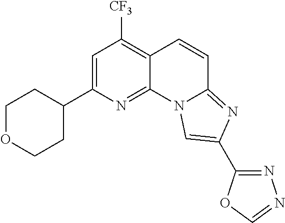 [SMILES: FC(F)(F)c1cc(C2CCOCC2)nc2c1ccc1nc(-c3nnco3)cn12] | 2-[2-(oxan-4-yl)-4- (trifluoromethyl)imidazo[1,2- a]1,8-naphthyridin-8-yl]-1,3,4- oxadiazole |
| 59 | 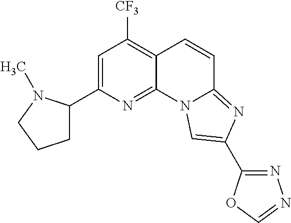 [SMILES: CN1CCCC1c1cc(C(F)(F)F)c2ccc3nc(-c4nnco4)cn3c2n1] | 2-[2-(1-methylpyrrolidin-2-yl)-4- (trifluoromethyl)imidazo[1,2- a]1,8-naphthyridin-8-yl]-1,3,4- oxadiazole |
| 60 | 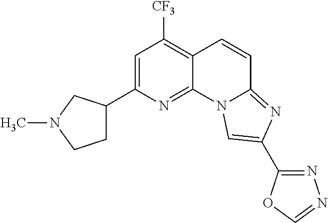 [SMILES: CN1CCC(c2cc(C(F)(F)F)c3ccc4nc(-c5nnco5)cn4c3n2)C1] | 2-[2-(1-methylpyrrolidin-3-yl)-4- (trifluoromethyl)imidazo[1,2- a]1,8-naphthyridin-8-yl]-1,3,4- oxadiazole |
| 61 | 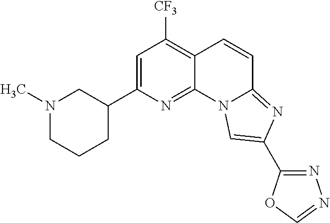 [SMILES: CN1CCCC(c2cc(C(F)(F)F)c3ccc4nc(-c5nnco5)cn4c3n2)C1] | 1-methyl-3-[8-(1,3,4-oxadiazol- 2-yl)-4- (trifluoromethyl)imidazo[1,2- a]1,8-naphthyridin-2- yl]piperidine |
| 62 | 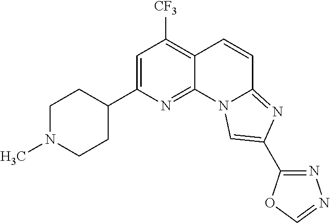 [SMILES: CN1CCC(c2cc(C(F)(F)F)c3ccc4nc(-c5nnco5)cn4c3n2)CC1] | 1-methyl-4-[8-(1,3,4-oxadiazol- 2-yl)-4- (trifluoromethyl)imidazo[1,2- a]1,8-naphthyridin-2- yl]piperidine |
| 63 | 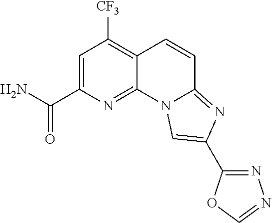 [SMILES: NC(=O)c1cc(C(F)(F)F)c2ccc3nc(-c4nnco4)cn3c2n1] | 8-(1,3,4-oxadiazol-2-yl)-4- (trifluoromethyl)imidazo[1,2- a]1,8-naphthyridine-2- carboxamide |
| 64 | 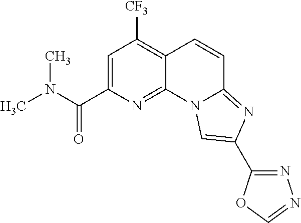 [SMILES: CN(C)C(=O)c1cc(C(F)(F)F)c2ccc3nc(-c4nnco4)cn3c2n1] | N,N-dimethyl-8-(1,3,4- oxadiazol-2-yl)-4- (trifluoromethyl)imidazo[1,2- a]1,8-naphthyridine-2- carboxamide |
| 65 | 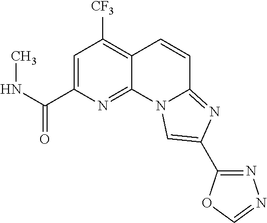 [SMILES: CNC(=O)c1cc(C(F)(F)F)c2ccc3nc(-c4nnco4)cn3c2n1] | N-methyl-8-(1,3,4-oxadiazol-2- yl)-4- (trifluoromethyl)imidazo[1,2- a]1,8-naphthyridine-2- carboxamide |
| 66 | 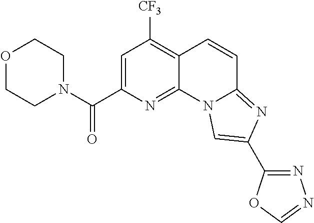 [SMILES: O=C(c1cc(C(F)(F)F)c2ccc3nc(-c4nnco4)cn3c2n1)N1CCOCC1] | 4-{[8-(1,3,4-oxadiazol-2-yl)-4- (trifluoromethyl)imidazo[1,2- a]1,8-naphthyridin-2- yl]carbonyl}morpholine |
| 67 | 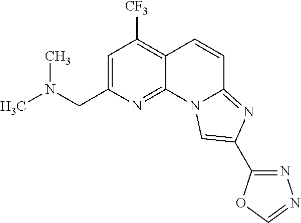 [SMILES: CN(C)Cc1cc(C(F)(F)F)c2ccc3nc(-c4nnco4)cn3c2n1] | dimethyl({[8-(1,3,4-oxadiazol-2- yl)-4- (trifluoromethyl)imidazo[1,2- a]1,8-naphthyridin-2- yl]methyl})amine |
| 68 | 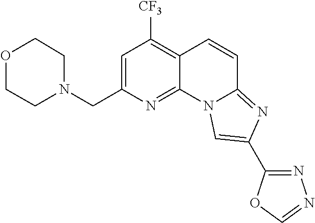 [SMILES: FC(F)(F)c1cc(CN2CCOCC2)nc2c1ccc1nc(-c3nnco3)cn12] | 4-{[8-(1,3,4-oxadiazol-2-yl)-4- (trifluoromethyl)imidazo[1,2- a]1,8-naphthyridin-2- yl]methyl}morpholine |
| 69 | 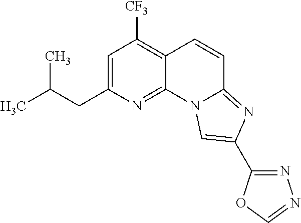 [SMILES: CC(C)Cc1cc(C(F)(F)F)c2ccc3nc(-c4nnco4)cn3c2n1] | 2-[2-(2-methylpropyl)-4- (trifluoromethyl)imidazo[1,2- a]1,8-naphthyridin-8-yl]-1,3,4- oxadiazole |
| 70 | 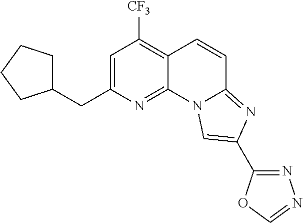 [SMILES: FC(F)(F)c1cc(CC2CCCC2)nc2c1ccc1nc(-c3nnco3)cn12] | 2-[2-(cyclopentylmethyl)-4- (trifluoromethyl)imidazo[1,2- a]1,8-naphthyridin-8-yl]-1,3,4- oxadiazole |
| 71 | 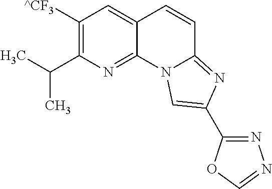 [SMILES: CC(C)c1nc2c(ccc3nc(-c4nnco4)cn32)cc1C(F)(F)F] | 2-[2-(propan-2-yl)-3- (trifluoromethyl)imidazo[1,2- a]1,8-naphthyridin-8-yl]-1,3,4- oxadiazole |
| 72 | 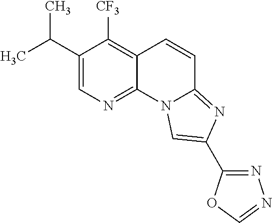 [SMILES: CC(C)c1cnc2c(ccc3nc(-c4nnco4)cn32)c1C(F)(F)F] | 2-[3-(propan-2-yl)-4- (trifluoromethyl)imidazo[1,2- a]1,8-naphthyridin-8-yl]-1,3,4- oxadiazole |
| 73 | 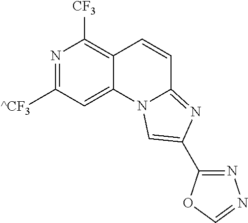 [SMILES: FC(F)(F)c1cc2c(ccc3nc(-c4nnco4)cn32)c(C(F)(F)F)n1] | 2-[2,4- bis(trifluoromethyl)imidazo[1,2- a]1,6-naphthyridin-8-yl]-1,3,4- oxadiazole |
| 74 | 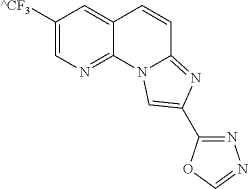 [SMILES: FC(F)(F)c1cnc2c(ccc3nc(-c4nnco4)cn32)c1] | 2-[3- (trifluoromethyl)imidazo[1,2- a]1,8-naphthyridin-8-yl]-1,3,4- oxadiazole |
| 75 | 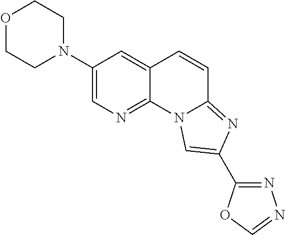 [SMILES: c1nnc(-c2cn3c(ccc4cc(N5CCOCC5)cnc43)n2)o1] | 4-[8-(1,3,4-oxadiazol-2- yl)imidazo[1,2-a]1,8- naphthyridin-3-yl]morpholine |
| 76 | 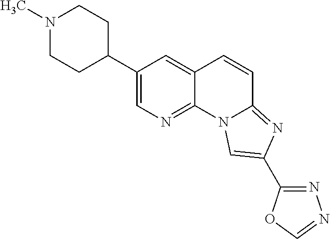 [SMILES: CN1CCC(c2cnc3c(ccc4nc(-c5nnco5)cn43)c2)CC1] | 1-methyl-4-[8-(1,3,4-oxadiazol- 2-yl)imidazo[1,2-a]1,8- naphthyridin-3-yl]piperidine |
| 77 | 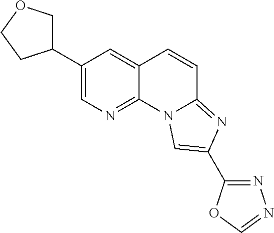 [SMILES: c1nnc(-c2cn3c(ccc4cc(C5CCOC5)cnc43)n2)o1] | 2-[3-(oxolan-3-yl)imidazo[1,2- a]1,8-naphthyridin-8-yl]-1,3,4- oxadiazole |
| 78 | 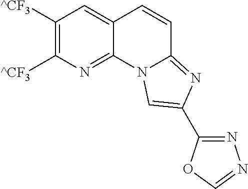 [SMILES: FC(F)(F)c1cc2ccc3nc(-c4nnco4)cn3c2nc1C(F)(F)F] | 2-[2,3- bis(trifluoromethyl)imidazo[1,2- a]1,8-naphthyridin-8-yl]-1,3,4- oxadiazole |
| 79 | 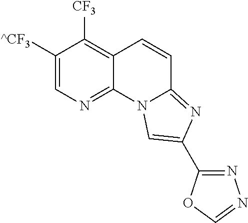 [SMILES: FC(F)(F)c1cnc2c(ccc3nc(-c4nnco4)cn32)c1C(F)(F)F] | 2-[3,4- bis(trifluoromethyl)imidazo[1,2- a]1,8-naphthyridin-8-yl]-1,3,4- oxadiazole |
| 80 | 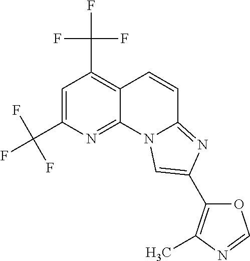 [SMILES: Cc1ncoc1-c1cn2c(ccc3c(C(F)(F)F)cc(C(F)(F)F)nc32)n1] | 5-[2,4- bis(trifluoromethyl)imidazo[1,2- a]1,8-naphthyridin-8-yl]-4- methyl-1,3-oxazole |
| 81 | 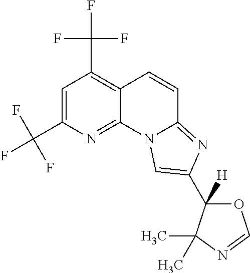 [SMILES: CC1(C)N=CO[C@H]1c1cn2c(ccc3c(C(F)(F)F)cc(C(F)(F)F)nc32)n1] | (5R)-5-[2,4- bis(trifluoromethyl)imidazo[1,2- a]1,8-naphthyridin-8-yl]-4,4- dimethyl-4,5-dihydro-1,3- oxazole |
| 82 | 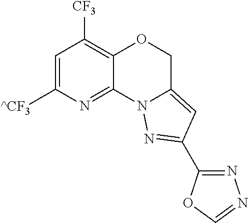 [SMILES: FC(F)(F)c1cc(C(F)(F)F)c2c(n1)-n1nc(-c3nnco3)cc1CO2] | 4-(1,3,4-oxadiazol-2-yl)-10,12- bis(trifluoromethyl)-8-oxa- 2,3,13- triazatricyclo[7.4.0.0^{2, 6}]trideca- 1(13),3,5,9,11-pentaene |
| 83 | 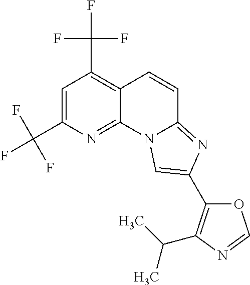 [SMILES: CC(C)c1ncoc1-c1cn2c(ccc3c(C(F)(F)F)cc(C(F)(F)F)nc32)n1] | 5-[2,4- bis(trifluoromethyl)imidazo[1,2- a]1,8-naphthyridin-8-yl]-4- (propan-2-yl)-1,3-oxazole |
| 84 | 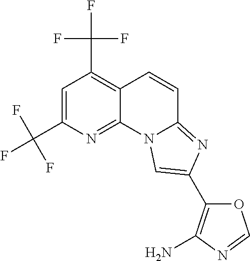 [SMILES: Nc1ncoc1-c1cn2c(ccc3c(C(F)(F)F)cc(C(F)(F)F)nc32)n1] | 5-[2,4- bis(trifluoromethyl)imidazo[1,2- a]1,8-naphthyridin-8-yl]-1,3- oxazol-4-amine |
| 85 | 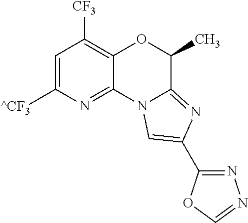 [SMILES: C[C@@H]1Oc2c(C(F)(F)F)cc(C(F)(F)F)nc2-n2cc(-c3nnco3)nc21] | (7S)-7-methyl-4-(1,3,4- oxadiazol-2-yl)-10,12- bis(trifluoromethyl)-8-oxa- 2,5,13- triazatricyclo[7.4.0.0^{2, 6}]trideca- 1(13),3,5,9,11-pentaene |
| 86 | 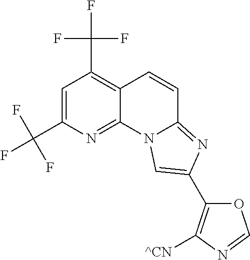 [SMILES: N#Cc1ncoc1-c1cn2c(ccc3c(C(F)(F)F)cc(C(F)(F)F)nc32)n1] | 5-[2,4- bis(trifluoromethyl)imidazo[1,2- a]1,8-naphthyridin-8-yl]-1,3- oxazole-4-carbonitrile |
| 87 | 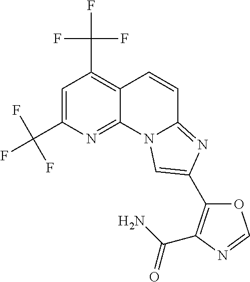 [SMILES: NC(=O)c1ncoc1-c1cn2c(ccc3c(C(F)(F)F)cc(C(F)(F)F)nc32)n1] | 5-[2,4- bis(trifluoromethyl)imidazo[1,2- a]1,8-naphthyridin-8-yl]-1,3- oxazole-4-carboxamide |
| 88 | 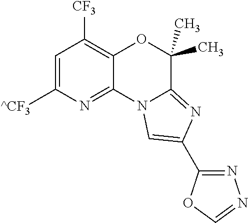 | 7,7-dimethyl-4-(1,3,4- oxadiazol-2-yl)-10,12- bis(trifluoromethyl)-8-oxa- 2,5,13- triazatricyclo[7.4.0.0^{2, 6}]trideca- 1(13),3,5,9,11-pentaene |
| 89 | 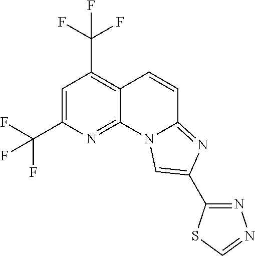 [SMILES: FC(F)(F)c1cc(C(F)(F)F)c2ccc3nc(-c4nncs4)cn3c2n1] | 2-(2,4- bisfluoromethyl)imidazo[1,2- a][1,8]naphthyridin-8-yl)-1,3,4- thiadiazole |

The compounds of Table 1 were synthesized according to the Synthetic Methods, General Schemes, and the Examples described below. The compounds of Table 2 can be synthesized by one of skill in the art by following the Synthetic Methods, General Schemes, and the Examples described below.

In certain embodiments, the compound(s) of the present invention, or a pharmaceutically acceptable salt thereof, are chosen from the compounds set forth in Tables 1 and 2. In other embodiments, the compounds of the present invention, or a pharmaceutically acceptable salt thereof, are chosen from the compounds set forth in Table 1. In still other embodiments, the compounds of the present invention, or a pharmaceutically acceptable salt thereof, are chosen from the compounds set forth in Table 2.

### Synthetic Methods

The methods of synthesis for the provided chemical entities employ readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, the methods of this invention may employ protecting groups which prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers or diastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementalso (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

Unless specified to the contrary, the reactions described herein take place at atmospheric pressure, generally within a temperature range from −78° C. to 200° C. Further, except as employed in the Examples or as otherwise specified, reaction times and conditions are intended to be approximate, e.g., taking place at about atmospheric pressure within a temperature range of about −78° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith, including, for example, benzene, toluene, acetonitrile, tetrahydrofuranyl (“THF”), dimethylformamide (“DMF”), chloroform, methylene chloride (or dichloromethane), diethyl ether, methanol, N-methylpyrrolidone (“NMP”), pyridine and the like.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

### EXAMPLES

The following examples serve to more fully describe the manner of making and using the above-described invention. It is understood that these examples in no way serve to limit the true scope of the invention, but rather are presented for illustrative purposes. In the examples below and the synthetic schemes above, the following abbreviations have the following meanings. If an abbreviation is not defined, it has its generally accepted meaning.
aq.=aqueous
μL=microliters
μM=micromolar
NMR=nuclear magnetic resonance
boc=tert-butoxycarbonyl
br=broad
Cbz=benzyloxycarbonyl
d=doublet
δ=chemical shift
° C.=degrees celcius
DCM=dichloromethane
dd=doublet of doublets
DMEM=Dulbeco's Modified Eagle's Medium
DMF=N,N-dimethylformamide
DMSO=dimethylsulfoxide
EtOAc=ethyl acetate
g=gram
h or hr=hours
HCV=hepatitus C virus
HPLC=high performance liquid chromatography
Hz=hertz
IU=International Units
IC₅₀=inhibitory concentration at 50% inhibition
J=coupling constant (given in Hz unless otherwise indicated)
m=multiplet
M=molar
M+H⁺=parent mass spectrum peak plus H*
mg=milligram
mL=milliliter
mM=millimolar
mmol=millimole
MS=mass spectrum
nm=nanomolar
ppm=parts per million
q.s.=sufficient amount
s=singlet
sat.=saturated
t=triplet
TFA=trifluoroacetic acid

### General Synthesis Schemes

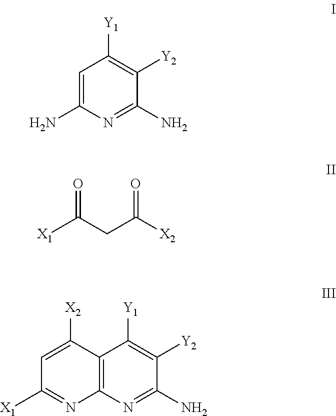

1,8-napthyridines of the general type III can be prepared from the corresponding 1,6-bisamino pyridines of general formula I and a corresponding diketone of general formula II. For example, those skilled in the art will recognize that treatment of I (Y₁═Y₂═H) with II (X₁═X₂═CF₃) in the presence of a suitable solvent (for example acetic acid) and heat (for example 80° C.) will give the corresponding napthyridine III (Y₁═Y₂═H; X₁═X₂═CF₃). Similarly, treatment of I (Y₁═Y₂═H) with II (X₁═OEt, X₂═CF₃) in the presence of solvent (diphenyl ether) and heat (for example 130° C. for 5 hours followed by 210° C. for 16 hours) affords III (X₁═OH, X₂═CF₃, Y₁═Y₂═H). Those skilled in the art will recognize this constitutes a general approach toward the preparation of molecules of general formula III of many different substitutions.

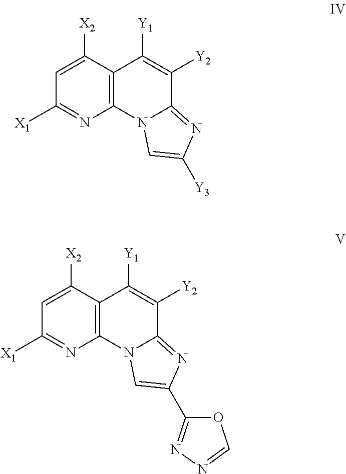

The corresponding 1,8-napthyridines of general formula III may be treated with an alkylating agent (for example -bromopyruvate) in solvent (for example DMF) with heat (for example 80° C.) to afford tricyclic structures of general formula IV (where Y₃═CO₂Et if -ethylbromopyruvate is used as an alkylating agent). Those skilled in the art will recognize alternate alkylating agents (preferably -halo ketones, including, for example, -bromoacetophenone or 2-bromo-1-(furan-2-yl)ethanone) may be employed in this transformation to afford compounds of formula IV where Y₃=phenyl or furyl respectively. Additionally, one skilled in the art will recognize when an alkylating agent is used to afford molecules of general formula IV with Y₃═CO₂Et, the ester functionality may be converted to any of a number of other structures (including, for example, oxazoles or oxadiazoles). For example, by treatment with hydrazine in solvent (for example ethanol) with heat (for example 80° C.) followed by subsequent exposure to a formate ester (for example trimethylorthoformate) with acid (for example p-toluenesulfonic acid) provides molecules of the general formula V. Alternatively, for molecules of general formula IV (Y₃═CO₂Et) may be readily converted to the corresponding aldehyde by treatment with a reducing agent (for example DIBALH) in solvent (for example toluene) with reduced temperature (for example −78° C.). Subsequent conversion to the corresponding oxazole (by treatment with the TOSMIC reagent, for example) can be readily accomplished using protocols well-known to those skilled in the art. Those skilled in the art will recognize an ester functionality may be transformed using standard conditions to numerous other heterocyclic rings.

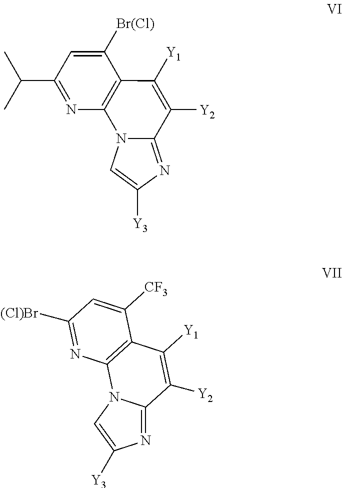

Those skilled in the art will recognize that molecules of general formula IV or V (wherein either X₁ or X₂ or both ═OH) may be converted to the corresponding halides (for example X₁ or X₂ or both ═Cl or Br) via treatment with a halogenating reagents (for example POCl₃ or POBr₃) in solvent (for example acetonitrile) with heat (for example 80° C.) to give, for example, molecules of general formula VI or VII. Aryl halides VI and VII may be transformed using well known chemistries (for example Suzuki, Stille, Negishi, or SNAR displacement chemistries) to afford molecules of the general formula IV or V wherein either X₁ or X₂ or both may be substituted with alkyl, aryl, amino, hydroxyl, or heteraryl functionalities. For example, treatment of molecules of general formula VI using Suzuki conditions including a vinyl boronic acid (for example cyclopentenyl boronic acid), a base (for example potassium carbonate) and a catalyst (for example PdCl₂(dppf)-CH₂Cl₂) in solvent (for example dioxane) followed by reduction of the corresponding olefin with a catalyst (for example palladium on carbon) in solvent (for example THF) under an atmosphere of hydrogen can afford molecules of the general formula IV or V where X₂=cyclopentyl.

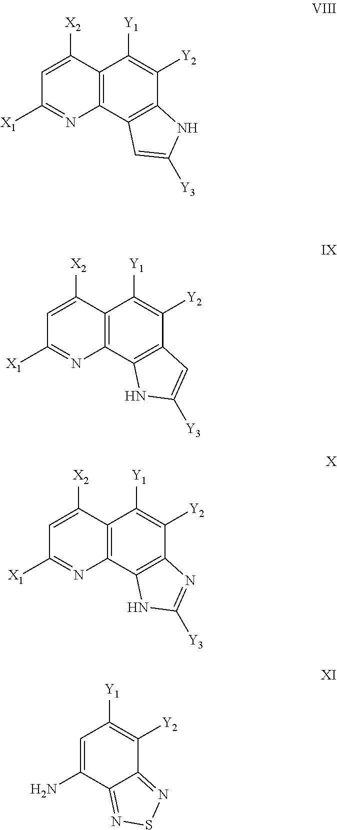

Those skilled in the art will recognize numerous related core structures (including, for example general structures VIII, IX, and X) may be prepared in a manner analogous to that described for the general preparation of structures of general formula IV. For example, treatment of the appropriate indoles with a diketone of general formula II (for example 1, 1,1,5,5,5-hexafluoropentane-2,4-dione) in solvent (for example acetic acid) affords molecules of general formula VIII and XI. Those skilled in the art will recognize molecules of general formula XI serve as more nucleophllic masked amino benzimidazoles, which when treated with a diketone of general formula II (for example 1,1,1,5,5,5-hexafluoropentane-2,4-dione) can be converted to molecules of general formula X using transformations well known to those skilled in the art.

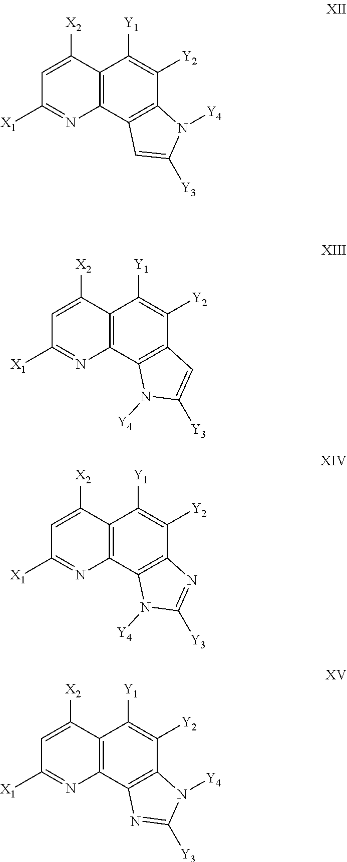

Further substitutions of molecules with general formula VIII, IX, or X with a variety of acylating or alkylating agents are possible using standard conditions known to those skilled in the art. For example, molecules of general formula XII or XIII (where Y₄=acyl group) can be obtained directly from the corresponding indoles by treatment with a base (for example triethylamine) in solvent (for example dichloromethane) and an acylating agent (for example cyclobutanecarbonyl chloride). Similarly, molecules of general formula XII or XIII (where Y₄=alkyl group, for example benzyl) can be obtained via treatment of VIII or IX with a base (for example potassium carbonate) in solvent (for example DMF or MeCN) with an alkylating agent (for example benzyl bromide) and heat (for example 80° C.). Those skilled in the art will recognize treatment of molecules with general formula X using any of the above conditions will afford mixtures of the corresponding acylated or alkylated molecules of general formula XIV or XV. Molecules of general formula XIV or XV can be readily separated using methods well known to those skilled in the art (for example high pressure liquid chromatography).

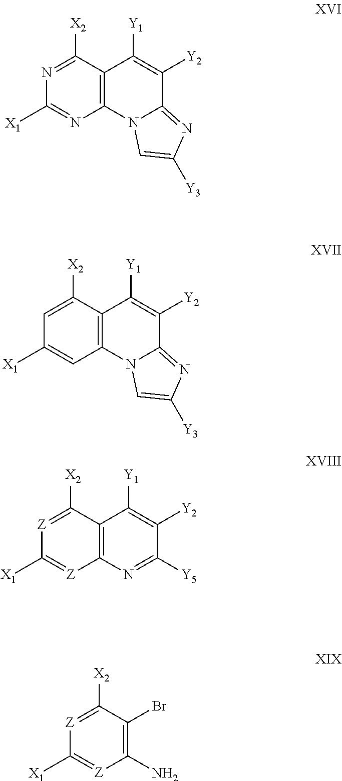

Those skilled in the art will further recognize additional core structures, for example molecules of general formula XVI can be prepared using analogous chemistries. For example treatment of compounds of general formula I with an electron deficient triazine (for example 2, 4,6-tris(trifluoromethyl)-1,3,5-triazine) in solvent, followed by alkylation and derivatization in a manner analogous to that described above, affords molecules of general formula XVI. Similarly, treatment of a functionalized aryl amine of general formula XIX (where Z may be carbon or nitrogen) with an olefin (for example acrolein or acrylonitrile) in the presence of a catalyst (for example Pd(OAc)₂) and ligand (for example triphenylphosphine) followed by exposure to an acid or base (for example acetic acid or piperidine) affords structures of general formula XVIII (where Y₅═O or NH₂). Those skilled in the art will recognize conversion of Y₅═O to the corresponding amino group can be readily accomplished first by treatment with a chlorination reagent (for example POCl₃), subsequent displacement of the derived chloride by an amine (for example p-methoxybenzylamine) and then finally by exposure to acid (for example trifluoroacetic acid). Once in hand, molecules of general formula XVI or XVII may be functionalized in a manner analogous to that described above for related core structures.

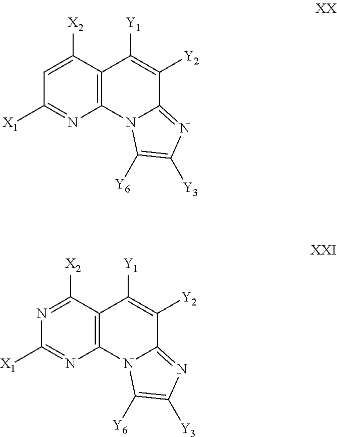

Direct functionalization of molecules of general formula XVI and IV to afford XXI and XX, respectively (for example Y₆═Cl or Br) can be accomplished via direct treatment of XVI or IV with a halogenating reagent (for example NCS or NBS) in solvent (for example DMF or chloroform). Those skilled in the art will recognize that for XX and XXI where Y₆=Br or Cl, a number of additional transformations are possible. For example, treatment of XX (Y₆=Br) under Negishi conditions including a catalyst (for example tetrakistriphenylphosphine palladium) and an organometalic reagent (for example dimethyl zinc) in a solvent (for example THF) with heat (for example 60° C.) will afford molecules of general structure XX wherein Y₆=Me.

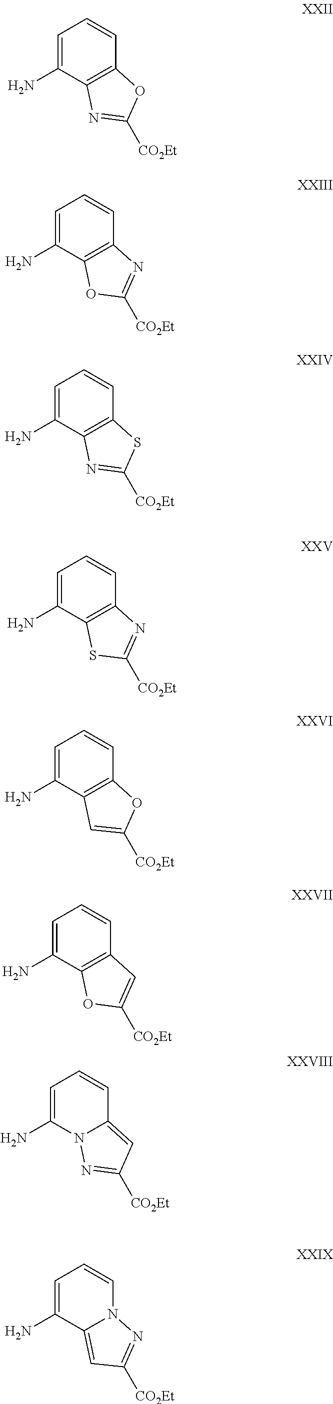

One skilled in the art will recognize numerous related tricyclic core structures may be synthesized via substitution of bicycles XXII-XXIX (or other related bicycles) through a reaction sequence analogous to that described above for the synthesis of VIII, IX or X. One skilled in the art will recognize the various transformations described above may be combined in different combinations or in a different order such that the functional groups present on any given molecule are compatible with the reaction conditions.

### Example 1

### 2-[2,4-bis(trifluoromethyl)imidazol-[1,2-a]1,8-naphthyridin-8-yl]-1,3,4-oxdadlazole

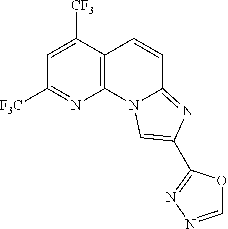 [SMILES: FC(F)(F)c1cc(C(F)(F)F)c2ccc3nc(-c4nnco4)cn3c2n1]

### Step A

### 5,7-bis(trifluoromethyl)-1,8-naphthyridin-2-amine

A mixture of pyridine-2,6-diamine (12 g, 110 mmol) and 1,1,1,5,5,5-hexafluoropentane-2,4-dione (25.2 g, 121 mmol) dissolved in acetic acid (80 mL) was heated at 120° C. under nitrogen for 1 hour. After cooling to room temperature, the reaction mixture was concentrated and then diluted with ice water. The resulting solid was filtered and washed with water to give 5,7-bis(trifluoromethyl)-1,8-naphthyridin-2-amine (23.98 g, 85 mmol, 78% yield) as a grey solid. ES LC-MS m/z=282.10 (M+H)⁺.

### Step B

### ethyl 2,4-bis(trifluoromethyl)imidazo[1,2-a][1,8]naphthyridine-8-carboxylate

To a solution of 20 g 5,7-bis(trifluoromethyl)-1,8-naphthyridin-2-amine in N,N-dimethylformamide (80 mL) was added ethyl 3-bromo-2-oxopropanoate (22.4 mL, 177 mmol) (2.5 eq) and the reaction mixture was heated at 68° C. under nitrogen for 3 h. The mixture was cooled room temperature, diluted with large quality of water and the resulting solid was filtered, and washed with water to give ethyl 2,4-bis(trifluoromethyl)imidazo[1,2-a][1,8]naphthyridine-8-carboxylate (13.55 g, 35.9 mmol, 32.7% yield) as a yellow brown solid, yield 50.5%. ES LC-MS m/z=378.20 (M+H)⁺,

### Step C

### 2,4-bis(trifluoromethyl)imidazo[1,2-a][1,8]naphthyridine-8-carbohydrazide

A solution of ethyl 2,4-bis(trifluoromethyl)imidazo[1,2-a][1,8]naphthyridine-8-carboxylate (25.5 g, 67.6 mmol) and hydrazine (42.4 mL, 1352 mmol) in ethanol (200 mL) was stirred at 65° C. for 2 hours. The mixture was cooled room temperature, and the precipitate was filtered off and washed with water to give 2,4-bis(trifluoromethyl)imidazo[1,2-a][1,8]naphthyridine-8-carbohydrazide (20.2 g, 55.6 mmol, 82% yield). ES LC-MS m/z=364.20 (M+H)⁺.

### Step D

### 2-[2,4-bis(trifluoromethyl)imidazo[1,2-a]1,8-naphthyridin-8-yl]-1,3,4-oxadiazole

A solution of 2,4-bis(trifluoromethyl)imidazo[1,2-a][1,8]naphthyridine-8-carbohydrazide (19.5 g, 53.7 mmol) and tosic acid (5.11 g, 26.8 mmol) in trimethylorthoformate (5.93 ml, 53.7 mmol) was stirred with heating at 70° C. for 4 hours. The solution was cooled to room temperature and most of the solvent was evaporated. The resulting slurry was filtered and the filter cake was washed with water to give 2-[2,4-bis(trifluoromethyl)imidazo[1,2-a]1,8-naphthyridin-8-yl]-1,3,4-oxadiazole (12.4 g, 33.2 mmol, 61.9% yield). ¹H NMR (400 MHz, DMSO-d₆) ppm 8.00 (dd, 1H) 8.14 (d, J=9.76 Hz, 1H) 8.53 (s, 1H) 9.23 (s, 1H) 9.46 (s, 1H);). ES LC-MS m/z=374.15 (M+H)⁺

### Example 2

### 2-(2-cyclopentyl-4-(trifluoromethyl)imidazo[1,2-a][1,8]naphthyridin-8-yl)-1,3,4-oxadiazole

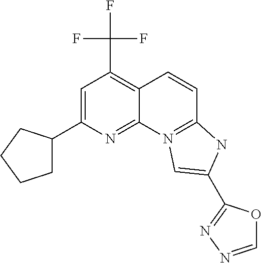

### Step A

### 7-amino-4-(trifluoromethyl)-1,8-naphthyridin-2(1H)-one

A mixture of ethyl 4,4,4-trifluoro-3-oxobutanoate (14.2 g, 77 mmol) and 2,6-diaminopyridine (6 g, 55 mmol) in diphenyl ether (80 mL) was heated to 130° C. for 2 h, and then 190° C. for 18 h. The reaction was cooled to rt and diluted with hexanes, solids filtered and dried to afford the title compound (12.2 g, 97%). LC-MS: ESI (M+H)⁺ m/z=230.13.

### Step B

### ethyl 2-oxo-4-(trifluoromethyl)-1,2-dihydroimidazo[1,2-a]-1,8-naphthyridine-8-carboxylate

To a suspension of 7-amino-4-(trifluoromethyl)-1,8-naphthyridin-2(1H)-one (12.2 g, 53.2 mmol) in anhydrous DMF (180 mL) was added ethyl 3-bromo-2-oxopropanoate (11.4 g, 58.6 mmol) and the mixture heated to 60° C. for 18 h under nitrogen. The solvent was removed in vacuo and the residue partitioned between ethyl acetate and water. The aqueous layer was extracted with ethyl acetate and the combined organic layers dried (MgSO₄) and concentrated in vacuo. The residue was triturated in dichloromethane and the solids filtered and dried to afford the title compound (5.97 g, 34% yield). LC-MS: ESI (M+H)⁺ m/z=326.19.

### Step C

### 8-(1,3,4-oxadiazol-2-yl)-4-(trifluoromethyl)imidazo[1,2-a]-1,8-naphthyridin-2(1H)-one

To a suspension of ethyl 2-oxo-4-(trifluoromethyl)-1,2-dihydroimidazo[1,2-a]-1,8-naphthyridine-8-carboxylate (2 g, 6.2 mmol) in ethanol was added hydrazine (3.9 g, 123 mmol) and the reaction heated to reflux for 18 h under nitrogen. The reaction was cooled to room temperature, and the solids were filtered and dried. The solids were suspended in triethyl orthoformate (25 mL), and p-toluenesulfonic acid monohydrate (0.59 g, 3.1 mmol) was added and the reaction heated to 85° C. for 2 h. The reaction mixture was filtered without cooling and the solids dried to afford the title compound (1.48 g, 75% yield). LC-MS: ESI (M+H)⁺ m/z=321.94.

### Step D

### 2-chloro-8-(1,3,4-oxadiazol-2-yl)-4-(trifluoromethyl)imidazo[1,2-a]-1,8-naphthyridine

A mixture of 8-(1,3,4-oxadiazol-2-yl)-4-(trifluoromethyl)imidazo[1,2-a]-1,8-naphthyridin-2(1H)-one (1.28 g. 4.0 mmol) and phosphorus oxytrichloride (13 mL) was heated to 100° C. under nitrogen for 1 h. The POCl₃ was removed in vacuo and the residue stirred with water for 5 min and neutralized with potassium carbonate until the solution gave blue pH paper. The solution was extracted twice with dichloromethane and the organic layer dried (MgSO₄) and concentrated in vacuo. The residue was triturated with ether and the solids filtered and dried to afford the title compound (774 mg, 57% yield). LC-MS: ESI (M+H)⁺ m/z=340.12.

### Step E

### 2-(2-cyclopentyl-4-(trifluoromethyl)imidazo[1,2-a][1,8]naphthyridin-8-yl)-1,3,4-oxadiazole

A mixture of 2-chloro-8-(1,3,4-oxadiazol-2-yl)-4-(trifluoromethyl)imidazo[1,2-a]-1,8-naphthyridine (85 mg, 0.25 mmol) and PdCl₂(dppf)-CH₂Cl₂ (20 mg, 0.025 mmol) in anhydrous dioxane (2 mL) was degassed with nitrogen. To the solution was added cyclopentylzinc bromide as a 0.5 M solution in THF (0.6 mL) and the reaction heated to 80° C. in a sealed tube for 1 h, then 100° C. for 1 h. The reaction was treated with water and the resulting mixture partitioned between ethyl acetate and water. The organic layer was washed with brine, dried (MgSO₄) and concentrated in vacuo. The residue was purified by silica gel chromatography eluting with 20-100% hexanes/ethyl acetate to afford the title compound (5 mg, 5% yield). LC-MS: ESI (M+H)⁺ m/z=374.29. ¹H NMR (400 MHz, DMSO-d₆) d ppm 9.43 (s, 1H), 9.13-9.29 (m, 1H), 8.03 (s, 1H), 7.79-7.95 (m, 2H), 3.45-3.68 (m, 1H), 2.15 (br. s., 2H), 1.82-2.08 (m, 3H), 1.60-1.81 (m, 2H), 1.23 (br. s., 1H).

### Example 3

### 2-[2-(propan-2-yl)-4-(trifluoromethyl)imidazo[1,2-a]1,8-naphthyridin-8-yl]-1,3,4-oxadiazole

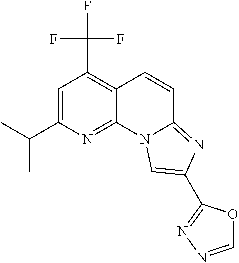 [SMILES: CC(C)c1cc(C(F)(F)F)c2ccc3nc(-c4nnco4)cn3c2n1]

Prepared from 2-chloro-8-(1,3,4-oxadiazol-2-yl)-4-(trifluoromethyl)imidazo[1,2-a]-1,8-naphthyridine in a manner similar as described in example 2, step E. LC-MS: ESI (M+H)⁺ m/z=348.25. ¹H NMR (400 MHz, DMSO-d₆) d ppm 9.43 (s, 1H), 9.23 (s, 1H), 8.05 (s, 1H), 7.78-7.96 (m, 2H), 3.37-3.48 (m, 1H), 1.33-1.50 (m, 6H).

### Example 4

### 5-[2,4-bis(trifluoromethyl)imidazo[1,2-a]1,8-naphthyridin-8-yl]-1,3-oxazole

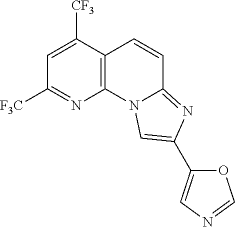 [SMILES: FC(F)(F)c1cc(C(F)(F)F)c2ccc3nc(-c4cnco4)cn3c2n1]

### Step A

### 2,4-bis(trifluoromethyl)imidazo[1,2-a][1,8]naphthyridine-8-carbaldehyde

To a solution of ethyl 2,4-bis(trifluoromethyl)imidazo[1,2-a][1,8]naphthyridine-8-carboxylate (500 mg, 1.325 mmol) in dichloromethane (15 mL) stirred under nitrogen at −78° C. was added DIBAL-H (1.0M solution) (3.98 mL, 3.98 mmol) dropwise over 30 minutes. After 2 hours at −78° C., the reaction was quenched with methanol at −78° C. Then the reaction mixture was allowed to warm to 0° C. and treated with a saturated solution of Rochelle's salt (100 mL). The resulting mixture was extracted with DCM (emulsion formed was filtered over Celite to remove white gummy precipitate). The combined extracts were concentrated under vacuum and the residue was purified via silica gel chromatography (0-5% MeOH/DCM) to give 2,4-bis(trifluoromethyl)imidazo[1,2-a][1,8]naphthyridine-8-carbaldehyde (293 mg, 0.835 mmol, 63.0% yield) as a light brown solid. ES LC-MS m/z=334.20 (M+H)⁺,

### Step B

### 5-[2,4-bis(trifluoromethyl)imidazo[1,2-a]1,8-naphthyridin-8-yl]-1,3-oxazole

To a mixture of 2,4-bis(trifluoromethyl)imidazo[1,2-a][1,8]naphthyridine-8-carbaldehyde (100 mg, 0.300 mmol) and TOSMIC reagent (58.6 mg, 0.300 mmol) in methanol (4 mL) was added K₂CO₃ (41.5 mg, 0.300 mmol). The solution was refluxed for 2 hours, and the solvent was evaporated under reduced pressure. The residue was poured into ice water and extracted with DCM. The organic layer was washed consecutively with 1% HCl, followed by water, and concentrated to dryness. The crude material was purified via silica gel chromatography (0-5% MeOH/DCM) to give 5-[2,4-bis(trifluoromethyl)imidazo[1,2-a]1,8-naphthyridin-8-yl]-1,3-oxazole (84.1 mg, 0.215 mmol, 71.5% yield) as a yellow solid.: ¹H NMR (400 MHz, DMSO-d₆, δ ppm 7.80 (s, 1H) 7.93 (dd, J=9.85, 1.85 Hz, 1H) 8.08 (d, J=9.76 Hz, 1H) 8.47 (s, 1H) 8.57 (s, 1H) 8.96 (s, 1H); ES LC-MS m/z=373.22 (M+H)⁺.

### Example 5

### 2-[2-cyclopropyl-4-(trifluoromethyl)imidazo[1,2-a]1,8-naphthyridin-8-yl]-1,3,4-oxadiazole

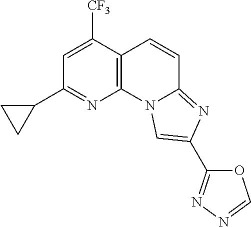 [SMILES: FC(F)(F)c1cc(C2CC2)nc2c1ccc1nc(-c3nnco3)cn12]

To a mixture of 2-(2-chloro-4-(trifluoromethyl)imidazo[1,2-a][1,8]naphthyridin-8-yl)-1,3,4-oxadiazole (34 mg, 0.100 mmol) and Pd(Ph₃P)₄ (11.57 mg, 10.01 μmol) dissolved in N,N-dimethylformamide (2 mL) was added cyclopropylzinc(II) bromide (0.400 mL, 0.200 mmol) dropwise. The reaction mixture was heated at 60° C. for 45 minutes under nitrogen, and the crude reaction mixture was purified via reverse phase HPLC to give 2-[2-cyclopropyl-4-(trifluoromethyl)imidazo[1,2-a]1,8-naphthyridin-8-yl]-1,3,4-oxadiazole (11.6 mg, 0.032 mmol, 31.9% yield) as a yellow solid. ¹H NMR (400 MHz, DMSO-d₆ δ: ppm 1.18-1.32 (m, 2H) 1.31-1.41 (m, 2H) 2.52-2.62 (m, 1H) 7.84 (s, 2H) 8.12 (s, 1H) 9.17 (s, 1H) 9.42 (s, 1H); ES LC-MS m/z=346.24 (M+H)⁺.

### Example 6

### 2-[2-(thiophen-3-yl)-4-(trifluoromethyl)imidazo[1,2-a]1,8-naphthyridin-8-yl]-1,3,4-oxadiazole

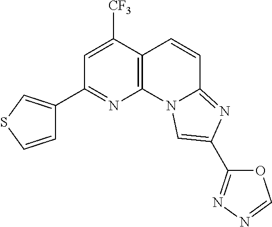 [SMILES: FC(F)(F)c1cc(-c2ccsc2)nc2c1ccc1nc(-c3nnco3)cn12]

To a mixture of 2-(2-chloro-4-(trifluoromethyl)imidazo[1,2-a][1,8]naphthyridin-8-yl)-1,3,4-oxadiazole (100 mg, 0.294 mmol), thiophen-3-ylboronic acid (75 mg, 0.589 mmol) and PdCl₂(dppf)-CH₂Cl₂ adduct (24.04 mg, 0.029 mmol) dissolved in N,N-dimethylacetamide (3 mL) was added Na₂CO₃ (0.883 mL, 0.883 mmol) and the reaction mixture was heated at 80° C. under nitrogen for 1 hour. The reaction mixture was cooled to room temperature, diluted with water, and extracted with DCM. The combined organic layer was washed consecutively with water, followed by saturated NaCl, and then concentrated to dryness. The residue was purified via silica gel chromatography (0-5% MeOH/DCM) to give 2-[2-(thiophen-3-yl)-4-(trifluoromethyl)imidazo[1,2-a]1,8-naphthyridin-8-yl]-1,3,4-oxadiazole (44 mg, 0.108 mmol, 36.7% yield) as a light yellow solid. ¹H NMR (400 MHz, DMSO-d₆ δ: ppm 7.79 (dd, 1H) 7.88 (s, 2H) 8.29 (dd, J=5.07, 0.98 Hz, 1H) 8.52 (s, 1H) 8.96 (d, J=1.76 Hz, 1H) 9.44 (s, 1H) 9.59 (s, 1H); ES LC-MS m/z=388.20 (M+H)⁺.

### Example 7

### 2-[2-methyl-4-(trifluoromethyl)imidazo[1,2-a]1,8-naphthyridin-8-yl]-1,3,4-oxadiazole

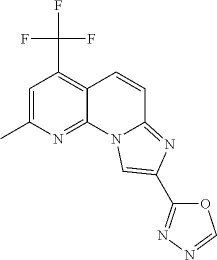 [SMILES: Cc1cc(C(F)(F)F)c2ccc3nc(-c4nnco4)cn3c2n1]

Prepared from 2-chloro-8-(1,3,4-oxadiazol-2-yl)-4-(trifluoromethyl)imidazo[1,2-a]-1,8-naphthyridine in a manner similar as described in example 2, step E. LC-MS: ESI (M+H)⁺ m/z=320.22. ¹H NMR (400 MHz, DMSO-d₆) d ppm 9.42 (s, 1H), 9.16 (s, 1H), 8.06 (s, 1H), 7.82-7.96 (m, 2H), 2.84 (s, 3H).

### Example 8

### 2-[2,4-bis(trifluoromethyl)-7H-pyrrolo[2,3-h]quinolin-8-yl]-1,3,4-oxadiazole

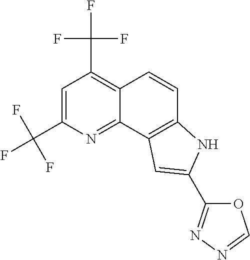 [SMILES: FC(F)(F)c1cc(C(F)(F)F)c2ccc3[nH]c(-c4nnco4)cc3c2n1]

### Step A

### ethyl 4-amino-1H-indole-2-carboxylate

To a solution of ethyl 4-nitro-1H-indole-2-carboxylate (1.7 g, 7.3 mmol) in ethanol was added Raney nickel and the reaction hydrogenated at 60 psi at room temperature for 1.5 h. The reaction was filtered through celite and concentrated in vacuo to afford the title compound (1.38 g, 93% yield). LC-MS: ESI (M+H)⁺ m/z=205.46.

### Step B

### ethyl 2,4-bis(trifluoromethyl)-7H-pyrrolo[2,3-h]quinoline-8-carboxylate

A solution of ethyl 4-amino-1H-indole-2-carboxylate (1.0 g, 4.9 mmol) and 1,1,1,5,5,5-hexafluoropentane-2,4-dione (1.5 g, 7.3 mmol) in acetic acid (23 mL) was heated to 100° C. for 3 h. The reaction was cooled to room temperature, diluted with ethyl acetate, washed with water, 10% aqueous potassium carbonate solution and brine, dried (MgSO₄) and concentrated in vacuo. The residue was triturated in methanol and the solids were filtered and dried to afford the title compound (1.17 g, 64% yield). LC-MS: ESI (M+H)⁺ m/z=376.92.

### Step C

### 2-[2,4-bis(trifluoromethyl)-7H-pyrrolo[2,3-h]quinolin-8-yl]-1,3,4-oxadiazole

Ethyl 2,4-bis(trifluoromethyl)-7H-pyrrolo[2,3-h]quinoline-8-carboxylate (1.17 g, 3.1 mmol) was suspended in ethanol (30 mL) and hydrazine (1.95 mL, 62.2 mmol) was added and the reaction heated to reflux for 18 h. The reaction was cooled to room temperature and the solids were filtered and dried. The solids were suspended in triethyl orthoformate (18 mL) and p-toluenesulfonic acid monohydrate (296 mg, 1.56 mmol) was added and the reaction heated to 85° C. for 1.5 h, and the reaction mixture filtered without cooling. The solids were dried to afford the title compound (990 mg, 86% yield). LC-MS: ESI (M+H)⁺ m/z=372.97. ¹H NMR (400 MHz, DMSO-d₆) d ppm 13.47 (br. s., 1H), 9.46 (s, 1H), 8.26 (s, 1H), 8.10-8.19 (m, 1H), 8.02 (d, J=9.2 Hz, 1H), 7.89 (s, 1H).

### Example 9

### 2-[9-methyl-2,4-bis(trifluoromethyl)imidazo[1,2-a]1,8-naphthyridin-8-yl]-1,3,4-oxadiazole

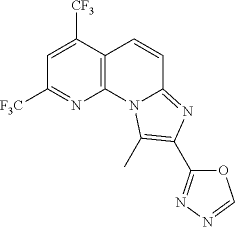 [SMILES: Cc1c(-c2nnco2)nc2ccc3c(C(F)(F)F)cc(C(F)(F)F)nc3n12]

### Step A

### 2-(9-bromo-2,4-bis(trifluoromethyl)imidazo[1,2-a][1,8]naphthyridin-8-yl)-1,3,4-oxadiazole

A solution of 2-(2,4-bis(trifluoromethyl)imidazo[1,2-a][1,8]naphthyridin-8-yl)-1,3,4-oxadiazole (1.5 g, 4.02 mmol) and NBS (1.431 g, 8.04 mmol) in N,N-dimethylformamide (4 mL) was stirred with heating at 60° C. for 1 hour. Water was added and the precipitate was filtered off to give 2-(9-bromo-2,4-bis(trifluoromethyl)imidazo[1,2-a][1,8]naphthyridin-8-yl)-1,3,4-oxadiazole (1.69 g, 3.55 mmol, 88% yield). ES LC-MS m/z=452.13 (Br⁷⁹, M+H)⁺, ES LC-MS m/z=454.10 (Br⁸¹, M+H)⁺.

### Step B

### 2-[9-methyl-2,4-bis(trifluoromethyl)imidazo[1,2-a]1,8-naphthyridin-8-yl]-1,3,4-oxadiazole

A solution of 2-(9-bromo-2,4-bis(trifluoromethyl)imidazo[1,2-a][1,8]naphthyridin-8-yl)-1,3,4-oxadiazole (100 mg, 0.221 mmol), 2,4,6-trimethyl-1,3,5,2,4,6-trioxatriborinane (278 mg, 0.221 mmol), PdCl₂(dppf)-CH₂Cl₂ adduct (18.06 mg, 0.022 mmol) and sodium carbonate (0.332 mL, 0.664 mmol, 1.0 M solution) in N,N-dimethylacetamide (5.0 mL) was heated at 60° C. for 1 hour. The crude reaction mixture was purified via reverse phase HPLC to give 2-[9-methyl-2,4-bis(trifluoromethyl)imidazo[1,2-a]1,8-naphthyridin-8-yl]-1,3,4-oxadiazole (7.2 mg, 0.018 mmol, 7.99% yield): ¹H NMR (400 MHz, DMSO-d₆, δ ppm 3.35 (s, 3H) 7.91 (d, J=9.76 Hz, 1H) 8.08 (d, J=9.76 Hz, 1H) 8.50 (s, 1H) 9.43 (s, 1H); ES LC-MS m/z=388.24 (M+H)⁺.

### Example 10

### 2-[2-ethoxy-4-(trifluoromethyl)imidazo[1,2-a]1,8-naphthyridin-8-yl]-1,3,4-oxadiazole

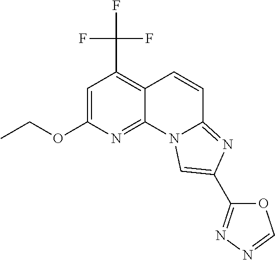 [SMILES: CCOc1cc(C(F)(F)F)c2ccc3nc(-c4nnco4)cn3c2n1]

To a solution of 2-chloro-8-(1,3,4-oxadiazol-2-yl)-4-(trifluoromethyl)imidazo[1,2-a]-1,8-naphthyridine (example 2, step D) (50 mg, 0.15 mmol) in ethanol (1 mL) was added sodium ethoxide (21 wt % in ethanol, 0.07 mL, 0.18 mmol) and the reaction stirred at room temperature for 45 min and then at 50° C. for 30 min. The reaction was cooled to room temperature, poured into ethyl acetate and washed with water, dried (MgSO₄) and concentrated in vacuo. The residue was purified by silica gel chromatography eluting with 50-100% hexanes/ethyl acetate to afford the title compound (19 mg, 31% yield). LC-MS: ESI (M+H)⁺ m/z=349.83. ¹H NMR (400 MHz, DMSO-d₆) d ppm 9.42 (s, 1H), 9.21 (s, 1H), 7.72-7.90 (m, 2H), 7.56 (s, 1H), 4.71 (q, J=7.0 Hz, 2H), 1.46 (t, J=7.0 Hz, 3H).

### Example 11

### 4-(1,3,4-oxadiazol-2-yl)-10,12-bis(trifluoromethyl)-2,5,11,13-tetraazatricyclo[7.4.0.0²,⁶]trideca-1(9),3,5,7,10,12-hexaene

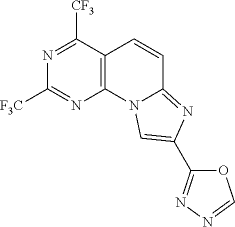 [SMILES: FC(F)(F)c1nc(C(F)(F)F)c2ccc3nc(-c4nnco4)cn3c2n1]

### Step A

### 2,4-bis(trifluoromethyl)pyrido[2,3-d]pyrimidin-7-amine

A solution of pyridine-2,6-diamine (1.5 g, 13.75 mmol) in AcOH (64.8 ml) was cooled to 0 deg and treated by the drop wise addition of 2,4,6-tris(trifluoromethyl)-1,3,5-triazine (3.89 ml, 13.75 mmol). The bath was removed and the reaction was heated to 80° C. overnight. After cooling to room temperature, the solvents were removed under reduced pressure and the residue was taken up in DCM and basified with 1N NaOH. The combined organics were washed with saturated NaHCO₃ (3×), brine, dried over Na₂SO₄, filtered, and concentrated to give 2,4-bis(trifluoromethyl)pyrido[2,3-d]pyrimidin-7-amine (3.77 g, 13.36 mmol, 97% yield) as a red solid. ES LC-MS m/z=283.11 (M+H)⁺.

### Step B

### ethyl 2,4-bis(trifluoromethyl)imidazo[1′,2′:1,6]pyrido[2,3-d]pyrimidine-8-carboxylate

A solution of 2,4-bis(trifluoromethyl)pyrido[2,3-d]pyrmidin-7-amine (2.0 g, 7.09 mmol) in DMF (33.2 ml) was treated with ethyl borompyruvate (2.230 ml, 17.72 mmol). The reaction was heated to 80° C. overnight. The black reaction was concentrated under reduced pressure to remove most of the DMF. The residue was diluted with H₂O and the solids were filtered to give ethyl 2,4-bis(trifluoromethyl)imidazo[1′,2′:1,6]pyrido[2,3-d]pyrimidine-8-carboxylate (2.45 g, 6.48 mmol, 91% yield) as a brown solid. ES LC-MS m/z=379.14 (M+H)⁺.

### Step C

### 2,4-bis(trifluoromethyl)imidazo[1′,2′:1,6]pyrido[2,3-d]pyrimidine-8-carbohydrazide

A solution of ethyl 2,4-bis(trifluoromethyl)imidazo[1′,2′:1,6]pyrido[2,3-d]pyrimidine-8-carboxylate (0.5 g, 1.322 mmol) and hydrazine (0.830 ml, 26.4 mmol) in EtOH (5.78 ml) was heated to reflux for 30 minutes The reaction was concentrated under reduced pressure to give 2,4-bis(trifluoromethyl)imidazo[1′,2′:1,6]pyrido[2,3-d]pyrimidine-8-carbohydrazide (0.481 g, 1.321 mmol, 100% yield) as a dark red/brown oil. ES LC-MS m/z=365.1 (M+H)⁺.

### Step D

### 2-(2,4-bis(trifluoromethyl)imidazo[1′,2′:1,6]pyrido[2,3-d]pyrimidin-8-yl)-1,3,4-oxadiazole

A solution of 2,4-bis(trifluoromethyl)imidazo[1′,2′:1,6]pyrido[2,3-d]pyrimidine-8-carbohydrazide (0.481 g, 1.321 mmol), TsOH (0.100 g, 0.528 mmol), and triethyl orthoformate (8.80 ml, 52.8 mmol) was heated at 80° C. under nitrogen overnight. After cooling to room temperature, the solvents were removed under reduced pressure and the residue was treated by water. The solution was extracted with EtOAc. The combined extracts were washed with brine, dried over Na₂SO₄, filtered, and concentrated. The residue was taken up in DMF and purified by reverse phase chromatography (10-90% ACN/H₂O+formic acid), then lyophilized to give 4-(1,3,4-oxadiazol-2-yl)-10,12-bis(trifluoromethyl)-2,5,11,13-tetraazatricyclo[7.4.0.0²,⁶]trideca-1(9),3,5,7,10,12-hexaene (0.0436 g, 0.117 mmol, 8.82% yield) as a solid. ¹H NMR (400 MHz, DMSO-d₆) δ ppm 9.51 (s, 3H), 9.39 (s, 1H), 8.07-8.28 (m, 2H), ES LC-MS m/z=375.2 (M+H)⁺.

### Example 12

### 2-[2-(furan-3-yl)-4-(trifluoromethyl)imidazo[1,2-a]1,8-naphthyridin-8-yl]-1,3,4-oxadiazole

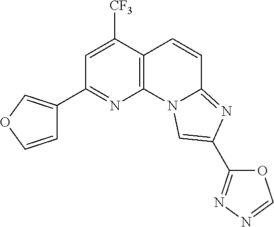 [SMILES: FC(F)(F)c1cc(-c2ccoc2)nc2c1ccc1nc(-c3nnco3)cn12]

To a mixture of 2-(2-chloro-4-(trifluoromethyl)imidazo[1,2-a][1,8]naphthyridin-8-yl)-1,3,4-oxadiazole (50 mg, 0.147 mmol) and furan-3-ylboronic acid (32.9 mg, 0.294 mmol) dissolved in 1,4-dioxane (2 mL) was added potassium phosphate tribasic (94 mg, 0.442 mmol) and PdCl₂(dppf)-CH₂Cl₂ adduct (12.02 mg, 0.015 mmol). The reaction vessel was sealed under nitrogen and heated in a Biotage Microwave Initiator at 160° C. for 30 minutes. This reaction mixture was submitted to the microwave conditions 7 times to ensure full conversion of the starting materials. The mixture was concentrated and the residue was purified via reverse phase HPLC to give 2-[2-(furan-3-yl)-4-(trifluoromethyl)imidazo[1,2-a]1,8-naphthyridin-8-yl]-1,3,4-oxadiazole (10.2 mg, 0.026 mmol, 17.73% yield) as a yellow solid. ¹H NMR (400 MHz, DMSO-d₆ δ: ppm 7.62 (s, 1H) 7.82-8.00 (m, 3H) 8.41 (s, 1H) 9.02 (s, 1H) 9.45 (s, 1H) 9.58 (s, 1H); ES LC-MS m/z=272.23 (M+H)⁺,

### Example 13

### 2-[2-ethyl-4-(trifluoromethyl)imidazo[1,2-a]1,8-naphthyridin-8-yl]-1,3,4-oxadiazole

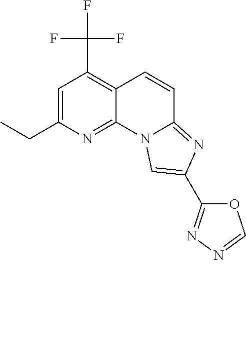 [SMILES: CCc1cc(C(F)(F)F)c2ccc3nc(-c4nnco4)cn3c2n1]

Prepared from 2-chloro-8-(1,3,4-oxadiazol-2-yl)-4-(trifluoromethyl)imidazo[1,2-a]-1,8-naphthyridine in a manner similar as described in example 2, step E. LC-MS: ESI (M+H)⁺ m/z=334.18. ¹H NMR (400 MHz, DMSO-d₆) d ppm 9.43 (s, 1H), 9.20 (s, 1H), 8.05 (s, 1H), 7.79-7.96 (m, 2H), 3.13 (q, J=7.4 Hz, 2H), 1.43 (t, J=7.5 Hz, 3H).

### Example 14

### 2-[1-benzyl-6,8-bis(trifluoromethyl)-1H-pyrrolo[3,2-h]quinolin-2-yl]-1,3,4-oxadiazole

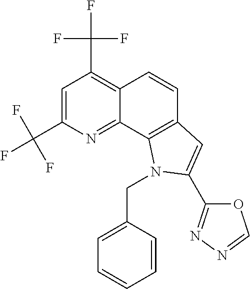 [SMILES: FC(F)(F)c1cc(C(F)(F)F)c2ccc3cc(-c4nnco4)n(Cc4ccccc4)c3c2n1]

### Step A

### ethyl 7-amino-1H-indole-2-carboxylate

A solution of ethyl 7-nitro-1H-indole-2-carboxylate (3.84 g, 16.40 mmol) in tetrahydrofuran (175 mL) was treated dropwise with sodium hydrosulfite (sodium dithionite) (14.26 g, 82 mmol) as a solution in water (175 mL). The mixture was maintained with stirring for 4 hours, diluted with ethyl acetate, and the organic layer washed three times with water. The organic layer was separated, dried over sodium sulfate, filtered, and concentrated to afford ethyl 7-amino-1H-indole-2-carboxylate (1.27 g, 6.22 mmol, 37.9% yield) as a yellow solid.

### Step B

### ethyl 6,8-bis(trifluoromethyl)-1H-pyrrolo[3,2-h]quinoline-2-carboxylate

A solution of ethyl 7-amino-1H-indole-2-carboxylate (3.40 g, 16.65 mmol) and 1,1,1,5,5,5-hexafluoropentane-2,4-dione (3.53 mL, 24.97 mmol) in acetic acid (60 mL) was maintained in a sealed pressure tube at 11° C. for 3 hours. The mixture was cooled, concentrated, suspended in DCM, and washed with saturated sodium bicarbonate. The organic layer was separated, dried over sodium sulfate, filtered, concentrated, and purified by column chromatography to afford ethyl 6,8-bis(trifluoromethyl)-1H-pyrrolo[3,2-h]quinoline-2-carboxylate (3.7 g, 9.83 mmol, 59.1% yield) as a yellow solid. LC-MS: ESI (M+H)⁺ m/z=377.22.

### Step C

### 2-(1,3,4-oxadiazol-2-yl)-6,8-bis(trifluoromethyl)-1H-pyrrolo[3,2-h]quinoline

Prepared in a manner similar as described in example 8 step C. LC-MS: ESI (M+H)⁺ m/z=373.01.

### Step D

### 2-(1-benzyl-6,8-bis(trifluoromethyl)-1H-pyrrolo[3,2-h]quinolin-2-yl)-1,3,4-oxadiazole

To a solution of 2-(1,3,4-oxadiazol-2-yl)-6,8-bis(trifluoromethyl)-1H-pyrrolo[3,2-h]quinoline (50 mg, 0.13 mmol) and potassium carbonate (37 mg, 0.27 mmol) in anhydrous DMF (1 mL) was added benzyl bromide (35 mg, 0.2 mmol) and the reaction stirred at room temperature for 1 h. The reaction was poured into ethyl acetate, washed with water, brine, dried (MgSO₄) and concentrated in vacuo. The residue was purified by silica gel chromatography eluting with 5-50% hexanes/ethyl acetate to afford the title compound (50 mg, 79% yield). LC-MS: ESI (M+H)⁺ m/z=463.08. ¹H NMR (400 MHz, DMSO-d₆) δ ppm 9.47 (s, 1H), 8.37 (d, J=9.0 Hz, 1H), 8.28 (s, 1H), 7.91 (dd, J=9.0, 2.0 Hz, 1H), 7.82 (s, 1H), 7.04-7.26 (m, 3H), 6.93 (d, J=7.2 Hz, 2H), 6.86 (s, 2H).

### Example 15

### 2-[6,8-bis(trifluoromethyl)-1H-pyrrolo[3,2-h]quinolin-2-yl]-1,3,4-oxadiazole

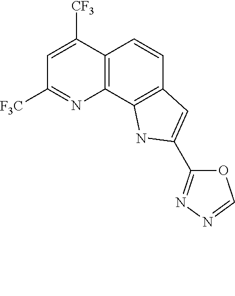 [SMILES: FC(F)(F)c1cc(C(F)(F)F)c2ccc3cc(-c4nnco4)[nH]c3c2n1]

### Step A

### ethyl 6,8-bis(trifluoromethyl)-1H-pyrrolo[3,2-h]quinoline-2-carboxylate

To a mixture of ethyl 7-nitro-1H-indole-2-carboxylate (1 g, 4.27 mmol) in methanol (10 mL) and ethyl acetate (10.00 mL) was added Pd/C (100 mg, 0.094 mmol) and the reaction mixture was hydrogenated for 7 hours at room temperature under 50-60 psi H₂ gas. The reaction mixture was filtered through a pad of celite and the filtrate was concentrated to dryness to give. ethyl 7-amino-1H-indole-2-carboxylate (848 mg) as a brown solid. A mixture of the crude ethyl 7-amino-1H-indole-2-carboxylate (848 mg, 4.15 mmol) and 1,1,1,5,5,5-hexafluoropentane-2,4-dione (0.888 g, 4.27 mmol) in acetic acid (10.00 mL) was heated at 120° C. under nitrogen for 1 hour. The reaction mixture was concentrated to remove the acetic acid, and the residue was diluted with water and DCM and basified to pH 8-9 with concentrated ammonium hydroxide. The organic layers were separated, washed consecutively with water and saturated NaCl, and concentrated to dryness. The residue was purified via silica gel chromatography (0-20% Hexane/EtOAc) to give ethyl 6,8-bis(trifluoromethyl)-1H-pyrrolo[3,2-h]quinoline-2-carboxylate (945 mg, 2.51 mmol, 58.8% yield) as a yellow solid. ES LC-MS m/z=376.99 (M+H)⁺,

### Step B

### 2-[6,8-bis(trifluoromethyl)-1H-pyrrolo[3,2-h]quinolin-2-yl]-1,3,4-oxadiazole

A mixture of ethyl 6,8-bis(trifluoromethyl)-1H-pyrrolo[3,2-h]quinoline-2-carboxylate (200 mg, 0.532 mmol) and hydrazine (0.334 mL, 10.63 mmol) in ethanol (5 mL) was refluxed under nitrogen for 20 hours. The reaction mixture was concentrated to dryness to give, 6,8-bis(trifluoromethyl)-1H-pyrrolo[3,2-h]quinoline-2-carbohydrazide (190 mg) as a light yellow solid, which was used directly in the following step. A mixture of crude 6,8-bis(trifluoromethyl)-1H-pyrrolo[3,2-h]quinoline-2-carbohydrazide (190 mg, 0.525 mmol) and TsOH (50 mg, 0.263 mmol) in triethylorthoformate (6 mL, 36.0 mmol) was heated at 80° C. under nitrogen for 1 hour. The reaction mixture was concentrated to dryness and the residue was purified via reverse phase HPLC to give 2-[6,8-bis(trifluoromethyl)-1H-pyrrolo[3,2-h]quinolin-2-yl]-1,3,4-oxadiazole (45 mg, 0.115 mmol, 21.61% yield) as a light yellow solid. ¹H NMR (400 MHz, DMSO-d₆ δ: ppm 7.62 (s, 1H) 7.84 (dd, J=8.98, 1.95 Hz, 1H) 8.16-8.47 (m, 2H) 9.47 (s, 1H) 13.99 (s, 1H); ES LC-MS m/z=473.22 (M+H)⁺.

### Example 16

### 2-[1-methyl-6,8-bis(trifluoromethyl)-1H-pyrrolo[3,2-h]quinolin-2-yl]-1,3,4-oxadiazole

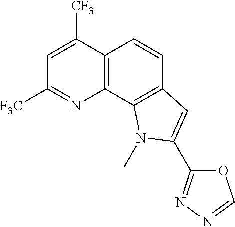 [SMILES: Cn1c(-c2nnco2)cc2ccc3c(C(F)(F)F)cc(C(F)(F)F)nc3c21]

To a mixture of 2-(6,8-bis(trifluoromethyl)-1H-pyrrolo[3,2-h]quinolin-2-yl)-1,3,4-oxadiazole (20 mg, 0.054 mmol) and K₂CO₃ (15 mg, 0.109 mmol) in N,N-dimethylformamide (1 mL) was added dimethyl sulfate (30 μL, 0.314 mmol) and the reaction mixture was heated at 60° C. under nitrogen for 30 minutes. The reaction mixture was cooled to room temperature and the crude mixture was purified via reverse phase HPLC to give to give 22-[1-methyl-6,8-bis(trifluoromethyl)-1H-pyrrolo[3,2-h]quinolin-2-yl]-1,3,4-oxadiazole (11.4 mg, 0.028 mmol, 52.2% yield) as a light yellow solid. ¹H NMR (400 MHz, CDCl3 δ: ppm 5.03 (s, 3H) 7.48 (s, 1H) 7.89 (dd, J=8.89, 1.66 Hz, 1H) 7.95-8.15 (m, 2H) 8.56 (s, 1H); ES LC-MS m/z=387.21 (M+H)⁺.

### Example 17

### 2-[2-phenyl-(trifluoromethyl)imidazo[1,2-a]1,8-naphthyridin-8-yl]-1,3,4-oxadiazole

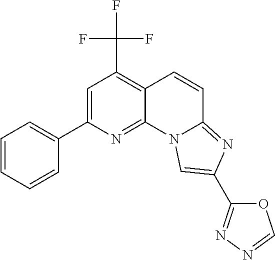 [SMILES: FC(F)(F)c1cc(-c2ccccc2)nc2c1ccc1nc(-c3nnco3)cn12]

A solution of 2-chloro-8-(1,3,4-oxadiazol-2-yl)-4-(trifluoromethyl)imidazo[1,2-a]-1,8-naphthyridine (50 mg, 0.15 mmol), PdCl₂(dppf)-CH₂Cl₂ (12 mg, 0.015 mmol), phenylboronic acid (21 mg, 0.18 mmol) and potassium acetate (58 mg, 0.59 mmol) in dioxane (1.5 mL) was degassed with nitrogen and heated to 100° C. in a sealed tube for 1 h. The reaction was cooled to room temperature, poured into ethyl acetate and washed with water. The organic layer was concentrated to half volume, and the mixture filtered and solids dried to afford the title compound (42 mg, 70% yield). LC-MS: ESI (M+H)⁺ m/z=382.11. ¹H NMR (400 MHz, DMSO-d₆) d ppm 9.57 (s, 1H), 9.45 (s, 1H), 8.49-8.77 (m, 3H), 7.86-8.02 (m, 2H), 7.49-7.80 (m, 3H).

### Example 18

### 2-[9-chloro-2,4-bis(trifluoromethyl)imidazo[1,2-a]1,8-naphthyridin-8-yl]-1,3,4-oxadiazole

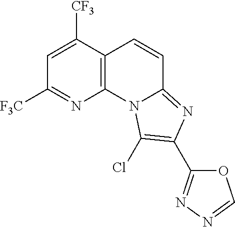 [SMILES: FC(F)(F)c1cc(C(F)(F)F)c2ccc3nc(-c4nnco4)c(Cl)n3c2n1]

A solution of 2-(2,4-bis(trifluoromethyl)imidazo[1,2-a][1,8]naphthyridin-8-yl)-1,3,4-oxadiazole (165 mg, 0.442 mmol) and 1-chloropyrrolidine-2,5-dione (236 mg, 1.768 mmol) in N,N-dimethylformamide (4 mL) was stirred at 60° C. for 2 hours. Water was added and the precipitate was filtered off to give 2-[9-chloro-2,4-bis(trifluoromethyl)imidazo[1,2-a]1,8-naphthyridin-8-yl]-1,3,4-oxadiazole (145 mg, 0.338 mmol, 76% yield).).: ¹H NMR (400 MHz, DMSO-d₆. δ ppm 7.99 (dd, 1H) 8.11 (d, J=9.87 Hz, 1H) 8.55 (s, 1H) 9.50 (s, 1H); ES LC-MS m/z=408.24 (M+H)⁺.

### Example 19

### 3-[8-(1,3,4,4-oxadiazol-2-yl)-4-(trifluoromethyl)imidazo[1,2-a]1,8-naphthyridin-2-yl]pyridine

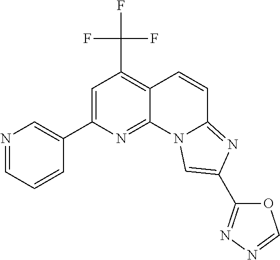 [SMILES: FC(F)(F)c1cc(-c2cccnc2)nc2c1ccc1nc(-c3nnco3)cn12]

A solution of 2-(2-chloro-4-(trifluoromethyl)imidazo[1,2-a][1,8]naphthyridin-8-yl)-1,3,4-oxadiazole (50 mg, 0.147 mmol), pyridin-3-ylboronic acid (36.2 mg, 0.294 mmol), sodium carbonate (46.8 mg, 0.442 mmol), Pd₂(dba)₃ (13.48 mg, 0.015 mmol), and tricyclohexylphosphine (10.32 mg, 0.037 mmol) in 1,4-dioxane (4 mL)/water (2 mL) was maintained with stirring at 80° C. for 4 hours. The mixture was cooled, poured into ethyl acetate, and washed with water. The organic layer was separated, dried over sodium sulfate, filtered, concentrated, and purified by reverse phase hplc to afford 2-(2-(pyridin-3-yl)-4-(trifluoromethyl)imidazo[1,2-a][1,8]naphthyridin-8-yl)-1,3,4-oxadiazole (4.1 mg, 10.72 μmol, 7.29% yield) as a yellow solid. LC-MS: ESI (M+H)⁺ m/z=383. ¹H NMR (400 MHz, CHLOROFORM-d/CD₃OD Mixture) ppm 7.59 (dd, J=7.90, 4.78 Hz, 1H) 7.86 (d, J=9.76 Hz, 1H) 7.95 (dd, J=9.76, 1.56 Hz, 1H) 8.30 (s, 1H) 8.57-8.65 (m, 2H) 8.84 (dd, J=4.78, 1.46 Hz, 1H) 9.39 (s, 1H) 9.48 (d, J=1.76 Hz, 1H).

### Example 20

### 2-[2-(difluoromethoxy)-4-(trifluoromethyl)imidazo[1,2-a]1,8-naphthyridin-8-yl]-1,3,4-oxadiazole

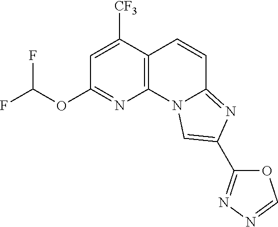 [SMILES: FC(F)Oc1cc(C(F)(F)F)c2ccc3nc(-c4nnco4)cn3c2n1]

A mixture of 8-(1,3,4-oxadiazol-2-yl)-4-(trifluoromethyl)imidazo[1,2-a][1,8]naphthyridin-2-ol (50 mg, 0.156 mmol), sodium 2-chloro-2,2-difluoroacetate (59.3 mg, 0.389 mmol) and Cs₂CO₃ (71.0 mg, 0.218 mmol) were dissolved in N,N-dimethylformamide (2 mL) was heated at 90° C. under nitrogen for 2 hours. The reaction mixture was purified via reverse phase HPLC to give 2-[2-(difluoromethoxy)-4-(trifluoromethyl)imidazo[1,2-a]1,8-naphthyridin-8-yl]-1,3,4-oxadiazole (22.2 mg, 0.057 mmol, 36.5% yield) as a light yellow solid. ¹H NMR (400 MHz, DMSO-d₆ δ: ppm 7.89-7.93 (m, 3H) 8.45 (t, 1H) 9.46 (s, 1H) 9.52 (s, 1H); ES LC-MS m/z=372.23 (M+H)⁺.

### Example 21

### 2-[f-cyclobutanecarbonyl-6,8-bis(trifluoromethyl)-1H-pyrrolo[3,2-h]quinolin-2-yl]-1,3,4-oxadiazole

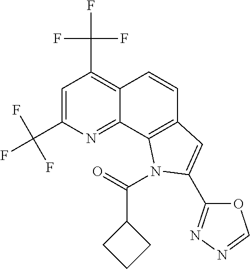 [SMILES: O=C(C1CCC1)n1c(-c2nnco2)cc2ccc3c(C(F)(F)F)cc(C(F)(F)F)nc3c21]

To a solution of 2-(1,3,4-oxadiazol-2-yl)-6,8-bis(trifluoromethyl)-1H-pyrrolo[3,2-h]quinoline (example 14, step C) (50 mg, 0.13 mmol) and triethylamine (0.04 mL, 0.27 mmol) in anhydrous DMF (1 mL) was added cyclobutanecarbonyl chloride (21 mg, 0.18 mmol) and the reaction stirred at room temperature for 2 h. Additional cyclobutanecarbonyl chloride was added (21 mg, 0.18 mmol) and the reaction stirred for an additional 1 h. The reaction was poured into ethyl acetate and washed with water, brine, and dried (MgSO₄) and concentrated in vacuo. The residue was purified by reverse-phase HPLC eluting with 10-90% acetonitrile/water/0.1% formic acid to afford the title compound (18 mg, 27% yield). LC-MS: ESI (M+H)⁺ m/z=455.10. ¹H NMR (400 MHz, DMSO-d₆) d ppm 9.51 (s, 1H), 8.31-8.48 (m, 2H), 7.99 (dd, J=9.0, 2.0 Hz, 1H), 7.82 (s, 1H), 4.05-4.27 (m, 1H), 2.52-2.71 (m, 2H), 2.14 (m, J=12.3, 8.4, 8.4, 3.9 Hz, 2H), 1.74-2.01 (m, 2H).

### Example 22

### 1-[2-(1,3,4-oxadiazol-2-yl)-6,8-bis(trifluoromethyl)-1H-pyrrolo[3,2-h]quinolin-1-yl]ethan-1-one

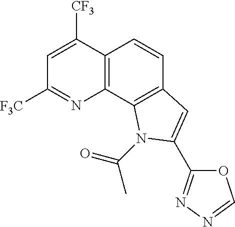 [SMILES: CC(=O)n1c(-c2nnco2)cc2ccc3c(C(F)(F)F)cc(C(F)(F)F)nc3c21]

To a mixture of 2-(6,8-bis(trifluoromethyl)-1H-pyrrolo[3,2-h]quinolin-2-yl)-1,3,4-oxadiazole (50 mg, 0.134 mmol) and TEA (0.112 mL, 0.806 mmol) in N,N-dimethylformamide (2 mL) was added acetyl chloride (0.048 mL, 0.672 mmol) dropwise and the reaction mixture was heated overnight at 60° C. The reaction mixture was cooled, diluted with water and extracted with DCM. The organics were separated, concentrated to dryness and the residue was purified via silica gel chromatography (0-5% MeOH/DCM) to give 1-[2-(1,3,4-oxadiazol-2-yl)-6,8-bis(trifluoromethyl)-1H-pyrrolo[3,2-h]quinolin-1-yl]ethan-1-one (20.6 mg, 0.047 mmol, 35.2% yield) as a white solid. ¹H NMR (400 MHz, CDCl₃ δ: ppm 3.15 (s, 3H) 7.50 (s, 1H) 7.97 (dd, J=8.98, 1.95 Hz, 1H) 8.04-8.18 (m, 2H) 8.53 (s, 1H); ES LC-MS m/z=415.21 (M+H)⁺.

### Example 23

### 2-[4-chloro-2-(propan-2-yl)imidazo[1,2-a]1,8-naphthyridin-8-yl]-1,3,4-oxadiazole

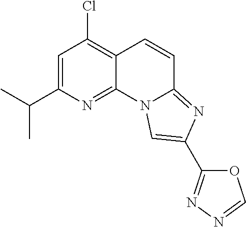 [SMILES: CC(C)c1cc(Cl)c2ccc3nc(-c4nnco4)cn3c2n1]

### Step A

### 7-amino-2-(1-methylethyl)-1,8-naphthyridin-4(1H)-one

A solution of pyridine-2,6-diamine (5 g, 45.8 mmol) and ethyl 4-methyl-3-oxopentanoate (11.09 mL, 68.7 mmol) in diphenyl ether (50 mL) was maintained at 150° C. overnight and then warmed to 250° C. for another 24 hours. The mixture was cooled to room temperature and product allowed to crystallize out over 5 hours. The supernatant was poured off and the solids were triturated with DCM/MeOH and the solids collected via vacuum filtration to afford 7-amino-2-isopropyl-1,8-naphthyridin-4(1H)-one (3.3 g, 16.24 mmol, 35.4% yield) as a yellow solid. LC-MS: ESI (M+H)⁺ m/z=222.45.

### Step B

### ethyl 2-(1-methylethyl)-4-oxo-1,4-dihydroimidazo[1,2-a]-1,8-naphthyridine-8-carboxylate

To a solution of 7-amino-2-(1-methylethyl)-1,8-naphthyridin-4(1H)-one (2.8 g, 13.8 mmol) in anhydrous DMF (40 mL) was added ethyl 3-bromo-2-oxopropanoate (4.0 g, 20.7 mmol) and the reaction stirred at 60° C. for 18 h. The reaction was cooled to room temperature and poured into ethyl acetate, washed with water, brine, dried (MgSO₄) and concentrated in vacuo. The residue was triturated in ether and filtered, the solids dried. The filtrate was concentrated in vacuo and the residue purified by silica gel chromatography eluting with 0-10% ethyl acetate/methanol. The eluent was combined with the filtered solids to afford the title compound (800 mg, 19% yield). LC-MS: ESI (M+H)⁺ m/z=299.82.

### Step C

### 2-(1-methylethyl)-8-(1,3,4-oxadiazol-2-yl)imidazo[1,2-a]-1,8-naphthyridin-4(1H)-one

To a solution of ethyl 2-(1-methylethyl)-4-oxo-1,4-dihydroimidazo[1,2-a]-1,8-naphthyridine-8-carboxylate (922 mg, 3.1 mmol) in ethanol (25 mL) was added hydrazine (1.9 mL, 61.6 mmol) and the reaction heated to 85° C. overnight. The reaction was cooled to room temperature, the solvent removed in vacuo and the residue dried. To the residue was added triethyl orthoformate (20 mL) and p-toluenesulfonic acid monohydrate (586 mg, 3.1 mmol) and the reaction heated to 110° C. for 1 h. The reaction was cooled to room temperature, poured into ethyl acetate, washed with saturated sodium bicarbonate solution, and dried (MgSO₄) and concentrated in vacuo. The residue was triturated in ether and solids were filtered and dried to afford the title compound (175 mg, 19% yield). LC-MS: ESI (M+H)⁺ m/z=296.24.

### Step D

### 2-[4-chloro-2-(propan-2-yl)imidazo[1,2-a]1,8-naphthyridin-8-yl]-1,3,4-oxadiazole

A mixture of 2-(1-methylethyl)-8-(1,3,4-oxadiazol-2-yl)imidazo[1,2-a]-1,8-naphthyridin-4(1H)-one (175 mg, 0.59 mmol) and phosphorus oxytrichloride (4 mL) was heated to 100° C. for 30 min. The reaction was cooled to room temperature and the volatiles removed in vacuo. The residue was stirred with water for 10 min and neutralized with potassium carbonate. The solution was extracted twice with dichloromethane and the organic layer dried (MgSO₄) and concentrated in vacuo. The residue was purified by silica gel chromatography eluting with 50-100% hexanes/ethyl acetate to afford the title compound (54 mg, 29% yield). LC-MS: ESI (M+H)⁺ m/z=314.25. ¹H NMR (400 MHz, DMSO-d₆) d ppm 9.41 (s, 1H), 9.14 (s, 1H), 7.93-8.04 (m, 1H), 7.86-7.93 (m, 1H), 7.83 (d, J=9.8 Hz, 1H), 3.21-3.31 (m, 1H), 1.27-1.46 (m, 6H).

### Example 24

### 2-[6,8-bis(trifluoromethyl)imidazo[1,2-a]quinolin-2-yl]-1,3,4-oxadiazole

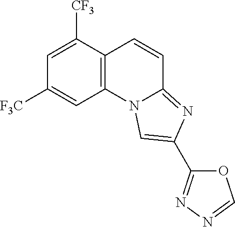 [SMILES: FC(F)(F)c1cc(C(F)(F)F)c2ccc3nc(-c4nnco4)cn3c2c1]

### Step A

### (E)-methyl 3-(2-amino-4,6-bis(trifluoromethyl)phenyl)acrylate

A pressure tube was treated by the addition of 2-bromo-3,5-bis(trifluoromethyl)aniline (9.0 g, 29.2 mmol) and ACN (44.8 ml), followed by the addition of PdOAc₂ (0.656 g, 2.92 mmol), P(o-tol)₃ (1.779 g, 5.84 mmol), and purged with nitrogen. TEA (20.36 ml, 146 mmol) and methyl acrylate (7.90 ml, 88 mmol) were then added. The tube was flushed with nitrogen, sealed tightly, and heated to 100° C. for 5 hours. The reaction was filtered through GF/F, washing with DCM. The filtrate was treated with water, extracted with DCM (3×), washed with brine, dried over Na₂SO₄, filtered, and concentrated onto celite. The residue was purified by silica gel chromatography (10-30% EtOAc/Hexanes) to give (E)-methyl 3-(2-amino-4,6-bis(trifluoromethyl)phenyl)acrylate (5.90 g, 18.84 mmol, 64.5% yield) as a yellow solid. ES LC-MS m/z=314.1 (M+H)⁺.

### Step B

### 5,7-bis(trifluoromethyl)quinolin-2(1H)-one

A solution of (E)-methyl 3-(2-amino-4,6-bis(trifluoromethyl)phenyl)acrylate (4.0 g, 12.77 mmol) in toluene (64.1 ml) was treated by the addition of piperidine (6.83 ml, 69.0 mmol). The reaction was then heated to reflux and stirred for 48 hours. After cooling to room temperature, additional piperidine (6.5 mL) was added and heating was continued for 4 hours. A small amount of the reaction was taken out and transferred to a round bottom flask. The reaction was cooled to room temperature and then concentrated under reduced pressure. The residue was taken up in EtOAc and water. The combined organics were washed with brine, dried over Na₂SO₄, filtered, and concentrated. The residue was taken up in DCM, the solids were filtered to give pure product (0.538 g) and the filtrate was loaded onto celite and purified by silica gel chromatography (30% EtOAc/Hexane) to give additional product (1.326 g). The batches were combined to give 5,7-bis(trifluoromethyl)quinolin-2(1H)-one (1.86 g, 52%). ES LC-MS m/z=282.1 (M+H)⁺.

### Step C

### 2-chloro-5,7-bis(trifluoromethyl)quinoline

A solution of 5,7-bis(trifluoromethyl)quinolin-2(1H)-one (1.0 g, 3.56 mmol) was treated with POCl₃ (6.30 ml, 67.6 mmol) and the reaction was heated to 110° C. for 1 hour. After cooling to room temperature, the reaction was concentrated and the residue was taken up in EtOAc and washed with water (3×), brine, dried MgSO₄, filtered, and concentrated to give 2-chloro-5,7-bis(trifluoromethyl)quinoline (1.0256 g, 3.42 mmol, 96% yield) as a solid. ES LC-MS m/z=300.4 (M+H)⁺.

### Step D

### N-(4-methoxybenzyl)-5,7-bis(trifluoromethyl)quinolin-2-amine

A solution of 2-chloro-5,7-bis(trifluoromethyl)quinoline (1.026 g, 3.42 mmol),4-methoxybenzylamine (0.492 ml, 3.77 mmol), and DIEA (0.897 ml, 5.14 mmol) in DMF (15.73 ml) was heated to 60° C. for 4 hours. After cooling to room temperature, the reaction was concentrated and the residue was taken up in EtOAc, washed with water (3×), brine, dried over MgSO₄, filtered, and concentrated to give N-(4-methoxybenzyl)-5,7-bis(trifluoromethyl)quinolin-2-amine (1.34 g, 3.35 mmol, 98% yield). ES LC-MS m/z=401.2 (M+H)⁺.

### Step E

### 5,7-bis(trifluoromethyl)quinolin-2-amine

A solution of N-(4-methoxybenzyl)-5,7-bis(trifluoromethyl)quinolin-2-amine (1.34 g, 3.35 mmol) in TFA (16.74 ml) was heated to 140° C. in the microwave for 20 minutes. The solvents were then removed under reduced pressure, the residue was taken up in DCM, washed with saturated NaHCO₃ (3×), brine, dried over Na₂SO₄, filtered, and concentrated to give 5,7-bis(trifluoromethyl)quinolin-2-amine (1.081 g, 3.86 mmol, quantitative yield) as a solid. ES LC-MS m/z=281.1 (M+H)⁺.

### Step F

### ethyl 6,8-bis(trifluoromethyl)imidazo[1,2-a]quinoline-2-carboxylate

A solution of 5,7-bis(trifluoromethyl)quinolin-2-amine (1.512 g, 5.40 mmol) in DMF (25.3 ml) was treated with ethyl bromopyruvate (1.697 ml, 13.49 mmol). The reaction was heated to 80° C. overnight. The black reaction was concentrated under reduced pressure to remove most of the DMF. The residue was diluted with H₂O and was extracted with EtOAc. The combine organics were washed with 5% LiCl (3×), brine, dried MgSO₄, filtered, and concentrated onto celite. The residue was purified by silica gel chromatography (0-3% MeOH/DCM) to give ethyl 6,8-bis(trifluoromethyl)imidazo[1,2-a]quinoline-2-carboxylate (1.069 g, 2.84 mmol, 52.6% yield). ES LC-MS m/z=377.1 (M+H)⁺.

### Step G

### 6,8-bis(trifluoromethyl)imidazo[1,2-a]quinoline-2-carbohydrazide

A solution of ethyl 6,8-bis(trifluoromethyl)imidazo[1,2-a]quinoline-2-carboxylate (0.510 g, 1.355 mmol) and hydrazine (0.851 ml, 27.1 mmol) in EtOH (5.93 ml) was heated to reflux for 2 hours. The reaction was concentrated under reduced pressure to give 6,8-bis(trifluoromethyl)imidazo[1,2-a]quinoline-2-carbohydrazide (0.491 g, 1.355 mmol, 100% yield). ES LC-MS m/z=363.14 (M+H)⁺.

### Step H

### 2-[6,8-bis(trifluoromethyl)imidazo[1,2-a]quinolin-2-yl]-1,3,4-oxadiazole

A solution of 6,8-bis(trifluoromethyl)imidazo[1,2-a]quinoline-2-carbohydrazide (0.491 g, 1.355 mmol), TsOH (0.103 g, 0.542 mmol), and triethyl orthoformate (9.03 ml, 54.2 mmol) was heated at 80° C. under nitrogen overnight. The reaction was treated by additional of TsOH (0.103 g, 0.542 g) and continued to heat for an additional 90 minutes. The reaction was concentrated and the residue was diluted with water and sonicated. The brown solids were filtered (551 mg) and then were diluted with DCM and loaded onto celite. The residue was purified by silica gel chromatography (3% MeOH/DCM). The fractions containing the product were combined and concentrated. The residue was taken up in ACN and the solids were filtered to give pure product (0.0053 g). The filtrate was purified by reverse phase chromatography to give additional product (0.0064 g). The batches were combined to give 2-[6,8-bis(trifluoromethyl)imidazo[1,2-a]quinolin-2-yl]-1,3,4-oxadiazole (0.011 g, 2.2%). ¹H NMR (400 MHz, DMSO-d₆) δ ppm 9.98 (s, 1H), 9.47 (s, 1H), 9.45 (s, 1H), 8.29 (s, 1H), 7.94-8.11 (m, 2H), ES LC-MS m/z=373.1 (M+H)⁺.

### Example 25

### 8-(furan-2-yl)-2,4-bis(trifluoromethyl)imidazo[1,2-a], 8-naphthyridine

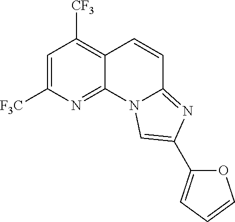 [SMILES: FC(F)(F)c1cc(C(F)(F)F)c2ccc3nc(-c4ccco4)cn3c2n1]

### Step A

### 5,7-bis(trifluoromethyl)-1,8-naphthyridin-2-amine

A mixture of pyridine-2,6-diamine (10 g, 91 mmol), 1,1,1,5,5,5-hexafluoropentane-2,4-dione (19 g, 91 mmol) in H₃PO₄ (100 mL) was stirred at 95° C. overnight. After cooling to room temperature, the mixture was poured into ice/water mixture. The pH of the aqueous phase was adjusted to 7 with the addition of ammonium hydroxide. The solid formed was collected by vacuum filtration, washed with water, and dried under reduced pressure. The crude product was recrystallized in EtOH to provide 5,7-bis(trifluoromethyl)-1,8-naphthyridin-2-amine (8 g, 28 mmol, 30% of yield) as a green solid: ES LC-MS m/z=282 (M+H)⁺.

### Step B

### 8-(furan-2-yl)-2,4-bis(trifluoromethyl)imidazo[1,2-a]1,8-naphthyridine

A mixture of 5,7-bis(trifluoromethyl)-1,8-naphthyridin-2-amine (100 mg, 0.356 mmol) and 2-bromo-1-(furan-2-yl)ethanone (88 mg, 0.427 mmol) was refluxed in EtOH (5 mL) overnight. The mixture was cooled to room temperature and EtOH was removed under reduced pressure. The residue was taken up with EtOAc (15 mL), washed with saturated NaHCO₃ (10 mL). The organic phase was dried over Na₂SO₄, filtered and concentrated. The residue was purified with column chromatography (silica gel, 0-10% of EtOAc in petroleum ether) to obtain 8-(furan-2-yl)-2,4-bis(trifluoromethyl)imidazo[1,2-a]1,8-naphthyridine (50 mg, 0.13 mmol, 38% of yield) as a yellow solid: ¹H NMR (300 MHz, CDCl₃) δ ppm 8.79 (s, 1H), 8.11 (s, 1H), 7.98-7.87 (m, 2H), 7.58 (s, 1H), 7.02 (d, 1H), 6.59 (d, 1H); ES LC-MS m/z=372.0 (M+H)⁺.

### Example 26

### 2-{2,4-dimethylimidazo[1,2-a]1,8-naphthyridin-8-yl}-1,3,4-oxadiazole

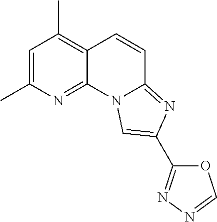 [SMILES: Cc1cc(C)c2ccc3nc(-c4nnco4)cn3c2n1]

### Step A

### 5,7-dimethyl-1,8-naphthyridin-2-amine

A mixture of pyridine-2,6-diamine (2 g, 18.3 mmol), pentane-2,4-dione (1.83, 18.3 mmol) and H₂SO₄ (0.25 mL) in glacial acetic acid (10 mL) was refluxed for 8 hours. After cooling to room temperature, the mixture was poured into a mixture of ice/water. The pH of the aqueous phase was adjusted to 7 with the addition of ammonium hydroxide. The brown solid formed was collected with filtration, washed with water, dried and recrystallized in EtOH to provide 5,7-dimethyl-1,8-naphthyridin-2-amine (1 g, 5.7 mmol, 32%) as a brown solid: ¹H NMR (300 MHz, DMSO-d₆) δ ppm 8.04 (d, 1H), 6.91 (s, 1H), 6.74 (d, 1H), 6.59 (s, br, 2H), 2.49 (s, 3H), 2.48 (s, 3H); ES LC-MS m/z=174.0 (M+H)⁺.

### Step B

### ethyl 2,4-dimethylimidazo[1,2-a][1,8]naphthyridine-8-carboxylate

A mixture of 5,7-dimethyl-1,8-naphthyridin-2-amine (900 mg, 5.2 mmol) and ethyl 3-bromo-2-oxopropanoate (1.15 g, 5.7 mmol) was refluxed in EtOH (10 mL) under nitrogen overnight. After cooling to room temperature, the mixture was concentrated and the residue was purified by silica gel chromatography (silica gel, 20% to 50% of EtOAc/petroleum ether) to provide ethyl 2,4-dimethylimidazo[1,2-a][1,8]naphthyridine-8-carboxylate (420 mg, 1.56 mmol, 30% of yield) as a yellow solid: ES LC-MS m/z=270.0 (M+H)⁺.

### Step C

### 2,4-dimethylimidazo[1,2-a][1,8]naphthyridine-8-carbohydrazide

To a solution of ethyl 2,4-dimethylimidazo[1,2-a][1,8]naphthyridine-8-carboxylate (420 mg, 1.56 mmol) in EtOH (5 mL) was added hydrazine hydrate (780 mg, 15.6 mmol) at 0° C. The mixture was stirred at room temperature overnight. The yellow solid formed was collected by vacuum filtration, washed with EtOH and dried under reduced pressure to provide 2,4-dimethylimidazo[1,2-a][1,8]naphthyridine-8-carbohydrazide (300 mg, 1.17 mmol, 75% of yield) as yellow solid which was used in the next step without further purification. ES LC-MS m/z=256.1 (M+H)⁺.

### Step D

### 2-{2,4-dimethylimidazo[1,2-a]1,8-naphthyridin-8-yl}-1,3,4-oxadiazole

A mixture of 2,4-dimethylimidazo[1,2-a][1,8]naphthyridine-8-carbohydrazide (200 mg, 0.78 mmol) and trimethyl orthoformate (166 mg, 1.57 mmol) was refluxed in EtOH (5 mL) overnight. After cooling to room temperature, the mixture was concentrated in vacuo. The residue was recrystallized in EtOH to provide 2-{2,4-dimethylimidazo[1,2-a]1,8-naphthyridin-8-yl}-1,3,4-oxadiazole (60 mg, 0.22 mmol, 29% of yield) as a light yellow solid: ¹H NMR (300 MHz, CD₃OD) δ ppm 9.10-9.08 (m, 2H), 7.96 (d, 1H), 7.54 (d, 1H), 7.36 (s, 1H), 2.68 (s, 6H). ES LC-MS m/z=266.1 (M+H)⁺.

### Example 27

### 2-[2,4-bis(trifluoromethyl)imidazo[1,2-a]1,8-naphthyridin-8-yl]-1,3-oxazole

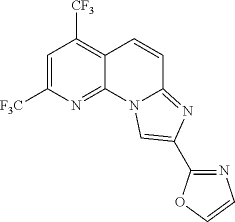 [SMILES: FC(F)(F)c1cc(C(F)(F)F)c2ccc3nc(-c4ncco4)cn3c2n1]

### Step A

### ethyl 2,4-bis(trifluoromethyl)imidazo[1,2-a][1,8]naphthyridine-8-carboxylate

A mixture of 5,7-bis(trifluoromethyl)-1,8-naphthyridin-2-amine (1.5 g, 5.34 mmol) and ethyl 3-bromo-2-oxopropanoate (1.25 g, 6.4 mmol) was refluxed in EtOH (15 mL) for 4 hours. After cooling down to room temperature, the yellow solid was collected via vacuum filtration and washed with EtOH to afford ethyl 2,4-bis(trifluoromethyl)imidazo[1,2-a][1,8]naphthyridine-8-carboxylate (745 mg, 1.97 mmol, 37%) as yellow solid: ¹H NMR (300 MHz, CDCl₃) δ ppm 9.15 (s, 1H), 8.14 (s, 1H), 7.94-7.92 (m, 2H), 4.52 (q, 2H), 1.48 (t, 3H); ES LC-MS m/z=378.1 (M+H)⁺.

### Step B

### 2,4-bis(trifluoromethyl)imidazo[1,2-a][1,8]naphthyridine-8-carboxylic acid

To a solution of ethyl 2,4-bis(trifluoromethyl)imidazo[1,2-a][1,8]naphthyridine-8-carboxylate (400 mg, 1.06 mmol) in THF (15 mL) and water (15 mL) was added lithium hydroxide monohydrate (223 mg, 5.31 mmol). The mixture was stirred at room temperature for 1 hour. THF was removed under reduced pressure. The aqueous layer was acidified to pH 2-3 with the addition of 1M HCl, extracted with EtOAc (20 mL×2). The combined organic layer was washed with brine (50 mL), dried over Na₂SO₄, filtered and concentrated. The crude 2,4-bis(trifluoromethyl)imidazo[1,2-a][1,8]naphthyridine-8-carboxylic acid (320 mg, 0.92 mmol, 86% of crude yield) was used in the next step without further purification.

### Step C

### N-(2,2-dimethoxyethyl)-2,4-bis(trifluoromethyl)imidazo[1,2-a][1,8]naphthyridine-8-carboxamide

To a solution of 2,4-bis(trifluoromethyl)imidazo[1,2-a][1,8]naphthyridine-8-carboxylic acid (220 mg, 0.64 mmol) in DMF (20 mL) was added DIPEA (177 mg, 1.32 mmol), TBTU (205 mg, 0.64 mmol) and 2,2-dimethoxyethanamine (67 mg, 0.64 mmol). The resulting mixture was stirred at room temperature overnight. Water was added and the aqueous phase was extracted with EtOAc (50 mL×2). The combined organic phase s were washed with brine, dried over Na₂SO₄, filtered and concentrated to give a residue. The crude product was purified on column chromatography (20% of EtOAc/petroleum ether) to give N-(2,2-dimethoxyethyl)-2,4-bis(trifluoromethyl)imidazo[1,2-a][1,8]naphthyridine-8-carboxamide (220 mg, 80%) as a white solid. ES LC-MS m/z=436.1 (M+H)⁺.

### Step D

### N-(2-oxoethyl)-2,4-bis(trifluoromethyl)imidazo[1,2-a][1,8]naphthyridine-8-carboxamide

To a solution of N-(2,2-dimethoxyethyl)-2,4-bis(trifluoromethyl)imidazo[1,2-a][1,8]naphthyridine-8-carboxamide (200 mg, 0.46 mmol) in DCM (20 mL) was added trifluoroacetic acid (262.2 mg, 2.3 mmol) at room temperature. The mixture was stirred at r.t. for 2 hours. The solution was washed with saturated NaHCO₃. The aqueous phase was extracted with EtOAc (10 mL×2). The combined organic phase was dried over Na₂SO₄, filtered and concentrated to provide N-(2-oxoethyl)-2,4-bis(trifluoromethyl)imidazo[1,2-a][1,8]naphthyridine-8-carboxamide (120 mg, 0.31 mmol, 67% of yield) which was used in the next step without further purification.

### Step E

### 2-[2,4-bis(trifluoromethyl)imidazo[1,2-a]1,8-naphthyridin-8-yl]-1,3-oxazole

To a solution of N-(2-oxoethyl)-2,4-bis(trifluoromethyl)imidazo[1,2-a][1,8]naphthyridine-8-carboxamide (120 mg, 0.3 mmol) in DCM (20 mL) was added perchloroethane (141 mg, 0.6 mmol), PPh₃ (157.2 mg, 0.6 mmol) and Et₃N (151.5 mg, 1.5 mmol) at room temperature. The resulting mixture was stirred at r.t. overnight. The solvent was removed under vacuum and the residue was purified with column chromatography (silica gel, 20%-50% of EtOAc/petroleum ether) to provide 2-[2,4-bis(trifluoromethyl)imidazo[1,2-a]1,8-naphthyridin-8-yl]-1,3-oxazole (40 mg, 0.09 mmol, 35% of yield): ¹H NMR (300 MHz, CD₃OD) δ ppm 9.15 (s, 1H), 8.35 (s, 1H), 8.09-8.05 (m, 2H), 7.99 (m, 1H), 7.40 (d, 1H); ES LC-MS m/z=372.0 (M+H)⁺.

### Example 28

### 5-[6,8-bis(trifluoromethyl)-3H-imidazo[4,5-h]quinolin-2-yl]-1,3-oxazole

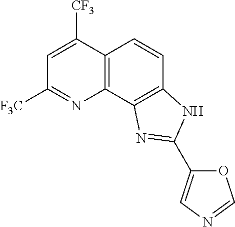 [SMILES: FC(F)(F)c1cc(C(F)(F)F)c2ccc3[nH]c(-c4cnco4)nc3c2n1]

### Step A

### 6,8-bis(trifluoromethyl)-[1,2,5]thiadiazolo[3,4-h]quinoline

A solution of 1,1,1,5,5,5-hexafluoropentane-2,4-dione (6.55 ml, 46.3 mmol) and benzo[c][1,2,5]thiadiazol-4-amine (5.0 g, 33.1 mmol) and AcOH (101 ml) was heated to 100° C. in a sealed tube overnight. The reaction was concentrated under reduced pressure and the residue was taken up in DCM and basified with saturated NaHCO₃. The combined organics were washed with saturated NaHCO₃ (3×), brine, dried over MgSO₄, filtered, and concentrated. The crude residue was loaded onto celite and purified by silica gel chromatography (0-30% EtOAc/Hexanes) to give 6,8-bis(trifluoromethyl)-[1,2,5]thiadiazolo[3,4-h]quinoline (7.51 g, 23.24 mmol, 70.3% yield) as a yellow solid. ES LC-MS m/z=323.9 (M+H)⁺.

### Step B

### 2,4-bis(trifluoromethyl)quinoline-7,8-diamine hydrochloride

A solution of 6,8-bis(trifluoromethyl)-[1,2,5]thiadiazolo[3,4-h]quinoline (7.51 g, 23.24 mmol) in MeOH (96 ml) was treated with cobalt(II) chloride hexahydrate (0.553 g, 2.324 mmol) and then NaBH₄ (1.319 g, 34.9 mmol) portionwise. The reaction was stirred at room temperature for 90 minutes. The reaction quenched by the addition of water. The black solids were filtered, rinsing with water. The aqueous layer was extracted with DCM. The combined organics were washed with brine, dried over Na₂SO₄, filtered, and concentrated. The black solids were rinsed with DCM. This organic phase was washed with brine, dried over Na₂SO₄, filtered, combined with the previously isolated batch, and concentrated. The dark residue was taken up in DCM and then treated by the addition of 4N in dioxanes HCl (20.33 ml, 81 mmol) to form a very fine solid. The solvents were removed under reduced pressure. The residue was taken up in Et₂O and MeOH and concentrated. The solid was triturated with Et₂O and filtered. The solid was then triturated with DCM and filtered to give 2,4-bis(trifluoromethyl)quinoline-7,8-diamine hydrochloride (1.88 g, 6.37 mmol, 27.4% yield) as a brownish yellow solid. ES LC-MS m/z=296.2 (M+H)⁺.

### Step C

### 5-[6,8-bis(trifluoromethyl)-3H-imidazo[4,5-h]quinolin-2-yl]-1,3-oxazole

A solution of 2,4-bis(trifluoromethyl)quinoline-7,8-diamine hydrochloride (0.150 g, 0.452 mmol) in NMP (1.822 ml) was treated by the addition of DIEA (0.197 ml, 1.131 mmol). The mixture was then treated by the addition of oxazole-5-carbaldehyde (0.044 g, 0.452 mmol) and sodium bisulfite (0.047 g, 0.452 mmol) and heated to 100° C. overnight. The reaction was treated with water and the solid was filtered. The solid was partially dissolved in DCM, the solids were filtered, rinsing with MeOH, and set aside. The filtrate was concentrated onto celite and purified by silica gel chromatography (0-3% MeOH/DCM). The fractions that contained the product were concentrated, taken up in DMSO and purified by reverse phase chromatography (10-90% ACN/H₂O+formic acid). Isolation and lyophilization give 55-[6,8-bis(trifluoromethyl)-3H-imidazo[4,5-h]quinolin-2-yl]-1,3-oxazole. ¹H NMR (400 MHz, DMSO-d₆) δ ppm 8.76 (s, 1H), 8.33 (s, 2H), 8.16 (s, 1H), 8.02-8.09 (m, 1H), ES LC-MS m/z=373.1 (M+H)⁺.

### Example 29

### 2-[2-chloro-4-(trifluoromethyl)imidazo[1,2-a]1,8-naphthyridin-8-yl]-1,3,4-oxadiazole

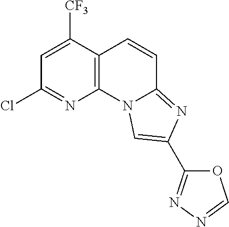 [SMILES: FC(F)(F)c1cc(Cl)nc2c1ccc1nc(-c3nnco3)cn12]

### Step A

### 7-amino-4-(trifluoromethyl)-1,8-naphthyridin-2-o

A mixture of pyridine-2,6-diamine (500 mg, 4.58 mmol) and ethyl 4,4,4-trifluoro-3-oxobutanoate (886 mg, 4.81 mmol) was heated until pyridine-2,6-diamine was completely dissolved. The mixture was cooled to 0° C. and concentrated H₂SO₄ (8 mL, 150 mmol) was added dropwise. The reaction mixture was then allowed to stand for 12 hours at 60° C., was poured into crushed ice and basified with 20% NaOH(aq) solution. The precipitate was filtered and washed with water to give (866 mg, Yield 82.9%) 7-amino-4-(trifluoromethyl)-1,8-naphthyridin-2-ol was afforded as a yellow solid. ES LC-MS m/z=230.02 (M+H)⁺,

### Step B

### methyl 2-hydroxy-4-(trifluoromethyl)imidazo[1,2-a][1,8]naphthyridine-8-carboxylate

A mixture of 7-amino-4-(trifluoromethyl)-1,8-naphthyridin-2-ol (1 g, 4.36 mmol) and methyl 3-bromo-2-oxopropanoate (1.185 g, 6.55 mmol) in N,N-dimethylformamide (10 mL) was heated at 60° C. for 8 hours under nitrogen. After cooling to room temperature, the reaction mixture was diluted with water and the filtrate filtered off and washed with water to give 560 mg (yield 41.2%) methyl 2-hydroxy-4-(trifluoromethyl)imidazo[1,2-a][1,8]naphthyridine-8-carboxylate was afforded as a yellow solid. ES LC-MS m/z=326.03 (M+H)⁺,

### Step C

### 2-hydroxy-4-(trifluoromethyl)imidazo[1,2-a][1,8]naphthyridine-8-carbohydrazide

To a solution of methyl 2-hydroxy-4-(trifluoromethyl)imidazo[1,2-a][1,8]naphthyridine-8-carboxylate (305 mg) dissolved in ethanol (8 mL) was added 20 eq hydrazine (640 μl, 20.39 mmol) and the reaction mixture was refluxed for 4 hours under nitrogen. The mixture was cooled to room temperature and concentrated to dryness in vacuum to give 2-hydroxy-4-(trifluoromethyl)imidazo[1,2-a][1,8]naphthyridine-8-carbohydrazide (228 mg) as a yellow solid. ES LC-MS m/z=312.09 (M+H)⁺,

### Step D

### 8-(1,3,4-oxadiazol-2-yl)-4-(trifluoromethyl)imidazo[1,2-a][1,8]naphthyridin-2-ol

A mixture of 100 mg 2-hydroxy-4-(trifluoromethyl)imidazo[1,2-a][1,8]naphthyridine-8-carbohydrazide and TsOH (40 mg, 0.210 mmol) (40 wt %) in triethylorthoformate (4 mL, 24.02 mmol) was heated at 80° C. for 1 hour. The mixture was cooled to room temperature, and was purified via reverse phase HPLC to give 8-(1,3,4-oxadiazol-2-yl)-4-(trifluoromethyl)imidazo[1,2-a][1,8]naphthyridin-2-ol (20 mg, 0.059 mmol, 1.35% yield) as a light brown solid. ES LC-MS m/z=322.22 (M+H)⁺,

### Step E

### 2-[2-chloro-4-(trifluoromethyl)imidazo[1,2-a]1,8-naphthyridin-8-yl]-1,3,4-oxadiazole

To a solution of 8-(1,3,4-oxadiazol-2-yl)-4-(trifluoromethyl)imidazo[1,2-a][1,8]naphthyridin-2-ol (100 mg, 0.311 mmol) dissolved in N,N-dimethylformamide (3 mL) at room temperature was added POCl₃ (0.058 mL, 0.623 mmol) dropwise. The reaction mixture was stirred at 80° C. for 5 hours, cooled to room temperature, and diluted with water. The brown precipitate was filtered off and purified via reverse phase HPLC to give 2-[2-chloro-4-(trifluoromethyl)imidazo[1,2-a]1,8-naphthyridin-8-yl]-1,3,4-oxadiazole (8.3 mg, 0.023 mmol, 7.46% yield) as a light yellow solid. ¹H NMR (400 MHz, DMSO-d₆ δ: ppm 7.91 (dd, J=9.76, 1.76 Hz, 1H) 7.98-8.02 (m, 1H) 8.29 (s, 1H) 9.16 (s, 1H) 9.44 (s, 1H); ES LC-MS m/z=340.16 (M+H)⁺.

### Example 30

### 2-[2,4-bis(propan-2-yl)imidazo[1,2-a]1,8-naphthyridin-8-yl]-1,3,4-oxadiazole

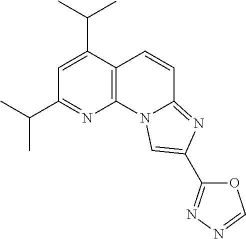 [SMILES: CC(C)c1cc(C(C)C)c2ccc3nc(-c4nnco4)cn3c2n1]

### Step A

### 7-amino-2-isopropyl-1,8-naphthyridin-4(1H)-one

Pyridine-2,6-diamine (15.0 g, 137 mmol) and ethyl 4-methyl-3-oxopentanoate (30.6 mL, 190 mmol) were added to diphenyl ether (150 mL). The mixture was heated at 150° C. for 4 hours. The mixture was then heated to 230° C. and excess ethyl 4-methyl-3-oxopentanoate was distilled off using a short path condenser. After ˜30 minutes, the short path condenser was replaced with a reflux condenser and the mixture continued to heat at 230° C. overnight. The mixture was allowed to cool to room temperature. Solids began to precipitate. Ethyl ether was added and then hexanes until a free-flowing solid was observed. The mixture was cooled to 0° C. in an ice-bath and the solids collected by filtration. The solids were washed with cold ether and dried to give the title compound (14.3 g, 47%) as tan solids. ES LC-MS m/z=204 (M+H)⁺.

### Step B

### 5-bromo-7-isopropyl-1,8-naphthyridin-2-amine

7-amino-2-isopropyl-1,8-naphthyridin-4(1H)-one (6.00 g, 29.5 mmol) was slurried in acetonitrile (60 mL) and phosphorus oxybromide (16.1 g, 56.1 mmol) added. An exotherm was observed. The mixture was heated to 80° C. for 3 hours, then allowed to cool to room temperature and stirred overnight. The mixture was poured into ice and made basic with saturated sodium bicarbonate. The mixture was extracted 3 times with ethyl acetate. The combined organic layers were washed with brine, dried over sodium sulfate, concentrated, and the residue dried under vacuum to give the title compound (5.2 g, 60%) as a rust-colored solid. ES LC-MS m/z=266, 268 (M+H)⁺.

### Step C

### ethyl 4-bromo-2-isopropylimidazo[1,2-a][1,8]naphthyridine-8-carboxylate

5-bromo-7-isopropyl-1,8-naphthyridin-2-amine (5.3 g, 20 mmol) and ethyl bromopyruvate (5.01 mL, 39.8 mmol) in ethanol (200 mL) were heated to 80° C. for 2 hours. N,N-diisopropylethylamine (13.9 mL, 80.0 mmol) was added and the reaction continued to heat at 80° C. for 2 hours. The mixture was allowed to cool to room temperature and was concentrated. The residue was purified by silica chromatography eluting with a gradient of 0% to 30% ethyl acetate in dichloromethane. Fractions were concentrated to give the title compound (2.83 g, 39%) as a pale yellow solid. ES LC-MS m/z=362, 364 (M+H)⁺.

### Step D

### lithium 4-bromo-2-isopropylimidazo[1,2-a][1,8]naphthyridine-8-carboxylate

Ethyl 4-bromo-2-isopropylimidazo[1,2-a][1,8]naphthyridine-8-carboxylate (2.8 g, 7.7 mmol) was dissolved in tetrahydrofuran (20 mL) and methanol (20 mL) before a solution of lithium hydroxide monohydrate (0.39 g, 9.3 mmol) in water (20 mL) was added. The mixture was stirred at room temperature overnight and concentrated. The residue was co-evaporated 2 times with toluene and concentrated to give the title compound (2.79 g, >99%) as a tan solid. ES LC-MS m/z=334, 336 (M+H)⁺.

### Step E

### 4-bromo-2-isopropylimidazo[1,2-a][1,8]naphthyridine-8-carbohydrazide

Thionyl chloride (50 mL, 685 mmol) was added to lithium 4-bromo-2-isopropylimidazo[1,2-a][1,8]naphthyridine-8-carboxylate (2.7 g, 7.5 mmol) and the mixture heated at 80° C. for 1 hour. The mixture was concentrated and the residue co-evaporated 2 times with toluene. The residue was dissolved in tetrahydrofuran (40 mL) and added to a stirring solution of hydrazine (4.7 mL, 150 mmol) and N,N-diisopropylethylamine (3.91 mL, 22.39 mmol) in tetrahydrofuran (40 mL). After stirring for 1 hour at room temperature, the mixture was concentrated, the residue quenched with water, and the mixture extracted 2 times with dichloromethane. The combined organic layers were washed with brine, dried over sodium sulfate, and concentrated to give the title compound (2.43 g, 82% pure, 77%). ES LC-MS m/z=348, 350 (M+H)⁺.

### Step F

### 2-(4-bromo-2-isopropylimidazo[1,2-a][1,8]naphthyridin-8-yl)-1,3,4-oxadiazole

4-bromo-2-isopropylimidazo[1,2-a][1,8]naphthyridine-8-carbohydrazide (2.43 g, 5.72 mmol), p-toluenesulfonic acid monohydrate (1.09 g, 5.72 mmol), and triethyl orthoformate (95 ml, 570 mmol) were heated at 80° C. for 2 hours. The mixture was allowed to cool to room temperature and was concentrated. The residue was purified by silica chromatography eluting with a gradient of 0% to 100% ethyl acetate in dichloromethane. Fractions were concentrated to give the title compound (1.4 g, 65%) as a pale yellow solid. ES LC-MS m/z=358, 360 (M+H)⁺.

### Step G

### 2-(2-isopropyl-4-(prop-1-en-2-yl)imidazo[1,2-a][1,8]naphthyridin-8-yl)-1,3,4-oxadiazole

2-(4-bromo-2-isopropylimidazo[1,2-a][1,8]naphthyridin-8-yl)-1,3,4-oxadiazole (75 mg, 0.19 mmol), potassium phosphate (164 mg, 0.771 mmol), potassium trifluoro(prop-1-en-2-yl)borate (57.0 mg, 0.385 mmol), and PdCl₂(dppf)-CH₂Cl₂ adduct (15.7 mg, 0.019 mmol) in 1,4-dioxane (2 mL) and water (0.500 mL) were degassed with nitrogen for 5 minutes before being heated at 90° C. for 3 hours. The mixture was allowed to cool to room temperature and was quenched with water. The mixture was extracted 2 times with ethyl acetate. The combined organic layers were washed with brine, dried over sodium sulfate, concentrated, and the residue purified by silica chromatography eluting with a gradient of 0% to 100% ethyl acetate in dichloromethane. Fractions were concentrated to give the title compound (40 mg, 61%) as an off-white solid. ES LC-MS m/z=320 (M+H)⁺. ¹H NMR (400 MHz, DMSO-d₆) ppm 9.40 (s, 1H), 9.12 (s, 1H), 7.86 (d, 1H), 7.69 (d, 1H), 7.52 (s, 1H), 5.61 (t, 1H), 5.15 (s, 1H), 3.18-3.31 (m, 1H), 2.22 (s, 3H), 1.39 (d, 6H).

### Step H

### 2-[2,4-bis(propan-2-yl)imidazo[1,2-a]1,8-naphthyridin-8-yl]-1,3,4-oxadiazole

2-(2-isopropyl-4-(prop-1-en-2-yl)imidazo[1,2-a][1,8]naphthyridin-8-yl)-1,3,4-oxadiazole (32 mg, 0.100 mmol), 10% palladium on carbon (Degussa) (10.66 mg, 10.02 μmol), and acetic acid (0.011 mL, 0.200 mmol) in ethanol (1 mL) and tetrahydrofuran (1 mL) were hydrogenated under balloon pressure for 5 hours. The catalyst was filtered off over celite and the filtrate concentrated. The residue was purified by silica chromatography eluting with a gradient of 0% to 100% ethyl acetate in dichloromethane. Fractions were concentrated to give the title compound (23 mg, 71%) as a white solid. LC-MS m/z=322 (M+H)⁺. ¹H NMR (400 MHz, DMSO-d₆) ppm 9.39 (s, 1H), 9.10 (s, 1H), 8.10 (d, 1H), 7.70 (d, 1H), 7.55 (s, 1H), 3.66-3.90 (m, 1H), 3.21-3.31 (m, 1H), 1.31-1.43 (m, 12H).

### Example 31

### 2-[4-phenyl-2-(propan-2-yl)imidazo[1,2-a]1,8-naphthyridin-8-yl]-1,3,4-oxadiazole

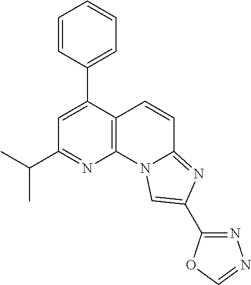 [SMILES: CC(C)c1cc(-c2ccccc2)c2ccc3nc(-c4nnco4)cn3c2n1]

2-(4-bromo-2-isopropylimidazo[1,2-a][1,8]naphthyridin-8-yl)-1,3,4-oxadiazole (51 mg, 0.13 mmol), potassium phosphate tribasic (111 mg, 0.524 mmol), phenylboronic acid (31.9 mg, 0.262 mmol), and PdCl2 (dppf)-CH₂Cl₂ adduct (10.7 mg, 0.013 mmol) in 1,4-dioxane (2 mL) and water (0.500 mL) were degassed with nitrogen for 5 minutes before being heated at 90° C. for 3 hours. The mixture was allowed to cool to room temperature and was quenched with water. The mixture was extracted 2 times with ethyl acetate. The combined organic layers were washed with brine, dried over sodium sulfate, concentrated, and the residue purified by silica chromatography eluting with a gradient of 0% to 100% ethyl acetate in dichloromethane. Fractions were concentrated to give the title compound (32 mg, 69%). LC-MS m/z=356 (M+H)⁺. ¹H NMR (400 MHz, DMSO-d₆) ppm 9.41 (s, 1H), 9.18 (s, 1H), 7.67 (d, 2H), 7.55-7.66 (m, 6H), 3.35 (s, 1H), 1.43 (d, 6H).

### Administration and Formulation

In further embodiments, there is provided a pharmaceutical composition comprising a pharmaceutically acceptable diluent and a therapeutically effective amount of a compound of Formula (I) or a pharmaceutically acceptable salt thereof. The chemical entities are administered at a therapeutically effective dosage, e.g., a dosage sufficient to provide treatment for the disease.

The compounds of the present invention can also be supplied in the form of a pharmaceutically acceptable salt. The terms “pharmaceutically acceptable salt” refer to salts prepared from pharmaceutically acceptable inorganic and organic acids and bases.

Pharmaceutically acceptable inorganic bases include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary salts include aluminum, calcium, lithium, magnesium, potassium, sodium and zinc. Particularly preferred are the ammonium, calcium, magnesium, potassium, and sodium salts.

Salts derived from pharmaceutically acceptable organic non-toxic bases include salts of primary, secondary, and tertiary amines, including in part, trimethylamine, diethylamine, N,N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Illustrative pharmaceutically acceptable acid addition salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, 3-hydroxybutyric, galactaric and galacturonic acids. Preferred pharmaceutically acceptable salts include the salts of hydrochloric acid and trifluoroacetic acid. All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The salt may precipitate from solution and be collected by filtration or may be recovered by evaporation of the solvent. The degree of ionisation in the salt may vary from completely ionised to almost non-ionised. Lists of suitable salts are found in Remington's Pharmaceutical Sciences, 17th ed., Mack Publishing Company, Easton, Pa., 1985, p. 1418, the disclosure of which is hereby incorporated by reference only with regards to the lists of suitable salts.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used, the route and form of administration, and other factors. The drug can be administered more than once a day, such as once or twice or three times a day.

Therapeutically effective amounts of the chemical entities described herein may range from approximately 0.01 to 200 mg per kilogram body weight of the recipient per day; such as about 0.01-100 mg/kg/day, for example, from about 0.1 to 50 mg/kg/day. Thus, for administration to a 70 kg person, the dosage range may be about 7-3500 mg per day.

In addition, the amount of the chemical entity in a composition can vary within the full range employed by those skilled in the art. Typically, the composition will contain, on a weight percent (wt %) basis, from about 0.01-99.99 wt % of at least one chemical entity described herein based on the total composition, with the balance being one or more suitable pharmaceutical excipients. In certain embodiments, the at least one chemical entity described herein is present at a level of about 1-80 wt %.

In certain embodiments, the chemical entities will be administered as pharmaceutical compositions by any one of the following routes: oral, systemic (e.g., transdermal, intranasal or by suppository), sublingually, subcutaneously, topically, intrapulmonarilly, vaginally, rectally, or intraocularly, or parenteral (e.g., intramuscular, intravenous or subcutaneous) administration. In other embodiments, oral administration with a convenient daily dosage regimen that can be adjusted according to the degree of disorder or disease may be used. The choice of administration route and/or formulation depends on various factors such as the mode of drug administration and bioavailability of the drug substance.

In one embodiment, the compounds of the present invention may be administered topically to the diseased area on the skin or mucous membranes of a subject. In another embodiment, the compounds of the present invention may be administered topically to the diseased area on the skin or mucous membranes of a subject so that the topical administration allows for the compound to penetrate into the subject's skin layer keratinocyte cells.

In some embodiments, the compositions are comprised of, in general, at least one chemical entity described herein in combination with at least one pharmaceutically acceptable excipient. Acceptable excipients are non-toxic, aid administration, and do not adversely affect the therapeutic benefit of at least one chemical entity described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition will comprise from about 0.2 to 2% of the active agent in solution.

In one embodiment, the compounds of the present invention can be formulated into dermatological topical delivery formulations. Pharmaceutical formulations adapted for topical administration may be formulated as ointments, creams, suspensions, lotions, powders, solutions, pastes, gels, sprays, aerosols or oils. For treatments of external tissues, such as skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

In addition to the compounds of the present invention, the compositions herein may additionally include an organic solvent, an adhesive, plasticizer, and a water swellable polymer. The organic solvent may be one or more of dimethylsulfoxide (DMSO), N,N′-dimethylacetamide (DMA), N′N′-dimethylformamide (DMF), dioxane, tetraglycol, or the like.

Appropriate adhesives for use in the invention include, but are not limited to, polyvinyl alcohol, polyethylene oxides, polyethylene glycols of molecular weight 3350 and higher, hydroxypropylcellulose, and povidone. Polyvinyl alcohol is preferred. The adhesive is typically present in an amount from about 10 to 75% by weight, preferably about 45-55% by weight, and most preferably about 50% by weight of the composition.

The compositions herein may optionally also include a plasticizer. Suitable plasticizers are typically high-boiling, water-soluble organic compounds containing hydroxyl, amide, or amino groups. Such plasticizers include, but are not limited to, soy, egg or synthetic lecithin, ethylene glycol, tetraethylene, hexamethylene, nonaethylene glycol, formamide, ethanolamine salts, water, glycerin, or combinations thereof. Such plasticizers are well known in the art. A plasticizer is therefore preferably included in the formulation to provide these benefits. The plasticizer is typically present in the composition in an amount ranging from about 0.4-2.0% by weight, with about 1-2% by weight being preferred, and about 0.9% by weight being most preferred.

The composition may also include a water swellable polymer which acts as an extender, and serves to thicken the composition. Such water swellable polymers are well known in the art and include, but are not limited to, microcrystalline cellulose, hydroxypropyl cellulose, hydroxypropyl methylcellulose, methyl cellulose, methyl ethyl cellulose, sodium carboxymethylcellulose, gums, carboxyvinyl polymer, hydroxyethyl cellulose, cornstarch, casein, urea, dextrin, and fume silica. The filler is typically present in an amount from about 1-10% by weight, preferably about 3-6% by weight, with about 4.67% by weight being most preferred.

The present invention is further directed to a method of treating warts by applying the pharmaceutical composition(s) topically to the location on the skin where the warts are present. The method of the invention comprises topically applying to a wart on an individual a therapeutically effective amount of the compositions of the invention. The composition may be applied using an applicator, for example, a swab, sponge, finger cot or a toothpick. While some compositions of this invention can be adhesive in and of themselves, in another embodiment of the invention, the method further comprises occluding the wart with an occluding agent to aid the composition's absorption into the wart, protect the composition from rubbing off, and also further keratolytic activity. Many occluding agents are known to those skilled in the art. These include, but are not limited to, bandages, plastic wrap, and adhesive tape, for example, duct tape.

The compositions of the invention may further include a variety of substances, including suitable stabilizers, buffers, thickeners, lubricants, wetting, and dissolving agents as well as colorings, moisturizers, preservatives, and fragrances. These minors are added in small amounts and are conventionally known in pharmaceutical formulation work to enhance elegance. Such minors typically comprise less than about 1% of the overall composition.

In still other embodiments, the compounds of the present invention can be formulated into dermatological delivery formulations, such as a stick-gel, which can be used to target the delivery of the compound directly onto the site of action. For example, if the compounds of the present invention are intended to be used as a treatment for papillomavirus induced warts, then the compound(s) may be formulated into a stick-gel that can apply the compounds in a formulation directly to the surface of the wart. In still other embodiments, the stick-gel application formulation can be based on a PSAs (Pressure Sensitive Adhesives) concept. PSAs, unlike structural adhesives or sealants, differ in that the adhesive-substrate interface does not resist separation when the adhesive is peeled off. In other words, PSAs are intended to show adhesive failure, especially when skin is the substrate, whereas this would be a major fatal flaw for cement and glue. Developing a suitable PSA-Gel for a targeted adherend to treat a skin common wart, takes the following two critical adhesive attributes into consideration: surface activity and visco-elastic properties.

As such, these attributes are associated to the three steps of adhesion process. The first step involves contact between the adhesive and the surface. This dynamic step is known as “bonding or sticking” and is dependent on wetting behavior and quick spreadability of the adhesive composition. The second step “adhering” relies on the capacity of the adhesive to remain in contact with surface. This is important for treating warts where the active should be adherent to the warts long enough to exert its intended action. Flowability and creep resistance are the physical characteristics that contribute to maintain the established bond and stick. During this more static phase, the adhesion will build up if the adhesive-to-surface interactions increase (e.g., interpenetration). The third step “debonding” is also dynamic. It consists in separating the adhesive-stick from the surface by means of a peel release process. The peel adhesion property of the adhesive composition will direct the force required to break the bond in an adhesive failure mode.

The formulation composition to achieve all these attributes can comprise suitable hydrophilic polymers incorporated into a gel matrix containing the active drug in solution. Large organic macromolecules that are either natural or synthetic hydrophilic polymers (e.g., hydroxy propyl methyl cellulose, ethyl cellulose, etc.) on the other hand, exist as randomly coiled chains that entangle with each other to form the gel structure. The nature of the solvent determines whether the gel is a hydrogel (water based) or an organogel (nonaqueous solvent). For example, gels prepared with hydroxyethyl cellulose containing water are hydrogels, whereas gels prepared with polyethylene-containing mineral oil (Plastibase) are organogels. Another class of gels, called thermally sensitive gels, are prepared from poloxamers. In addition to hydrophilic polymers, silicones are versatile materials permitting the design of various transdermal and topical drug delivery forms. The substantivity to skin can be adjusted from hours to one week in duration. Moreover, the hydrophobic, highly open, and mobile dimethylsiloxane network allows for the preparation of semi-occlusive matrices, permeable to many molecules including the compound(s) of the present invention.

In other embodiments of the present invention, there is provided sustained release of certain compounds described herein from silicone pressure sensitive adhesive matrices. This capability can also be expanded to other types of silicone matrices including fillerless or reinforced elastomers. As such, modulation of the release of certain compounds of the present invention could enhance drug targeting and therapeutic effectiveness. The silicone formulations could include a loosely cross-linked fillerless elastomer dispersion (Dow Corning® 9040 Silicone Elastomer Blend), a fully cross-linked fillerless elastomer (Dow Corning® 7-9800 A&B Soft Skin Adhesive), a rubber film-forming dispersion (Dow Corning® 7-5300 Film-In-Place Coating), and/or a viscoelastic system (Dow Corning® PSA 7-4502 and 7-4602 pressure sensitive adhesive. In certain embodiments, the compound(s) of the present invention could be formulated in the different silicone and polymer matrices along with the following excipients: surfactants, citric-sodium bicarbonates, and/or carbomer 974.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDIs typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation. Likewise, compressed gases may be used to disperse a chemical entity described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

Recently, pharmaceutical compositions have been developed for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The following examples serve to more fully describe the manner of making and using the above-described invention. It is understood that these examples in no way serve to limit the true scope of the invention, but rather are presented for illustrative purposes.

### BIOLOGICAL EXAMPLES

### Example 32

### Anti-Hepatitis C—Replicon Assay

A number of assays have been published to assess a compound's potential efficacy (activity) against the Hepatitis C virus (HCV). A general method that assesses the gross increase of HCV virus in culture was disclosed in U.S. Pat. No. 5,738,985 to Miles, et al. In vitro assays have been reported in Ferrari, et al. Jnl. of Vir., 73:1649-1654, (1999); Ishii, et al., Hepatology, 29:1227-1235, (1999); Lohmann, et al., J. Biol. Chem., 274:10807-10815, (1999); and Yamashita, et al., J. Biol. Chem., 273:15479-15486, (1998).

In the present application, the following method was used to assay for HCV activity.

Compounds were assayed for activity against HCV using the genotype 1a and 1b subgenomic replicon model systems. Stable cell lines bearing the genotype 1a and 1b replicons were used for screening of compounds. Both replicons are bicistonic and contain the firefly luciferase gene. The ET cell line is stably transfected with RNA transcripts harboring a I₃₈₉luc-ubi-neo/NS3-3′/ET replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The genotype 1a replicon is a stable cell line licensed from Apath LLC, modified to contain the firefly luciferase gene. The cells were grown in DMEM, supplemented with 10% fetal calf serum, 2 mM Glutamine, Penicillin (100 IU/mL)/Streptomycin (100 μg/mL), 1× nonessential amino acids, and 250-500 μg/mL G418 (“Geneticin”). They were all available through Life Technologies (Bethesda, Md.). The cells were plated at 0.5×10⁴ cells/well in 384 well plates containing compounds. The final concentration of compounds ranged between 0.03 pM to 50 μm and the final DMSO concentration of 0.5-1%.

Luciferase activity was measured 48 hours later by adding a Steady glo (Promega, Madison, Wis.). Percent inhibition of replication data was plotted relative to no compound control. Under the same condition, cytotoxicity of the compounds was determined using cell titer glo (Promega, Madison, Wis.). IC₅₀s were determined from a 10 point dose response curve using 3-4-fold serial dilution for each compound, which spans a concentration range>1000 fold. BioAssay determines the level of inhibition for each compound by normalizing cross-talk corrected plate values against the negative (low or background, cells with no compound present) and positive (high DMSO, no cells) controls to determine Percent Inhibition:

$$\frac{100 * ( 1 - ( \begin{matrix}Cross \text{-} talk corrected value - \\ Compound Positive Control Mean\end{matrix} ) )}{\begin{matrix}DMSO Negative Control Mean - \\ Compound Positive Control Mean\end{matrix}}$$

These normalized values are exported to IC₅₀ where they are plotted against the molar compound concentrations using the standard four parameter logistic equation:

$$y = A + \frac{B - A}{1 + {[ \frac{{10}^{X}}{{10}^{c}} ]}^{D}}$$

$$Where \text{:}$$

$$\begin{matrix}A = minimum y & D = slope factor \\ B = maximum y & x = {log}_{10} compound concentration [ M ] \\ C = {log}_{10} {EC}_{50} & \end{matrix}$$

### Example 33

### ISG56 Luciferase Reporter Assay

Compounds of the present invention were tested against a HEK (Human Embryonic Kidney) 293 cell line that was stably transfected with a firefly luciferase reporter gene under the control of the ISG56 (Interferon-Stimulated Gene 56) promoter ISRE (Interferon-Stimulated Response Element). While the ISRE is in the opposite orientation of the wild type promoter, literature¹ cites that the response elements are pallindromic and function properly in either orientation. ¹Reich, N., Evans, B., Levy, D., Fahey, D., Knight, E., Damell, J. E. (1987) Interferon-induced transcription of a gene encoding a 15-kDa protein depends on an upstream enhancer element. See, Proc. Natl. Acad. Sci. 84, (6394-6398).

In preparation for the assay, test compounds were serially diluted 3-fold in DMSO from a typical top concentration of 5 mM and plated at 0.2 μL in a 384-well, polystyrene, tissue culture treated plate with lid (Greiner Bio-One North America, Inc., Monroe, N.C.) to generate 11-point dose response curves in the assay. Low control wells (0% response) contained 0.2 μL of DMSO alone, and high control wells (100% response) contained 0.2 μL of a small molecule control test compound.

Frozen stocks of the transfected HEK 293 cells were washed and recovered in DMEM I Ham's F-12 media (Invitrogen Corporation, Carlsbad, Calif.) supplemented with 10% v/v qualified Australian fetal bovine serum (FBS) (Invitrogen Corporation, Carlsbad, Calif.), 1× GlutaMAX™ (Invitrogen Corporation, Carlsbad, Calif.), 1×MEM non-essential amino acids (NEAA) (Invitrogen Corporation, Carlsbad, Calif.) and 500 μg/ml Geneticin® (Invitrogen Corporation, Carlsbad, Calif.). The cells were diluted to 500,000 cells/mL in the supplemented DMEM/Ham's F-12 media, and 20 μL of the cell suspension were added to each well of the previously prepared 384-well compound plate, resulting in 10,000 cells/well. The plate, with lid, were placed in a 37° C., 5% CO₂ humidified incubator for 24 hours.

Following incubation, the plates were removed and placed on the bench top without lids to equilibrate to room temperature for 30 minutes. Steady-Glo® (Promega Corporation, Madison, Wis.) was prepared according to the manufacturer's instructions, and 10 μL were added to each plate well. After a twenty minute incubation at room temperature, luminescence was read on a ViewLux™ (PerkinElmer Inc., Waltham, Mass.).

The data for dose responses were plotted as % activation versus compound concentration following normalization using the formula 100*((U−C1)/(C2−C1)), where U was the unknown value, C1 was the average of the low (0% response) control wells and C2 was the average of the high (100% response) control wells. Curve fitting was performed with the equation y=A+((B−A)/(1+(10^{x}/10^{C})^{D})), where A was the minimum response, B was the maximum response, C was the log(EC₅₀) and D was the Hill slope. The results for each test compound were recorded as pEC50 values (—C in the above equation) and as max response values at a given concentration.

As shown below, the tested compounds were found to inhibit the activity of the replicon with pEC₅₀ values of about 9 or less. Preferably, the compounds will exhibit pEC₅₀ values of about 8 or less, in some embodiments, about 7 or less, and in some embodiments, about 6 or less. Further, compounds of the present disclosure, which were tested against more than one genotype of HCV replicon, were found to have similar inhibitory properties.

When tested in biological in vitro models, certain compounds of Table 1 were found to have pEC₅₀ values listed in Table 3.

TABLE 3

| | HCV Genotype | HCV Genotype | ISG56 |
|---|---|---|---|
| Compound | 1A | 1B | (Average Max |
| Number | Replicon | Replicon | Response) |
| (From Table 1) | pEC₅₀ (μM) | pEC₅₀ (μM) | % |
| 1 | 7.3 | 6.7 | 301.5 |
| 2 | 7.3 | 6.6 | 31 |
| 3 | 7 | 6.5 | 407.5 |
| 4 | 6.9 | 6.4 | 178.5 |
| 5 | 7 | 6.4 | 151.25 |
| 6 | 6.7 | 6.3 | 7.75 |
| 7 | 5.9 | 6.2 | — |
| 8 | 6.2 | 6.2 | 2.5 |
| 9 | 6.8 | 6.1 | 262 |
| 10 | 6 | 6.1 | 10 |
| 11 | 6.6 | 6.1 | 292.75 |
| 12 | 6.4 | 6.1 | 13.5 |
| 13 | 6.4 | 6 | 346 |
| 14 | 5.1 | 6 | 10 |
| 15 | 6.4 | 6 | 63.5 |
| 16 | 6.6 | 6 | 35.5 |
| 17 | 6.3 | 5.9 | 31.5 |
| 18 | 6.6 | 5.8 | 27 |
| 19 | 4.9 | 5.8 | 9 |
| 20 | 6 | 5.8 | 13.5 |
| 21 | 6.2 | 5.7 | 70 |
| 22 | 6.3 | 5.7 | 60.5 |
| 23 | 5.9 | 5.5 | 109.5 |
| 24 | 6.2 | 5.5 | 5.75 |
| 25 | 5.4 | 5.5 | 6.5 |
| 26 | 5.2 | 5.3 | 8 |
| 27 | 5.6 | 5.2 | 29 |
| 28 | 5 | 5.1 | 11.5 |
| 29 | 5.2 | 5.1 | 10 |
| 30 | 6.5 | 6 | — |
| 31 | 6.4 | 5.9 | — |

### Example 34

### Activation of the JAK/STAT Pathway Monitored in HEKBlue IFN-α/β Cells (InvivoGen)

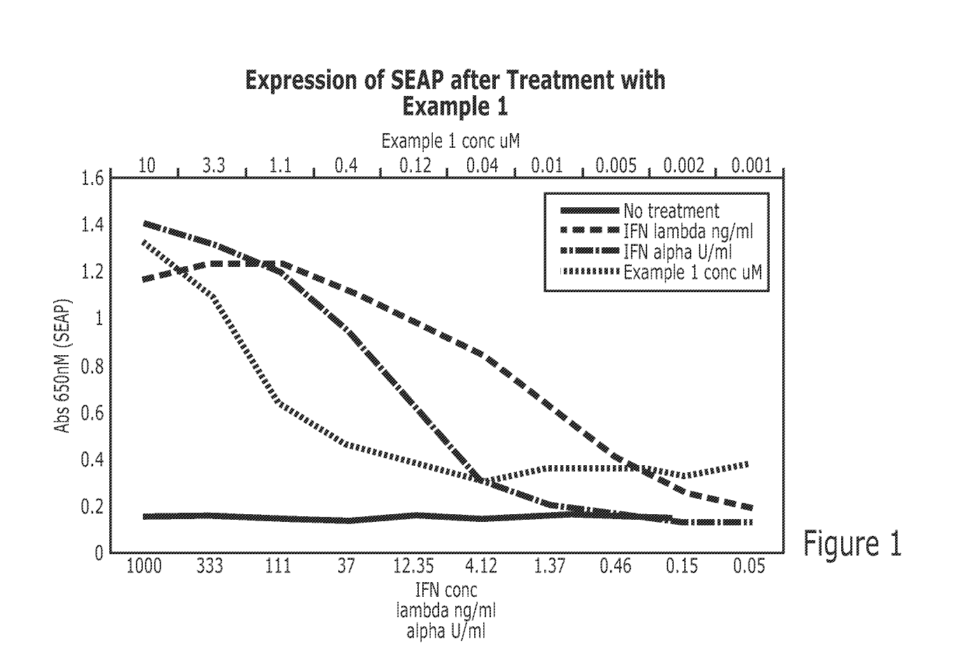

In this reporter cell line, the activation of the IFN mediated JAK/STAT pathway can be monitored by the level of the secreted alkaline phosphatase (SEAP), as shown in FIG. 1, the expression of which is under the control of the type I IFN inducible ISG54 promoter. The cells were incubated for 24 h in the presence of several treatments (Example 1, IFNα, and IFNλ3) and the supernatants were measured for the amount of the secreted alkaline phosphatase using QUANTI-Blue™ (InvivoGen) at the optical density of 650 nm. The treatment of Example 1 demonstrated the activation of the JAK/STAT pathway at an EC₅₀ of ˜1 μM. The activation of the pathway by IFNα (PBL) and IFNλ3 (R&D Systems Inc.) are shown as positive controls.

### Example 35

### Example 1 Induces STAT1 Phosphorylation

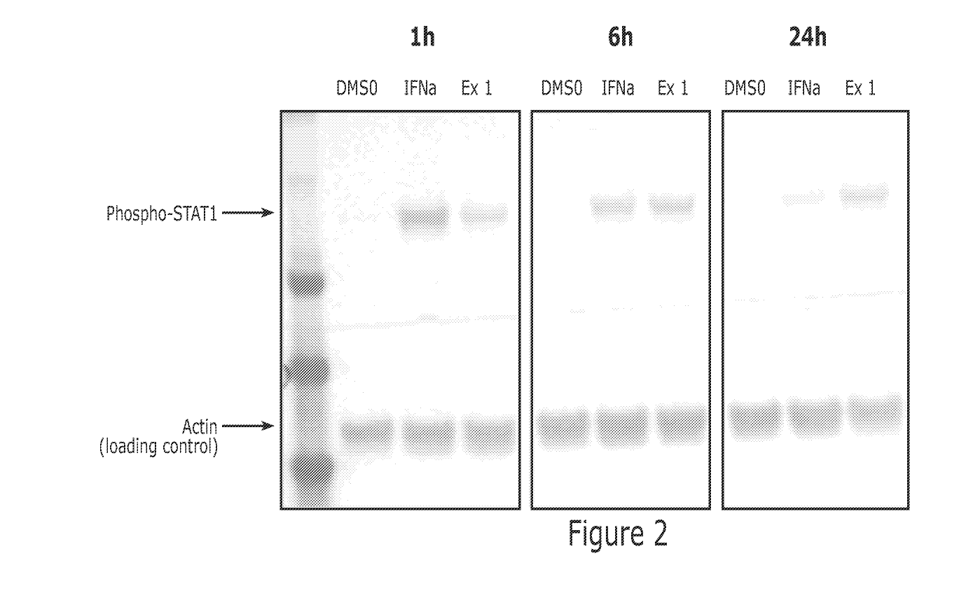

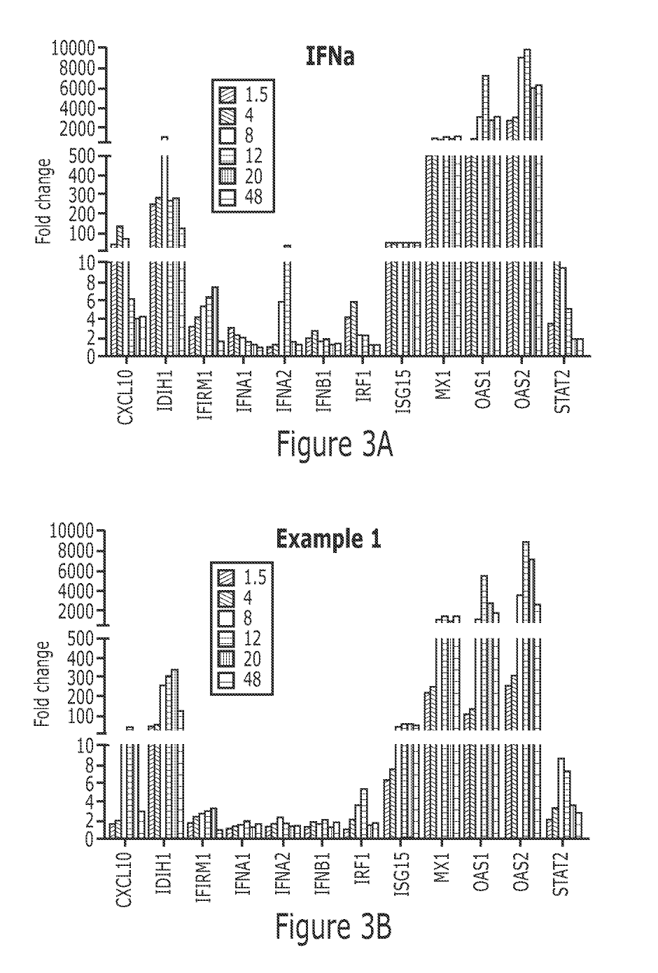

Cells harboring the hepatitis C virus replicon were treated with 2 uM of Example 1 for 1 h, 6 h, and 24 h, as shown in FIG. 2. IFNα (100 U/ml) and DMSO were included as controls. The total cell lysates were analyzed on a 4-20% SDS-PAGE gradient gel and followed by immunoblotting using anti-phopho STAT1 antibody (Cell Signaling). The level of actin was monitored as a loading control. The bands were visualized by the alkaline phosphatase activity conjugated with the secondary antibody (Promega) using ProtoBlot II AP System™ (Promega).

Treatment with IFNα or Example 1 induced STAT1 phosphorylation in a similar manner. However, the phosphorylated STAT1 was peaked at 1 h by the treatment of IFNα whereas the status of phosphor-STAT1 sustained up to 24 h with the treatment of Example 1.

### Example 36

### Confirmation of the Expression of the Interferon Stimulated Genes (ISGs) in HCV Replicon Cells

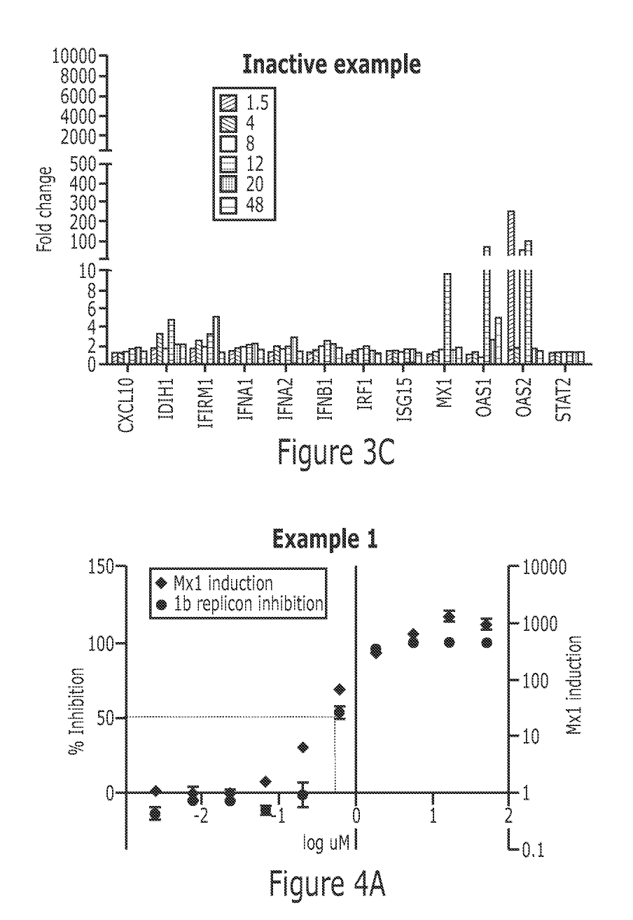

The up-regulation of interferon stimulated genes upon treatment with Example 1 was monitored by quantitative real time RT-PCR using specific primers for each gene, as shown in FIG. 3. The HCV replicon cells were treated with Example 1 (2 μM), an inactive analog compound (2 μM), or IFNα (100 U/ml) in a time course (1.5 h, 4 h, 8 h, 12 h, 20 h, and 48 h). Total RNA was isolated using RNeasy 96 kit (Qiagen) and converted to cDNA using High Capacity cDNA reverse transcription kit (Applied Biosystems). For real time PCR, the cDNA was used for PCR reactions using TaqMan Fast Universal PCR master mix (Applied Biosystems) and specific primers (Applied Biosystems). As housekeeping genes, actin and GAPDH were used for normalization. Data was calculated by the ΔΔCt method and fold change determined compared to DMSO-treated control samples.

The treatment of Example 1 gave rise to the induction of various known ISGs (ISG15, Mx1, OAS1, OAS2, CXCL10, IFIH1, and STAT2) in a time-dependent manner similar to the level observed by the treatment of IFNα. The maximum induction was observed at 8 h with the treatment of Example 1, which was slightly slower than 4 h detected by IFNα. Notably, there was no induction of IFNα and β mRNA (IFNA1, IFNA2, and IFNB1) suggesting that the mechanism of Example 1 is independent of the type I IFN production.

### Example 37

### Correlation of the Antiviral Activity with the Induction of ISG

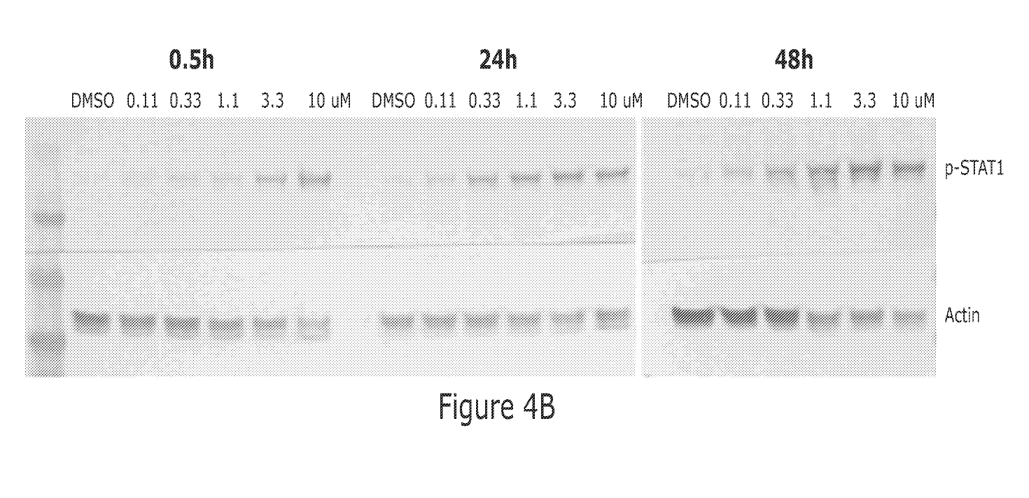

The activation of the JAK/STAT pathway was confirmed in a dose response of Example 1. The concentration of EC₅₀ in antiviral activity was similar to the concentration (˜0.2 μM) in which the onset of Mx1 induction (top panel) or phospho-STAT1 (bottom panel) was observed as shown in FIG. 4.

The top panel, as shown in FIG. 4, demonstrates the correlation of the antiviral activity in HCV replication and the induction of Mx1 mRNA. The replicon cells were treated with Example 1 in a dose response. After 48 hours, the cells were harvested for the measurement of the HCV replication by luminescence. The parallel replicate plate was harvested for RNA isolation and processed for a real time RT-PCR with Mx1 specific probe and primers (Applied Biosystems). The level of GAPDH was calculated at the same time and used for normalization (Applied Biosystems, GAPD endogenous control).

The bottom panel, as shown in FIG. 4, indicates the activation of phopho-STAT1 in a dose response of Example 1. The HCV replicon cell line was seeded in a 6 well plate in the presence of Example 1. The cells were harvested at 0.5 h, 24 h, and 48 h post treatment. The status of phosphor-STAT1 was observed by western blot as described above.

### Example 38

### Identification of the Key Factors in the JAK/STAT Pathway for the Antiviral Activity of Example 1 by Small Interfering RNA (siRNAs)

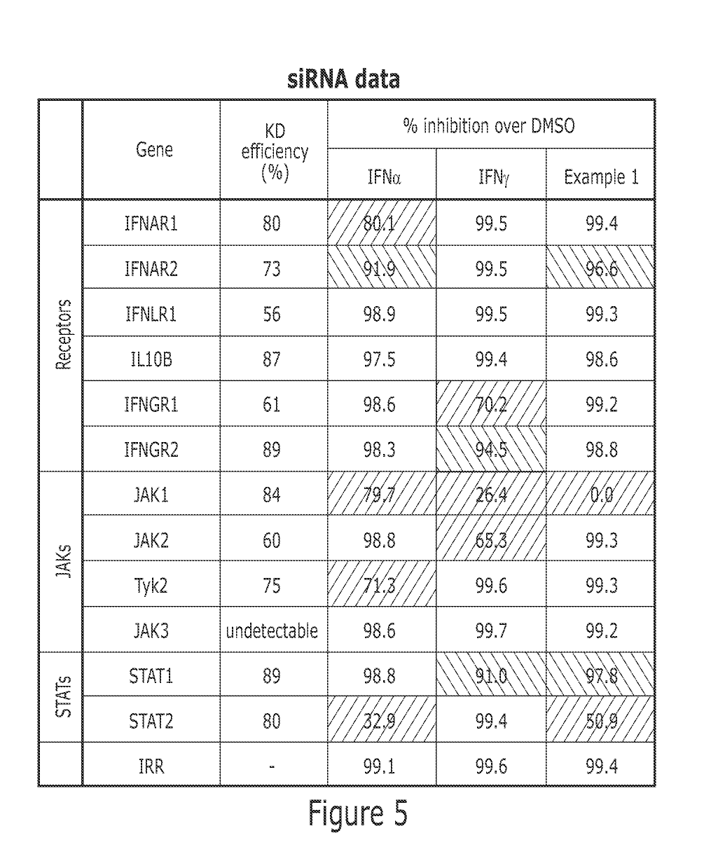

50 nM of siRNA against each gene (Dharmacon, on-target SMART pool: L-020209-00-0005 (IFNAR1), L-015411-00-0005 (IFNAR2), L-007981-00-0005 (IL28RA), L-007926-00-0005 (IL10RB), L-011-57-00-0005(IFNGR1), L-012713-00-0005 (IFNGR2), L-003145-00-0005 (JAK1), L-003146-00-0005 (JAK2), L-003182-00-0005 (Tyk2), L-003147-00-0 005 (JAK3), L-003543-00-0005 (STAT1), L-012064-00-0005 (STAT2)) was transfected in the 1b HCV replicon cells using lipofectamine RNAiMax™ (Invitrogen) according to the manufacturer's protocol. A scrambled irrelevant smart pool control siRNA was included as a control (IRR). After 3 days post transfection, the cells were treated with DMSO, IFNα (5 U/ml), IFNγ (100 U/ml), and Example 1 (2 μM) in triplicate for 30 h. The cells were harvested with Bright-Glo (Promega) and the HCV replication was measured by luminescence. For each gene, the % inhibition, as shown in FIG. 5, of HCV replication upon treatment was normalized based on the value of the DMSO treated cells. To measure the efficiency of the knockdown, total RNA was harvested from the siRNA-transfected cells at day 3 post transfection and analyzed by real time RT-PCR.

Among the key RNAs in the type I/II/III IFN pathways tested, the knockdown of IFNAR2, JAK1, STAT1, or STAT2 affected the antiviral activity of Example 1. Notably, the knockdown of JAK1 fully abolished the aniviral activity of Example 1 while the knockdown of other Janus kinases (JAKs) did not show any effect with Example 1, implying that JAK1 is closely related with the mechanism of Example 1 antiviral activity.

### Example 39

### Example 1 is a JAK1 Activator

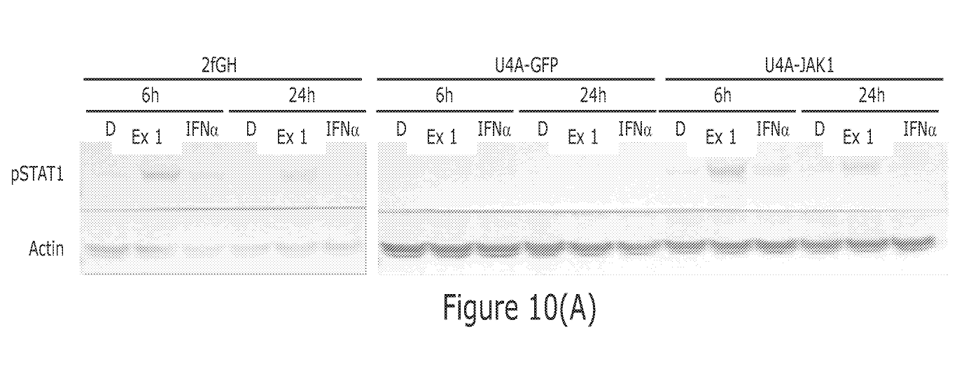

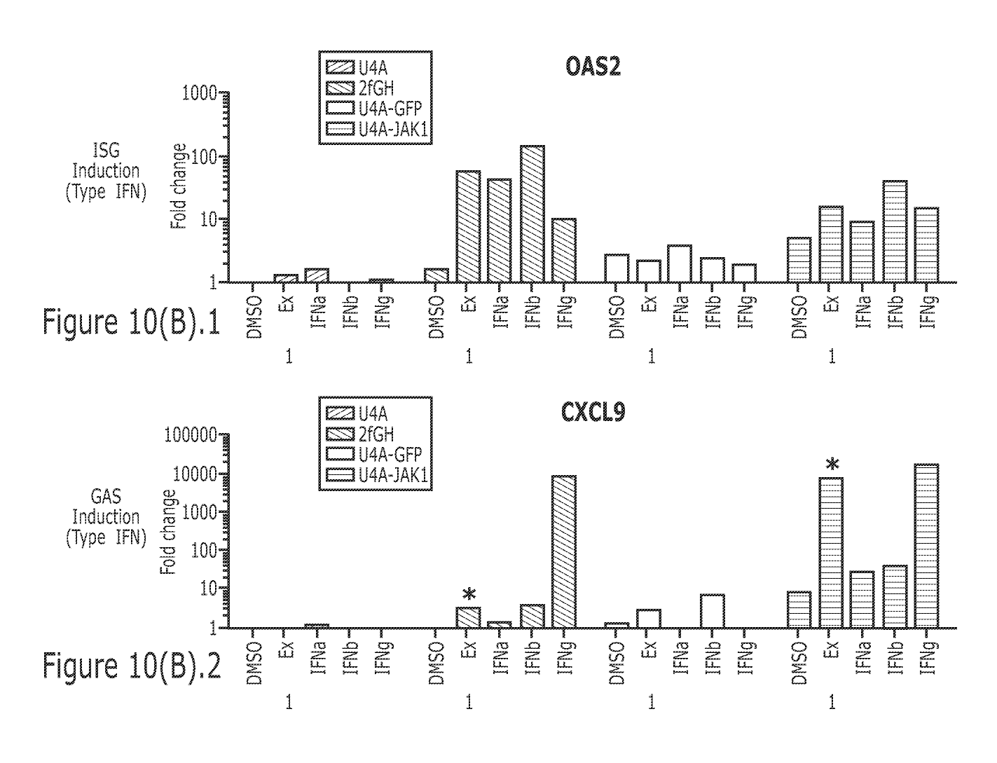

2fGH and U4A cells were obtained from the Cleveland Clinic. 2fGH is a human fibroblast cell line and U4A cell line is a derivative 2fGH harboring a defect in JAK1 expression (Muller, et al., Nature 366, 129-135 (1993)). Green fluoroscent protein (GFP) or human JAK1 was transduced in U4A cells by baculovirus mammalian expression system. After 24 hours post transduction, the cells were treated with DMSO, Example 1 (10 μM), IFNα (100 U/ml), IFNβ (100 U/ml), or IFNγ (100 U/ml). Untransduced U4A cells and 2fGH cells were included as controls for 6 or 18 hours. The cells were harvested at indicated time points and used for detecting phosphor-STAT1 by western blot (FIG. 10, panel A) or for mRNA analysis by Taqman quantitation (FIG. 10, panel B).

The total cell lysates were analyzed on a 4-20% SDS-PAGE gradient gel and followed by immunoblotting using anti-phopho STAT1 antibody (Cell Signaling). The level of actin was monitored as a loading control. The bands were visualized by the alkaline phosphatase activity conjugated with the secondary antibody (Promega) using ProtoBlot II AP system (Promega).

While p-STAT1 was not present in U4A cells (JAK1 deficient cell line) transduced with GFP upon treatment of Example 1 or IFNα, the activation of STAT1 was observed in U4A cells transduced with JAK1 upon treatment of Example 1 or IFNα indicating that the overexpression of human JAK1 resulted in the reconstitution of JAK/STAT pathway in U4A cells as shown in FIG. 10, Panel A. To confirm the induction of interferon stimulated genes (ISGs), the treated cells were processed for real time RT-PCR. cDNA was made using High Capacity cDNA Reverse Transcription Kit (Applied Biosystems). Gene expression was measured using TaqMan Fast Universal PCR Master Mix (Applied Biosystems) and gene specific probes and murine primers in HT7900 FAST System (Invitrogen). As housekeeping genes, actin and GAPDH were used for normalization. Data was calculated by the ΔΔCt method and fold change determined compared to non-treated control samples.

The gene expression of OAS2 and CXCL9 upon various treatments are shown in FIG. 10, Panel B). Example 1 induced a marked increase of mRNA for OAS2 similar to the level seen with IFNα or IFNβ in the parental cell line (2fGH). The expression of a gamma activated signal (GAS) gene, CXCL9, was only observed with IFNγ treatment in 2fGH cells. Interestingly, in U4A cells transduced with JAK1, the treatment with Example 1 gave rise to a marked up-regulation of CXCL9 similar to that of IFNγ treatment. The expression of OAS2 in U4A cells transduced with JAK1 was relatively comparable between treatments. This observation indicated that treatment with Example 1 was able to mimic type II IFNγ pathway in the context of U4A cells transduced with JAK1 implying JAK1 as a pivotal factor of the activity of Example 1 rather than IFN receptors.

### Example 40

### Confirmation of the Induction of ISGs in Mice In Vivo

Naive Balb/c mice were purchased from Charles River Laboratories (Wilmington, Mass.) and administered with murine IFNα2 (30 ug/kg) intravenously or administered with oral Example 1 (300 mg/kg in 30/70% solutol/polyethylene glycol 400). The mice were then euthanized by CO₂ inhalation at 0.5, 2, 6, 8, and 24 hours for sample collection. Four mice per dose group were tested.

For RNA isolation, the blood was collected in an RNAprotect™ tube (Qiagen) and processed with RNeasy Protect™ animal blood kit (Qiagen) according to the manufacturer's protocol. To preserve RNA, 30-200 mg of tissue pieces were stored in RNAlater™ solution (Invitrogen) until use. For RNA isolation, the thawed tissues were homogenized using a TissueLyser™ system (Qiagen) and processed with RNeasy 96 Universal Tissue Kit™ (Qiagen) according to the manufacturer's protocol. To remove DNA contamination, on-plate DNase digestion was included during the RNA purification.

For real time RT-PCR, cDNA was made using High Capacity cDNA Reverse Transcription Kit™ (Applied Biosystems). Gene expression was measured using TaqMan Fast Universal PCR Master Mix™ (Applied Biosystems) and gene specific probes and murine primers in HT7900 FAST System™ (Invitrogen). As housekeeping genes, actin and GAPDH were used for normalization. Data was calculated by the ΔΔCt method and fold change determined compared to non-treated control samples.

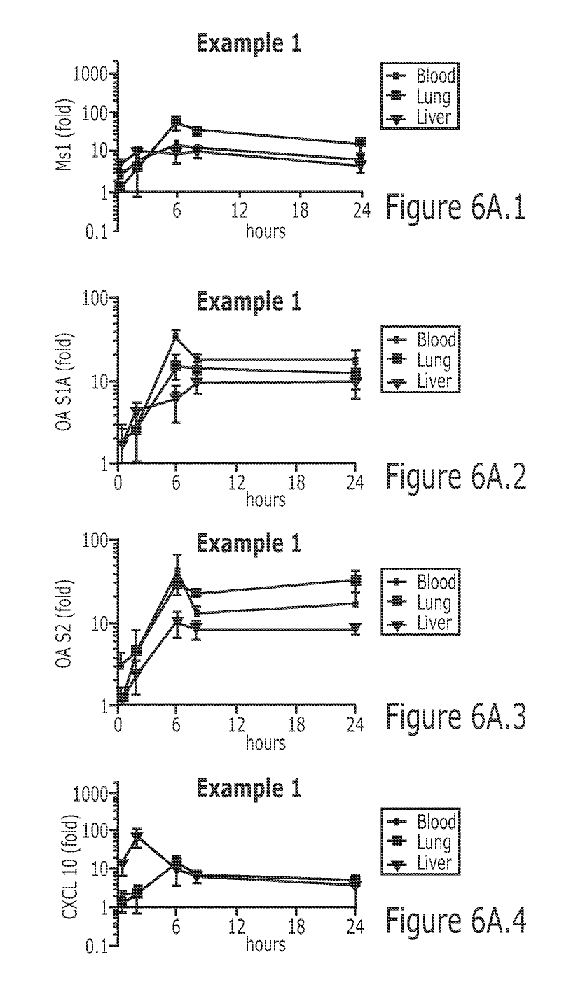

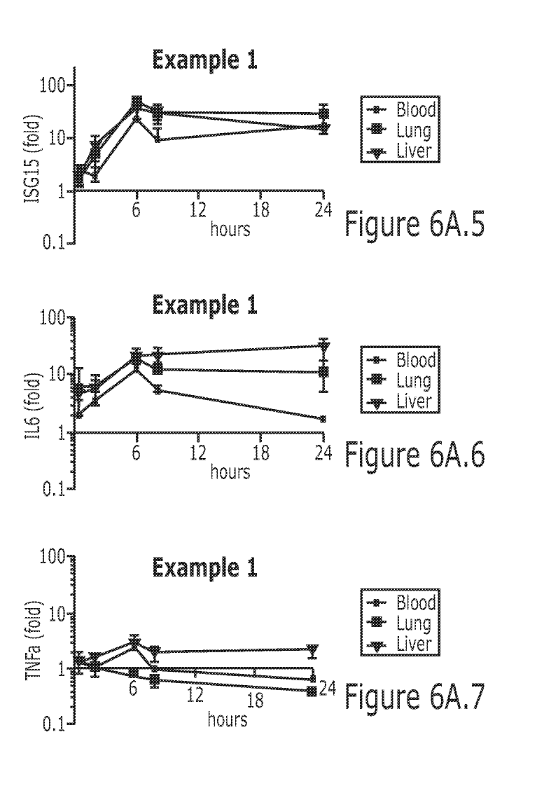

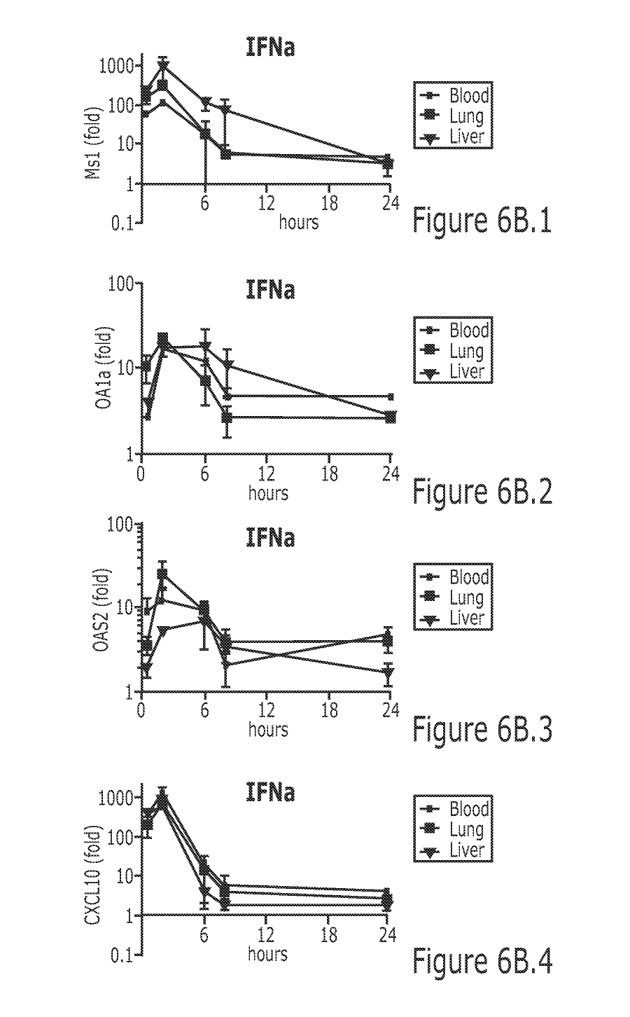

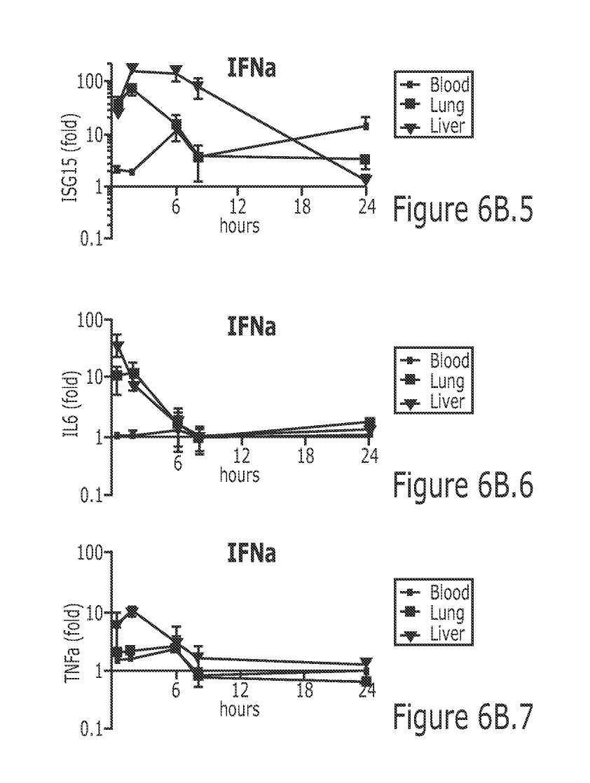

The gene expression of various ISGs and cytokines upon Example 1 (panel A) or murine IFNα (panel B) was shown in time course, as shown in FIG. 6. Example 1 induced a marked increase of mRNA for Mx1, OAS1A, OAS2, CXCL10, ISG15, and IL6 at 6 to 8 hours which correlates with the time to Cmax phamarcokinetically. Interestingly, the up-regulation of ISGs appeared to sustain at 24 hours whereas the induction by IFNα was evidently diminished by 24 h.

### Example 41

### Dose Response of ISGs (Induction In Vivo with Example 11

Naive male CD-1 mice were obtained from Charles Rivers Laboratories (Wilmington, Mass.) and administered with Example 11 in a dose response (0, 200, 600, and 1000 mg/kg; three mice per dose group) by oral gavage. The dose of 200 mg/kg was in 0.5% HPMC/0.1% Tween 80 whereas the rest of doses were in 30% solutol/70% PEG400. At 24 h, the blood and tissues were collected and processed as described above. The gene expressions of various ISGs and cytokines were monitored by real time RT-PCR (see above for the details).

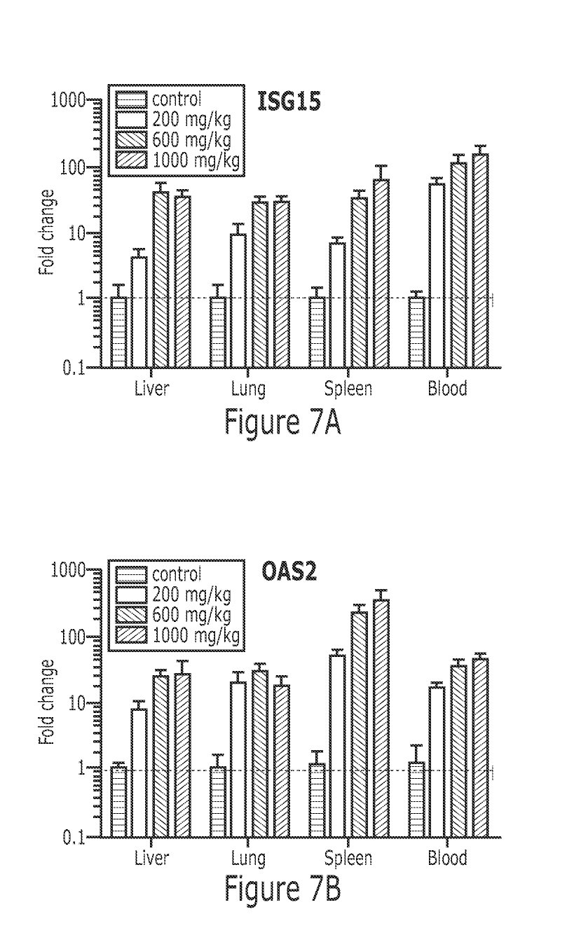

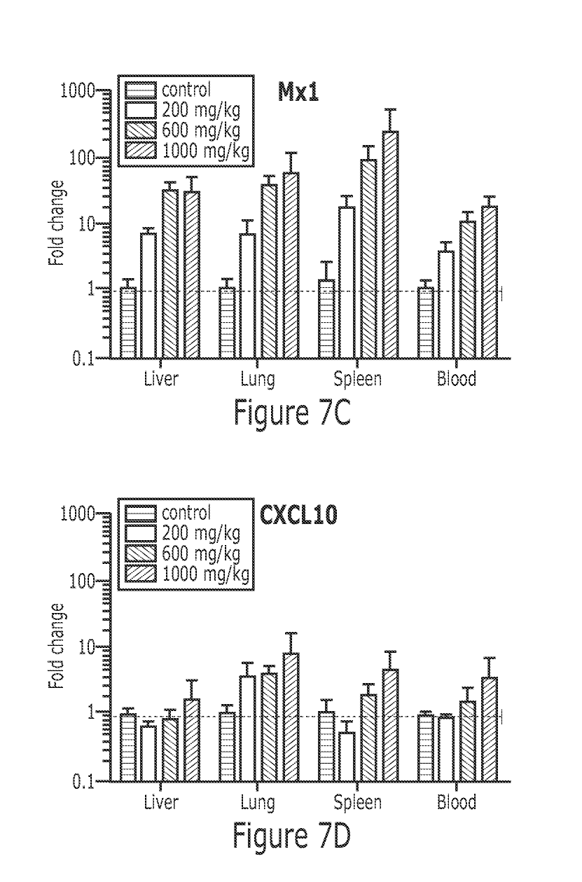

The induction of ISG appeared to be correlated with given doses in all tissues, as shown in FIG. 7.

### Example 42

### A Broad Spectrum of Antiviral Activity of Example 1

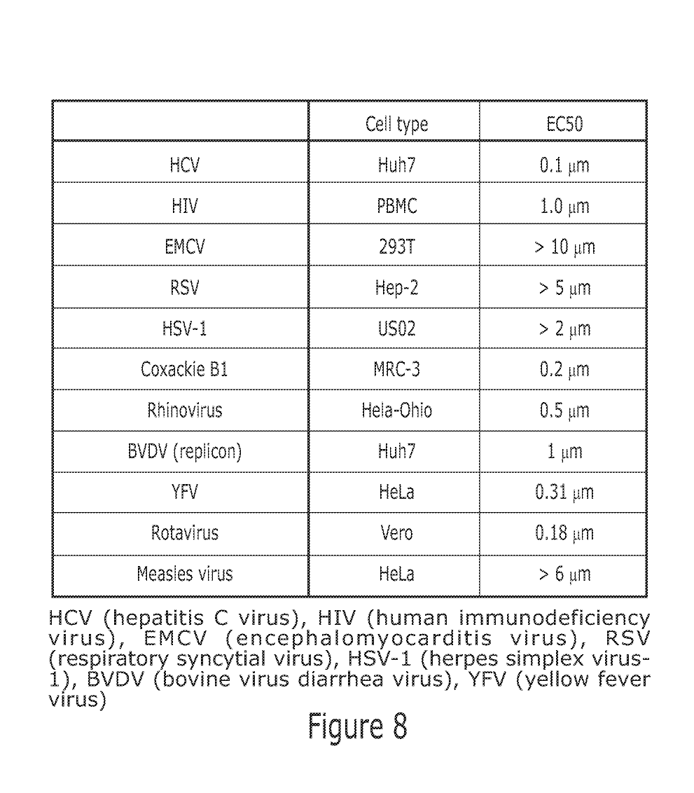

A broad spectrum of antiviral activity of Example 1 was accessed by testing it for potency against other viruses. (See FIG. 8). Selecting one such virus, the inhibition of the replication of respiratory syncytial virus (RSV), a negative strand RNA virus, by Example 1 was also demonstrated in a plaque assay. RSV (long form) was inoculated on the layer of HEp-2 cells at multiplicity of infection of <0.001. After 4 hour post infection, the inoculum was replaced with 0.3% agarose containing MEM and various concentration of Example 1 or IFNα. The cells were incubated for up to 5-6 days until the plaques were visible. The cells were then fixed with 3% formaldehyde and stained with neutral red for visualization.

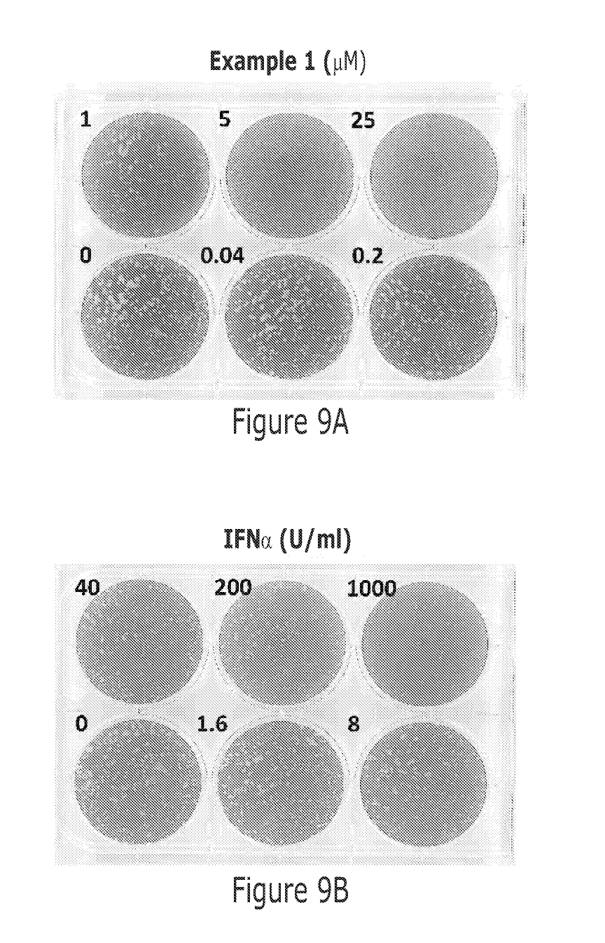

The treatment of Example 1 at 1 μM reduced the number of plaques, as shown in FIG. 9, compared to that observed with DMSO-only treated. With 5 μM of Example 1, no visible clear plaque was observed, suggesting that RSV replication was severely hindered by treatment of Example 1. Similarly, the treatment of IFNα at 1000 U/ml significantly reduced the number and size of the plaques.

### Example 43

### Induction of the JAK/STAT Pathway by Certain Compounds Described Herein in Human Skin Keratinocyte Cells

This example shows that certain compounds described herein can induce the JAK/STAT and Interferon pathway in human skin cells (keratinocytes), and therefore, potentially increase the antiviral capabilities of those cells. An induction of the antiviral capabilities of human keratinocytes could feasibly lead to a method for treating and/or preventing viral infections on or in human skin or mucous membranes, such as, for example, human papilloma infections causing common warts.

First, reconstructed human epidermis (“RHE”) consisting of cultured human keratinocyte cells were incubated in triplicate with media containing either the media alone, media+0.1% DMSO, media containing 10 μM of several putative JAK/STAT activator compounds described herein in Tables 1 and 2 (Example 1, Example 2, Example 11, and compound no. 89 (as a negative control)), or media containing 100 U/mL of a positive control Interferon-alpha (IFN-alpha) recombinant protein at 37° C. in a humidified atmosphere containing 5% CO₂, for 6 and 72 hours.

At the end of the incubation period, the RHE tissues were cut into two sections. One section was ¼ of the total size and the second section was of the total size. The smallest section (¼) was then used for RNA isolation and gene expression analysis of interferon-stimulated genes [ISG], such as MX1 and OAS2, and IL-6 by real-time quantitative PCR and the largest part (¾) was used for protein extraction and western blot analysis of Stat1 phosphorylation.

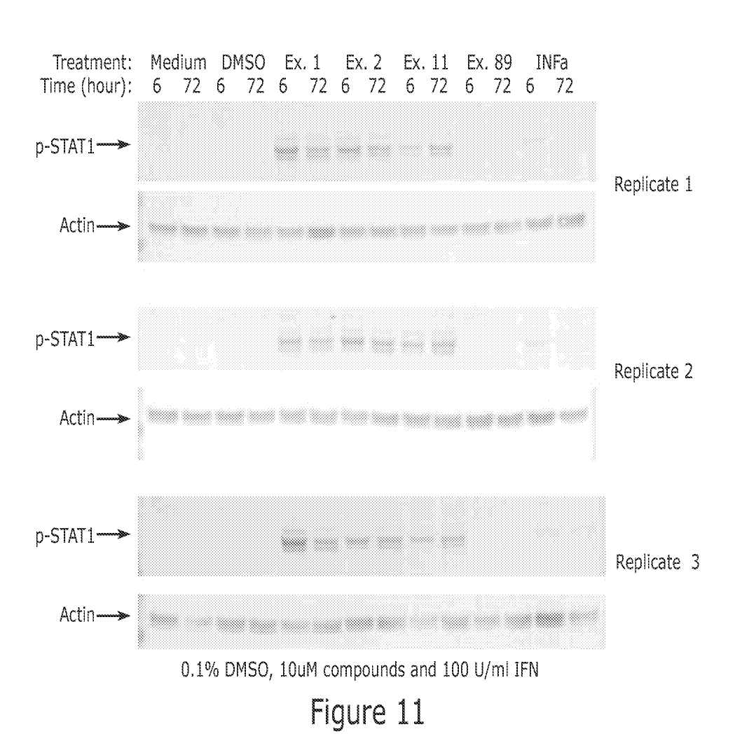

Western blot analysis in FIG. 11 showed Stat1 phosphorylation at 6 and 72 hrs in all the triplicate RHE cultures treated with the aforementioned putative JAK/Stat activators (Example 1, Example 2, and Example 11) as well as for the positive control, INF-alpha (FIG. 11). The negative controls (compound no. 89, media alone, and media+0.1% DMSO) did not show phosphorylation of Stat1 at any time-point tested.

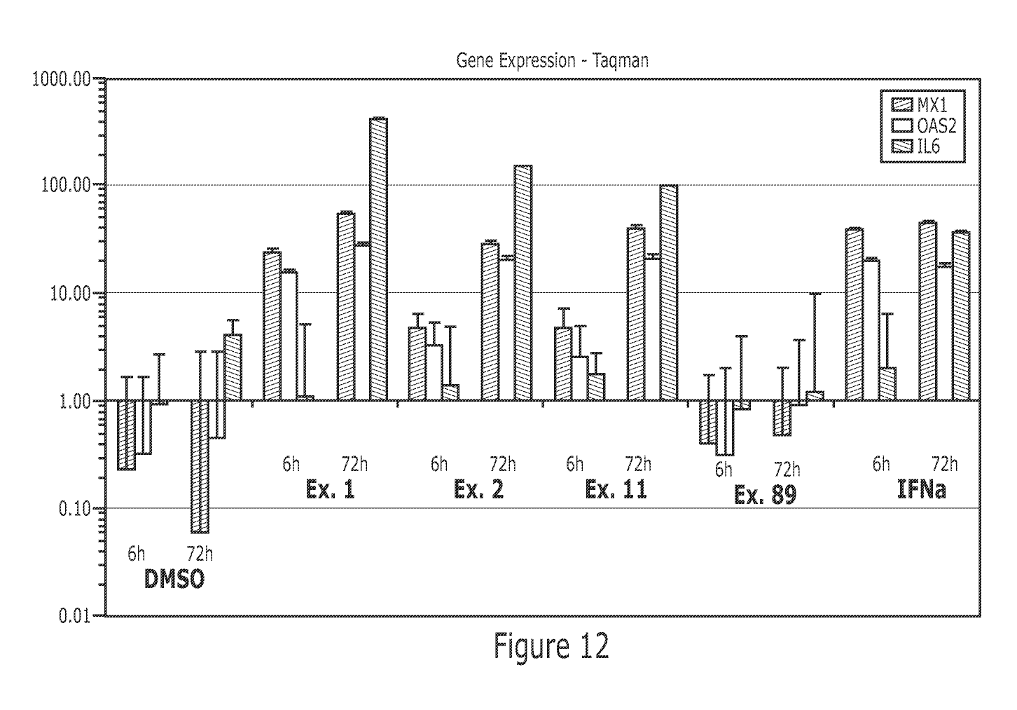

Gene expression analysis at 6 and 72 hrs post JAK/Stat activators (Example 1, Example 2, and Example 11) treatment show significant upregulation (>10-fold) of ISG expression, including MX1, OAS2, and IL-6, similar to IFNalpha (FIG. 12) in RHE. A closely related analog compound no. 89, which was negative in the JAK/STAT activator assay, was used as a negative control in this experiment. Taken together, these data demonstrate that human keratinocytes can be stimulated by JAK/Stat activators and have the potential to induce an anti-viral response in human skin cells.

### Example 44

### JAK/STAT Activators Induce Interferon Stimulated Gene (ISG) Expression in 1106 KERTr (E6/E7 Transformed) Human Keratinocytes

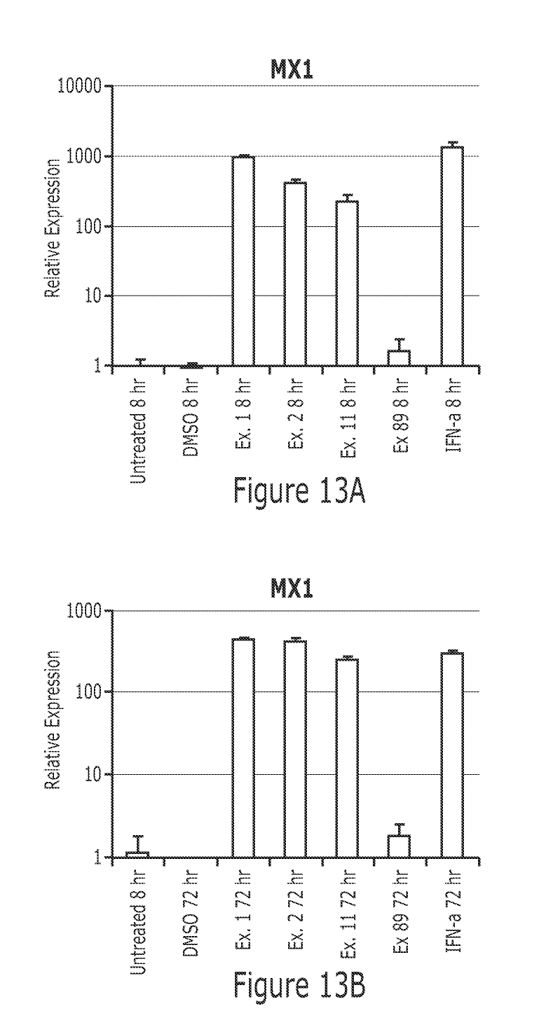

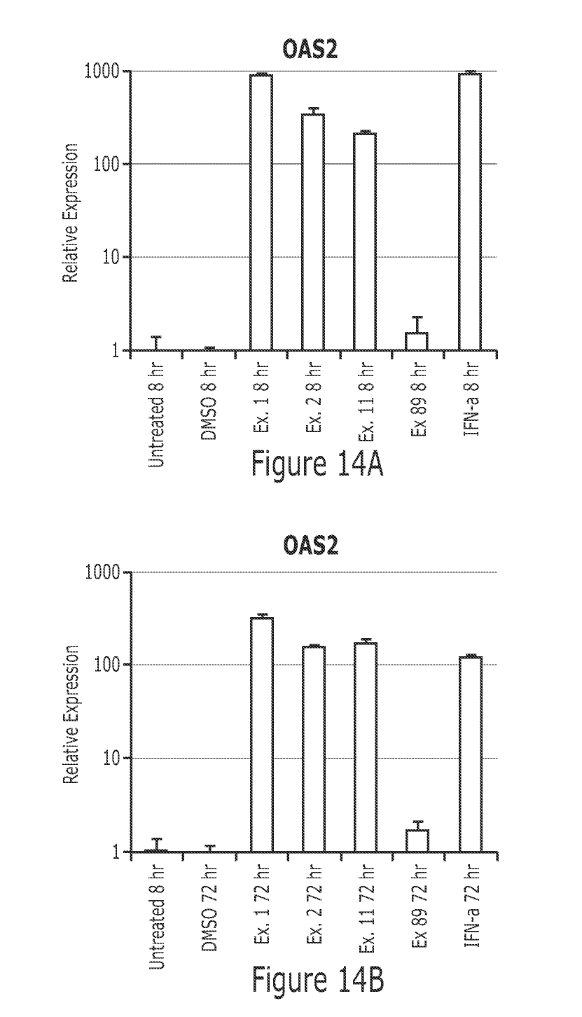

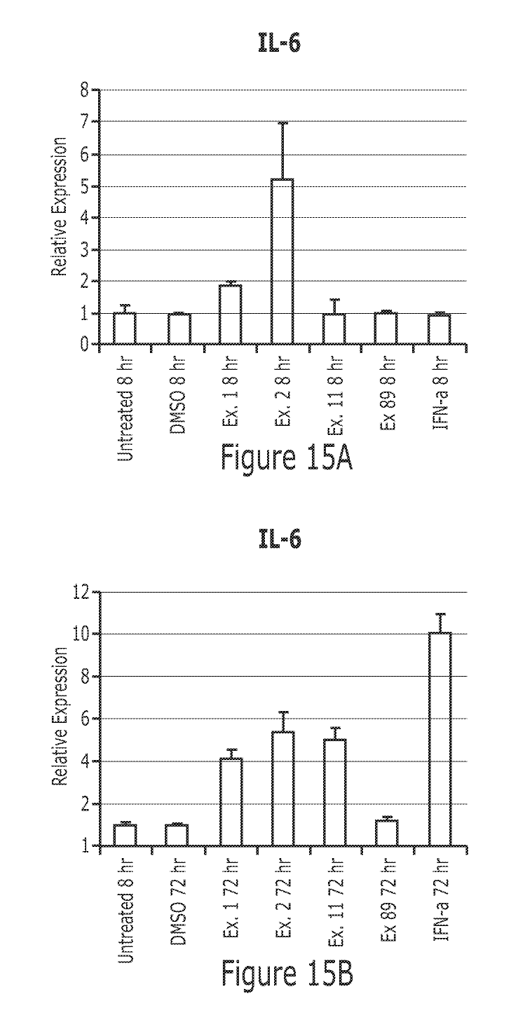

This example shows that certain JAK/STAT activators (compounds) of the present invention can induce Interferon Stimulated Gene (ISG) expression in 1106 KERTr (E6/E7 transformed) human keratinocytes. Keratinocytes expressing E6 and E7 from HPV type 18 were treated in triplicate with media containing either the media alone, media+0.1% DMSO, media containing 10 μM of each of the JAK/Stat activator (The JAK/Stat activators (Ex. 1, 2, 11, and 89 [inactive]), or media containing 100 U/mL of IFN-alpha recombinant protein at 37° C. in a humidified atmosphere containing 5% CO₂, for 68 and 72 hours. At the end of the incubation, cells were harvested for RNA isolation. Gene expression analysis at 8 and 72 hrs post JAK/Stat activators (Ex. 1, 2, and 11) treatment show significant upregulation (>100-fold) in FIGS. 13-15 of ISG expression, including MX1, OAS2, and IL-6, similar to IFNalpha (FIG. 12) in RHE. A closely related analog Ex. 89, which was negative in the JAK/STAT activator assay, was used as a negative control in this experiment. These results suggest that JAK/STAT activators can overcome the ISG inhibition by E6 and E7 and have potential therapeutic effect against human papillomavirus infection, and thereby, for example, a treatment for warts.

### Example 45

### Tablet Formulation

The following ingredients are mixed intimately and pressed into single scored tablets.

| Ingredient | Quantity per tablet (mg) |
|---|---|
| compound | 400 |
| cornstarch | 50 |
| croscarmellose sodium | 25 |
| lactose | 120 |
| magnesium stearate | 5 |

### Example 46

Capsule Formulation

The following ingredients are mixed intimately and loaded into a hard-shell gelatin capsule.

| Ingredient | Quantity per capsule (mg) |
|---|---|
| compound | 200 |
| Lactose, spray-dried | 148 |
| magnesium stearate | 2 |

### Example 47

Suspension Formulation

The following ingredients are mixed to form a suspension for oral administration.

| Ingredient | Amount | |
|---|---|---|
| compound | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. (quantity sufficient) to 100 mL |

### Example 48

### Injectable Formulation

The following ingredients are mixed to form an injectable formulation.

| Ingredient | Amount |
|---|---|
| compound | 0.2 mg-20 mg |
| sodium acetate buffer solution, | 0.4M 2.0 mL |
| HCl (1N) or NaOH (1N) | q.s. to suitable pH |
| water (distilled, sterile) | q.s. to 20 mL |

### Example 49

### Suppository Formulation

A suppository of total weight 2.5 g is prepared by mixing the compound with Witepsol® H-15 (triglycerides of saturated vegetable fatty acid; Riches-Nelson, Inc., New York), and has the following composition:

| Ingredient | Amount |
|---|---|
| compound | 500 mg |
| Witepsol ® H-15 | balance |

### Example 50

### Topical Formulation

The following ingredients are mixed into a dermatological formulation for topical administration of a compound of the present invention to a skin wart.

| Ingredient | Quantity per application (%) |
|---|---|
| Compound (Ex. 2) | 0.05% |
| Propylene glycol | 10.0% |
| Microcrystalline wax | 10.0% |
| Cetostearyl alcohol | 2.0% |
| Liquid paraffin | 32.5% |
| Isopropyl myristate | 7.5% |
| *Arlacel ™ 165 | 2.0% |
| Sorbitan monostearate | 1.0% |
| Dimethicone 360 | 2.5% |
| Imidurea | 0.2% |
| Dibasic sodium phosphate | 0.06% |
| Citric acid, Hydrous | 0.05% |
| Purified water | to 100.0% |

[*Glyceryl stearate and PEG 100 stearate]

Although the invention has been shown and described above with reference to some embodiments, those skilled in the art will readily appreciate that the specific experiments detailed are only illustrative of the invention. It should be understood that various modifications can be made without departing from the spirit of the invention.

For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims. Accordingly, the invention is limited only by the following claims. All publications, issued patents, patent applications, books and journal articles, cited in this application are each herein incorporated by reference in their entirety.

1. A compound having the structure according to Formula (I): 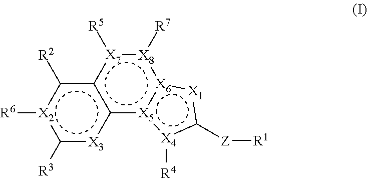
or a pharmaceutically acceptable salt thereof, wherein:
X₁, X₄, X₇, and X₈, are independently selected from N, NH, S, O, C, CH, or CH₂;
X₂, X₃, X₅, and X₆ are independently selected from N, C, or CH;
Z is selected from a bond, —C(O), or (C₁-C₆)alkylene;
R¹ is selected from the group consisting of hydrogen, —R¹², —R¹⁴, R⁹(R¹⁵)ₘ, —OR⁹(R¹⁵)ₘ, and halo;
R² is selected from the group consisting of hydrogen, (C₁-C₆)alkyl, (C₁-C₆)alkoxy, —R¹², —R¹⁴, C(O)R¹², —R⁹R², —R⁹R¹³, —R⁹R¹⁴, —C(O)R¹⁴, —R⁹(R¹⁵)ₘ, —OR⁹(R¹⁵)ₘ, —OR¹³, —R¹²S(O)₂, —S(O)₂R¹², halo, nitrile, sulfonamide, sulfone, sulfoxide, (C₄-C₁₄)aryl, and (C₃-C₁₂)cycloalkyl, wherein said R² group may be optionally substituted with one to three R¹¹ groups;
R³ is selected from the group consisting of hydrogen, (C₁-C₆)alkyl, (C₁-C₆)alkoxy, —R¹², —R¹⁴, C(O)R², —R⁹R¹², —R⁹R¹³, —R⁹R¹⁴, —C(O)R¹⁴, —R(R¹⁵)ₘ, —OR⁹(R¹⁵)ₘ, —OR¹³, halo, nitrile, sulfonamide, sulfone, sulfoxide, (C₄-C₁₄)aryl, and (C₃-C₁₂)cycloalkyl, wherein said R³ group may be optionally substituted with one to three R¹¹ groups;
R⁴ is optionally absent or is selected from the group consisting of hydrogen, (C₁-C₆)alkyl, —R(R¹⁵)ₘ, —OR(R)ₘ, —R⁹R¹⁰, —C(O)R⁹, —C(O)R¹³, halo, and (C₃-C₁₂)cycloalkyl;
R⁵ is selected from the group consisting of hydrogen, (C₁-C₆)alkyl, —C(O)R¹², —R⁹R¹², —R⁹(R¹⁵)ₘ, —OR⁹(R¹⁵)ₘ, —R¹⁴, halo, and nitrile;
R⁶ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R(R¹⁵)ₘ, —OR⁹(R¹⁵)ₘ, —OR¹³, halo, nitrile, sulfonamide, sulfone, sulfoxide, (C₄-C₁₄)aryl, and (C₃-C₁₂)cycloalkyl, wherein said R⁶ group may be optionally substituted with one to three R¹¹ groups;
R⁷ is selected from the group consisting of hydrogen, (C₁-C₆)alkyl, —R⁹(R¹⁵)ₘ, —OR(R¹⁵)ₘ, halo, —C(O)R¹², —R⁹R¹², nitrile, and —R¹⁴;
R⁸ is independently selected from the group consisting of hydrogen and (C₁-C₆)alkyl;
R⁹ is (C₁-C₆)alkyl;
R¹⁰ is (C₄-C₁₄)aryl;
R¹¹ is selected from the group consisting of (C₁-C₆)alkyl, dimethyl, sulfonamide, —OR⁸, —C(O)R¹², oxo, nitrile, —R¹², halo, —R⁹ (R¹⁵)ₘ, and —OR⁹(R¹⁵)ₘ;
R¹² is —NR^{x}R^{y}, wherein R^{x} and R^{y} are independently selected from the group consisting of hydrogen and (C₁-C₆)alkyl; wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring independently have one to four heteroatoms selected from N, S and O, and wherein said heterocyclic ring or heteroaryl ring may be also optionally substituted with one to three R¹¹ groups;
R¹³ is (C₃-C₁₂)cycloalkyl;
R¹⁴ is selected from the group consisting of (C₁-C₁₁)heteroaryl or (C₁-C₁₁)heterocyclic, wherein said (C₁-C₁₁)heterocyclic or (C₁-C₁₁)heteroaryl each may have one to three heteroatoms selected from N, S, or O, and wherein said (C₁-C₁₁)heteroaryl or (C₁-C₁₁)heterocyclic may also be optionally substituted by one to three independent R¹¹ groups;
R¹⁵ is halo; and
m is independently 0 or an integer from 1 to 3.

2. The compound according to claim 1, or a pharmaceutically acceptable salt thereof, wherein:
X₁, X₄, X₇, and X₈, are independently selected from N, NH, C, S, O, CH, or CH₂;
X₂, X₃, X₅, and X₆ are independently selected from N, C, or CH;
Z is selected from the group consisting of a bond, —C(O), and methylene;
R¹ is selected from the group consisting of hydrogen, —R¹², chloro, bromo, fluoro, difluoromethyl, trifluoromethyl, difluoromethoxy, trifluoromethoxy, oxazolyl, furanyl, oxolanyl, oxadiazolyl, oxazolidinyl, imidazolidinyl, imidazolyl, oxanyl, piperidinyl, morpholinyl, dihydropyranyl, pyranyl, tetrahydropyridinyl, pyridinyl, and pyrrolidinyl, wherein said R¹ group may be optionally substituted with one to three R¹¹ groups;
R² is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R², R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R² may be optionally substituted by one to three independent R¹¹ groups;
R³ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R³ may be optionally substituted by one to three independent R¹¹ groups;
R⁴ is optionally absent or is selected from the group consisting of hydrogen, methyl, ethyl, propyl, butyl, —C(O)R⁹, —(CO)R¹³, chloro, bromo, fluoro, difluoromethyl, trifluoromethyl, difluoromethoxy, trifluoromethoxy, cyclopropyl, cyclobutyl, cyclopentyl, and cyclohexyl;
R⁵ is selected from the group consisting of hydrogen, methyl, ethyl, propyl, butyl, —C(O)R¹², —R⁹R¹², nitrile, chloro, bromo, fluoro, difluoromethyl, trifluoromethyl, difluoromethoxy, trifluoromethoxy, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R⁶ may be optionally substituted by one to three independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl; and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, each independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, imidazolyl, thiophenyl, oxanyl, pyrrolidinyl, furanyl, morpholinyl, oxazolyl, oxadiazolyl, oxazolidinyl, dihydropyranyl, tetrahydropyridinyl, imidazolidinyl, and pyridinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

3. The compound according to claim 1, or a pharmaceutically acceptable salt thereof, wherein:
X₁, X₂, X₃, X₄, X₅, X₆ X₇, and X₈, are independently selected from N, NH, or CH;
Z is selected from a bond or methylene;
R¹ is selected from the group consisting of oxazolyl, oxanyl, oxolanyl, oxadiazolyl, oxazolidinyl, dihydropyranyl, tetrahydropyridinyl, pyrrolidinyl, morpholinyl, imidazolidinyl, and furanyl, wherein said R¹ group may be optionally substituted with one to two R¹¹ groups;
R² is selected from the group consisting of hydrogen, methyl, ethyl, propyl, isopropyl, butyl, isobutyl, methoxy, ethoxy, propoxy, chloro, bromo, fluoro, nitrile, difluoromethyl, trifluoromethyl, difluoromethoxy, trifluoromethoxy, cyclopropyl, cyclobutyl, cyclopentyl, cyclohexyl, imidazolyl, phenyl, and oxolanyl, wherein R² may be optionally substituted by one to two independent R¹¹ groups;
R³ is selected from the group consisting of hydrogen, methyl, ethyl, propyl, isopropyl, butyl, isobutyl, methoxy, ethoxy, propoxy, nitrile, difluoromethyl, trifluoromethyl, —C(O)R¹², oxanyl, oxolanyl, pyridinyl, phenyl, thiophenyl, piperidinyl, pyrrolidinyl, wherein R³ may be optionally substituted by one to two independent R¹¹ groups;
R⁴ is optionally absent or is selected from the group consisting of hydrogen, methoxy, ethoxy, propoxy, methyl, ethyl, propyl, butyl, nitrile, —C(O)R⁹, —(CO)R¹³, chloro, bromo, and fluoro;
R⁵ is selected from the group consisting of hydrogen, methyl, ethyl, propyl, butyl, —C(O)R², —R⁹R¹², nitrile, methoxy, ethoxy, propoxy, nitrile, chloro, bromo, fluoro, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, difluoromethoxy, trifluoromethoxy, piperidinyl, morpholinyl, oxolanyl, wherein R⁶ may be optionally substituted by one to two independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl; and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, each independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

4. The compound according to claim 1, or a pharmaceutically acceptable salt thereof, wherein:
X₁, X₂, X₃, X₄, X₅, X₆ X₇, and X₈, are independently selected from N or CH;
Z is selected from a bond or methylene;
R¹ is selected from the group consisting of oxazolyl, oxanyl, oxolanyl, oxadiazolyl, oxazolidinyl, dihydropyranyl, tetrahydropyridinyl, pyrrolidinyl, morpholinyl, imidazolidinyl, and furanyl, wherein said R¹ group may be optionally substituted with one to two R¹¹ groups;
R² is selected from the group consisting of hydrogen, methyl, ethyl, propyl, isopropyl, butyl, isobutyl, methoxy, ethoxy, propoxy, chloro, bromo, fluoro, nitrile, difluoromethyl, trifluoromethyl, difluoromethoxy, trifluoromethoxy, cyclopropyl, cyclobutyl, cyclopentyl, cyclohexyl, imidazolyl, phenyl, and oxolanyl, wherein R² may be optionally substituted by one to two independent R¹¹ groups;
R³ is selected from the group consisting of hydrogen, methyl, ethyl, propyl, isopropyl, butyl, isobutyl, methoxy, ethoxy, propoxy, nitrile, difluoromethyl, trifluoromethyl, —C(O)R¹², oxanyl, oxolanyl, pyridinyl, phenyl, thiophenyl, piperidinyl, pyrrolidinyl, wherein R³ may be optionally substituted by one to two independent R¹¹ groups;
R⁴ is optionally absent or is selected from the group consisting of hydrogen, methoxy, ethoxy, propoxy, methyl, ethyl, propyl, butyl, nitrile, —C(O)R⁹, —C(O)R¹³, chloro, bromo, and fluoro;
R⁵ is selected from the group consisting of hydrogen, methyl, ethyl, propyl, butyl, —C(O)R¹², —R⁹R¹², nitrile, methoxy, ethoxy, propoxy, nitrile, chloro, bromo, fluoro, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, difluoromethoxy, trifluoromethoxy, piperidinyl, morpholinyl, oxolanyl, wherein R⁶ may be optionally substituted by one to two independent R¹¹ groups;
R⁷ is selected from the group consisting of hydrogen, —C(O)R², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹⁰ is phenyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from hydrogen or methyl;
R¹³ is selected from the group consisting of cyclopropyl, cyclobutyl, cyclopentyl, and cyclohexyl;
R¹⁴ is selected from the group consisting of piperidinyl, oxolanyl, morpholinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

5. The compound according to claim 1, or a pharmaceutically acceptable salt thereof, wherein R¹ is selected from the group consisting of thiophenyl, furanyl, pyridinyl, tetrahydrofuranyl, tetrahydropyranyl, methylpyrrolidinyl, methylpiperdidinyl, 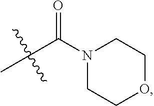 [SMILES: *C(=O)N1CCOCC1]
and methyl-morpholinyl.

6. The compound according to claim 1, or a pharmaceutically acceptable salt thereof, wherein R² is selected from the group consisting of morpholinyl, methylpiperidinyl, and tetrahydrofuranyl.

7. The compound according to claim 1, or a pharmaceutically acceptable salt thereof, wherein R³ is selected from the group consisting of tetrahydrofuranyl, piperidinyl, pyrrolidinyl, 1H-imidazolyl, propanyloxy, and carbonyl-morpholinyl.

8. The compound according to claim 1, or a pharmaceutically acceptable salt thereof, wherein R⁴ is pyrrolidinyl.

9. The compound according to claim 1, or a pharmaceutically acceptable salt thereof, wherein R⁵ is pyrrolidinyl.

10. The compound according to claim 1, or a pharmaceutically acceptable salt thereof, wherein R⁶ is selected from the group consisting of oxadiazolyl, furanyl, oxazolyl, methyl-pyrrolidyl, methyl-pyrrolidinol, methyl-morpholinyl, oxazolidinone, pyrrolidinone, imidazolidinone, imidazolidinedione, and methyl-oxazole.

11. The compound according to claim 1, or a pharmaceutically acceptable salt thereof, wherein:
X₁, X₂, X₃, X₄, X₅, X₆ X₇, and X₈ are selected from N and CH;
R¹ is selected from the group consisting of hydrogen, cyclopentyl, cyclopropyl, propan-2-yl, methyl, ethyl, 2-methylpropyl, thiophen-3-yl, furan-3-yl, pyridine-3-yl, ethoxy, phenyl, difluoromethoxy, chloride, tetrahydrofuran-(2 or 3)-yl, tetrahydropyran-(3 or 4)-yl, 1-methylpyrrolidin-(2 or 3)-yl, 1-methyl-(3 or 4)-piperdidinyl, carboxamide 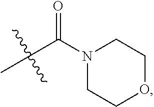 [SMILES: *C(=O)N1CCOCC1]
N,N-dimethyl-carboxamide, N-methyl-carboxamide, methyl-dimethylamine, 4-methyl-morpholinyl, 4-carbonyl-morpholinyl, cyclopentyl-methyl, and trifluoromethyl;
R² is selected from the group consisting of hydrogen, trifluoromethyl, propan-2-yl, morpholin-4-yl, 1-methylpiperidin-4-yl, and tetrahydrofuran-3-yl;
R³ is selected from the group consisting of hydrogen, trifluoromethyl, chloride, methyl, propan-2-yl, 2-methylpropyl, phenyl, cyclopropyl, cyclobutyl, cyclopentyl, cyclohexyl, tetrahydrofuran-(2 or 3)-yl, and piperidin-1-yl, pyrrolidin-1-yl, 1H-imidazol-(2 or 5)-yl, propan-2-yloxy, ethoxy, cyano, carboxamide, and carbonyl-morpholinyl;
R⁴ is optionally absent or is selected from the group consisting of hydrogen, pyrrolidin-1-yl, cyano, carboxamide, and dimethyl-methylamine;
R⁵ is selected from the group consisting of hydrogen, pyrrolidin-1-yl, cyano, carboxamide, and dimethyl-methylamine;
R⁶ is selected from the group consisting of hydrogen, 1,3,4-oxadiazol-2-yl, furan-2-yl, 1,3-oxazol-2-yl, methyl-dimethylamine, 1-methyl-pyrrolidyl, 1-methyl-pyrrolidin-3-ol, 4-methyl-morpholinyl, 3-(1,3-oxazolidin-2-one), 1-pyrrolidin-2-one, 1-imidazolidin-2-one, 1-imidazolidine-2,4-dione, 4-methyl-1,3-oxazol-5-yl, 4-(propan-2-yl)-1,3-oxazol-5-yl, 5-(4,4-dimethyl-4,5-dihydro-1,3-oxazol-5-yl), 5-(1,3-oxazol-4-amine), 5-(1,3-oxazole-4-carbonitrile), 5-(1,3-oxazole-4-carboxamide); and
R⁷ is selected from the group consisting of hydrogen and chloro.

12. The compound according to claim 1, or a pharmaceutically acceptable salt thereof, wherein:
X₁, X₂, X₃, X₄, X₅, X₆ X₇, and X₈ are selected from N or CH;
R¹ is selected from the group consisting of hydrogen, cyclopentyl, cyclopropyl, propan-2-yl, methyl, ethyl, 2-methylpropyl, thiophen-3-yl, furan-3-yl, pyridine-3-yl, ethoxy, phenyl, difluoromethoxy, chloride, tetrahydrofuran-(2 or 3)-yl, tetrahydropyran-(3 or 4)-yl, 1-methylpyrrolidin-(2 or 3)-yl, 1-methyl-(3 or 4)-piperdidinyl, carboxamide, 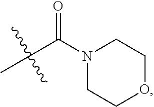 [SMILES: *C(=O)N1CCOCC1]
N,N-dimethyl-carboxamide, N-methyl-carboxamide, methyl-dimethylamine, 4-methyl-morpholinyl, 4-carbonyl-morpholinyl, cyclopentyl-methyl, and trifluoromethyl;
R² is selected from the group consisting of hydrogen, trifluoromethyl, propan-2-yl, morpholin-4-yl, 1-methylpiperidin-4-yl, and tetrahydrofuran-3-yl;
R³ is selected from the group consisting of hydrogen, trifluoromethyl, chloride, methyl, propan-2-yl, 2-methylpropyl, phenyl, cyclopropyl, cyclobutyl, cyclopentyl, cyclohexyl, tetrahydrofuran-(2 or 3)-yl, and piperidin-1-yl, pyrrolidin-1-yl, 1H-imidazol-(2 or 5)-yl, propan-2-yloxy, ethoxy, cyano, carboxamide, and carbonyl-morpholinyl;
R⁴ is optionally absent or is selected from the group consisting of hydrogen, pyrrolidin-1-yl, cyano, carboxamide, and dimethyl-methylamine;
R⁵ is selected from the group consisting of hydrogen, pyrrolidin-1-yl, cyano, carboxamide, and dimethyl-methylamine;
R⁶ is selected from the group consisting of hydrogen, 1,3,4-oxadiazol-2-yl, furan-2-yl, 1,3-oxazol-2-yl, methyl-dimethylamine, 1-methyl-pyrrolidyl, 1-methyl-pyrrolidin-3-ol, 4-methyl-morpholinyl, 3-(1,3-oxazolidin-2-one), 1-pyrrolidin-2-one, 1-imidazolidin-2-one, 1-imidazolidine-2,4-dione, 4-methyl-1,3-oxazol-5-yl, 4-(propan-2-yl)-1,3-oxazol-5-yl, 5-(4,4-dimethyl-4,5-dihydro-1,3-oxazol-5-yl), 5-(1,3-oxazol-4-amine), 5-(1,3-oxazole-4-carbonitrile), 5-(1,3-oxazole-4-carboxamide); and
R⁷ is selected from the group consisting of hydrogen and chloro.

13. A compound having the structure according to Formula (II): 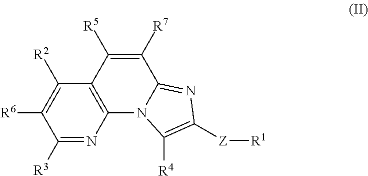
or a pharmaceutically acceptable salt thereof, wherein:
Z is selected from a bond, —C(O), or (C₁-C₆)alkylene;
R¹ is selected from the group consisting of hydrogen, —R¹², —R¹⁴, —R⁹ (R¹⁵)ₘ, —OR⁹(R¹⁵)ₘ, and halo;
R² is selected from the group consisting of hydrogen, (C₁-C₆)alkyl, (C₁-C₆)alkoxy, —R¹², —R⁴, C(O)R¹², —R⁹R¹², —R⁹R¹³, —R⁹R¹⁴, —C(O)R¹⁴, —R⁹(R¹⁵)ₘ, —OR⁹(R¹⁵)ₘ, —OR¹³, —R¹²S(O)₂, S(O)₂R¹² halo, nitrile, sulfonamide, sulfone, sulfoxide, (C₄-C₁₄)aryl, and (C₃-C₁₂)cycloalkyl, wherein said R² group may be optionally substituted with one to three R¹¹ groups;
R³ is selected from the group consisting of hydrogen, (C₁-C₆)alkyl, (C₁-C₆)alkoxy, —R¹², —R¹⁴, C(O)R¹², —R⁹R¹², —R⁹R¹³, —R⁹R¹⁴, —C(O)R¹⁴, —R⁹(R¹⁵)ₘ, —OR⁹(R¹⁵)ₘ, —OR¹³, halo, nitrile, sulfonamide, sulfone, sulfoxide, (C₄-C₁₄)aryl, and (C₃-C₁₂)cycloalkyl, wherein said R³ group may be optionally substituted with one to three R¹¹ groups;
R⁴ is selected from the group consisting of hydrogen, (C₁-C₆)alkyl, —R⁹(R¹⁵)ₘ, —OR⁹(R¹⁵)ₘ, —C(O)R⁹, —C(O)R¹³, halo, and (C₃-C₁₂)cycloalkyl;
R⁵ is selected from the group consisting of hydrogen, (C₁-C₆)alkyl, —C(O)R², —R⁹R¹², —R⁹(R¹⁵)ₘ, —OR⁹(R¹⁵)ₘ, —R¹⁴, halo, and nitrile;
R⁶ is selected from the group consisting of hydrogen, (C₁-C₆)alkyl, (C₁-C₆)alkoxy, —R², —R¹⁴, C(O)R¹², —R⁹R¹², —R⁹R¹³, —R⁹R¹⁴, —C(O)R¹⁴, —R⁹(R¹⁵)ₘ, —OR⁹(R¹⁵)ₘ, —OR¹³, halo, nitrile, sulfonamide, sulfone, sulfoxide, (C₄-C₁₄)aryl, and (C₃-C₁₂)cycloalkyl, wherein said R⁶ group may be optionally substituted with one to three R¹¹ groups;
R⁷ is selected from the group consisting of hydrogen, (C₁-C₆)alkyl, —R⁹(R¹⁵)ₘ, —OR⁹(R¹⁵)ₘ, halo, —C(O)R¹², —R⁹R¹², nitrile, and —R¹⁴;
R⁸ is independently selected from the group consisting of hydrogen and (C₁-C₆)alkyl;
R⁹ is (C₁-C₆)alkyl;
R¹¹ is selected from the group consisting of (C₁-C₆)alkyl, dimethyl, sulfonamide, —OR⁸, —C(O)R¹², oxo, nitrile, —R¹², halo, —R⁹(R¹⁵)ₘ, and —OR⁹(R¹⁵)ₘ;
R¹² is —NR^{x}R^{y}, wherein R^{x} and R^{y} are independently selected from the group consisting of hydrogen and (C₁-C₆)alkyl, and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring independently have one to four heteroatoms selected from N, S and O, and wherein said heterocyclic ring or heteroaryl ring may be also optionally substituted with one to three R¹¹ groups;
R¹³ is (C₃-C₁₂)cycloalkyl;
R¹⁴ is selected from the group consisting of (C₁-C₁₁)heteroaryl or (C₁-C₁₁)heterocyclic, wherein said (C₁-C₁₁)heterocyclic or (C₁-C₁₁)heteroaryl each may have one to three heteroatoms selected from N, S, or O, and wherein said (C₁-C₁₁)heteroaryl or (C₁-C₁₁)heterocyclic may also be optionally substituted by one to three independent R¹¹ groups;
R¹⁵ is halo; and
m is independently 0 or an integer from 1 to 3.

14. The compound according to claim 13, or a pharmaceutically acceptable salt thereof, wherein:
Z is selected from the group consisting of a bond, —C(O), and methylene;
R¹ is selected from the group consisting of hydrogen, —R¹², chloro, bromo, fluoro, difluoromethyl, trifluoromethyl, difluoromethoxy, trifluoromethoxy, oxazolyl, furanyl, oxolanyl, oxadiazolyl, oxazolidinyl, imidazolidinyl, imidazolyl, oxanyl, piperidinyl, morpholinyl, dihydropyranyl, pyranyl, tetrahydropyridinyl, pyridinyl, and pyrrolidinyl, wherein said R¹ group may be optionally substituted with one to three R¹¹ groups;
R² is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R² may be optionally substituted by one to three independent R¹¹ groups;
R³ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R³ may be optionally substituted by one to three independent R¹¹ groups;
R⁴ is selected from the group consisting of hydrogen, methyl, ethyl, propyl, butyl, —C(O)R⁹, —C(O)R¹³, chloro, bromo, fluoro, difluoromethyl, trifluoromethyl, difluoromethoxy, trifluoromethoxy, cyclopropyl, cyclobutyl, cyclopentyl, and cyclohexyl;
R⁵ is selected from the group consisting of hydrogen, methyl, ethyl, propyl, butyl, —C(O)R¹², —R⁹R¹², nitrile, chloro, bromo, fluoro, difluoromethyl, trifluoromethyl, difluoromethoxy, trifluoromethoxy, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, —R⁹R¹², —R⁹R¹³, —R⁹R¹⁴, —C(O)R¹², —C(O)R¹⁴, difluoromethoxy, trifluoromethoxy, —OR¹³, cyclopropyl, cyclobutyl, cyclopentyl, cyclohexyl, phenylmethyl, imidazolyl, phenyl, thiophenyl, piperidinyl, oxanyl, pyrrolidinyl, furanyl, morpholinyl, pyridinyl, oxolanyl, wherein R⁶ may be optionally substituted by one to three independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl; and wherein R^{x} and R^{y} can optionally join together along with the nitrogen to which they are joined to form a (C₁-C₁₁)heterocyclic ring or (C₁-C₁₁)heteroaryl ring, wherein said heterocyclic ring or said heteroaryl ring, independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, imidazolyl, thiophenyl, oxanyl, pyrrolidinyl, furanyl, morpholinyl, oxazolyl, oxadiazolyl, oxazolidinyl, dihydropyranyl, tetrahydropyridinyl, imidazolidinyl, and pyridinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

15. The compound according to claim 13, or a pharmaceutically acceptable salt thereof, wherein:
Z is selected from a bond or methylene;
R¹ is selected from the group consisting of oxazolyl, oxanyl, oxolanyl, oxadiazolyl, oxazolidinyl, dihydropyranyl, tetrahydropyridinyl, pyrrolidinyl, morpholinyl, imidazolidinyl, and furanyl, wherein said R¹ group may be optionally substituted with one to two R¹¹ groups;
R² is selected from the group consisting of hydrogen, methyl, ethyl, propyl, isopropyl, butyl, isobutyl, methoxy, ethoxy, propoxy, chloro, bromo, fluoro, nitrile, difluoromethyl, trifluoromethyl, difluoromethoxy, trifluoromethoxy, cyclopropyl, cyclobutyl, cyclopentyl, cyclohexyl, imidazolyl, phenyl, and oxolanyl, wherein R² may be optionally substituted by one to two independent R¹¹ groups;
R³ is selected from the group consisting of hydrogen, methyl, ethyl, propyl, isopropyl, butyl, isobutyl, methoxy, ethoxy, propoxy, nitrile, difluoromethyl, trifluoromethyl, —C(O)R¹², oxanyl, oxolanyl, pyridinyl, phenyl, thiophenyl, piperidinyl, pyrrolidinyl, wherein R³ may be optionally substituted by one to two independent R¹¹ groups;
R⁴ is selected from the group consisting of hydrogen, methoxy, ethoxy, propoxy, methyl, ethyl, propyl, butyl, nitrile, —C(O)R⁹, —C(O)R¹³, chloro, bromo, and fluoro;
R⁵ is selected from the group consisting of hydrogen, methyl, ethyl, propyl, butyl, —C(O)R¹², —R⁹R¹², nitrile, methoxy, ethoxy, propoxy, nitrile, chloro, bromo, fluoro, and pyrrolidinyl;
R⁶ is selected from the group consisting of hydrogen, methyl, ethyl, propyl, isopropyl, butyl, isobutyl, methoxy, ethoxy, propoxy, nitrile, chloro, bromo, fluoro, difluoromethyl, trifluoromethyl, difluoromethoxy, trifluoromethoxy, piperidinyl, morpholinyl, oxolanyl, wherein R⁶ may be optionally substituted by one to two independent R¹¹ groups;
R⁷ is selected from the group consisting of hydrogen, —C(O)R¹², —R⁹R¹², methyl, ethyl, propyl, butyl, chloro, bromo, fluoro, difluoromethyl, trifluoromethyl, difluoromethoxy, trifluoromethoxy, nitrile, and pyrrolidinyl;
R⁸ is independently selected from the group consisting of hydrogen, methyl, ethyl, propyl, butyl, and pentyl;
R⁹ is selected from the group consisting of methyl, ethyl, propyl, butyl, and pentyl;
R¹¹ is selected from the group consisting of methyl, dimethyl, ethyl, propyl, isopropyl, hydroxyl, oxo, nitrile, —C(O)R¹², and amino;
R¹² is —NR^{x}R^{y}, wherein R^{x} and R^{y} are independently selected from the group consisting of hydrogen and methyl; and wherein R^{x} and R¹ can optionally join together along with the nitrogen to which they are joined to form a (C₁-C₁₁)heterocyclic ring or (C₁-C₁₁)heteroaryl ring, wherein said heterocyclic ring or said heteroaryl ring, independently have one to four heteroatoms selected from N, S and O, and wherein said heterocyclic ring or heteroaryl ring may be also optionally substituted with one to three R¹¹ groups;
R¹³ is selected from the group consisting of cyclopropyl, cyclobutyl, cyclopentyl, and cyclohexyl;
R¹⁴ is selected from the group consisting of piperidinyl, oxolanyl, morpholinyl, wherein R¹⁴ may be optionally substituted by one to three independent R¹¹ groups;
R¹⁵ is selected from the group consisting of fluoro, bromo, and chloro; and
m is independently 0 or an integer from 1 to 3.

16. A compound having the structure according to Formula (X): 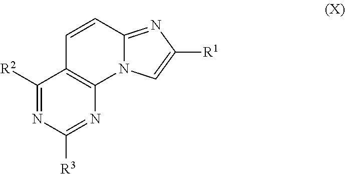
or a pharmaceutically acceptable salt thereof, wherein:
R¹ is selected from the group consisting of hydrogen and (C₁-C₁₁)heteroaryl;
R² is selected from the group consisting of hydrogen and (C₁-C₆)haloalkyl; and
R³ is selected from the group consisting of hydrogen and (C₁-C₆)haloalkyl.

17. The compound according to claim 16, or a pharmaceutically acceptable salt thereof, wherein R¹ is selected from the group consisting of hydrogen and oxadiazolyl.

18. The compound according to claim 16, or a pharmaceutically acceptable salt thereof, wherein:
R¹ is selected from the group consisting of hydrogen and oxadiazolyl;
R² is selected from the group consisting of hydrogen and trifluoromethyl; and
R³ is selected from the group consisting of hydrogen and trifluoromethyl.

19. The compound according to claim 16, or a pharmaceutically acceptable salt thereof, wherein:
R¹ is selected from the group consisting of hydrogen, and 1,3,4-oxadiazol-2-yl;
R² is selected from the group consisting of hydrogen, and trifluoromethyl; and
R³ is selected from the group consisting of hydrogen, and trifluoromethyl.

20. A compound having the structure according to Formula (XI): 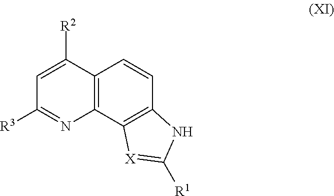
or a pharmaceutically acceptable salt thereof, wherein:
X is selected from the group consisting of N and CH;
R¹ is selected from the group consisting of hydrogen, and (C₁-C₁₁)heteroaryl;
R² is selected from the group consisting of hydrogen, and (C₁-C₆)haloalkyl; and
R³ is selected from the group consisting of hydrogen, and (C1-C₆)haloalkyl.

21. The compound according to claim 20, or a pharmaceutically acceptable salt thereof, wherein R¹ is selected from the group consisting of hydrogen, oxadiazolyl, and oxazolyl.

22. The compound according to claim 20, or a pharmaceutically acceptable salt thereof, wherein:
X is selected from the group consisting of N and CH:
R¹ is selected from the group consisting of hydrogen, 1,3,4-oxadiazol-2-yl, and 1,3-oxazol-5-yl;
R² is selected from the group consisting of hydrogen, and trifluoromethyl; and
R³ is selected from the group consisting of hydrogen, and trifluoromethyl.

23. A compound having the structure according to Formula (XIII): 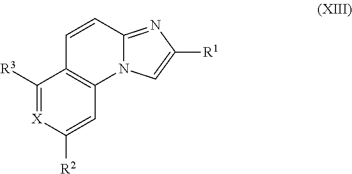
or a pharmaceutically acceptable salt thereof, wherein:
X is selected from the group consisting of N and CH;
R¹ is selected from the group consisting of hydrogen and (C₁-C₁₁)heteroaryl;
R² is selected from the group consisting of hydrogen and (C₁-C₆)haloalkyl; and
R³ is selected from the group consisting of hydrogen, and (C₁-C₆)haloalkyl.

24. The compound according to claim 23, or a pharmaceutically acceptable salt thereof, wherein: R¹ is selected from the group consisting of hydrogen and oxadiazolyl.

25. The compound according to claim 23, or a pharmaceutically acceptable salt thereof, wherein:
X is selected from the group consisting of nitrogen and carbon;
R¹ is selected from the group consisting of hydrogen and 1,3,4-oxadiazol-2-yl;
R² is selected from the group consisting of hydrogen and trifluoromethyl; and
R³ is selected from the group consisting of hydrogen and trifluoromethyl.

26. A compound having the structure according to Formula (XIV): 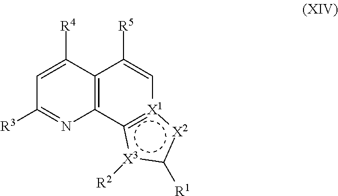
or a pharmaceutically acceptable salt thereof, wherein:
X¹ is selected from the group consisting of N and C;
X² is selected from the group consisting of S, C, and CH;
X³ is selected from the group consisting of N and O;
R¹ is selected from the group consisting of hydrogen, (C₁-C₁₁)heteroaryl, and (C₁-C₁₁)heterocycle;
R² is selected from the group consisting of hydrogen, benzyl, (C₁-C₆)alkyl, acetyl, and cycloalkylcarbonyl;
R³ is selected from the group consisting of hydrogen, and (C₁-C₆)haloalkyl;
R⁴ is selected from the group consisting of hydrogen, and (C₁-C₆)haloalkyl; and
R⁵ is hydrogen.

27. The compound according to claim 26, or a pharmaceutically acceptable salt thereof, wherein R¹ is oxadiazolyl.

28. The compound according to claim 26, or a pharmaceutically acceptable salt thereof, wherein:
X¹ is selected from the group consisting of N and C;
X² is selected from the group consisting of S, C, and CH;
X³ is selected from the group consisting of N and O;
R¹ is selected from the group consisting of hydrogen, 3,4-oxadiazol-2-yl, tetrahydropyran-(3 or 4)-yl, 1-methylpiperidin-(3 or 4)-yl, 3,6-dihydro-2H-pyran-4-yl, 5,6-dihydro-2H-pyran-3-yl, and 1-methyl-1,2,3,6-tetrahydropyridin-(4 or 5)-yl;
R² is selected from the group consisting of hydrogen, benzyl, methyl, acetyl, and cyclobutylcarbonyl;
R³ is selected from the group consisting of hydrogen, and trifluoromethyl;
R⁴ is selected from the group consisting of hydrogen, and trifluoromethyl; and
R⁵ is hydrogen.

29. A compound having the structure according to Formula (XV): 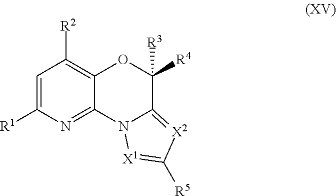
or a pharmaceutically acceptable salt thereof, wherein:
X¹ and X² are independently selected from the group consisting of N and CH;
R¹ is selected from the group consisting of hydrogen and (C₁-C₆)haloalkyl;
R² is selected from the group consisting of hydrogen and (C₁-C₆)haloalkyl;
R³ is selected from the group consisting of hydrogen and (C₁-C₆)alkyl;
R⁴ is selected from the group consisting of hydrogen and (C₁-C₆)alkyl; and
R⁵ is selected from the group consisting of hydrogen and (C₁-C₁₁)heteroaryl.

30. The compound according to claim 29, or a pharmaceutically acceptable salt thereof, wherein R⁵ is oxadiazolyl.

31. The compound according to claim 29, or a pharmaceutically acceptable salt thereof, wherein:
X¹ and X² are independently selected from the group consisting of N and CH;
R¹ is selected from the group consisting of hydrogen, and trifluoromethyl;
R² is selected from the group consisting of hydrogen, and trifluoromethyl;
R³ is selected from the group consisting of hydrogen, and methyl;
R⁴ is selected from the group consisting of hydrogen, and methyl; and
R⁵ is selected from the group consisting of hydrogen, and 1,3,4-oxadiazol-2-yl.

32. A compound having the structure according to Formula (XVI): 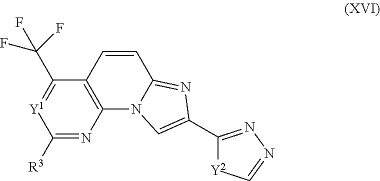
or a pharmaceutically acceptable salt thereof, wherein:
Y¹ is selected from the group consisting of N and CH;
Y² is selected from the group consisting of O and S; and
R³ is selected from the group consisting of trifluoromethyl and cyclopentyl.

33. A compound having the structure according to Formula (XVII): 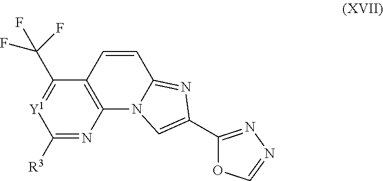
or a pharmaceutically acceptable salt thereof, wherein:
Y¹ is selected from the group consisting of N and CH;
R³ is selected from the group consisting of trifluoromethyl and cyclopentyl.

34. A compound having the structure: 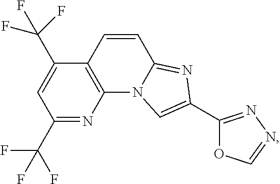 [SMILES: FC(F)(F)c1cc(C(F)(F)F)c2ccc3nc(-c4nnco4)cn3c2n1]
or a pharmaceutically acceptable salt thereof.

35. A compound having the structure: 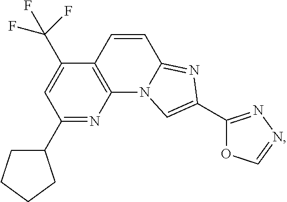 [SMILES: FC(F)(F)c1cc(C2CCCC2)nc2c1ccc1nc(-c3nnco3)cn12]
or a pharmaceutically acceptable salt thereof.

36. A compound having the structure: 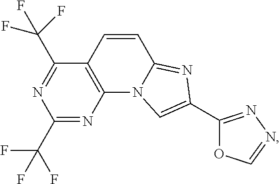 [SMILES: FC(F)(F)c1nc(C(F)(F)F)c2ccc3nc(-c4nnco4)cn3c2n1]
or a pharmaceutically acceptable salt thereof.

37. A compound selected from the group consisting of:
2-[2,4-bis(trifluoromethyl)imidazo[1,2-a]1,8-naphthyridin-8-yl]-1,3,4-oxadiazole,
2-[2-cyclopentyl-4-(trifluoromethyl)imidazo[1,2-a]1,8-naphthyridin-8-yl]-1,3,4-oxadiazole,
2-[2-(propan-2-yl)-4-(trifluoromethyl)imidazo[1,2-a]1,8-naphthyridin-8-yl]-1,3,4-oxadiazole,
5-[2,4-bis(trifluoromethyl)imidazo[1,2-a]1,8-naphthyridin-8-yl]-1,3-oxazole,
2-[2-cyclopropyl-4-(trifluoromethyl)imidazo[1,2-a]1,8-naphthyridin-8-yl]-1,3,4-oxadiazole,
2-[2-(thiophen-3-yl)-4-(trifluoromethyl)imidazo[1,2-a]1,8-naphthyridin-8-yl]-1,3,4-oxadiazole,
2-[2-methyl-4-(trifluoromethyl)imidazo[1,2-a]1,8-naphthyridin-8-yl]-1,3,4-oxadiazole,
2-[2,4-bis(trifluoromethyl)-7H-pyrrolo[2,3-h]quinolin-8-yl]-1,3,4-oxadiazole,
2-[9-methyl-2,4-bis(trifluoromethyl)imidazo[1,2-a]1,8-naphthyridin-8-yl]-1,3,4-oxadiazole,
2-[2-ethoxy-4-(trifluoromethyl)imidazo[1,2-a]1,8-naphthyridin-8-yl]-1,3,4-oxadiazole,
4-(1,3,4-oxadiazol-2-yl)-10,12-bis(trifluoromethyl)-2,5,11,13-tetraazatricyclo[7.4.0.0²,⁶]trideca-1(9),3,5,7,10,12-hexaene,
2-[2-(furan-3-yl)-4-(trifluoromethyl)imidazo[1,2-a]1,8-naphthyridin-8-yl]-1,3,4-oxadiazole,
2-[2-ethyl-4-(trifluoromethyl)imidazo[1,2-a]1,8-naphthyridin-8-yl]-1,3,4-oxadiazole,
2-[1-benzyl-6,8-bis(trifluoromethyl)-1H-pyrrolo[3,2-h]quinolin-2-yl]-1,3,4-oxadiazole,
2-[6,8-bis(trifluoromethyl)-1H-pyrrolo[3,2-h]quinolin-2-yl]-1,3,4-oxadiazole,
2-[1-methyl-6,8-bis(trifluoromethyl)-1H-pyrrolo[3,2-h]quinolin-2-yl]-1,3,4-oxadiazole,
2-[2-phenyl-4-(trifluoromethyl)imidazo[1,2-a]1,8-naphthyridin-8-yl]-1,3,4-oxadiazole,
2-[9-chloro-2,4-bis(trifluoromethyl)imidazo[1,2-a]1,8-naphthyridin-8-yl]-1,3,4-oxadiazole,
3-[8-(1,3,4-oxadiazol-2-yl)-4-(trifluoromethyl)imidazo[1,2-a]1,8-naphthyridin-2-yl]pyridine,
2-[2-(difluoromethoxy)-4-(trifluoromethyl)imidazo[1,2-a]1,8-naphthyridin-8-yl]-1,3,4-oxadiazole,
2-[1-cyclobutanecarbonyl-6,8-bis(trifluoromethyl)-1H-pyrrolo[3,2-h]quinolin-2-yl]-1,3,4-oxadiazole,
1-[2-(1,3,4-oxadiazol-2-yl)-6,8-bis(trifluoromethyl)-1H-pyrrolo[3,2-h]quinolin-1-yl]ethan-1-one,
2-[4-chloro-2-(propan-2-yl)imidazo[1,2-a]1,8-naphthyridin-8-yl]-1,3,4-oxadiazole,
2-[6,8-bis(trifluoromethyl)imidazo[1,2-a]quinolin-2-yl]-1,3,4-oxadiazole,
8-(furan-2-yl)-2,4-bis(trifluoromethyl)imidazo[1,2-a]1,8-naphthyridine,
2-{2,4-dimethylimidazo[1,2-a]1,8-naphthyridin-8-yl}-1,3,4-oxadiazole,
2-[2,4-bis(trifluoromethyl)imidazo[1,2-a]1,8-naphthyridin-8-yl]-1,3-oxazole,
5-[6,8-bis(trifluoromethyl)-3H-imidazo[4,5-h]quinolin-2-yl]-1,3-oxazole,
2-[2-chloro-4-(trifluoromethyl)imidazo[1,2-a]1,8-naphthyridin-8-yl]-1,3,4-oxadiazole,
2-[2,4-bis(propan-2-yl)imidazo[1,2-a]1,8-naphthyridin-8-yl]-1,3,4-oxadiazole,
2-[4-phenyl-2-(propan-2-yl)imidazo[1,2-a]1,8-naphthyridin-8-yl]-1,3,4-oxadiazole,
2-[6,8-bis(trifluoromethyl)-[1,3]oxazolo[5,4-h]quinolin-2-yl]-1,3,4-oxadiazole,
2-[6,8-bis(trifluoromethyl)furo[3,2-h]quinolin-2-yl]-1,3,4-oxadiazole,
2-[6,8-bis(trifluoromethyl)-[1,3]thiazolo[5,4-h]quinolin-2-yl]-1,3,4-oxadiazole,
{[2,4-bis(trifluoromethyl)imidazo[1,2-a]1,8-naphthyridin-8-yl]methyl}dimethylamine,
1-{[2,4-bis(trifluoromethyl)imidazo[1,2-a]1,8-naphthyridin-8-yl]methyl}pyrrolidine,
1-{[2,4-bis(trifluoromethyl)imidazo[1,2-a]1,8-naphthyridin-8-yl]methyl}pyrrolidin-3-ol,
4-{[2,4-bis(trifluoromethyl)imidazo[1,2-a]1,8-naphthyridin-8-yl]methyl}morpholine,
1-[2,4-bis(trifluoromethyl)imidazo[1,2-a]1,8-naphthyridin-8-yl]pyrrolidin-2-one,
3-[2,4-bis(trifluoromethyl)imidazo[1,2-a]1,8-naphthyridin-8-yl]-1,3-oxazolidin-2-one,
1-[2,4-bis(trifluoromethyl)imidazo[1,2-a]1,8-naphthyridin-8-yl]imidazolidin-2-one,
1-[2,4-bis(trifluoromethyl)imidazo[1,2-a]1,8-naphthyridin-8-yl]imidazolidine-2,4-dione,
2-(oxan-4-yl)-6,8-bis(trifluoromethyl)-1H-pyrrolo[3,2-h]quinoline,
2-(oxan-3-yl)-6,8-bis(trifluoromethyl)-1H-pyrrolo[3,2-h]quinoline,
4-[6,8-bis(trifluoromethyl)-1H-pyrrolo[3,2-h]quinolin-2-yl)-1-methylpiperidine,
3-(6,8-bis(trifluoromethyl)-1H-pyrrolo[3,2-h]quinolin-2-yl]-1-methylpiperidine,
2-(3,6-dihydro-2H-pyran-4-yl)-6,8-bis(trifluoromethyl)-1H-pyrrolo[3,2-h]quinoline,
2-(5,6-dihydro-2H-pyran-3-yl)-6,8-bis(trifluoromethyl)-1H-pyrrolo[3,2-h]quinoline,
4-[6,8-bis(trifluoromethyl)-1H-pyrrolo[3,2-h]quinolin-2-yl]-1-methyl-1,2,3,6-tetrahydropyridine,
5-[6,8-bis(trifluoromethyl)-1H-pyrrolo[3,2-h]quinolin-2-yl]-1-methyl-1,2,3,6-tetrahydropyridine,
4-(1,3,4-oxadiazol-2-yl)-10,12-bis(trifluoromethyl)-2,5,8,13-tetraazatricyclo[7.4.0.0²,⁶]trideca-1(13),3,5,7,9,11-hexaene,
4-(1,3,4-oxadiazol-2-yl)-10,12-bis(trifluoromethyl)-2,5,7,13-tetraazatricyclo[7.4.0.0²,⁶]trideca-1(13),3,5,7,9,11-hexaene,
2-[5-(pyrrolidin-1-yl)-2,4-bis(trifluoromethyl)imidazo[1,2-a]1,8-naphthyridin-8-yl]-1,3,4-oxadiazole,
8-(1,3,4-oxadiazol-2-yl)-2,4-bis(trifluoromethyl)imidazo[1,2-a]1,8-naphthyridine-5-carbonitrile,
8-(1,3,4-oxadiazol-2-yl)-2,4-bis(trifluoromethyl)imidazo[1,2-a],1,8-naphthyridine-5-carboxamide,
dimethyl({[8-(1,3,4-oxadiazol-2-yl)-2,4-bis(trifluoromethyl)imidazo[1,2-a]1,8-naphthyridin-5-yl]methyl})amine,
2-[6-(pyrrolidin-1-yl)-2,4-bis(trifluoromethyl)imidazo[1,2-a]1,8-naphthyridin-8 yl]-1,3,4-oxadiazole,
8-(1,3,4-oxadiazol-2-yl)-2,4-bis(trifluoromethyl)imidazo[1,2-a]1,8-naphthyridine-6-carbonitrile,
8-(1,3,4-oxadiazol-2-yl)-2,4-bis(trifluoromethyl)imidazo[1,2-a]1,8-naphthyridine-6-carboxamide,
dimethyl({[8-(1,3,4-oxadiazol-2-yl)-2,4-bis(trifluoromethyl)imidazo[1,2-a]1,8-naphthyridin-6-yl]methyl})amine,
2-[5-(pyrrolidin-1-yl)-2-(trifluoromethyl)imidazo[1,2-a]1,8-naphthyridin-8-yl]-1,3,4-oxadiazole,
8-(1,3,4-oxadiazol-2-yl)-2-(trifluoromethyl)imidazo[1,2-a]1,8-naphthyridine-5-carbonitrile,
8-(1,3,4-oxadiazol-2-yl)-2-(trifluoromethyl)imidazo[1,2-a]1,8-naphthyridine-5-carboxamide,
dimethyl({[8-(1,3,4-oxadiazol-2-yl)-2-(trifluoromethyl)imidazo[1,2-a]1,8-naphthyridin-6-yl]methyl})amine,
2-[6-(pyrrolidin-1-yl)-2-(trifluoromethyl)imidazo[1,2-a]1,8-naphthyridin-8-yl]-1,3,4-oxadiazole,
8-(1,3,4-oxadiazol-2-yl)-2-(trifluoromethyl)imidazo[1,2-a]1,8-naphthyridine-6-carbonitrile,
8-(1,3,4-oxadiazol-2-yl)-2-(trifluoromethyl)imidazo[1,2-a]1,8-naphthyridine-6-carboxamide,
dimethyl({[8-(1,3,4-oxadiazol-2-yl)-2-(trifluoromethyl)imidazo[1,2-a]1,8-naphthyridin-6-yl]methyl})amine,
2-[4-cyclopropyl-2-(propan-2-yl)imidazo[1,2-a]1,8-naphthyridin-8-yl]-1,3,4-oxadiazole,
2-[4-cyclobutyl-2-(propan-2-yl)imidazo[1,2-a]1,8-naphthyridin-8-yl]-1,3,4-oxadiazole,
2-[4-cyclopentyl-2-(propan-2-yl)imidazo[1,2-a]1,8-naphthyridin-8-yl]-1,3,4-oxadiazole,
2-[4-cyclohexyl-2-(propan-2-yl)imidazo[1,2-a]1,8-naphthyridin-8-yl]-1,3,4-oxadiazole,
2-[4-(oxolan-2-yl)-2-(propan-2-yl)imidazo[1,2-a]1,8-naphthyridin-8-yl]-1,3,4-oxadiazole,
2-[4-(oxolan-3-yl)-2-(propan-2-yl)imidazo[1,2-a]1,8-naphthyridin-8-yl]-1,3,4-oxadiazole,
2-[4-(1H-imidazol-5-yl)-2-(propan-2-yl)imidazo[1,2-a]1,8-naphthyridin-8-yl]-1,3,4-oxadiazole,
2-[4-(1H-imidazol-2-yl)-2-(propan-2-yl)imidazo[1,2-a]1,8-naphthyridin-8-yl]-1,3,4-oxadiazole,
1-[8-(1,3,4-oxadiazol-2-yl)-2-(propan-2-yl)imidazo[1,2-a]1,8-naphthyridin-4-yl]piperidine,
2-[2-(propan-2-yl)-4-(pyrrolidin-1-yl)imidazo[1,2-a]1,8-naphthyridin-8-yl]-1,3,4-oxadiazole,
2-[2-(propan-2-yl)-4-(propan-2-yloxy)imidazo[1,2-a]1,8-naphthyridin-8-yl]-1,3,4-oxadiazole,
2-[4-ethoxy-2-(propan-2-yl)imidazo[1,2-a]1,8-naphthyridin-8-yl]-1,3,4-oxadiazole,
8-(1,3,4-oxadiazol-2-yl)-2-(propan-2-yl)imidazo[1,2-a]1,8-naphthyridine-4-carbonitrile,
8-(1,3,4-oxadiazol-2-yl)-2-(propan-2-yl)imidazo[1,2-a]1,8-naphthyridine-4-carboxamide,
4-{[8-(1,3,4-oxadiazol-2-yl)-2-(propan-2-yl)imidazo[1,2-a]1,8-naphthyridin-4-yl]carbonyl}morpholine,
2-[4-(2-methylpropyl)-2-(propan-2-yl)imidazo[1,2-a]1,8-naphthyridin-8-yl]-1,3,4-oxadiazole,
2-[2-(oxolan-2-yl)-4-(trifluoromethyl)imidazo[1,2-a]1,8-naphthyridin-8-yl]-1,3,4-oxadiazole,
2-[2-(oxolan-3-yl)-4-(trifluoromethyl)imidazo[1,2-a]1,8-naphthyridin-8-yl]-1,3,4-oxadiazole,
2-[2-(oxan-3-yl)-4-(trifluoromethyl)imidazo[1,2-a]1,8-naphthyridin-8-yl]-1,3,4-oxadiazole,
2-[2-(oxan-4-yl)-4-(trifluoromethyl)imidazo[1,2-a]1,8-naphthyridin-8-yl]-1,3,4-oxadiazole,
2-[2-(1-methylpyrrolidin-2-yl)-4-(trifluoromethyl)imidazo[1,2-a]1,8-naphthyridin-8-yl]-1,3,4-oxadiazole,
2-[2-(1-methylpyrrolidin-3-yl)-4-(trifluoromethyl)imidazo[1,2-a]1,8-naphthyridin-8-yl]-1,3,4-oxadiazole,
1-methyl-3-[8-(1,3,4-oxadiazol-2-yl)-4-(trifluoromethyl)imidazo[1,2-a]1,8-naphthyridin-2-yl]piperidine,
1-methyl-4-[8-(1,3,4-oxadiazol-2-yl)-4-(trifluoromethyl)imidazo[1,2-a]1,8-naphthyridin-2-yl]piperidine,
8-(1,3,4-oxadiazol-2-yl)-4-(trifluoromethyl)imidazo[1,2-a]1,8-naphthyridine-2-carboxamide,
N,N-dimethyl-8-(1,3,4-oxadiazol-2-yl)-4-(trifluoromethyl)imidazo[1,2-a]1,8-naphthyridine-2-carboxamide,
N-methyl-8-(1,3,4-oxadiazol-2-yl)-4-(trifluoromethyl)imidazo[1,2-a]1,8-naphthyridine-2-carboxamide,
4-{[8-(1,3,4-oxadiazol-2-yl)-4-(trifluoromethyl)imidazo[1,2-a]1,8-naphthyridin-2-yl]carbonyl}morpholine,
dimethyl({[8-(1,3,4-oxadiazol-2-yl)-4-(trifluoromethyl)imidazo[1,2-a]1,8-naphthyridin-2-yl]methyl})amine,
4-{[8-(1,3,4-oxadiazol-2-yl)-4-(trifluoromethyl)imidazo[1,2-a]1,8-naphthyridin-2-yl]methyl}morpholine,
2-[2-(2-methylpropyl)-4-(trifluoromethyl)imidazo[1,2-a]1,8-naphthyridin-8-yl]-1,3,4-oxadiazole,
2-[2-(cyclopentylmethyl)-4-(trifluoromethyl)imidazo[1,2-a]1,8-naphthyridin-8-yl]-1,3,4-oxadiazole,
2-[2-(propan-2-yl)-3-(trifluoromethyl)imidazo[1,2-a]1,8-naphthyridin-8-yl]-1,3,4-oxadiazole,
2-[3-(propan-2-yl)-4-(trifluoromethyl)imidazo[1,2-a]1,8-naphthyridin-8-yl]-1,3,4-oxadiazole,
2-[2,4-bis(trifluoromethyl)imidazo[1,2-a]1,6-naphthyridin-8-yl)]-1,3,4-oxadiazole,
2-[3-(trifluoromethyl)imidazo[1,2-a]1,8-naphthyridin-8-yl]-1,3,4-oxadiazole,
4-[8-(1,3,4-oxadiazol-2-yl)imidazo[1,2-a]1,8-naphthyridin-3-yl]morpholine,
1-methyl-4-[8-(1,3,4-oxadiazol-2-yl)imidazo[1,2-a]1,8-naphthyridin-3-yl]piperidine,
2-[3-(oxolan-3-yl)imidazo[1,2-a]1,8-naphthyridin-8-yl]-1,3,4-oxadiazole,
2-[2,3-bis(trifluoromethyl)imidazo[1,2-a]1,8-naphthyridin-8-yl]-1,3,4-oxadiazole,
2-[3,4-bis(trifluoromethyl)imidazo[1,2-a]1,8-naphthyridin-8-yl]-1,3,4-oxadiazole,
5-[2,4-bis(trifluoromethyl)imidazo[1,2-a]1,8-naphthyridin-8-yl]-4-methyl-1,3-oxazole,
(5R)-5-[2,4-bis(trifluoromethyl)imidazo[1,2-a]1,8-naphthyridin-8-yl]-4,4-dimethyl-4,5-dihydro-1,3-oxazole,
4-(1,3,4-oxadiazol-2-yl)-10,12-bis(trifluoromethyl)-8-oxa-2,3,13-triazatricyclo[7.4.0.0²,⁶]trideca-1(13),3,5,9,11-pentaene,
5-[2,4-bis(trifluoromethyl)imidazo[1,2-a]1,8-naphthyridin-8-yl]-4-(propan-2-yl)-1,3-oxazole,
5-[2,4-bis(trifluoromethyl)imidazo[1,2-a]1,8-naphthyridin-8-yl]-1,3-oxazol-4-amine,
(7S)-7-methyl-4-(1,3,4-oxadiazol-2-yl)-10,12-bis(trifluoromethyl)-8-oxa-2,5,13-triazatricyclo[7.4.0.0²,⁶]trideca-1(13),3,5,9,11-pentaene,
5-[2,4-bis(trifluoromethyl)imidazo[1,2-a]1,8-naphthyridin-8-yl]-1,3-oxazole-4-carbonitrile,
5-[2,4-bis(trifluoromethyl)imidazo[1,2-a]1,8-naphthyridin-8-yl]-1,3-oxazole-4-carboxamide,
7,7-dimethyl-4-(1,3,4-oxadiazol-2-yl)-10,12-bis(trifluoromethyl)-8-oxa-2,5,13-triazatricyclo[7.4.0.0²,⁶]trideca-1(13),3,5,9,11-pentaene,
and pharmaceutically acceptable salts thereof.

38. A compound selected from the group consisting of:
2-[2,4-bis(trifluoromethyl)imidazo[1,2-a]1,8-naphthyridin-8-yl]-1,3,4-oxadiazole,
2-[2-cyclopentyl-4-(trifluoromethyl)imidazo[1,2-a]1,8-naphthyridin-8-yl]-1,3,4-oxadiazole,
2-[2-(propan-2-yl)-4-(trifluoromethyl)imidazo[1,2-a]1,8-naphthyridin-8-yl]-1,3,4-oxadiazole,
5-[2,4-bis(trifluoromethyl)imidazo[1,2-a]1,8-naphthyridin-8-yl]-1,3-oxazole,
2-[2-cyclopropyl-4-(trifluoromethyl)imidazo[1,2-a]1,8-naphthyridin-8-yl]-1,3,4-oxadiazole,
2-[2-(thiophen-3-yl)-4-(trifluoromethyl)imidazo[1,2-a]1,8-naphthyridin-8-yl]-1,3,4-oxadiazole,
2-[2-methyl-4-(trifluoromethyl)imidazo[1,2-a]1,8-naphthyridin-8-yl]-1,3,4-oxadiazole,
2-[2,4-bis(trifluoromethyl)-7H-pyrrolo[2,3-h]quinolin-8-yl]-1,3,4-oxadiazole,
2-[9-methyl-2,4-bis(trifluoromethyl)imidazo[1,2-a]1,8-naphthyridin-8-yl]-1,3,4-oxadiazole,
2-[2-ethoxy-4-(trifluoromethyl)imidazo[1,2-a]1,8-naphthyridin-8-yl]-1,3,4-oxadiazole,
4-(1,3,4-oxadiazol-2-yl)-10,12-bis(trifluoromethyl)-2,5,11,13-tetraazatricyclo[7.4.0.0²,⁶]trideca-1(9),3,5,7,10,12-hexaene,
2-[2-(furan-3-yl)-4-(trifluoromethyl)imidazo[1,2-a]1,8-naphthyridin-8-yl]-1,3,4-oxadiazole,
2-[2-ethyl-4-(trifluoromethyl)imidazo[1,2-a]1,8-naphthyridin-8-yl]-1,3,4-oxadiazole,
2-[1-benzyl-6,8-bis(trifluoromethyl)-1H-pyrrolo[3,2-h]quinolin-2-yl]-1,3,4-oxadiazole,
2-[6,8-bis(trifluoromethyl)-1H-pyrrolo[3,2-h]quinolin-2-yl]-1,3,4-oxadiazole,
2-[1-methyl-6,8-bis(trifluoromethyl)-1H-pyrrolo[3,2-h]quinolin-2-yl]-1,3,4-oxadiazole,
2-[2-phenyl-4-(trifluoromethyl)imidazo[1,2-a]1,8-naphthyridin-8-yl]-1,3,4-oxadiazole,
2-[9-chloro-2,4-bis(trifluoromethyl)imidazo[1,2-a]1,8-naphthyridin-8-yl]-1,3,4-oxadiazole,
3-[8-(1,3,4-oxadiazol-2-yl)-4-(trifluoromethyl)imidazo[1,2-a]1,8-naphthyridin-2-yl]pyridine,
2-[2-(difluoromethoxy)-4-(trifluoromethyl)imidazo[1,2-a]1,8-naphthyridin-8-yl]-1,3,4-oxadiazole,
2-[1-cyclobutanecarbonyl-6,8-bis(trifluoromethyl)-1H-pyrrolo[3,2-h]quinolin-2-yl]-1,3,4-oxadiazole,
1-[2-(1,3,4-oxadiazol-2-yl)-6,8-bis(trifluoromethyl)-1H-pyrrolo[3,2-h]quinolin-1-yl]ethan-1-one,
2-[4-chloro-2-(propan-2-yl)imidazo[1,2-a]1,8-naphthyridin-8-yl]-1,3,4-oxadiazole,
2-[6,8-bis(trifluoromethyl)imidazo[1,2-a]quinolin-2-yl]-1,3,4-oxadiazole,
8-(furan-2-yl)-2,4-bis(trifluoromethyl)imidazo[1,2-a]1,8-naphthyridine,
2-{2,4-dimethylimidazo[1,2-a]1,8-naphthyridin-8-yl}-1,3,4-oxadiazole,
2-[2,4-bis(trifluoromethyl)imidazo[1,2-a]1,8-naphthyridin-8-yl]-1,3-oxazole,
5-[6,8-bis(trifluoromethyl)-3H-imidazo[4,5-h]quinolin-2-yl]-1,3-oxazole,
2-[2-chloro-4-(trifluoromethyl)imidazo[1,2-a]1,8-naphthyridin-8-yl]-1,3,4-oxadiazole,
2-[2,4-bis(propan-2-yl)imidazo[1,2-a]1,8-naphthyridin-8-yl]-1,3,4-oxadiazole,
2-[4-phenyl-2-(propan-2-yl)imidazo[1,2-a]1,8-naphthyridin-8-yl]-1,3,4-oxadiazole,
and pharmaceutically acceptable salts thereof.

39. A pharmaceutical composition comprising a pharmaceutically acceptable diluent and a therapeutically effective amount of a compound, or pharmaceutically acceptable salt thereof, according to claim 1.

40. (canceled)

41. A method for treating a common wart on a subject comprising administering to the subject a compound of claim 1.

42. A method for treating a common wart on a subject comprising contacting a compound of claim 1 to the common wart on the subject.

43. The method according to claim 41, wherein the compound is administered in a topical formulation.

44. The method according to claim 41, wherein the common wart is caused by a human papillomavirus.

45. The method according to claim 41, wherein the compound is formulated into a topical formulation for treating and/or preventing a dermatological condition resulting from a viral infection.

46. The method according to claim 45, wherein the compound is formulated into a topical formulation for preventing and/or treating a dermatological condition comprising warts.

47. A method for treating a wart on the skin or mucous membrane of a subject comprising contacting a compound having the structure: 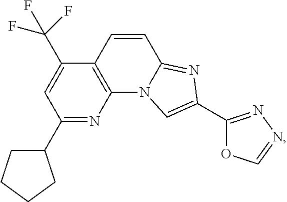 [SMILES: FC(F)(F)c1cc(C2CCCC2)nc2c1ccc1nc(-c3nnco3)cn12]
or a pharmaceutically acceptable salt thereof,
to the wart on the skin or mucous membrane of the subject.

48. A method for treating a wart on the skin or mucous membrane of a subject comprising contacting a compound having the structure: 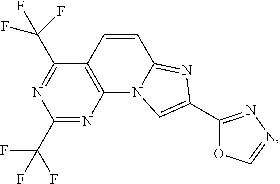 [SMILES: FC(F)(F)c1nc(C(F)(F)F)c2ccc3nc(-c4nnco4)cn3c2n1]
or a pharmaceutically acceptable salt thereof,
to the wart on the skin or mucous membrane of the subject.

49. A method for treating a viral infection in a subject that has been diagnosed with said viral infection or is at risk of developing said viral infection comprising administering to said subject, a compound of claim 1.

50. The method of claim 49, wherein said viral infection comprises one or more viruses from the Papillomavirus family.

51. The method of claim 50, wherein said viral infection comprises the human papillomavirus virus.

52. A method for enhancing the immune response in a subject that has been diagnosed with a viral infection or is at risk of developing said viral infection comprising administering to said subject, a compound claim 1.

53. A method for enhancing the immune response to a viral infection in a subject that is immunocompromised or is at risk of developing an immunocomprised immune system comprising administering to said subject, a compound of claim 1.

54. A method for upregulating the JAK/STAT immune pathway in a subject that has been diagnosed with a viral infection or is at risk of developing said viral infection comprising administering to said subject, a compound of claim 1.

55. A method of treating human papilloma virus associated skin diseases in a subject comprising administering to the subject a compound of claim 1.

56. The method according to claim 55, wherein the human papilloma virus associated skin disease comprises a disease that is selected from the group consisting of common warts, plantar warts, inguinal warts, venereal warts, and pre-cancerous lesions.

57. A method of treating high risk human papilloma virus infections in a subject comprising administering to the subject a compound of claim 1.

58. The method according to claim 57, wherein the high risk human papilloma virus infection in the subject comprises a site selected from the group consisting of the cervix, vulva, vagina, penis, oropharynx, and anus.

59. A method of topically treating human papilloma virus warts (verrucae) of the skin or mucous membranes of a subject comprising administering to the subject a compound of claim 1.

60. A method of treating precancerous and cancerous skin lesions in a subject comprising administering to the subject a compound of claim 1.

61. The method according to claim 60, wherein the skin lesion comprises actinic keratoses.

62. A method of treating a viral skin infection comprising molloscum contagiosum in a subject comprising administering to the subject a compound of claim 1.

63. A method for treating and/or preventing a viral infection in a subject comprising administering to the subject an activator of the subject's JAK/STAT pathway.

64. The method according to claim 63, wherein the activator is a chemical activator.

65. The method according to claim 63, wherein the activator is administered to the subject topically.

66. The method according to claim 64, wherein the chemical activator is a compound according to claim 1.

67. The method according to claim 63, wherein the viral infection is a viral infection of the subject's skin or mucous membranes.
